(19) United States
(12) Patent Application Publication
(10) Pub. No.: US 2002/0169155 A1
(43) Pub. Date: Nov. 14, 2002

(54) Chemokine receptor anagonists and methods of use therefor

(75) Inventors: Jay R. Luly, Wellesley, MA (US); Yoshisuke Nakasato, Shizuoka (JP); Etsuo Ohshima, Chiba (JP); Hiroki Sone, Aichi (JP); Osamu Kotera, Shizuoka (JP); Geraldine C.B. Harriman, Charlestown, RI (US); Kenneth G. Carson, Needham, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Millennium Pharmaceuticals, Inc., Cambridge, MA

(21) Appl. No.: 09/989,086

(22) Filed: Nov. 21, 2001

Related U.S. Application Data
(63) Continuation-in-part of application No. 09/627,886, filed on Jul. 28, 2000.
Application No. 09/627,886 is a continuation-in-part of application No. 09/362,837, filed on Jul. 28, 1999.
Application No. 09/362,837 is a continuation-in-part of application No. 09/235,102, filed on Jan. 21, 1999, now Pat. No. 6,329,385.
Application No. 09/235,102 is a continuation-in-part of application No. 09/148,823, filed on Sep. 4, 1998.

(51) Int. Cl. A61K 31/655; A61K 31/535; A61K 31/55; A61K 31/495

(52) U.S. Cl. 514/212.01; 514/211.01; 514/218; 514/151; 514/231.2; 514/252.12; 540/544; 540/575; 540/604; 544/164; 544/170; 544/383; 544/382

(57) ABSTRACT

Disclosed are novel compounds and a method of treating a disease associated with aberrant leukocyte recruitment and/or activation. The method comprises administering to a subject in need an effective amount of a compound represented by:

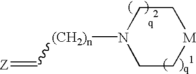

or physiologically acceptable salt thereof.

### RELATED APPLICATIONS

This application is a continuation-in-part of U.S. Ser. No. 09/627,886, filed Jul. 28, 2000, which is a continuation-in-part of U.S. Ser. No. 09/362,837, filed Jul. 28, 1999, which is a continuation-in-part of U.S. Ser. No. 09/235,102, filed Jan. 21, 1999, which is a continuation-in-part of U.S. Ser. No. 09/148,823, filed Sep. 4, 1998; the entire teachings of all above-referenced applications are incorporated herein by reference.

### BACKGROUND OF THE INVENTION

Chemoattractant cytokines or chemokines are a family of proinflammatory mediators that promote recruitment and activation of multiple lineages of leukocytes and lymphocytes. They can be released by many kinds of tissue cells after activation. Continuous release of chemokines at sites of inflammation mediates the ongoing migration of effector cells in chronic inflammation. The chemokines characterized to date are related in primary structure. They share four conserved cysteines, which form disulfide bonds. Based upon this conserved cysteine motif, the family is divided into two main branches, designated as the C—X—C chemokines (α-chemokines), and the C—C chemokines (β-chemokines), in which the first two conserved cysteines are separated by an intervening residue, or adjacent respectively (Baggiolini, M. and Dahinden, C. A., Immunology Today, 15:127-133 (1994)).

The C—X—C chemokines include a number of potent chemoattractants and activators of neutrophils, such as interleukin 8 (IL-8), PF4 and neutrophil-activating peptide-2 (NAP-2). The C—C chemokines include RANTES (Regulated on Activation, Normal T Expressed and Secreted), the macrophage inflammatory proteins 1α and 1 β(MIP-1β and MIP-1 β), eotaxin and human monocyte chemotactic proteins 1-3 (MCP-1, MCP-2, MCP-3), which have been characterized as chemoattractants and activators of monocytes or lymphocytes but do not appear to be chemoattractants for neutrophils. Chemokines, such as RANTES and MIP-1α, have been implicated in a wide range of human acute and chronic inflammatory diseases including respiratory diseases, such as asthma and allergic disorders.

The chemokine receptors are members of a superfamily of G protein-coupled receptors (GPCR) which share structural features that reflect a common mechanism of action of signal transduction (Gerard, C. and Gerard, N. P., Annu Rev. Immunol., 12:775-808 (1994); Gerard, C. and Gerard, N. P., Curr. Opin. Immunol., 6:140-145 (1994)). Conserved features include seven hydrophobic domains spanning the plasma membrane, which are connected by hydrophilic extracellular and intracellular loops. The majority of the primary sequence homology occurs in the hydrophobic transmembrane regions with the hydrophilic regions being more diverse. The first receptor for the C—C chemokines that was cloned and expressed binds the chemokines MIP-1 α and RANTES. Accordingly, this MIP-1α/RANTES receptor was designated C—C chemokine receptor 1 (also referred to as CCR-1; Neote, K., et al., Cell, 72:415-425 (1993); Horuk, R. et al., WO 94/11504, May 26, 1994; Gao, J. -I. et al., J. Exp. Med., 177:1421-1427 (1993)). Three receptors have been characterized which bind and/or signal in response to RANTES: CCR3 mediates binding and signaling of chemokines including eotaxin, RANTES, and MCP-3 (Ponath et al., J. Exp. Med., 183:2437 (1996)), CCR4 binds chemokines including RANTES, MIP-1α, and MCP-1 (Power, et al., J. Biol. Chem., 270:19495 (1995)), and CCR5 binds chemokines including MIP-1α, RANTES, and MIP-1β (Samson, et al., Biochem. 35: 3362-3367 (1996)). RANTES is a chemotactic chemokine for a variety of cell types, including monocytes, eosinophils, and a subset of T-cells. The responses of these different cells may not all be mediated by the same receptor, and it is possible that the receptors CCR1, CCR4 and CCR5 will show some selectivity in receptor distribution and function between leukocyte types, as has already been shown for CCR3 (Ponath et al). In particular, the ability of RANTES to induce the directed migration of monocytes and a memory population of circulating T-cells (Schall, T. et al., Nature, 347:669-71 (1990)) suggests this chemokine and its receptor(s) may play a critical role in chronic inflammatory diseases, since these diseases are characterized by destructive infiltrates of T cells and monocytes.

Many existing drugs have been developed as antagonists of the receptors for biogenic amines, for example, as antagonists of the dopamine and histamine receptors. No successful antagonists have yet been developed to the receptors for the larger proteins such as chemokines and C5a. Small molecule antagonists of the interaction between C—C chemokine receptors and their ligands, including RANTES and MIP-1α, would provide compounds useful for inhibiting harmful inflammatory processes “triggered” by receptor ligand interaction, as well as valuable tools for the investigation of receptor-ligand interactions.

### SUMMARY OF THE INVENTION

It has now been found that a class of small organic molecules are antagonists of chemokine receptor function and can inhibit leukocyte activation and/or recruitment. An antagonist of chemokine receptor function is a molecule which can inhibit the binding and/or activation of one or more chemokines, including C—C chemokines such as RANTES, MIP-1α, MCP-2, MCP-3 and MCP-4 to one or more chemokine receptors on leukocytes and/or other cell types. As a consequence, processes and cellular responses mediated by chemokine receptors can be inhibited with these small organic molecules. Based on this discovery, a method of treating a disease associated with aberrant leukocyte recruitment and/or activation is disclosed as well as a method of treating a disease mediated by chemokine receptor function. The method comprises administering to a subject in need an effective amount of a compound or small organic molecule which is an antagonist of chemokine receptor function. Compounds or small organic molecules which have been identified as antagonists of chemokine receptor function are discussed in detail hereinbelow, and can be used for the manufacture of a medicament for treating or for preventing a disease associated with aberrant leukocyte recruitment and/or activation. The invention also relates to the disclosed compounds and small organic molecules for use in treating or preventing a disease associated with aberrant leukocyte recruitment and/or activation. The invention also includes pharmaceutical compositions comprising one or more of the compounds or small organic molecules which have been identified herein as antagonists of chemokine function and a suitable pharmaceutical carrier. The invention further relates to novel compounds which can be used to treat an individual with a disease associated with aberrant leukocyte recruitment and/or activation and methods for their preparation.

### BRIEF DESCRIPTION OF THE DRAWINGS

FIG. 1 is a schematic showing the preparation of the compounds represented by Structural Formula (I).

FIG. 2 is a schematic showing the preparation of the compounds represented by Compound (VI-b).

FIG. 3 is a schematic showing the preparation of the compounds represented by Structural Formula (I)

FIG. 4 is a schematic showing the preparation of the compounds represented by Structural Formula (I), wherein Z is represented by Structural Formula (III) and wherein Ring A and/or Ring B in Z is substituted with R⁴⁰.

FIG. 5 is a schematic showing the preparation of the compounds represented by Structural Formula (1), wherein Z is represented by Structural Formula (III) and wherein Ring A and/or Ring B in Z is substituted with —(O)ᵤ—(CH₂)ₜ—COOR²⁰, —(O)ᵤ—(CH₂)ₜ—OC(O)R²⁰, —(O)ᵤ—(CH₂)ₜ—C(O)—NR²¹R²² or —(O)ᵤ—(CH₂)ₜ—NHC(O)O—R²⁰.

FIGS. 6A-6Z show the structures of exemplary compounds of the present invention.

FIG. 7 shows the preparation of compounds represented by Structural Formula (I), where in Z is represented by Structural Formulas (III) and wherein Ring A or Ring B in Z is substituted with R⁴⁰.

FIG. 8A is a schematic showing the preparation of 4-(4-chlorophenyl)-4-fluoropiperidine.

FIG. 8B is a schematic showing the preparation of 4-4-azido-4-(4-chlorophenyl)piperidine.

FIG. 8C is a schematic showing the preparation of 4-(4-chlorophenyl)-4-methylpiperidine.

FIG. 9A is a schematic showing the preparation of compounds represented by Structural Formulas (I), (VIII) and (VIII) wherein R¹ is an amine.

FIG. 9B is a schematic showing the preparation of compounds represented by Structural Formulas (I), (VIII) and (VIII) wherein R¹ is an alkylamine.

FIG. 9C is a schematic showing the preparation of 2-(4-chlorophenyl)-1-(N-methyl)ethylamine.

FIG. 9D is a schematic showing the preparation of 3-(4-chlorophenyl)-3-chloro-1-hydroxypropane.

FIG. 9E is a schematic showing the preparation of 3-(4-chlorophenyl)-1-N-methylaminopropane.

FIG. 10A is a schematic showing the preparation of 3-(4-chlorophenyl)-3-hydroxyl-3-methyl-1-N-methylaminopropane.

FIG. 10B is a schematic showing the preparation of 1-(4-chlorobenzoyl)-1,3-propylenediamine.

FIG. 10C is a schematic showing three procedures for the preparation of compounds represented by Structural Formulas (I),(VII), (VIII), (IX) and (XI) wherein Z is represented by Structural Formula (III) and wherein Ring A or Ring B in Z is substituted with R⁴⁰. In FIG. 10C, R⁴⁰ is represented by —(O)ᵤ—(CH₂)ₜ—C(O)—NR²¹R²², u is one, t is zero.

FIG. 10D is a schematic showing the preparation of 4-(4-chlorophenyl)-4-pyridine.

FIGS. 11A-11T show the structures of exemplary compounds of the present invention.

FIG. 12 is a schematic showing preparation of compounds of formula (VI-c).

FIG. 13 is a schematic showing preparation of compounds of formula (VI-e).

FIG. 14 is a schematic showing a procedure for the preparation of Examples 434 and 435.

FIG. 15 is a schematic showing a procedure for the preparation of Examples 436-438.

FIG. 16 is a schematic showing a procedure for the preparation of compounds of formula (I-f).

FIG. 17 is a schematic showing a procedure for the preparation of Examples 441 and 442.

FIG. 18 is a schematic showing a procedure for the preparation of Example 443.

FIG. 19 is a schematic showing a procedure for the preparation of Examples 315, 455, 338 and 446.

FIG. 20 shows the structures of exemplary compounds of the invention.

### DETAILED DESCRIPTION OF THE INVENTION

The present invention relates to small molecule compounds which are modulators of chemokine receptor function. In a preferred embodiment, the small molecule compounds are antagonists of chemokine receptor function. Accordingly, processes or cellular responses mediated by the binding of a chemokine to a receptor can be inhibited (reduced or prevented, in whole or in part), including leukocyte migration, integrin activation, transient increases in the concentration of intracellular free calcium [Ca⁺⁺]ᵢ, and/or granule release of proinflammatory mediators.

The invention further relates to a method of treatment, including prophylactic and therapeutic treatments, of a disease associated with aberrant leukocyte recruitment and/or activation or mediated by chemokines or chemokine receptor function, including chronic inflammatory disorders characterized by the presence of RANTES, MIP-1α, MCP-2, MCP-3 and/or MCP-4 responsive T cells, monocytes and/or eosinophils, including but not limited to diseases such as arthritis (e.g., rheumatoid arthritis), atherosclerosis, arteriosclerosis, restenosis, ischemia/reperfusion injury, diabetes mellitus (e.g., type 1 diabetes mellitus), psoriasis, multiple sclerosis, inflammatory bowel diseases such as ulcerative colitis and Crohn's disease, rejection of transplanted organs and tissues (i.e., acute allograft rejection, chronic allograft rejection), graft versus host disease, as well as allergies and asthma. Other diseases associated with aberrant leukocyte recruitment and/or activation which can be treated (including prophylactic treatments) with the methods disclosed herein are inflammatory diseases associated with Human Immunodeficiency Virus (HIV) infection, e.g., AIDS associated encephalitis, AIDS related maculopapular skin eruption, AIDS related interstitial pneumonia, AIDS related enteropathy, AIDS related periportal hepatic inflammation and AIDS related glomerulo nephritis. The method comprises administering to the subject in need of treatment an effective amount of a compound (i.e., one or more compounds) which inhibits chemokine receptor function, inhibits the binding of a chemokine to leukocytes and/or other cell types, and/or which inhibits leukocyte migration to, and/or activation at, sites of inflammation.

The invention further relates to methods of antagonizing a chemokine receptor, such as CCR1, in a mammal comprising administering to the mammal a compound as described herein.

According to the method, chemokine-mediated chemotaxis and/or activation of pro-inflammatory cells bearing receptors for chemokines can be inhibited. As used herein, “pro-inflammatory cells” includes but is not limited to leukocytes, since chemokine receptors can be expressed on other cell types, such as neurons and epithelial cells.

While not wishing to be bound by any particular theory or mechanism, it is believed that compounds of the invention are antagonists of the chemokine receptor CCR1, and that therapeutic benefits derived from the method of the invention are the result of antagonism of CCR1 function. Thus, the method and compounds of the invention can be used to treat a medical condition involving cells which express CCR1 on their surface and which respond to signals transduced through CCR1, as well as the specific conditions recited above.

In one embodiment, the antagonist of chemokine receptor function is represented by Structural Formula (I):

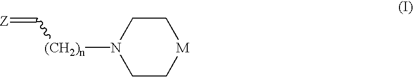

and physiologically acceptable salts thereof.

Z is a cycloalkyl or non-aromatic heterocyclic ring group fused to one, two or more aromatic rings, wherein each ring in Z is independently substituted or unsubstituted.

n is an integer, such as an integer from one to about four. Preferably, n is one, two or three. More preferably n is two. In alternative embodiments, other aliphatic or aromatic spacer groups (L) can be employed for (CH₂)ₙ.

M is >NR² or >CR¹R². M is preferably >C(OH)R².

R¹ is —H, —OH, —N₃, a halogen, an aliphatic group, a substituted aliphatic group, an aminoalkyl group, —O-(aliphatic group), —O-(substituted aliphatic group), —SH, —S-(aliphatic group), —S-substituted aliphatic group), —OC(O)-(aliphatic group), —O—C(O)-(substituted aliphatic group), —C(O)O-(aliphatic group), —C(O)O-(substituted aliphatic group), —COOH, —CN, —CO—NR³R⁴, —NR³R⁴; or R¹ can be a covalent bond between the ring atom at M and an adjacent carbon atom in the ring which contains M. R¹ is preferably —H or —OH.

R² is —H, —OH, a halogen, an acyl group, a substituted acyl group, —NR⁵R⁶, an aliphatic group, a substituted aliphatic group, an aromatic group, a substituted aromatic group, a benzyl group, a substituted benzyl group, a non-aromatic heterocyclic group, a substituted non-aromatic heterocyclic group, —O-(substituted or unsubstituted aromatic group) or —O-(substituted or unsubstituted aliphatic group). R² is preferably an aromatic group or a substituted aromatic group.

R³, R⁴, R⁵ and R⁶ are independently —H, an acyl group, a substituted acyl group, an aliphatic group, a substituted aliphatic group, an aromatic group, a substituted aromatic group, a benzyl group, a substituted benzyl group, a non-aromatic heterocyclic group or a substituted non-aromatic heterocyclic group.

R¹ and R², R³ and R⁴, or R⁵ and R⁶ taken together with the atom to which they are bonded, can alternatively form a substituted or unsubstituted non-aromatic carbocyclic or heterocyclic ring.

In embodiments where M is >CR¹R² and R¹ is a covalent bond between the carbon atom at M and an adjacent carbon atom in the ring which contains M, the antagonist of chemokine function can be represented by Structural Formula (Ia).

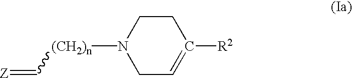

Z, n and R² are as described in Structural Formula (I).

In one preferred embodiment, Z is a tricyclic ring system comprising two carbocyclic aromatic groups fused to a five, six, seven or eight membered cycloalkyl group or to a non-aromatic heterocyclic ring. In one example, Z is represented by Structural Formula (II):

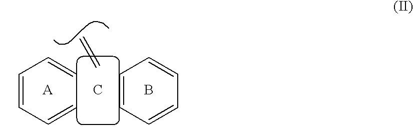

The phenyl rings in Structural Formula (II), labeled with an “A” and “B”, are referred to herein as “Ring A” and “Ring B”, respectively. The central ring, labeled with a “C”, is referred to as “Ring C” and can be, for example, a five, six, seven or eight membered non-aromatic carbocyclic ring (e.g., a cycloheptane or cyclooctane ring) or a non-aromatic heterocyclic ring. When Ring C is a non-aromatic heterocyclic ring, it can contain one or two heteroatoms such as nitrogen, sulfur or oxygen. In particular embodiments, Ring c is When Z is represented by Structural Formula (II), the tricyclic ring system can be connected to the remainder of the molecule by a covalent double bond between a carbon atom in Ring C and the carbon atom which, as depicted in Structural Formula (I), is bonded to Z.

Ring A and/or Ring B in Structural Formula (II) can be unsubstituted. Alternatively, Ring A and/or Ring B can have one or more substituents. Suitable substituents are as described hereinbelow. In one example, Ring A or Ring B is substituted with —(O)ᵤ—(CH₂)ₜ—C(O)OR²⁰, —(O)ᵤ—(CH₂)ₜ—OC(O)R²⁰, —(O)ᵤ—(CH₂)ₜ—C(O)—NR²¹R²² or —(O)ᵤ—(CH₂)ₜ—NHC(O)O—R²⁰.

u is zero or one.

t is an integer, such as an integer from zero to about three, and the methylene group —(CH₂)ₜ— can be substituted, as described herein for aliphatic groups, or unsubstituted.

R²⁰, R²¹ or R²² are independently —H, an aliphatic group, a substituted aliphatic group, an aromatic group, a substituted aromatic group or a non-aromatic heterocyclic group. Alternatively, R²¹ and R²², taken together with the nitrogen atom to which they are bonded, can form a non-aromatic heterocyclic ring.

Ring C optionally contains one or more substituents, as described hereinbelow. Examples of suitable tricyclic ring systems, Z, are provided by Structural Formula (III):

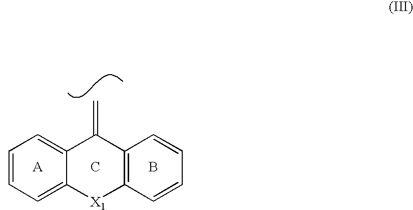

Ring A and Ring B in Structural Formula (III) are as described for Structural Formula (II).

X₁ is a bond, —O—, —S—, —CH₂—, —CH₂—CH₂—, —CH₂—S—, —S—CH₂—, —O—CH₂—, —CH₂—O—, —NR_{c}—CH₂—, —CH₂—NR_{c}—, —SO—CH₂—, —CH₂—SO—, —S(O)₂—CH₂—, —CH₂—S(O)₂—, —CH═CH—, —NR_{c}—CO— or —CO—NR_{c}—. Preferably X₁ is —CH₂—O—, —CH₂—CH₂—, —CH₂—S—, —NR_{c}—CO— or —CO—NR_{c}—.

R_{c} is hydrogen, an aliphatic group, a substituted aliphatic group, an aromatic group, a substituted aromatic group, a benzyl group or a substituted benzyl group.

In one example, R_{c} is —(CH₂)ₛ—COOR³⁰, —(CH₂)ₛ—C(O)—NR³¹R³² or —(CH₂)ₛ—NHC(O)—O—R³⁰, wherein s is an integer, such as an integer from one to about three;

R³⁰, R³¹ and R³² are independently —H, an aliphatic group, a substituted aliphatic group, an aromatic group, a substituted aromatic group or a non-aromatic heterocyclic group. Alternatively, R³¹ and R³², taken together with the nitrogen atom to which they are bonded, form a non-aromatic heterocyclic ring.

Other examples of suitable tricyclic ring systems for Z include benzodiazepines, benzooxazepines, benzooxazines, phenothiazines and groups represented by the following structural formulas:

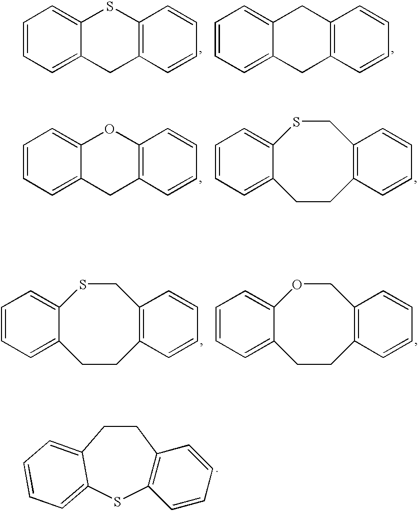

In another preferred embodiment, Z is a tricyclic ring system comprising two aromatic groups fused to a seven or eight membered cycloalkyl group or to a non-aromatic heterocyclic ring, wherein at least one of the aromatic groups is a heteroaryl group. In one example, Z is represented by Structural Formula (IV):

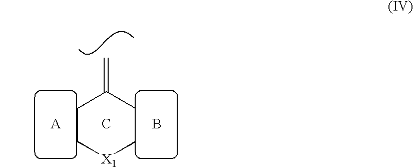

Ring A in Structural Formula (IV) can be a substituted or unsubstituted heteroaryl group. Ring B in Structural Formula (IV) can be a substituted or unsubstituted aromatic group, e.g., a heteroaryl group or carbocyclic aryl group. Suitable substituents are as described hereinbelow. In one example, Ring A and/or Ring B is substituted with —(O)ᵤ—(CH₂)ₜ—C(O)OR²⁰, —(O)ᵤ—(CH₂)ₜ—OC(O)R²⁰, —(O)ᵤ—(CH₂)ₜ—C(O)—NR²¹R²² or —(O)ᵤ—(CH₂)ₜ—NHC(O)O—R²⁰ as described above. u, t, R²⁰, R²¹, and R²² are as described above. X₁ and R_{c} can be as described above for Structural Formula (III).

In another embodiment of the present invention Z is represented by Structural Formula (IV), wherein Ring A is a pyridyl group and Ring B is an aromatic or heteroaromatic group. In one example, Z is represented by Structural Formula (IVa):

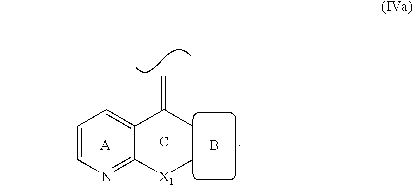

In this embodiment Ring A and Ring B are independently substituted or unsubstituted, and Ring B is preferably a phenyl group. X₁ and R_{c} can be as described above for Structural Formula (III).

In another embodiment, both Ring A and Ring B are pyridyl groups, and Z is represented by Structural Formula (IVb):

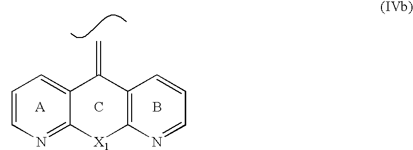

Ring A and Ring B can be independently substituted or unsubstituted as described above in Structural Formula (II), and X₁ can be as described above for Structural Formula (III).

In another embodiment of the present invention Z is represented by Structural Formula (V):

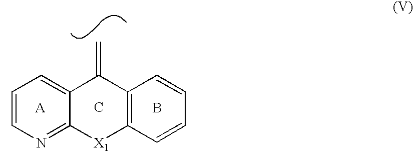

Ring A and Ring B can be independently substituted or unsubstituted as described above in Structural Formula (II), and X₁ can be as described above for Structural Formula (III).

In a preferred embodiment, Ring B in Structural Formula (V) is substituted para to the carbon atom of Ring B which is bonded to X₁ of Ring C, and Z is represented by Structural Formula (VI):

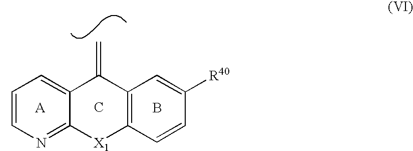

X₁ can be as described above in Structural Formula (II). Preferably X₁ is —CH₂—O—, —CH₂—CH₂— or —CH₂—S—.

R⁴⁰ is a substituent as described herein for aromatic groups. In one embodiment, R⁴⁰ is —OH, —COOH, a halogen, —NO₂, an aliphatic group, a substituted aliphatic group, an aromatic group, a substituted aromatic group, —NR²⁴R²⁵, —CONR²⁴R²⁵, —C(═NR⁶⁰)NR²¹R²², —Q-(aliphatic group), —Q-(substituted aliphatic group), —O-(aliphatic group), —O-(substituted aliphatic group),—O-(aromatic group), —O-(substituted aromatic group), an electron withdrawing group, —(O)ᵤ—(CH₂)ₜ—C(O)OR²⁰, —(O)ᵤ—(CH₂)ₜ—OC(O)R²⁰, —(O)ᵤ—(CH₂)ₜ—C(O)—NR²⁰R²² or —(O)ᵤ—(CH₂)ₜ—NHC(O)O—R²⁰. Q, R²⁰, R²¹, R²², R²⁴, R²⁵, R⁶⁰, u and t are as described herein.

Preferably R⁴⁰ is an aliphatic group, substituted aliphatic group, —O-(aliphatic group) or —O-(substituted aliphatic group). More preferably R⁴⁰ is an —O-alkyl, such as —O—CH₃, —O—C₂H₅, —O—C₃H₇ or —O—C₄H₉.

In another embodiment, R⁴⁰ can be represented by —(O)ᵤ—(CH₂)ₜ—C(O)—NR²¹R²², wherein u is one, t is zero, and R²¹ and R²² are as described herein. In this embodiment, R²¹ and R²² can each independently be —H, a substituted or unsubstituted aliphatic group, a substituted or unsubstituted aromatic group, or R²¹ and R²² taken together with the nitrogen atom to which they are bonded form a substituted or unsubstituted nonaromatic heterocyclic ring (e.g., pyrrolidine, piperidine, morpholine).

In another embodiment, R⁴⁰ can be represented by —(O)ᵤ—(CH₂)ₜ—C(O)—NR²¹R²², wherein u is zero, t is one to about three, and R²¹ and R²² are as described herein.

In another embodiment, R⁴⁰ can be represented by —(O)ᵤ—(CH₂)ₜ—C(O)—NR²¹R²², wherein both u and t are zero, and R²¹ and R²² are as described herein.

In another embodiment, R⁴⁰ is an aliphatic group (e.g., methyl, ethyl, propyl) that is substituted with —NR²⁴R²⁵ or —CONR²⁴R²⁵, wherein R²⁴ and R²⁵ are as described herein. For example, R⁴⁰ can be represented by

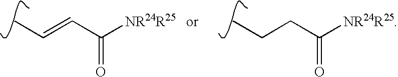

In another embodiment, R⁴⁰ is —O—C(O)—NR²¹R²⁶, wherein R²¹ is as described herein, R²⁶ can be —H, an aliphatic group, a substituted aliphatic group, an aromatic group, a substituted aromatic group, a non-aromatic heterocyclic group, —C(O)—O-(substituted or unsubstituted aliphatic group), —C(O)—O-(substituted or unsubstituted aromatic group), —S(O)₂-(substituted or unsubstituted aliphatic group), —S(O)₂-(substituted or unsubstituted aromatic group) or R²¹ and R²⁶, taken together with the nitrogen atom to which they are bonded, can form a substituted or unsubstituted non-aromatic heterocyclic ring.

In additional embodiments, R⁴⁰ can be —S(O)₂—NR²¹R²² or —N—C(O)—NR²¹R²², wherein R²¹ and R²² are as described herein.

In a preferred embodiment, the chemokine receptor antagonist can be represented by Structural Formula I wherein n is three, M is C(OH)R², R² is a phenyl group or a halophenyl group (e.g., 4-chlorophenyl) and Z is represented by Structural Formula (VI) wherein X₁ is —CH₂—O—. In one example of this embodiment, R⁴⁰ can be —O-(substituted aliphatic group), such as

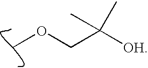

In particularly preferred embodiments, R⁴⁰ is

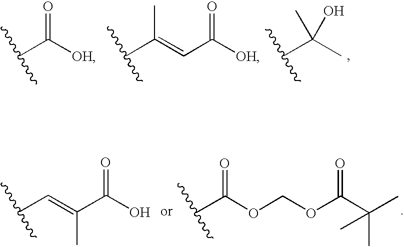

In other preferred embodiments, R⁴⁰ is a substituted aliphatic group, a substituted aromatic group, —O-substituted aliphatic group or —O-substituted aromatic group. Preferably the aliphatic or aromatic moiety of the substituted aliphatic group, substituted aromatic group, —O-substituted aliphatic group or —O-substituted aromatic group bears a substituent selected from the group consisting of —OH, —COOR—Q-aliphatic group or —Q-aromatic group substituent. Q is as described herein. Preferably, Q is —C(O)O—. For example, R⁴⁰ can be a linear, branched or cyclic aliphatic group that contains 1 to 6 carbon atoms, such as a C₁-C₆ alkyl group, a C₂-C₆ alkenyl, C₂-C₆ alkynyl, that is substituted with —OH, —COOH, —C(O)O—(C₁-C₆ aliphatic) or —C(O)O-(aromatic).

In another embodiment, the antagonist of chemokine activity can be represented by Structural Formula (VII):

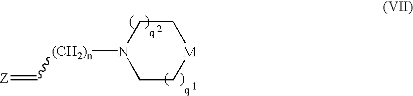

and physiologically acceptable salts thereof

n is as described in Structural Formula (I). Z is as described herein, preferably as described in Structural Formula (V) or (VI).

M is >NR², >CR¹R², —O—CR¹R²—O— or —CH₂-CR¹R²—O—.

R¹ and R² are as described in Structural Formula (I).

q¹ is an integer, such as an integer from zero to about three, and q² is an integer from zero to about one. The ring containing M can be substituted or unsubstituted.

Thus, the antagonist of chemokine function can be represent by, for example, Structural Formulas (VIIa)-(VIIk):

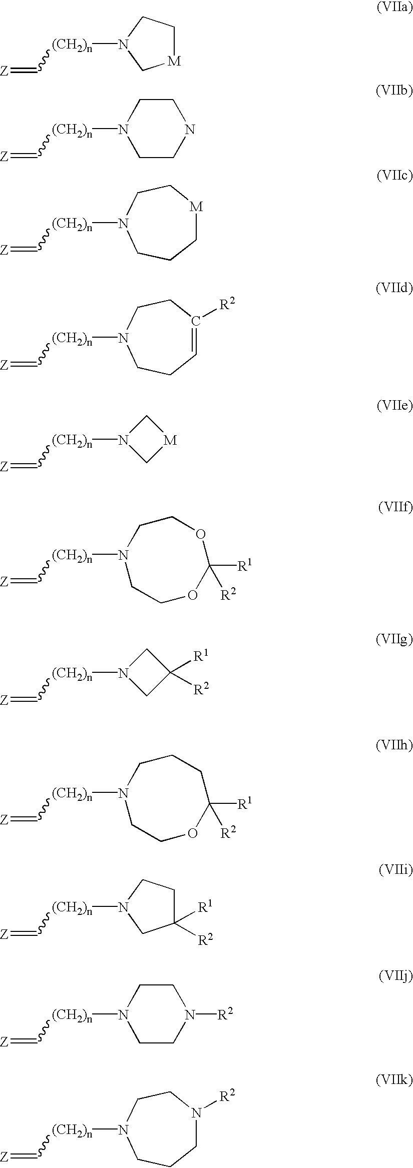

and physiologically acceptable salts thereof, wherein Z, n and M are as described in Structural Formula (VII), and the ring which contains M is substituted or unsubstituted. The ring containing M can have one or more suitable substituents which are the same or different. Suitable substituents for the ring which contains M and other nonaromatic heterocyclic rings are as described herein. For example, the ring containing M can be substituted with a methyl, ethyl, propyl, butyl or oxo group.

When the ring containing M is substituted, the compound can be represented by Stuctural Formula (VIII):

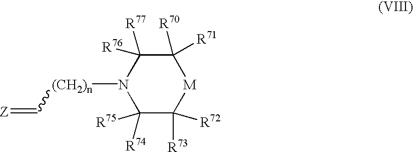

or physiologically acceptable salt thereof.

R⁷⁰, R⁷¹, R⁷², R⁷³, R⁷⁴, R⁷⁵, R⁷⁶ and R⁷⁷ are independently —H, —OH, —N₃, a halogen, an aliphatic group, a substituted aliphatic group, an aminoalkyl group, —O-(aliphatic group), —O-(substituted aliphatic group), —SH, —S-(aliphatic group), —S-(substituted aliphatic group), —OC(O)-(aliphatic group), —O—C(O)-(substituted aliphatic group), —C(O)O-(aliphatic group), —C(O)O-(substituted aliphatic group), —COOH, —CN, —CO—NR³R⁴, —NR³R⁴, an acyl group, a substituted acyl group, a benzyl group, a substituted benzyl group, a non-aromatic heterocyclic group, a substituted non-aromatic heterocyclic group, —O-(substituted or unsubstituted aromatic group), or any two of R⁷⁰, R⁷¹, R⁷², R⁷³, R⁷⁴, R⁷⁵, R⁷⁶ and R⁷⁷ taken together with the atoms to which they are bonded form a three to eight membered ring.

n is as described in Structural Formula (I). Z is as described herein, preferably as described in Structural Formula (V) or (VI). M is as described in Structural Formula (VII). Preferably, M is >NR² or >CR¹R².

In certain embodiments R⁷⁴, R⁷⁵, R⁷⁶ and R⁷⁷ are —H. In other embodiments, R⁷⁴, R⁷⁵, R⁷⁶ and R⁷⁷ are —H, and at least one of R⁷⁰, R⁷¹, R⁷² and R⁷³ is an aliphatic group or a substituted aliphatic group. Preferred aliphatic groups at R⁷⁰, R⁷¹, R⁷², R⁷³, R⁷⁴, R⁷⁵, R⁷⁶ and R⁷⁷ are C₁-C₆ alkyl. In more particular embodiments, the compound has the formula of Structural Formula VIII wherein R⁷⁰, R⁷³, R⁷⁴, R⁷⁵, R⁷⁶ and R⁷⁷ are —H, and at least one of R⁷¹ and R⁷² is —CH₃.

The nitrogen atom in the ring containing M can be a tertiary nitrogen as depicted in Structural Formula (IV), or the nitrogen atom can be quaternized with a suitable substituent, such as a C₁ to about C₆ or a C₁ to about C₃ substituted or unsubstituted aliphatic group. Compounds which comprise a quaternary nitrogen atom can also contain a counteranion such as chloride, bromide, iodide, acetate, perchlorate and the like.

The antagonist of chemokine function can be represented by Structural Formula (VII) wherein the heterocyclic ring containing M is substituted with a suitable bivalent group which is bonded to two atoms that are in the ring, thereby forming a bicyclic moiety. Suitable bivalent groups include, for example, substituted or unsubstituted bivalent aliphatic groups, such as a C₁-C₆ alkylene group.

The antagonist of chemokine receptor function can comprise a variety of bicyclic moieties. In one embodiment, the antagonist of chemokine receptor function can be represented by Structural Formula (VIII):

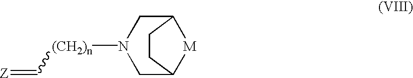

and physiologically acceptable salts thereof.

M is >NR², >CR¹R², —O—CR¹R²—O— or —CH₂—CR¹R²—O—. Preferably, M is >NR² or >CR¹R². R¹ and R² are as described in Structural Formula (I), and n and Z are as described in structural Formula (VII).

In another embodiment, the antagonist of chemokine receptor function is represented by Structural Formula (IX):

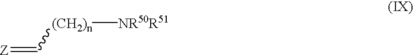

and physiologically acceptable salts thereof.

Z is as described herein, preferably as described in Structural Formula (V) or (VI).

n is an integer, such as an integer from one to about four. Preferably, n is one, two or three. More preferably n is two. In alternative embodiments, other aliphatic or aromatic spacer groups (L) can be employed for (CH₂)ₙ.

R⁵⁰ and R⁵¹ are each independently —H, an aliphatic group, a substituted aliphatic group, an aminoalkyl group, —NR³R⁴, an aromatic group, a substituted aromatic group, a benzyl group, a substituted benzyl group, a non-aromatic heterocyclic group, a substituted non-aromatic heterocyclic group or a covalent bond between the nitrogen atom an adjacent carbon atom.

R³ and R⁴ are independently —H, an acyl group, a substituted acyl group, an aliphatic group, a substituted aliphatic group, an aromatic group, a substituted aromatic group, a benzyl group, a substituted benzyl group, a non-aromatic heterocyclic group or a substituted non-aromatic heterocyclic group.

R³ and R⁴ taken together with the atom to which they are bonded, can alternatively form a substituted or unsubstituted non-aromatic carbocyclic or heterocyclic ring.

In a preferred embodiment R⁵⁰ is a substituted aliphatic group, such as a substituted C₁ to about C₁₂ alkyl group, and R⁵¹ is —H or a substituted or unsubstituted aliphatic group. More preferably, R⁵⁰ is a substituted linear or branched C₂ to about C₇ aliphatic group wherein one or more carbon atoms can be replaced by a heteroatom, such as nitrogen, oxygen or sulfur, and R⁵¹ is —H or a linear or branched C₁ to about C₆ or a C₁ to about C₃ aliphatic group wherein one or more carbon atoms can be replaced by a heteroatom. R⁵⁰ and R⁵¹ can be substituted with one or more suitable substituents, as described herein, Preferably an aromatic group (e.g., phenyl, 4-halophenyl). For example, R⁵⁰ can be selected from the group consisting of:

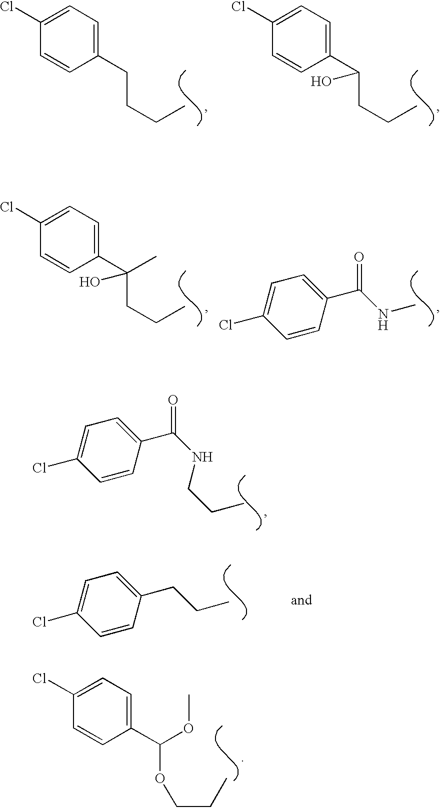

The activity of chemokine receptor antagonists represented by Structural Formula IX can be affected by the character of the nitrogen atom to which R⁵⁰ and R⁵¹ are bonded. It is believed that compounds in which said nitrogen atom is basic can have potent chemokine receptor antagonist activity. It is known that the basicity of a nitrogen atom can be decreased when the nitrogen atom is bonded to a carbonyl group, sulfonyl group or a sulfinyl group. Therefore, it is preferred that neither R⁵⁰ nor R⁵¹ comprise a carbonyl group, sulfonyl group or sulfinyl group that is directly bonded to the nitrogen atom.

In another aspect, the antagonist of chemokine receptor function is represented by Structural Formula (X):

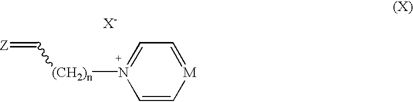

and physiologically acceptable salts thereof.

Z is a cycloalkyl or non-aromatic heterocyclic ring group fused to one, two or more aromatic rings, wherein each ring in Z is independently substituted or unsubstituted. Preferably, Z is as described in Structural Formula (VI).

n is an integer, such as an integer from one to about four. Preferably, n is one, two or three. More preferably n is two. In alternative embodiments, other aliphatic or aromatic spacer groups (L) can be employed for (CH₂)ₙ.

M is >NR₂ or >CR².

R² is —H, —OH, an acyl group, a substituted acyl group, —NR⁵R⁶, an aliphatic group, a substituted aliphatic group, an aromatic group, a substituted aromatic group, a benzyl group, a substituted benzyl group, a non-aromatic heterocyclic group, a substituted non-aromatic heterocyclic group, —O-(substituted or unsubstituted aromatic group) or —O-(substituted or unsubstituted aliphatic group). R² is preferably an aromatic group or a substituted aromatic group.

R⁵ and R⁶ are independently —H, an acyl group, a substituted acyl group, an aliphatic group, a substituted aliphatic group, an aromatic group, a substituted aromatic group, a benzyl group, a substituted benzyl group, a non-aromatic heterocyclic group or a substituted non-aromatic heterocyclic group.

R⁵ and R⁶ taken together with the atom to which they are bonded, can alternatively form a substituted or unsubstituted non-aromatic carbocyclic or heterocyclic ring.

X⁻ is a physiologically acceptable anion. Preferably, X⁻ is Cl⁻ or Br⁻.

The chemokine receptor antagonist described herein can be prepared and administered as active compounds or as prodrugs. Generally, prodrugs are analogues of pharmaceutical agents which can undergo chemical conversion by metabolic processes to become fully active. For example, A prodrug of the invention can be prepared by selecting appropriate groups for R⁴⁰. In one embodiment, a prodrug can be represented by Structural Formula (XI):

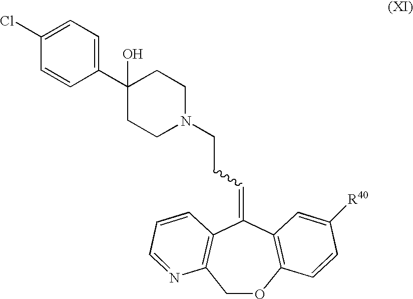

wherein, R⁴⁰ is Q-substituted aliphatic group, and the aliphatic group is substituted with —(O)ᵤ—(CH₂)ₜ—C(O)OR²⁰, wherein Q is —C(O)O—, u is one, t is zero and R²⁰ is a cyclic aliphatic group. For example, when the substituted aliphatic group is a substituted ethyl group, R⁴⁰ can be represented by:

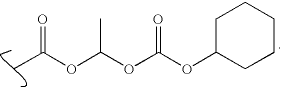

Such a prodrug can be converted to an active chemokine receptor antagonist represented by Structural Formula XI, wherein R⁴⁰ is —COOH.

Another embodiment of the present invention includes novel compounds employed in these methods.

The compounds disclosed herein can be obtained as E- and Z-configurational isomers. It is expressly pointed out that the invention includes compounds of the E-configuration and the Z-configuration around the double bond connecting Ring C of Z to the remainder of the molecule, and a method of treating a subject with compounds of the E-configuration, the Z-configuration, and mixtures thereof. Accordingly, in the structural formulas presented herein, the symbol:

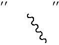

is used to represent both the E-configuration and the Z-configuration. Preferably Ring A and the alkylene chain bonded to Ring C are in the cis configuration. For example, the compounds can have the configuration of:

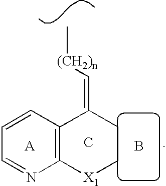

It is understood that one configuration can have greater activity than another. The desired configuration can be determined by screening for activity, employing the methods described herein.

Additionally, certain compounds of the invention may be obtained as different sterioisomers (e.g., diastereomers and enantiomers). It is pointed out that the invention includes all isomeric forms and racemic mixtures of the disclosed compounds and a method of treating a subject with both pure isomers and mixtures thereof, including racemic mixtures. Sterioisomers can be separated and isolated using any suitable method, such as chromatography. Again, it is understood that one sterioisomer may be more active than another. The desired isomer determined by screening.

Also included in the present invention are physiologically acceptable salts of the compounds represented by Structural Formulas (I) through (XI). Salts of compounds containing an amine or other basic group can be obtained, for example, by reacting with a suitable organic or inorganic acid, such as hydrogen chloride, hydrogen bromide, acetic acid, citric acid, perchloric acid and the like. Compounds with a quaternary ammonium group also contain a counteranion such as chloride, bromide, iodide, acetate, perchlorate and the like. Salts of compounds containing a carboxylic acid or other acidic functional group can be prepared by reacting with a suitable base, for example, a hydroxide base. Salts of acidic functional groups contain a countercation such as sodium, potassium, ammonium, calcium and the like.

As used herein, aliphatic groups include straight chained, branched or cyclic C₁-C₂₀ hydrocarbons which are completely saturated or which contain one or more units of unsaturation. Preferred aliphatic groups are C₁ to about C₁₀ hydrocarbons. More preferred are C₁ to about C₆ or C₁ to about C₃ hydrocarbons. One or more carbon atoms in an aliphatic group can be replaced with a heteroatom, such as nitrogen, oxygen or sulfur. For example, suitable aliphatic groups include substituted or unsubstituted linear, branched or cyclic C₁-C₂₀ alkyl, alkenyl or alkynyl groups.

An aminoalkyl group is an alkyl group substituted with —NR²⁴R²⁵, R²⁴ and R²⁵ are as described herein. Preferably the alkyl moiety comprises one to about twelve, more preferably one to about six carbon atoms. The alkyl moiety of an aminoalkyl group can be unsubstituted or substituted as described herein for aliphatic groups. Examples of suitable aminoalkyl groups include aminomethyl, 2-aminoethyl, 3-aminopropyl, 4-aminobutyl, dimethylaminoethyl, diethylaminomethyl, methylaminohexyl, aminoethylenyl and the like.

Aromatic groups include carbocyclic aromatic groups such as phenyl, 1-naphthyl, 2-naphthyl, 1-anthracyl and 2-anthracyl, and heterocyclic aromatic or heteroaryl groups such as N-imidazolyl, 2-imidazolyl, 4-imidazolyl, 5-imidazolyl, 2-thienyl, 3-thienyl, 2-furanyl, 3-furanyl, 2-pyrrolyl, 3-pyrrolyl, 2-pyridyl, 3-pyridyl, 4-pyridyl, 2-pyrimidyl, 4-pyrimidyl, 5-pyrimidyl, 3-pyridazinyl, 4-pyridazinyl, 3-pyrazolyl, 4-pyrazolyl, 5-pyrazolyl, 2-pyrazinyl, 2-thiazolyl, 4-thiazolyl, 5-thiazolyl, 5-tetrazolyl, 2-oxazolyl, 4-oxazolyl and 5-oxazolyl. Where these rings are fused, for example, to Ring C, the stated point of attachment can be either of the two fused bonds.

Aromatic groups also include fused polycyclic aromatic ring systems in which a carbocyclic aromatic ring or heteroaryl ring is fused to one or more other rings. Examples include tetrahydronaphthyl, 2-benzothienyl, 3-benzothienyl, 2-benzofuranyl, 3-benzofuranyl, 2-indolyl, 3-indolyl, 2-quinolinyl, 3-quinolinyl, 2-benzothiazolyl, 2-benzooxazolyl, 2-benzimidazolyl, 1-isoquinolinyl, 3-quinolinyl, 1-isoindolyl, 3-isoindolyl, acridinyl, 3-benzisoxazolyl, and the like. Also included within the scope of the term “aromatic group”, as it is used herein, is a group in which one or more carbocyclic aromatic rings and/or heteroaryl rings are fused to a cycloalkyl or non-aromatic heterocyclic ring, for example, benzocyclopentane, benzocyclohexane.

Non-aromatic heterocyclic rings are non-aromatic carbocyclic rings which include one or more heteroatoms such as nitrogen, oxygen or sulfur in the ring. The ring can be five, six, seven or eight-membered and/or fused to another ring, such as a cycloalkyl on aromatic ring. Examples include 1,3-dioxolan-2-yl, 3-1H-benzimidazol-2-one, 3-1-alkyl-benzimidazol-2-one, 3-1-methyl-benzimidazol-2-one, 2-tetrahydrofuranyl, 3-tetrahydrofuranyl, 2-tetrahyrothiophenyl, 3-tetrahyrothiophenyl, 2-morpholino, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-piperazinyl, 2-piperazinyl, 1-piperidinyl, 2-piperidinyl, 3-piperidinyl, 4-piperidinyl, 4-thiazolidinyl, diazolonyl, N-substituted diazolonyl, 1-phthalimidyl, 1-3-alkyl-phthalimidyl, benzoxane, benzopyrolidine, benzopiperidine, benzoxolane, benzothiolane, benzothiane, tetrahydrofuran-2-one-3-yl, 2,5-dihydro-5-oxo-4H-1 ,2,4-thiadiazol-3-yl, 2-oxo-3H-1,2,3,5-oxathiadiazol-4-yl,

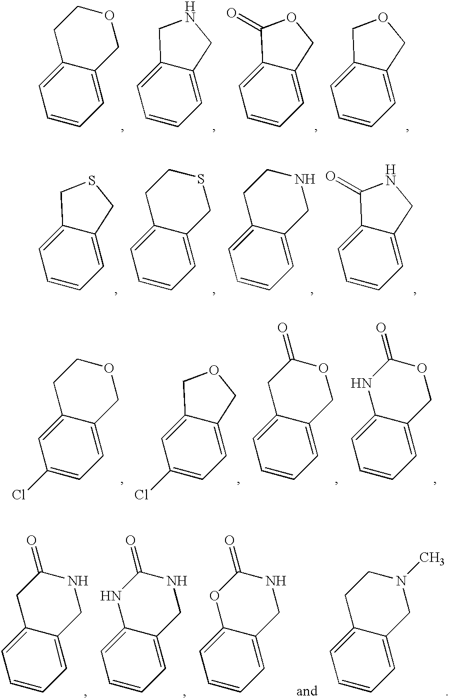

Suitable substituents on an aliphatic group, aromatic group (carbocyclic and heteroaryl), non-aromatic heterocyclic ring or benzyl group include, for example, an electron withdrawing group, a halogen (chloride, bromide, fluoride, iodide), azido, —CN, —COOH, —OH, —CONR²⁴R²⁵, —NR²⁴R²⁵, —OS(O)₂NR²⁴R²⁵, —S(O)₂NR²⁴R²⁵, —SO₃H, —S(O)₂NH₂, guanidino, ureido, oxalo, amidino, —C(═NR⁶⁰)NR²¹R²², ═NR⁶⁰, —(O)ᵤ—(CH₂)ₜ—C(O)OR²⁰, —(O)ᵤ—(CH₂)ₜ—OC(O)R²⁰, —(O)ᵤ—(CH₂)ₜ—C(O)—NR²¹R²², —(O)ᵤ—(CH₂)ₜ—NHC(O)O—R²⁰, —Q—H, —Q-(aliphatic group), —Q-(substituted aliphatic group), —Q-(aryl), —Q-(aromatic group), —Q-(substituted aromatic group), —Q-(CH₂)ₚ-(substituted or unsubstituted aromatic group) p is an integer from 1-5), —Q-(non-aromatic heterocyclic group) or —Q-(CH₂)ₚ-(non-aromatic heterocyclic group).

R²⁰, R²¹ and R²² are independently —H, an aliphatic group, a substituted aliphatic group, an aromatic group, a substituted aromatic group, a non-aromatic heterocyclic group, —NHC(O)—O-(aliphatic group), —NHC(O)—O-(aromatic group) or —NHC(O)—O-(non-aromatic heterocyclic group) and wherein R²¹ and R²², taken together with the nitrogen atom to which they are bonded, can form a substituted or unsubstituted non-aromatic heterocyclic ring.

R⁶⁰ is a —H, —OH, —NH₂, an aromatic group or a substituted aromatic group.

t is an integer from zero to about three, and the methylene group, —(CH₂)ₜ—, can be substituted, as described herein for aliphatic groups, or unsubstituted. u is zero or one.

Q is —O—, —S—, —S(O)—, —S(O)2—, —OS(O)2—, —C(O)—, —OC(O)—, —C(O)O—, —C(O)C(O)—O—, —O—C(O)C(O)—, —C(O)NH—, —NHC(O)—, —OC(O)NH—, —NHC(O)O—, —NH—C(O)—NH—, —S(O)₂NH—, —NHS(O)₂—, —N(R²³)—, —C(NR²³)NHNH—, —NHNHC(NR²³)—, —NR²⁴C(O)— or —NR²⁴S(O)₂—.

R²³ is —H, an aliphatic group, a benzyl group, an aryl group or non-aromatic heterocyclic group.

R²⁴ and R²⁵ are independently —H, —OH, an aliphatic group, a substituted aliphatic group, a benzyl group, an aryl group, non-aromatic heterocyclic group or R²⁴ and R²⁵ taken together with the nitrogen atom to which they are bonded can form a substituted or unsubstituted non-aromatic heterocyclic ring.

A substituted non-aromatic heterocyclic ring, benzyl group or aromatic group can also have an aromatic group, an aliphatic or substituted aliphatic group, as a substituent. When a non-aromatic ring (carbocyclic or heterocyclic) or an aromatic ring (carbocyclic aromatic or heteroaryl) is substituted with another ring, the two rings can be fused. A substituted aliphatic group can also have an oxo group, epoxy group, non-aromatic heterocyclic ring, benzyl group, substituted benzyl group, aromatic group or substituted aromatic group as a substituent. A substituted non-aromatic heterocyclic ring can also have ═O, ═S, ═NH or ═N (aliphatic, aromatic or substituted aromatic group) as a substituent. A substituted aliphatic, substituted aromatic, substituted non-aromatic heterocyclic ring or substituted benzyl group can have more than one substituent, which can be the same or different.

Acyl groups include substituted and unsubstituted aliphatic carbonyl, aromatic carbonyl, aliphatic sulfonyl and aromatic sulfonyl.

Suitable electron withdrawing groups include, for example, alkylimines, alkylsulfonyl, carboxamido, carboxylic alkyl esters, —CH═NH, —CN, —NO₂ and halogens.

In the structural formulas depicted herein, the single or double bond by which a chemical group or moiety is connected to the remainder of the molecule or compound is indicated by the following symbol:

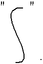

For example, the corresponding symbol in Structural Formulas (II), (III) and (IV) indicates the double bond by which the central ring of the tricyclic ring system is connected to the remainder of the molecule represented by Structural Formula (I).

A “subject” is preferably a bird or mammal, such as a human, but can also be an animal in need of veterinary treatment, e.g., domestic animals (e.g., dogs, cats, and the like), farm animals (e.g., cows, sheep, fowl, pigs, horses, and the like) and laboratory animals (e.g., rats, mice, guinea pigs, and the like).

An “effective amount” of a compound is an amount which results in the inhibition of one or more processes mediated by the binding of a chemokine to a receptor in a subject with a disease associated with aberrant leukocyte recruitment and/or activation. Examples of such processes include leukocyte migration, integrin activation, transient increases in the concentration of intracellular free calcium [Ca²⁺]ᵢ and granule release of proinflammatory mediators. Alternatively, an “effective amount” of a compound is a quantity sufficient to achieve a desired therapeutic and/or prophylactic effect, such as an amount which results in the prevention of or a decrease in the symptoms associated with a disease associated with aberrant leukocyte recruitment and/or activation.

The amount of compound administered to the individual will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. Typically, an effective amount of the compound can range from about 0.1 mg per day to about 100 mg per day for an adult. Preferably, the dosage ranges from about 1 mg per day to about 100 mg per day. An antagonist of chemokine receptor function can also be administered in combination with one or more additional therapeutic agents, e.g. theophylline, β-adrenergic bronchodilators, corticosteroids, antihistamines, antiallergic agents, immunosuppressive agents (e.g., cyclosporin A, FK-506, prednisone, methylprednisolone) and the like.

The compound can be administered by any suitable route, including, for example, orally in capsules, suspensions or tablets or by parenteral administration. Parenteral administration can include, for example, systemic administration, such as by intramuscular, intravenous, subcutaneous, or intraperitoneal injection. The compound can also be administered orally (e.g., dietary), transdermally, topically, by inhalation (e.g., intrabronchial, intranasal, oral inhalation or intranasal drops), or rectally, depending on the disease or condition to be treated. Oral or parenteral administration are preferred modes of administration.

The compound can be administered to the individual in conjunction with an acceptable pharmaceutical or physiological carrier as part of a pharmaceutical composition for treatment of HIV infection, inflammatory disease, or the other diseases discussed above. Formulation of a compound to be administered will vary according to the route of administration selected (e.g., solution, emulsion, capsule). Suitable carriers may contain inert ingredients which do not interact with the compound. Standard pharmaceutical formulation techniques can be employed, such as those described in Remington's Pharmaceutical Sciences, Mack Publishing Company, Easton, Pa. Suitable carriers for parenteral administration include, for example, sterile water, physiological saline, bacteriostatic saline (saline containing about 0.9% benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents”, John Wiley and Sons, 1986).

The activity of compounds of the present invention can be assessed using suitable assays, such as receptor binding assays and chemotaxis assays. For example, as described in the Exemplification Section, small molecule antagonists of RANTES and MIP-1α binding have been identified utilizing THP-1 cells which bind RANTES and chemotax in response to RANTES and MIP-1α as a model for leukocyte chemotaxis. Specifically, a high through-put receptor binding assay, which monitors ¹²⁵I-RANTES and ¹²⁵I-MIP-1α binding to THP-1 cell membranes, was used to identify small molecule antagonists which block binding of RANTES and MIP-1α. Compounds of the present invention can also be identified by virtue of their ability to inhibit the activation steps triggered by binding of a chemokine to its receptor, such as chemotaxis, integrin activation and granule mediator release. They can also be identified by virtue of their ability to block RANTES and MIP-1α mediated HL-60, T-cell, peripheral blood mononuclear cell, and eosinophil chemotactic response.

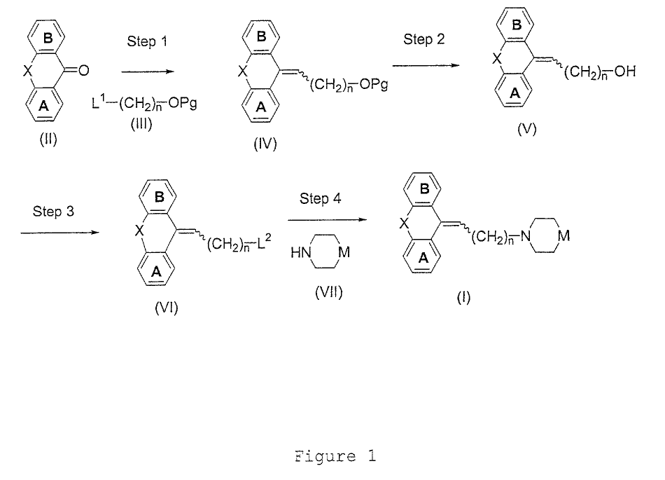

The compounds disclosed herein can be prepared accordingly to the schemes shown in FIGS. 1-5 and 7. The schemes are described in greater detail below.

FIG. 1 shows the preparation of compounds represented by Structural Formula (I). L¹ is PPh₃Cl, PPh₃Br, PPh₃I or (EtO)₂P(O), L² is a suitable leaving group such as halogen, p-toluene sulfonate, mesylate, alkoxy, and phenoxy; Pg is a suitable protecting group such as tetrahydropyranyl; and the other symbols are as defined above.

In Step 1 of FIG. 1, a Wittig reaction is carried out in a solvent such as ether, or tetrahydrofuran (THF) in the presence of a base such as sodium hydride, n-butyl lithium or lithium diisopropylamide (LDA) at 0° C. up to the reflux temperature for the solvent used for 5 minutes to 72 h. Compounds represented by Formula II in FIG. 1 can be prepared by methods disclosed in JP 61/152673, U.S. Pat. No. 5,089,496, WO 89/10369, WO 92/20681 and WO 93/02081, the entire teachings of which are incorporated herein by reference.

In Step 2 of FIG. 1, deprotection is carried out with an acid in a solvent such as methanol at room temperature up to the reflux temperature for the solvent used for 5 minutes to 72 h. Alternatively, a compound of represented by Formula V in FIG. 1 can be prepared directly from step 1 without isolating an intermediate. The reaction mixture obtained after the work up of the reaction described in step 1 can be dissolved in the solvent and reacted with the acid.

In Step 3 of FIG. 1, the hydroxy group can be converted to a leaving group by known methods. Compounds represented by Formula VI in FIG. 1 can be prepared by methods disclosed in J. Med. Chem., 1992 (35) 2074-2084 and JP 61/152673.

In Step 4 of FIG. 1, an alkylation reaction is carried out in a solvent such as acetone, methyl ethyl ketone, ethyl acetate, toluene, tetrahydrofuran (THF) or dimethylformamide (DMF) in the presence of a base such as potassium carbonate or sodium hydride and a catalyst such as an alkali metal iodide at room temperature up to the reflux temperature for the solvent used for 5 minutes to 72 h.

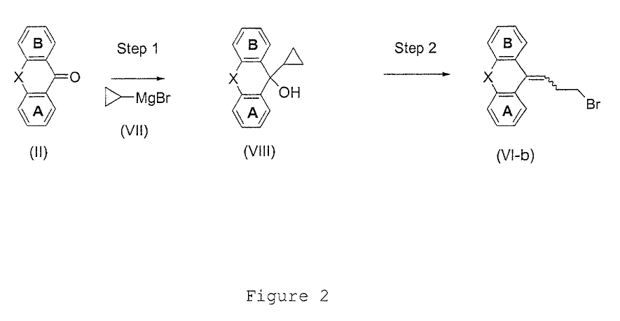

FIG. 2 shows the preparation of compounds represented by Compound (VI-b). In Step 1 of FIG. 2, a Grignard reaction may be carried out in a solvent such as ether, or tetrahydrofuran (THF) at 0° C. up to the reflux temperature for the solvent used for 5 minuets to 72 h. Compound VII is available commercially.

In Step 2 of FIG. 2, bromination may be carried out with brominate agents such as hydrobromic acid, bromotrimethylsilane or boron tribromide-methyl sulfide complex in a solvent such as acetic acid, dichloromethane or dichloroethane at room temperature up to the reflux temperature for the solvent used for 5 minutes to 72 h.

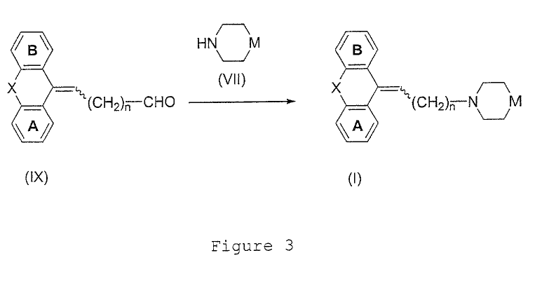

FIG. 3 shows the preparation of compounds represented by Structural Formula (I). In FIG. 3, a reductive amination may be carried out with reducing regents such as sodium cyanoborohydride, sodium acetoxyborohydride or sodium borohydride in a solvent such as methanol, ethanol, tetrahydrofuran (THF), dichloromethane or dichloroethane at room temperature up to the reflux temperature for the solvent used for 5 minutes to 72 h.

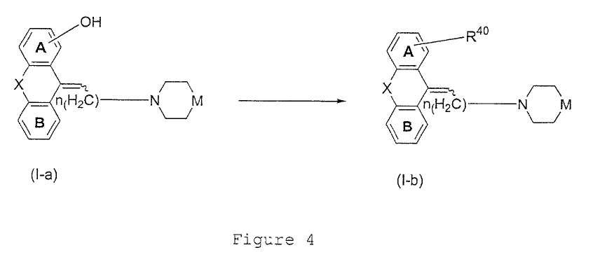

FIG. 4 shows the preparation of compounds represented by Structural Formula (I), where in Z is represented by Structural Formulas (III) and wherein Ring A and/or Ring B in Z is substituted with R⁴⁰. In FIG. 4, the alkylation reaction can be carried out in a solvent such as acetone, methyl ethyl ketone, ethyl acetate, toluene, tetrahydrofuran (THF) or dimethylformamide (DMF) in the presence of a base such as potassium carbonate or sodium hydride and a catalyst such as an alkali metal iodide at room temperature up to the reflux temperature for the solvent used for 5 minutes to 72 h.

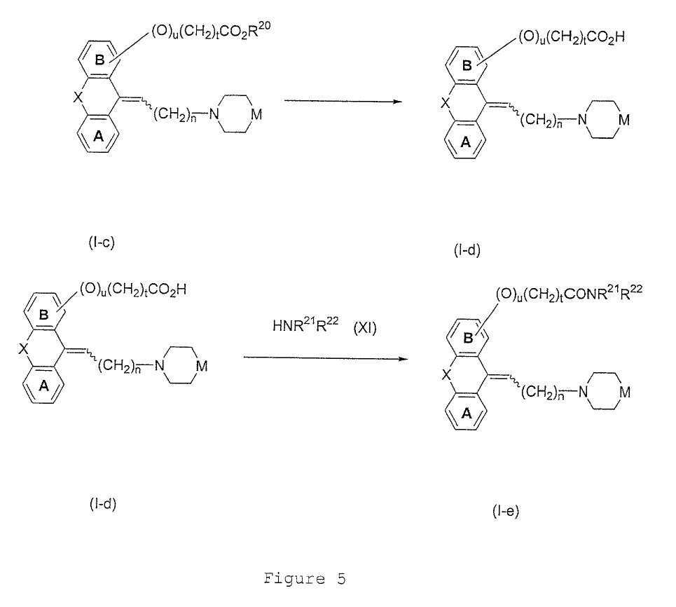

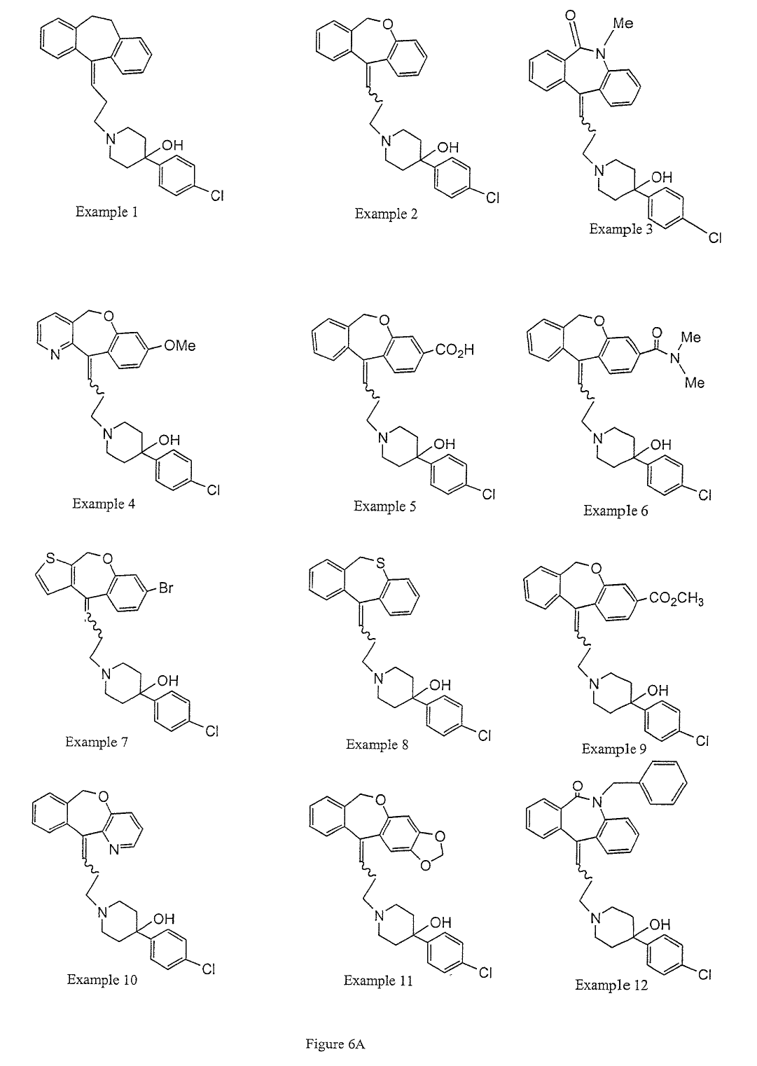

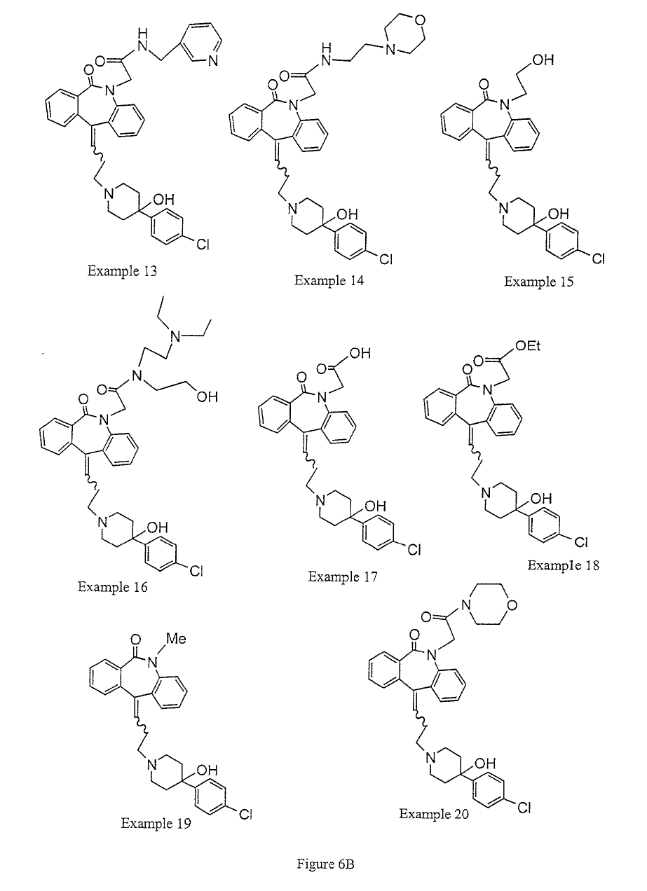

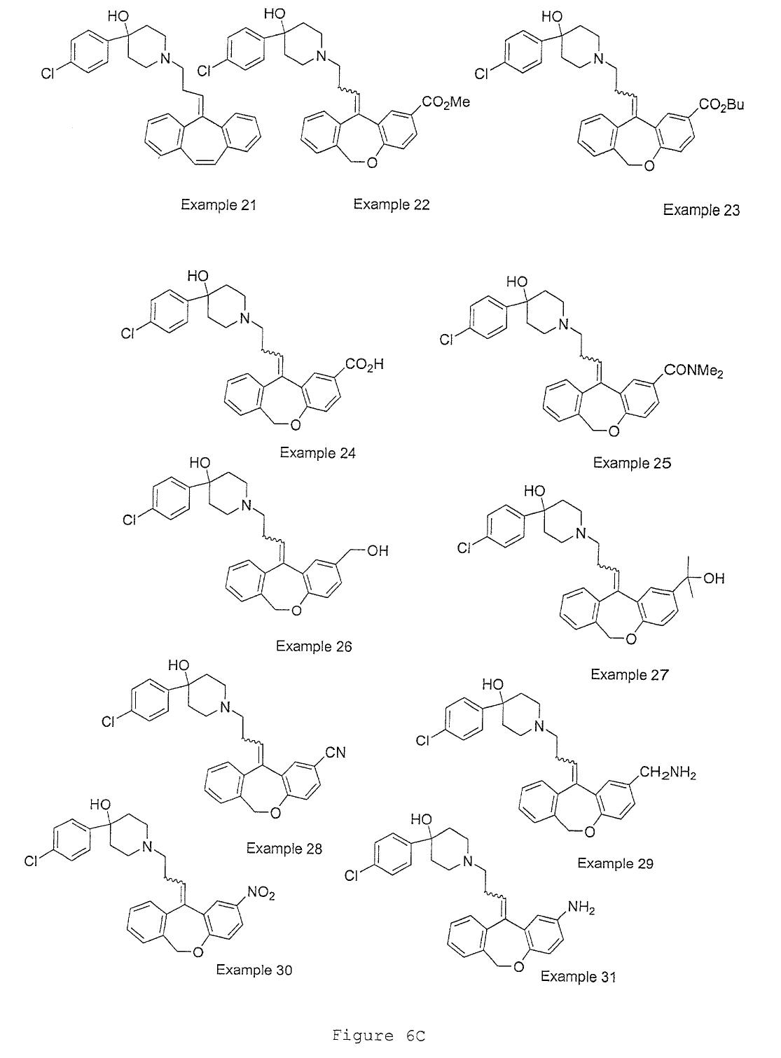

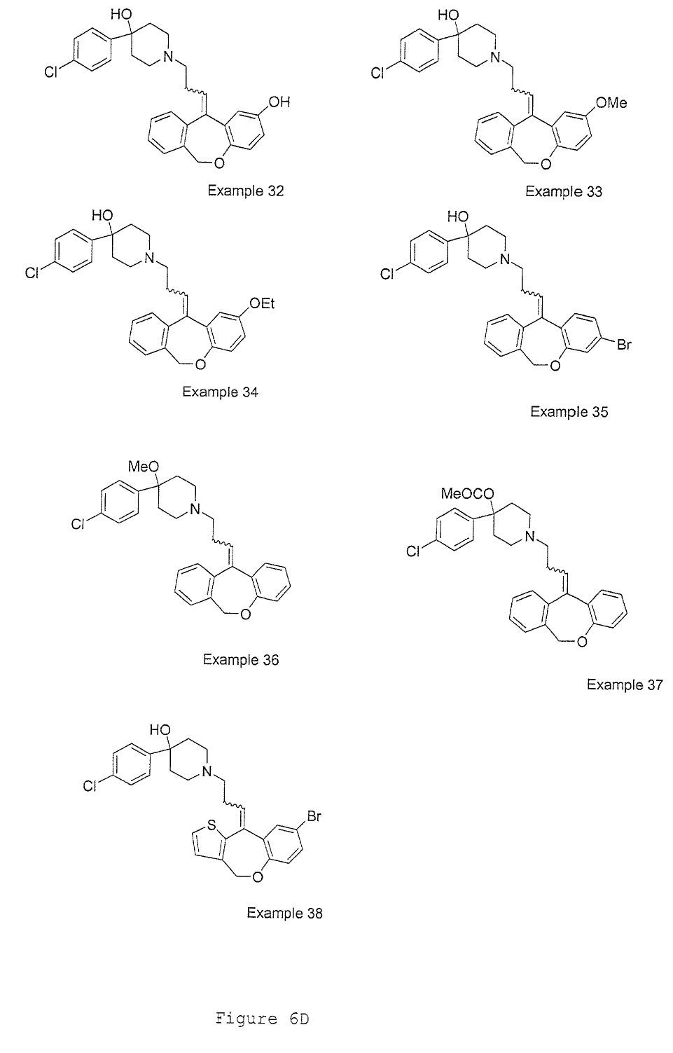

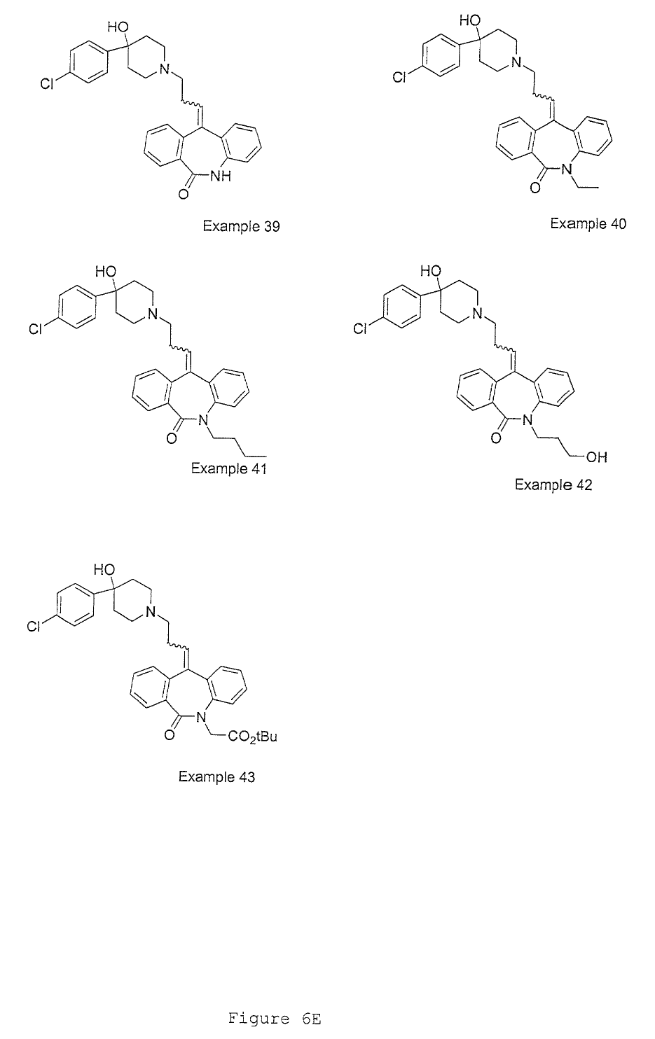

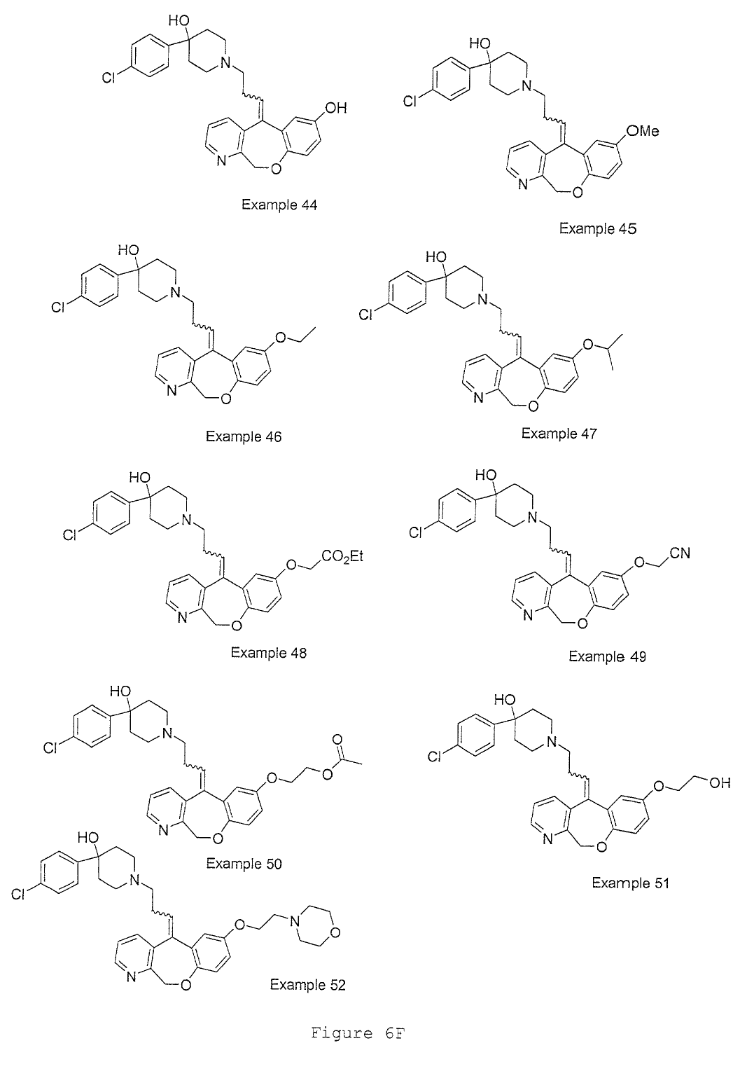

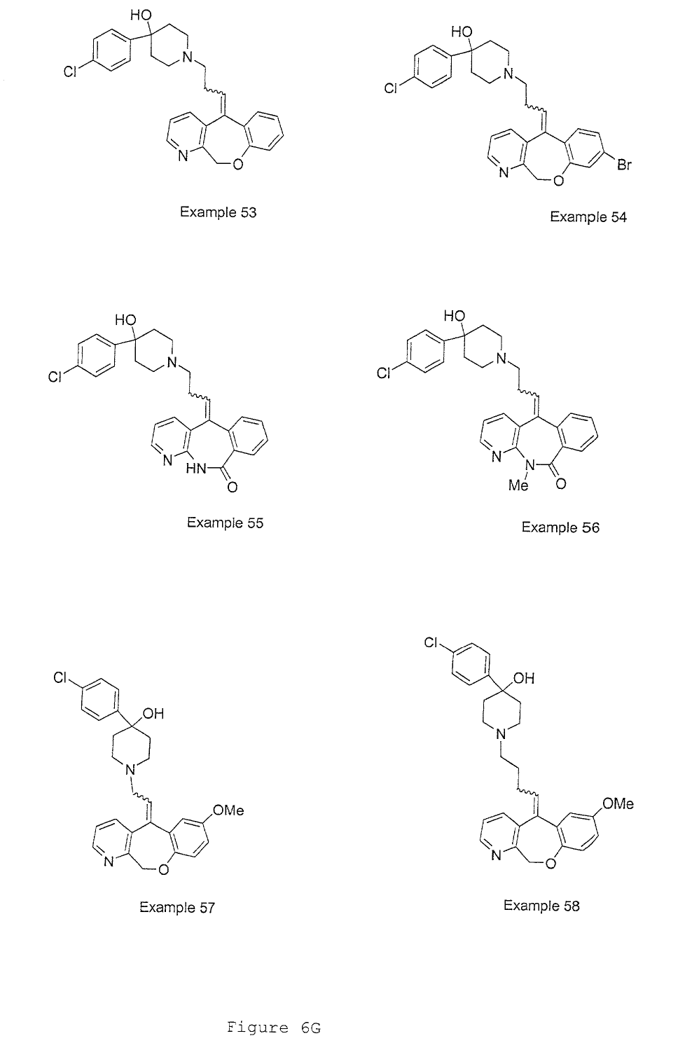

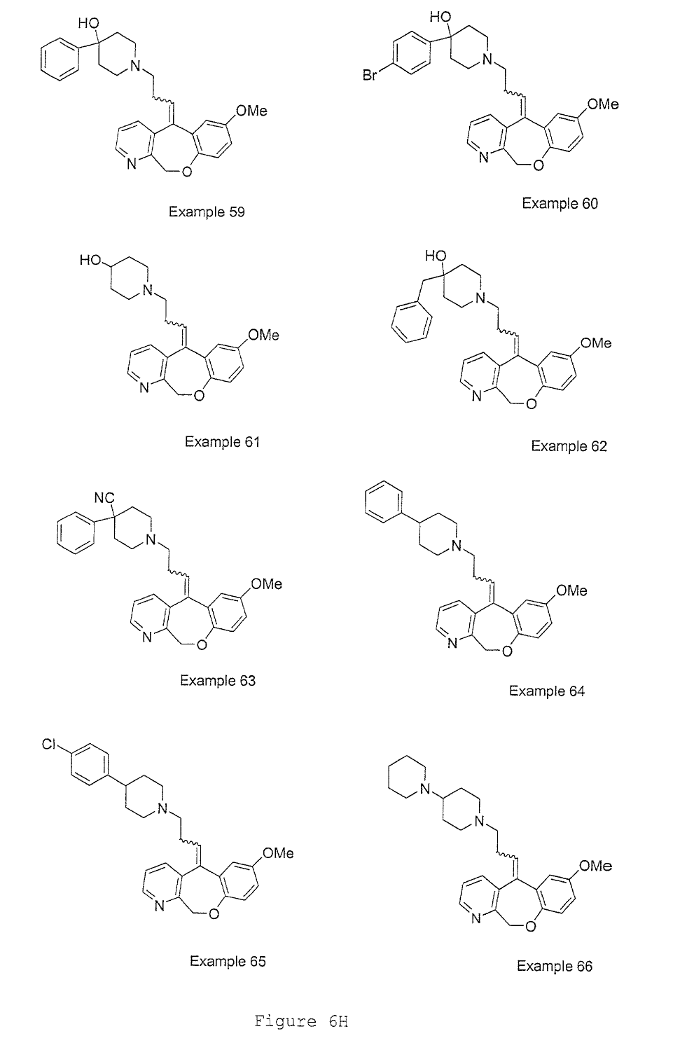

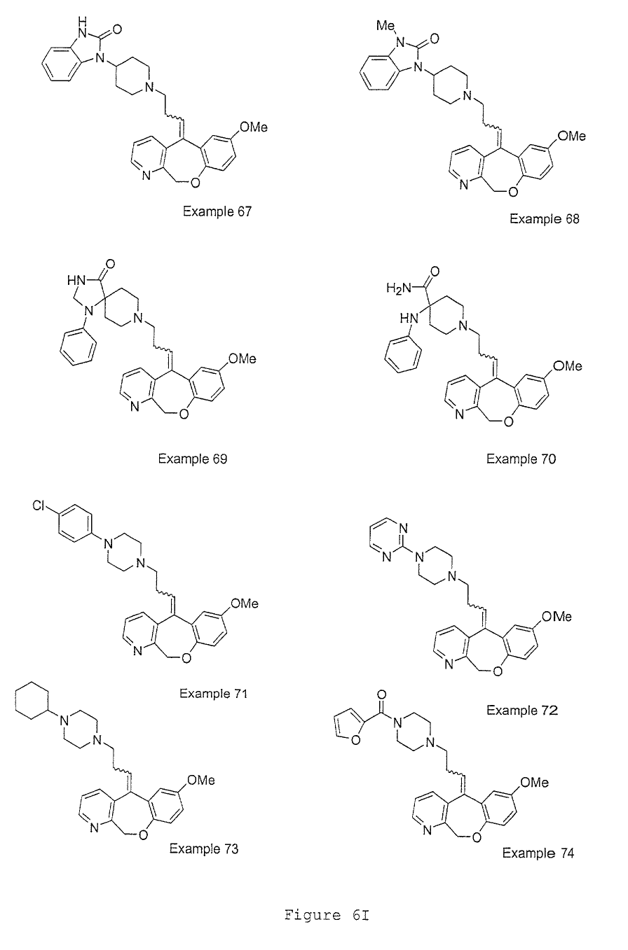

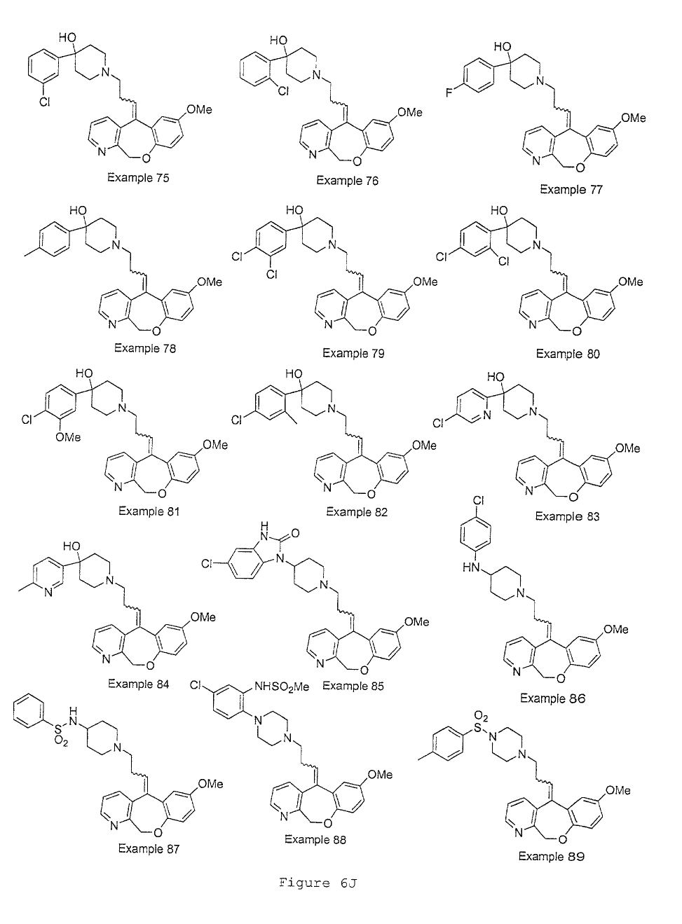

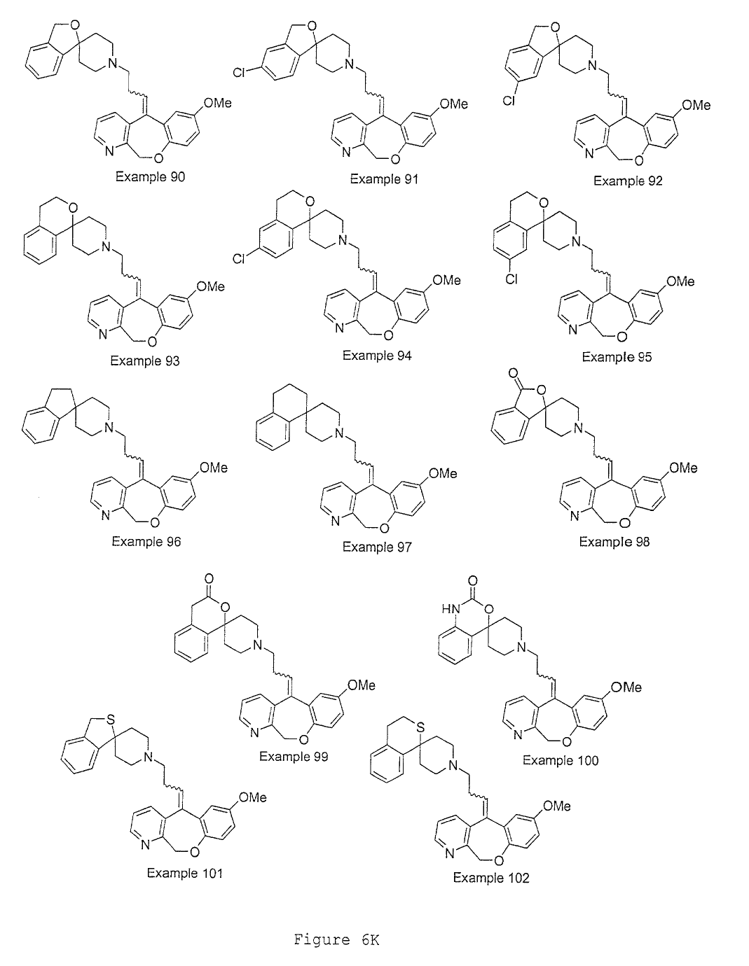

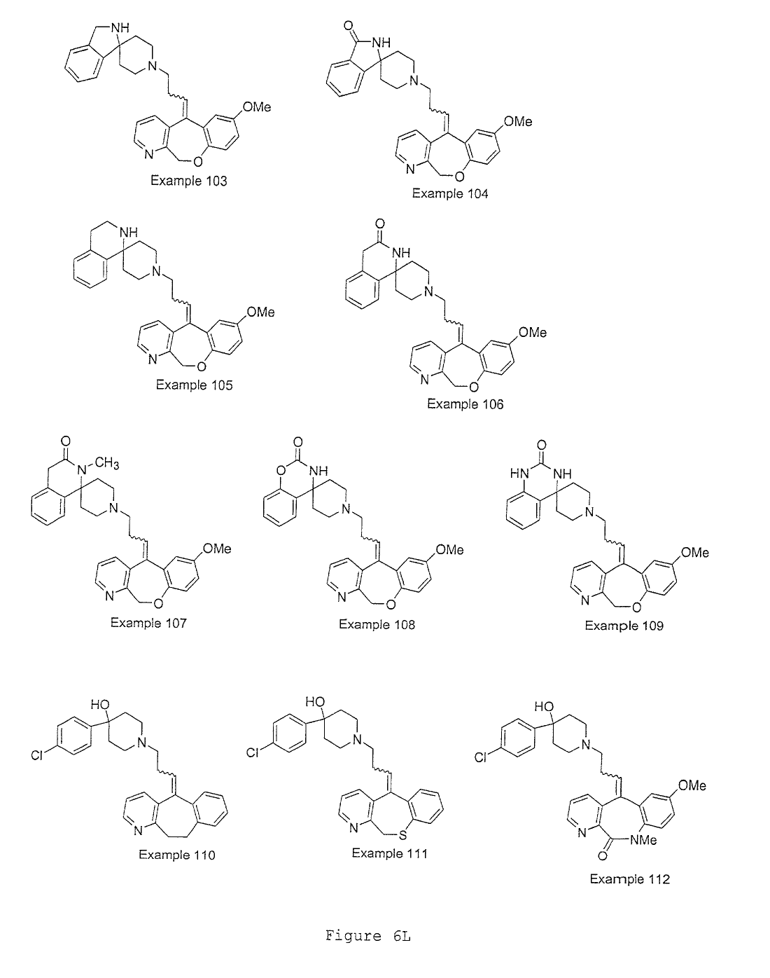

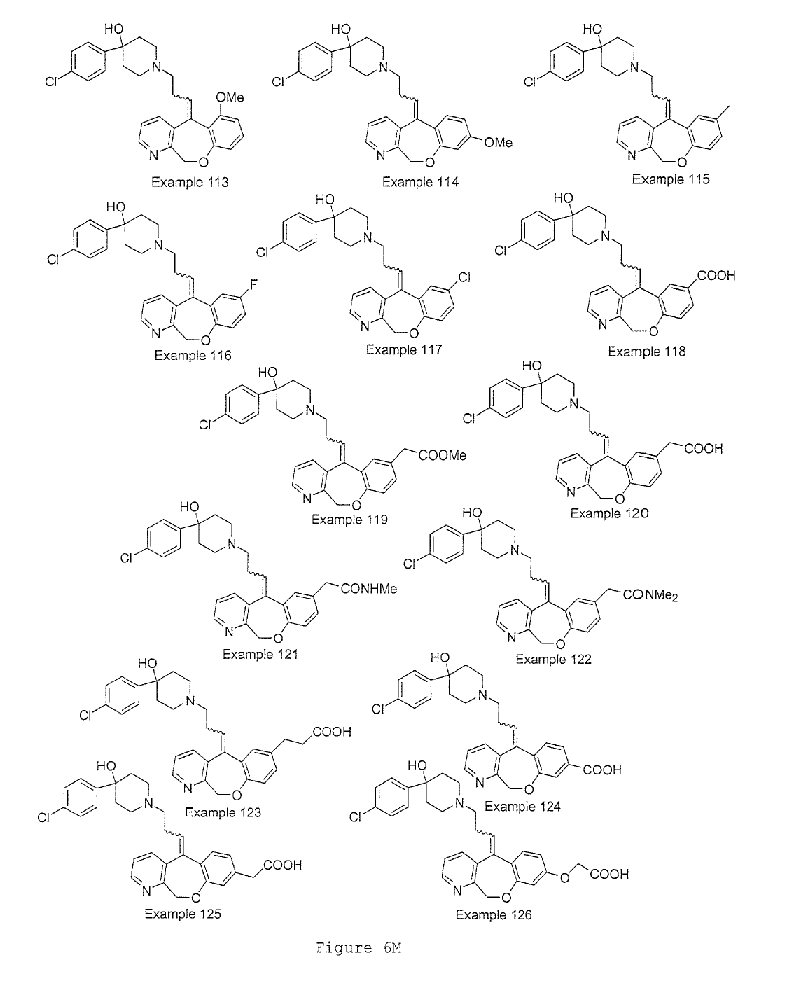

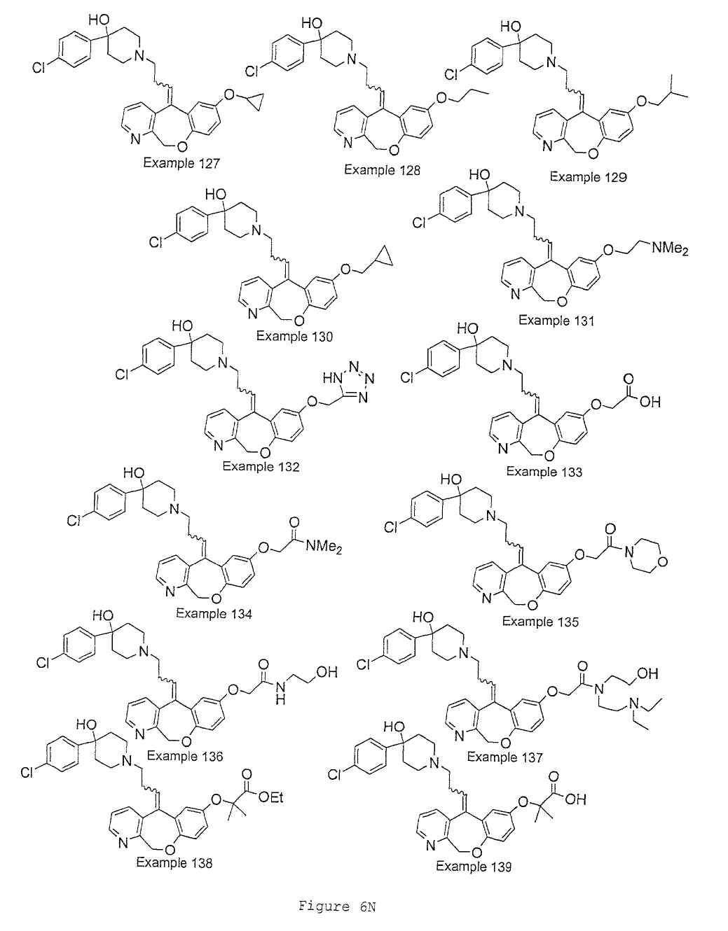

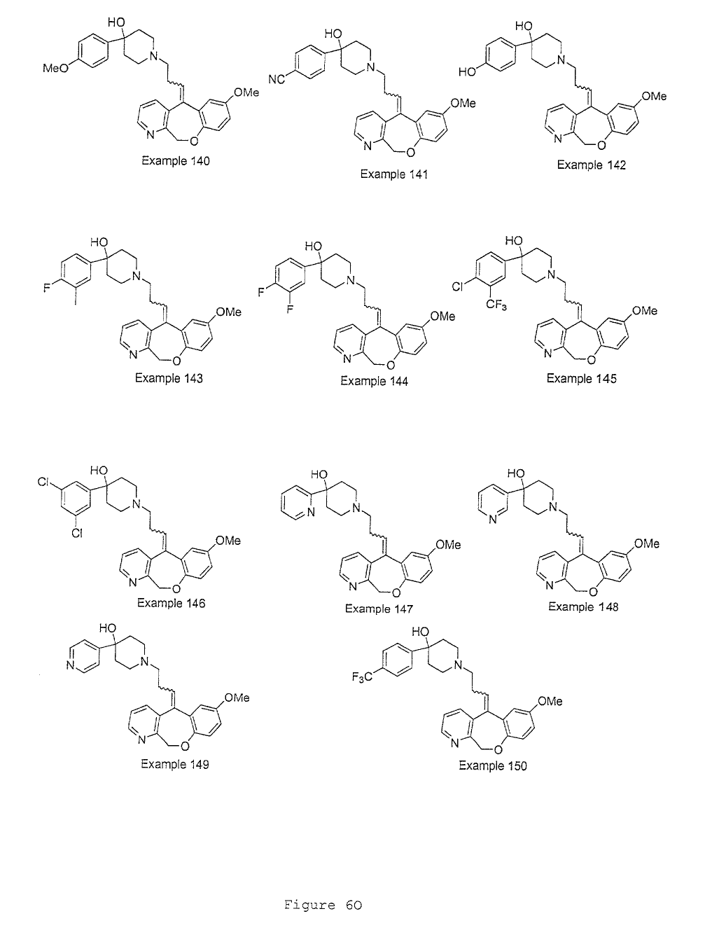

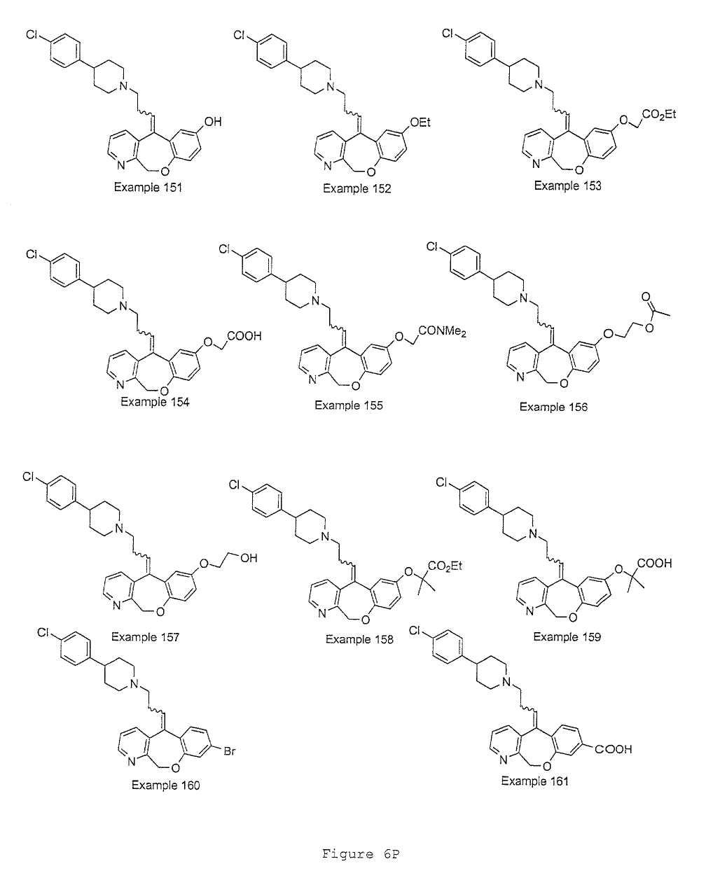

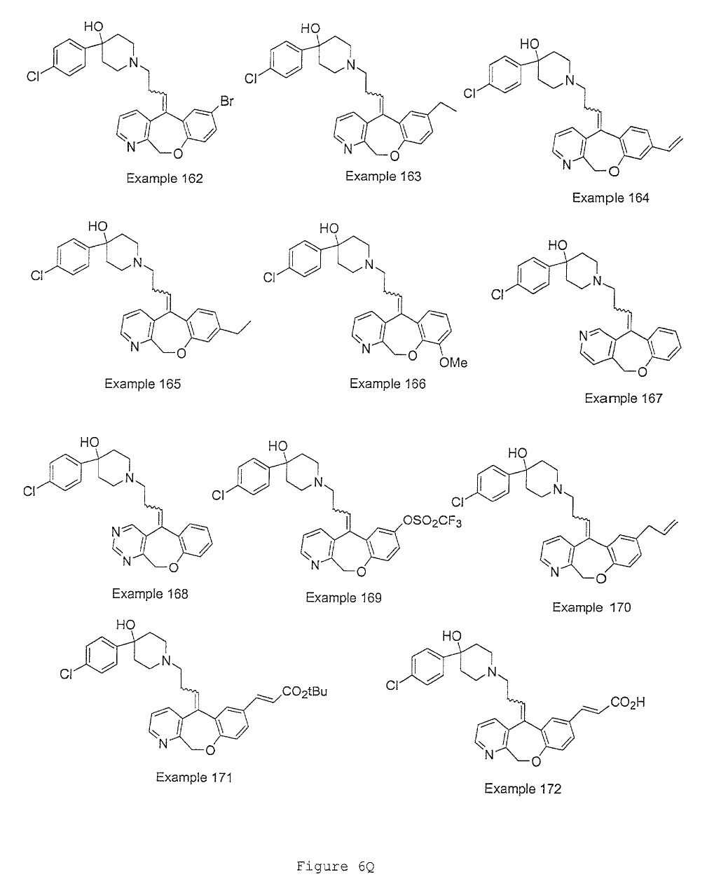

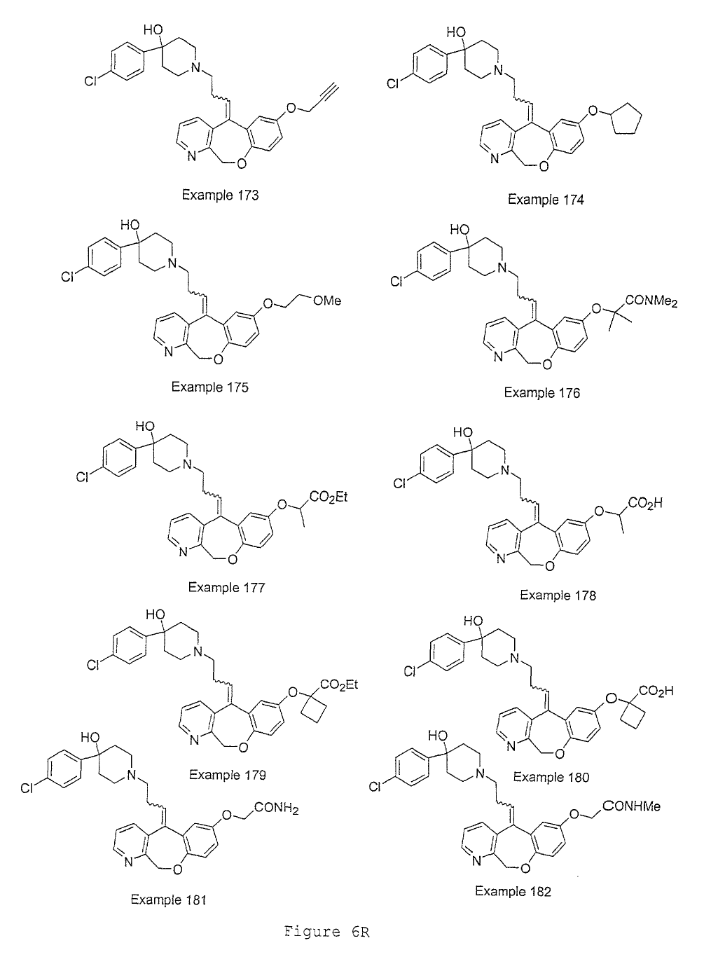

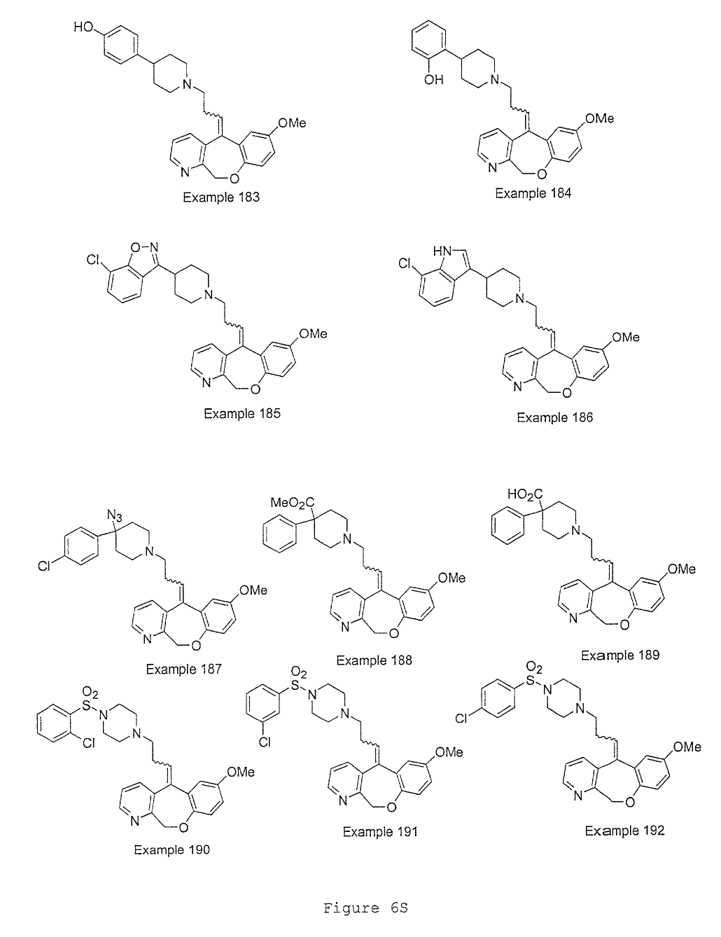

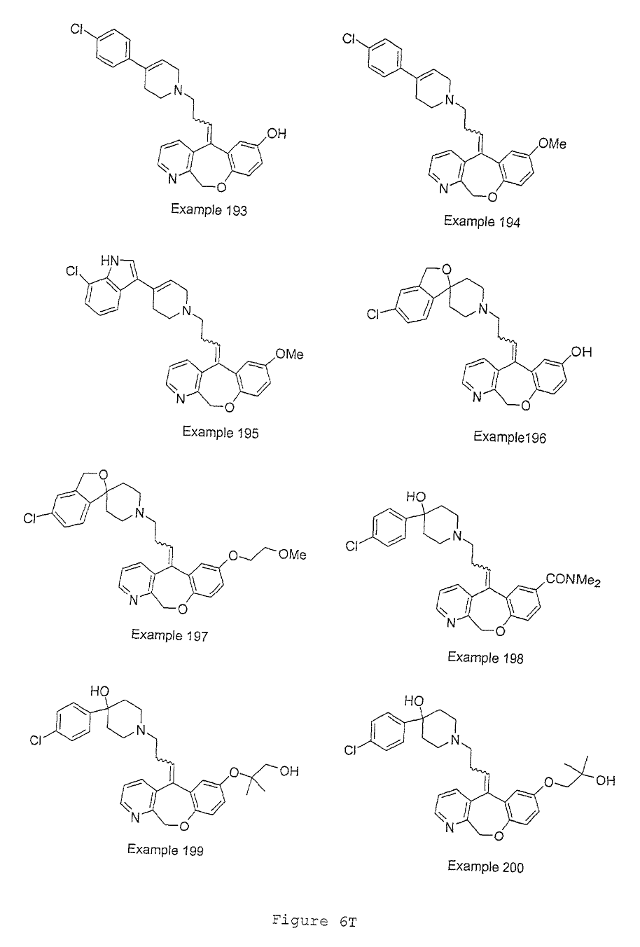

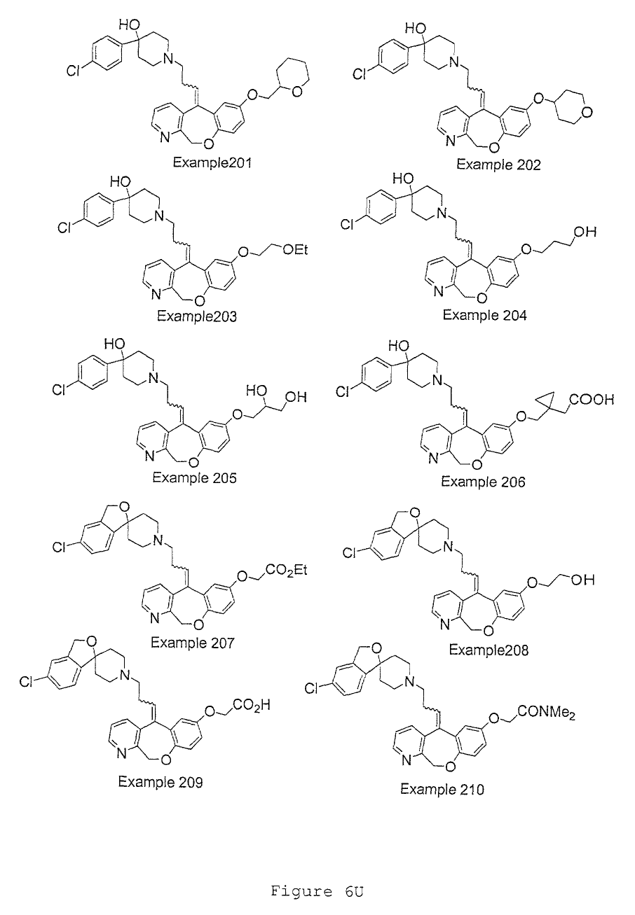

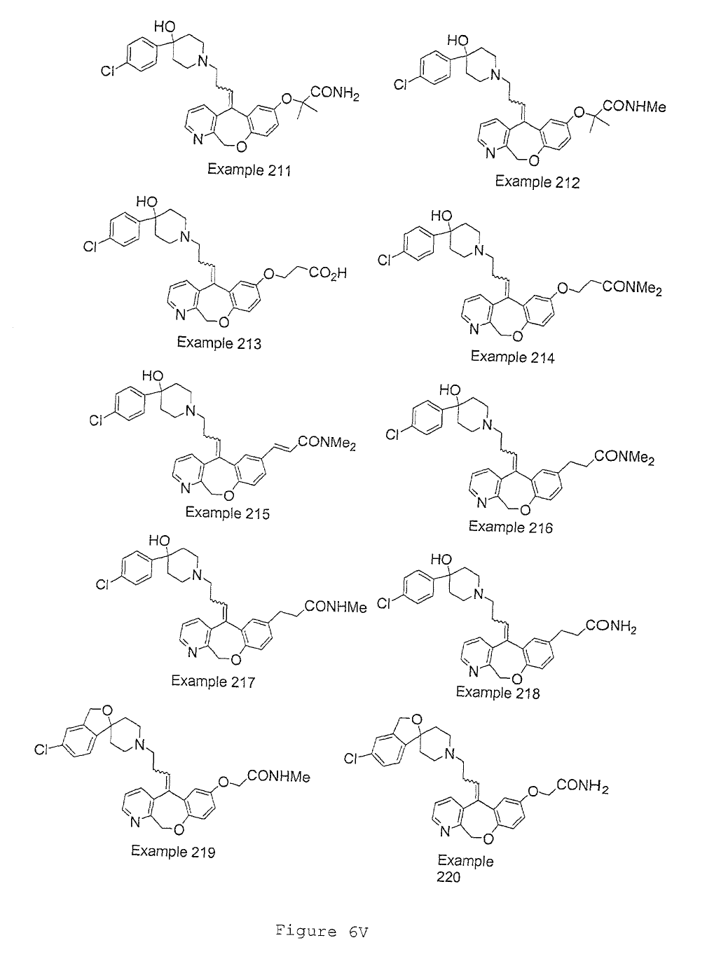

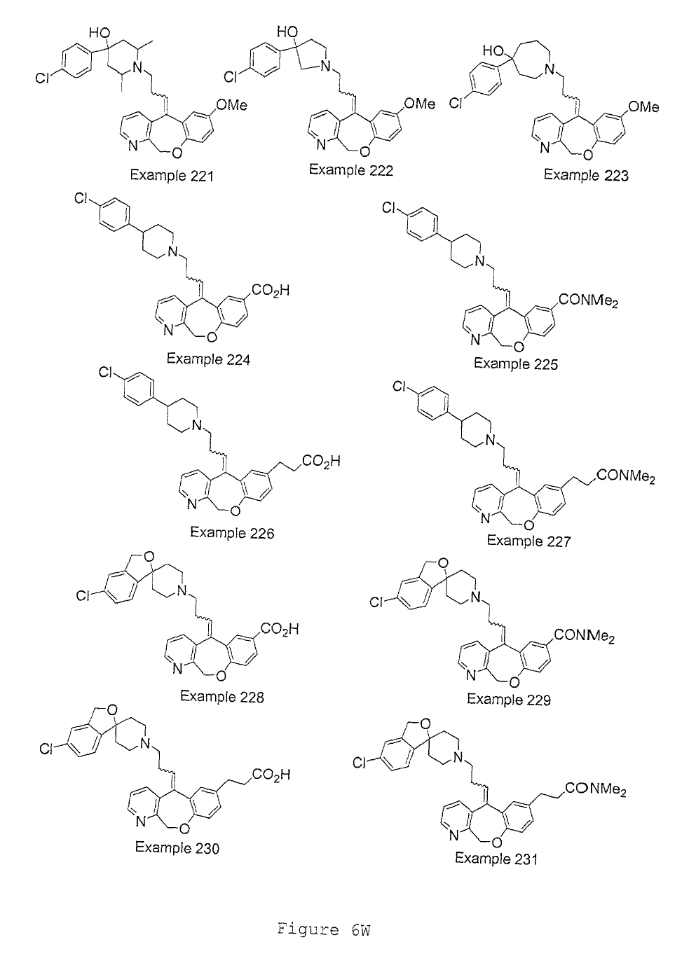

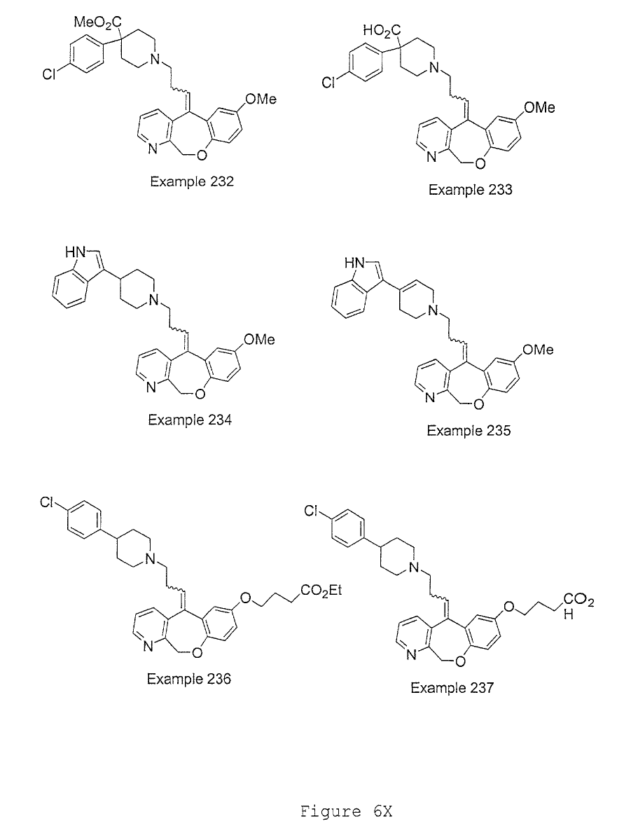

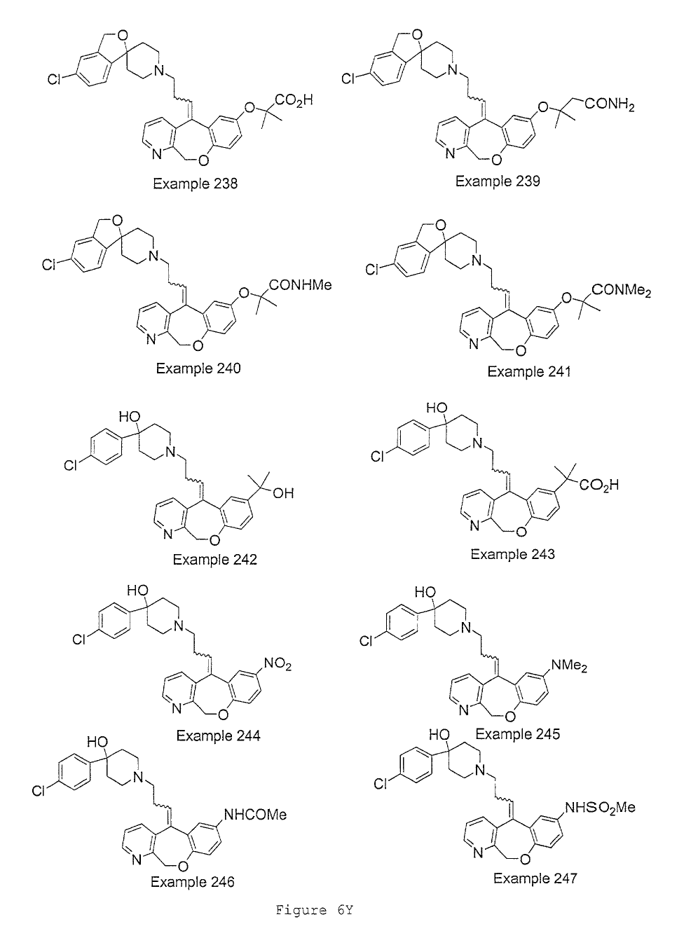

FIG. 5 is a schematic showing the preparation of the compounds represented by Structural Formula (I), wherein Z is represented by Structural Formulas (III) and wherein Ring A and/or Ring B in Z is substituted with —(O)ᵤ—(CH₂)ₜ—COOR²⁰, —(O)ᵤ—(CH₂)ₜ—OC(O)R²⁰, —(O)ᵤ—(CH₂)ₜ—C(O)—NR²¹R²² or —(O)ᵤ—(CH₂)ₜ—NHC(O)O—R²°. In FIG. 5, the hydrolysis reaction may be carried out in a mixture of aqueous alkali metal hydroxide solution and a solvent such as methanol, ethanol, tetrahydrofuran (THF) or dioxane at room temperature up to the reflux temperature for the solvent used for 5 minutes to 72 h. The acylation reaction can be carried out using dicyclohexylcarbodiimide (DCC) or (1-ethyl-3-(3-dimethylaminopropyl)carbodiimide (DEC) in a solvent such as tetrahydrofuran (THF), dimethylformamide (DMF) or methylene chloride in the presence of a base such as pyridine or triethylamine (when necessary) at temperatures of 0 to 100° C. for 5 minutes to 72 h.

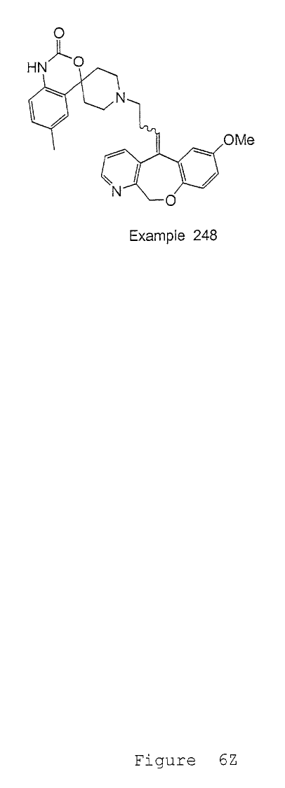

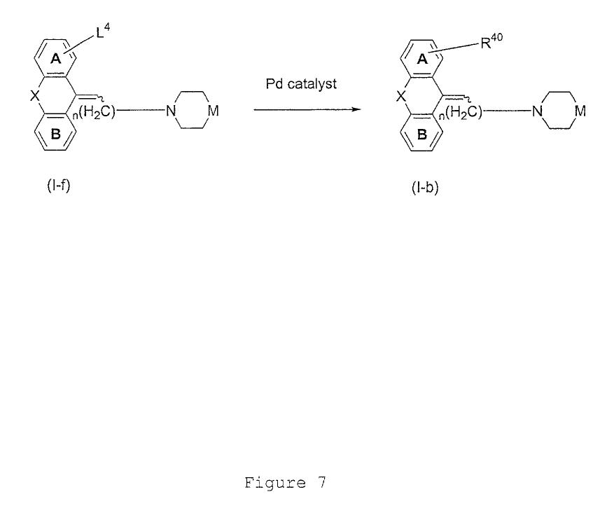

FIG. 7 shows the preparation of compounds represented by Structural Formula (I), wherein Z is represented by Structural Formulas (III) and wherein Ring A or Ring B in Z is substituted with R⁴⁰. L⁴ is a suitable leaving group such as halogen or trifluoromethylsulfonate. In FIG. 7, a palladium coupling reaction such as Stille coupling, Suzuki coupling, Heck reaction, or carboxylation using carbon monoxide may be carried out using a palladium catalyst such as tetrakis(triphenylphosphine)palladium, bis(triphenylphosphine)palladium chloride, and palladium acetate in a solvent such as tetrahydrofuran (THF), 1,4-dioxane, toluene, dimethylformamide (DMF), or dimethylsufoxide (DMSO) in the presence of additive (when necessary) such as triphenylphosphine, 1,1′-bis(diphenylphosphino)ferrocene, triethylamine, sodium bicarbonate, tetraethylammonium chloride, or lithium chloride at room temperature up to the reflux temperature for the solvent used for 5 minutes to 72 h.

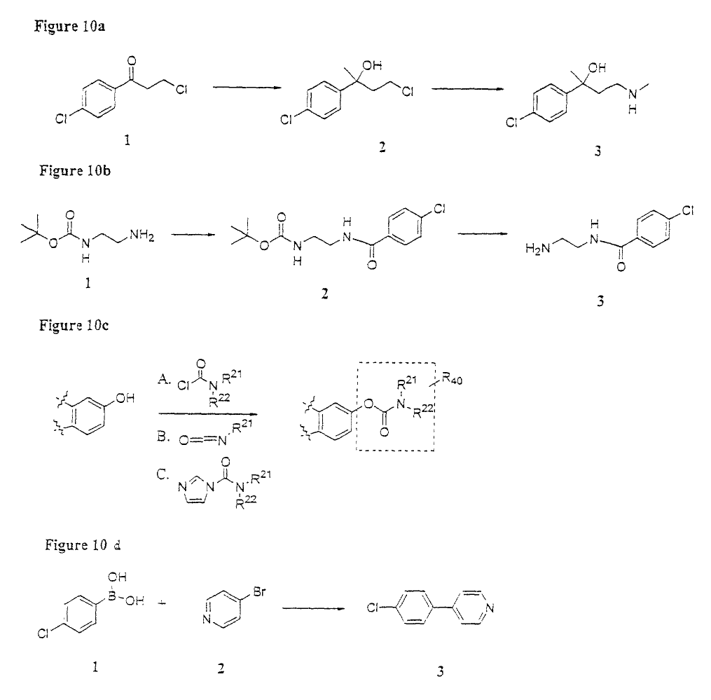

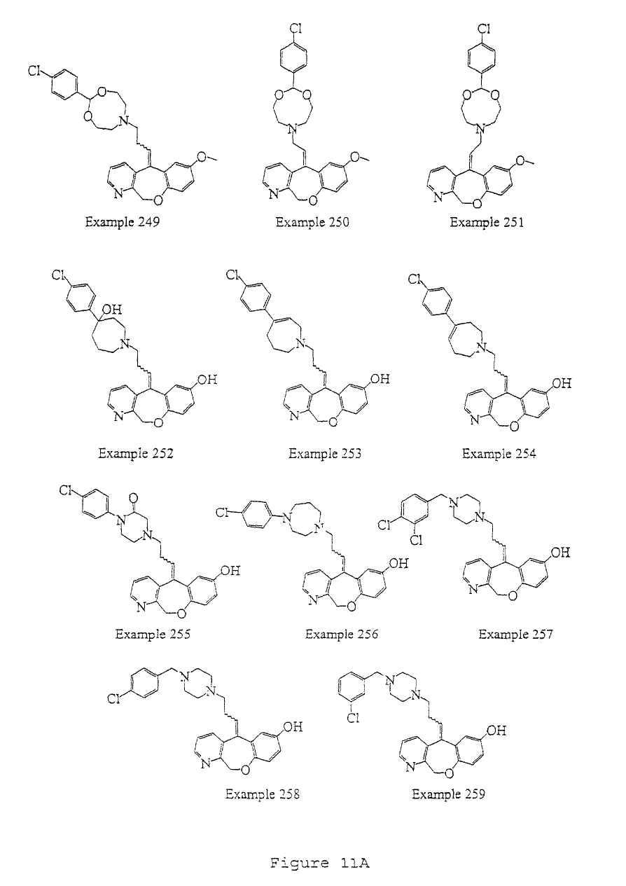

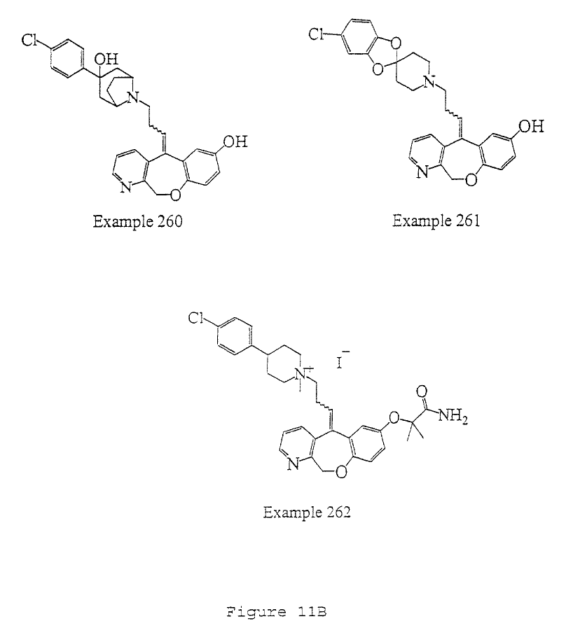

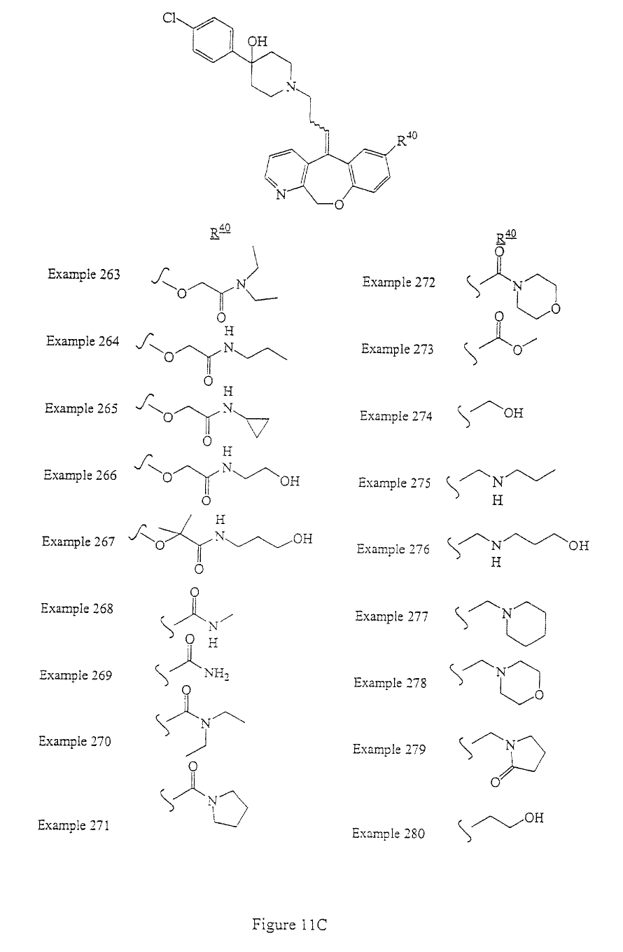

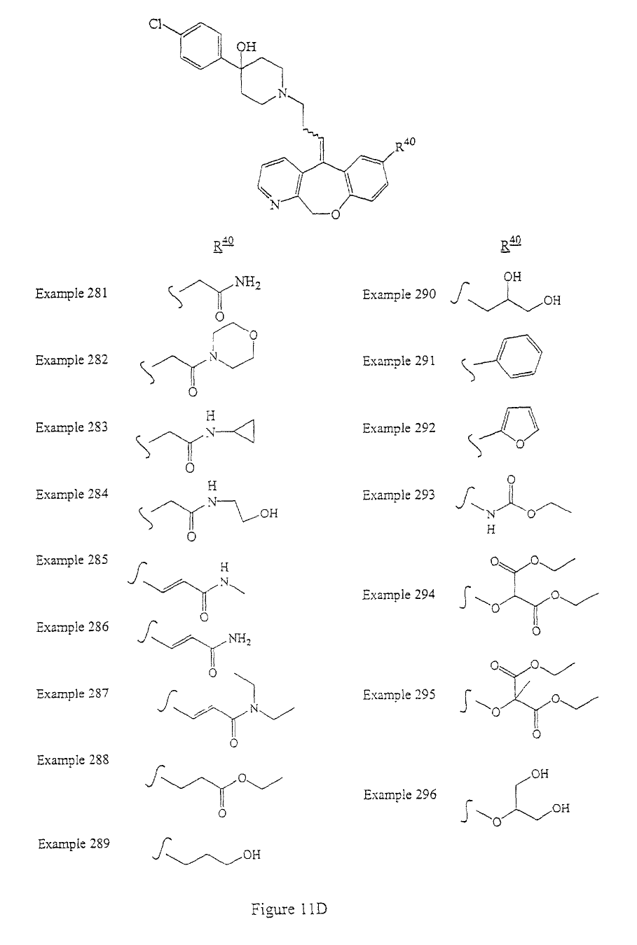

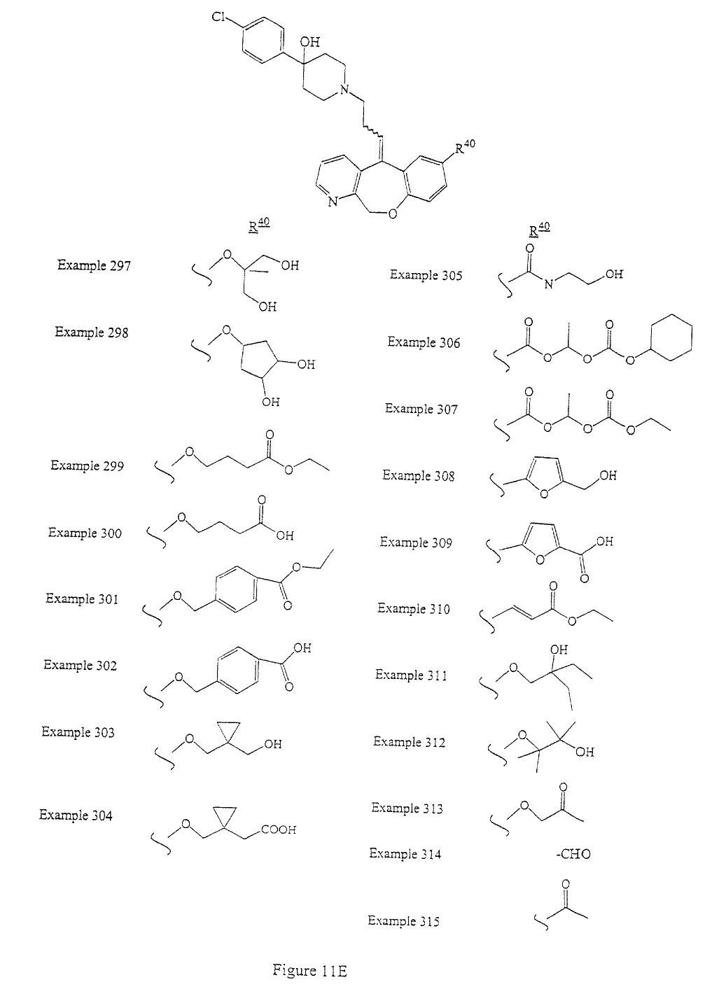

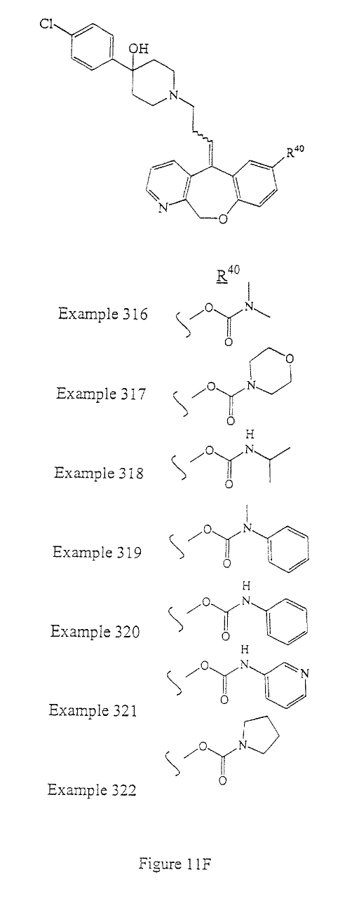

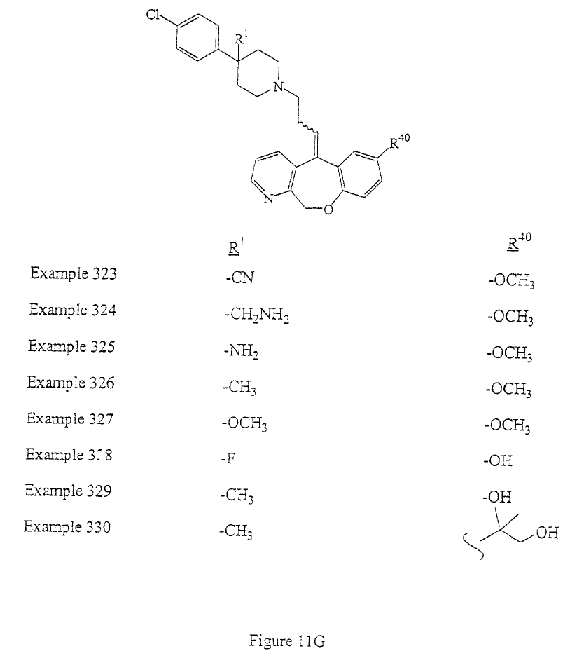

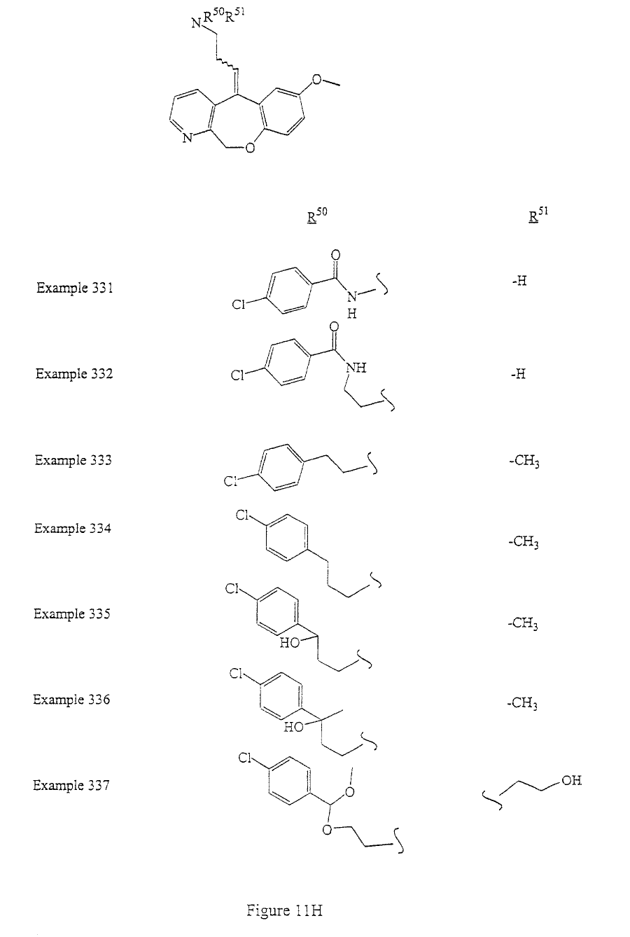

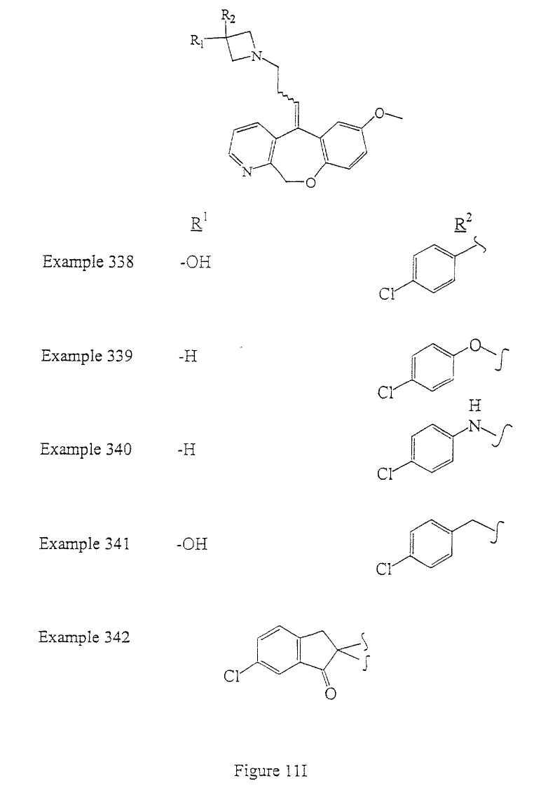

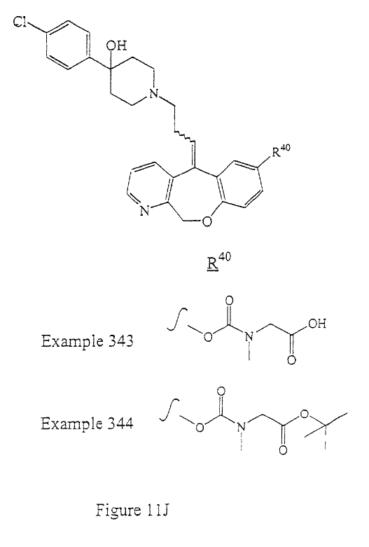

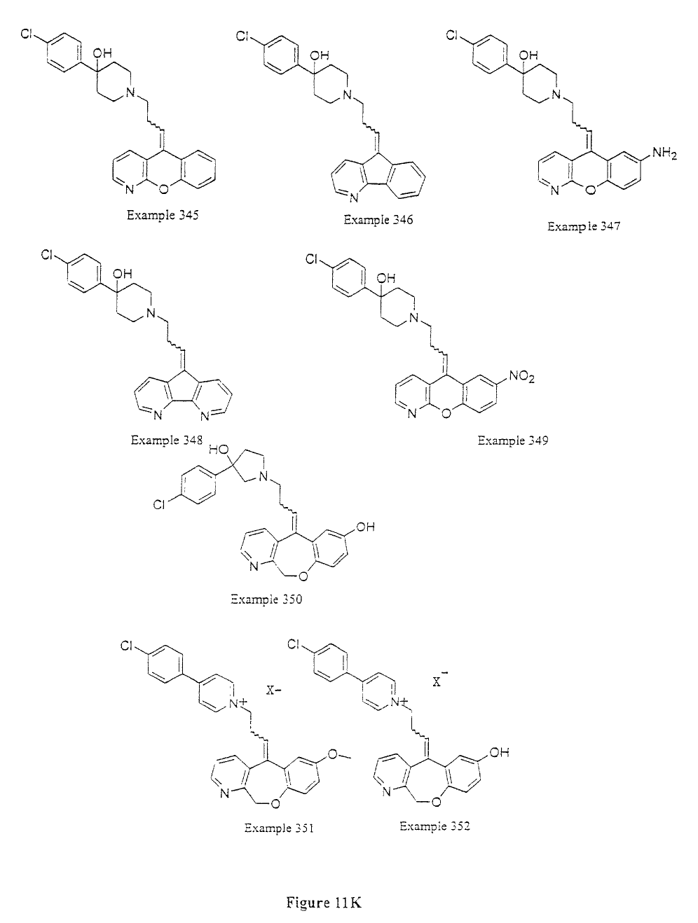

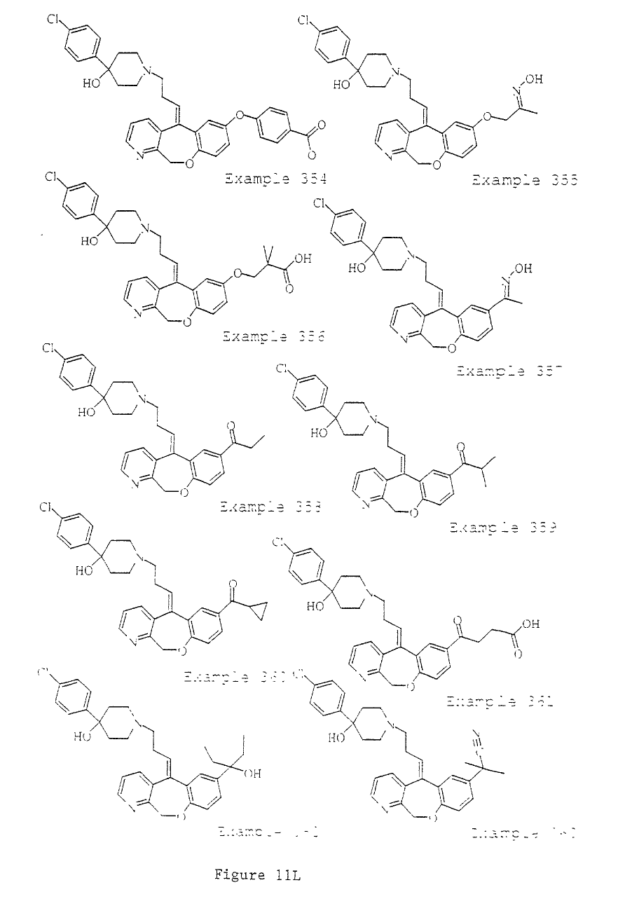

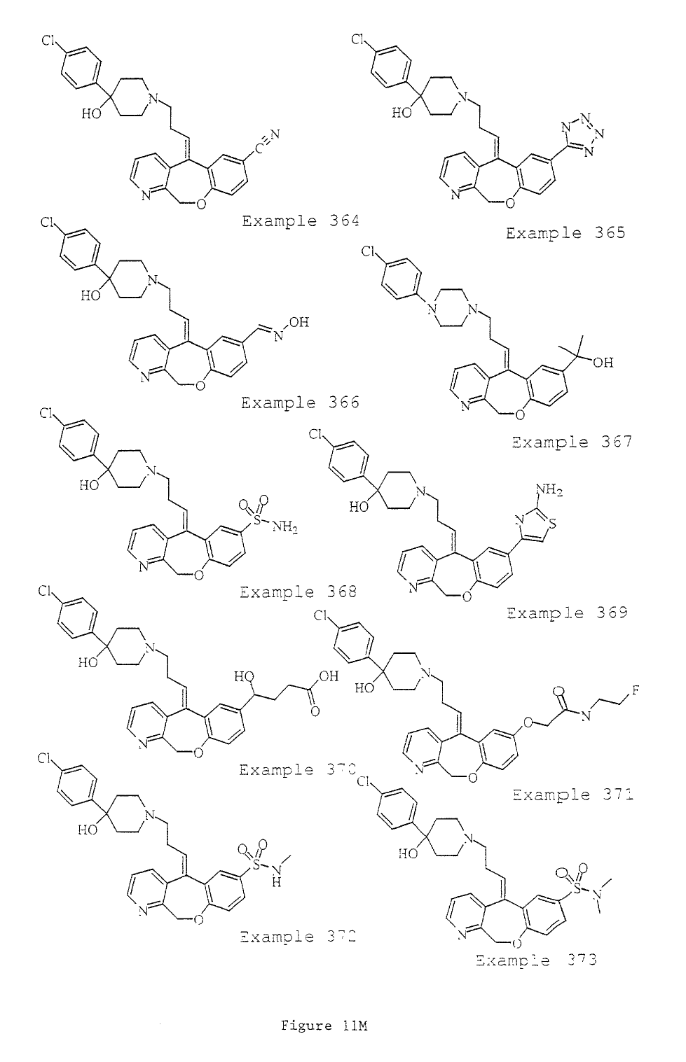

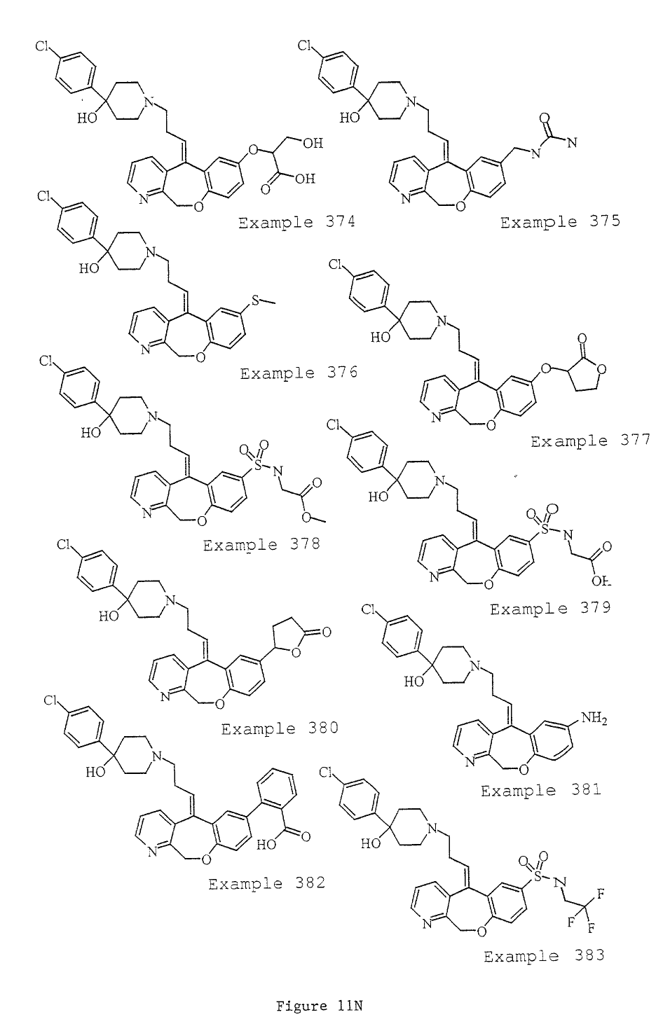

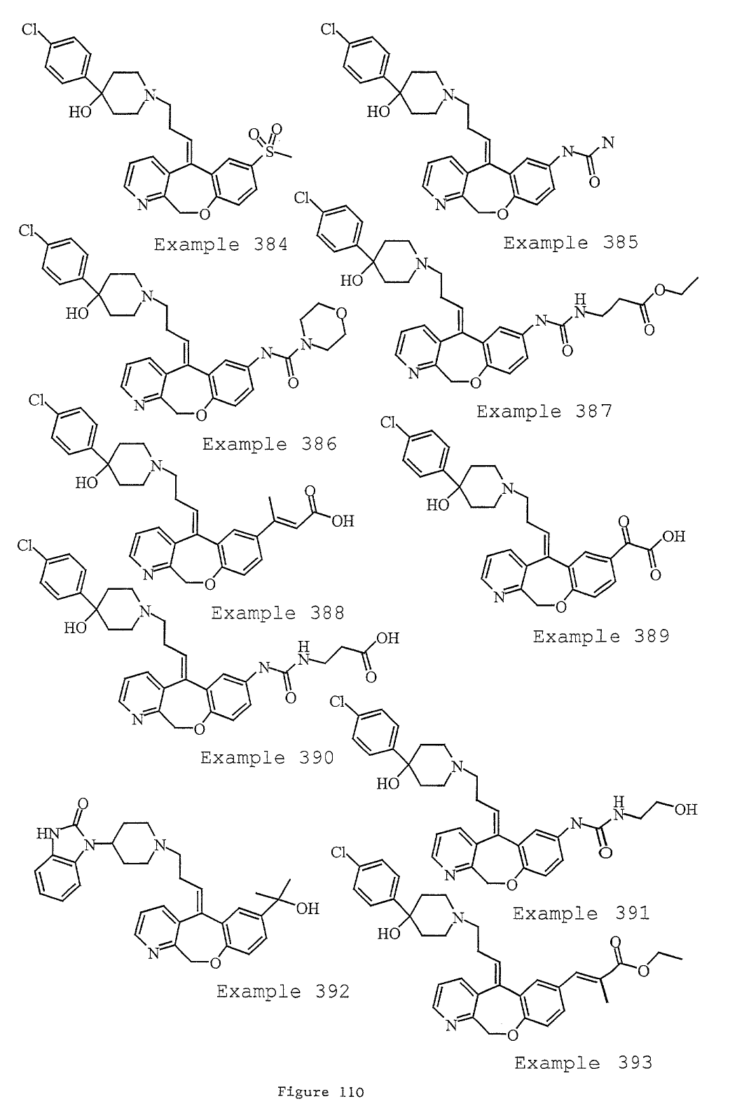

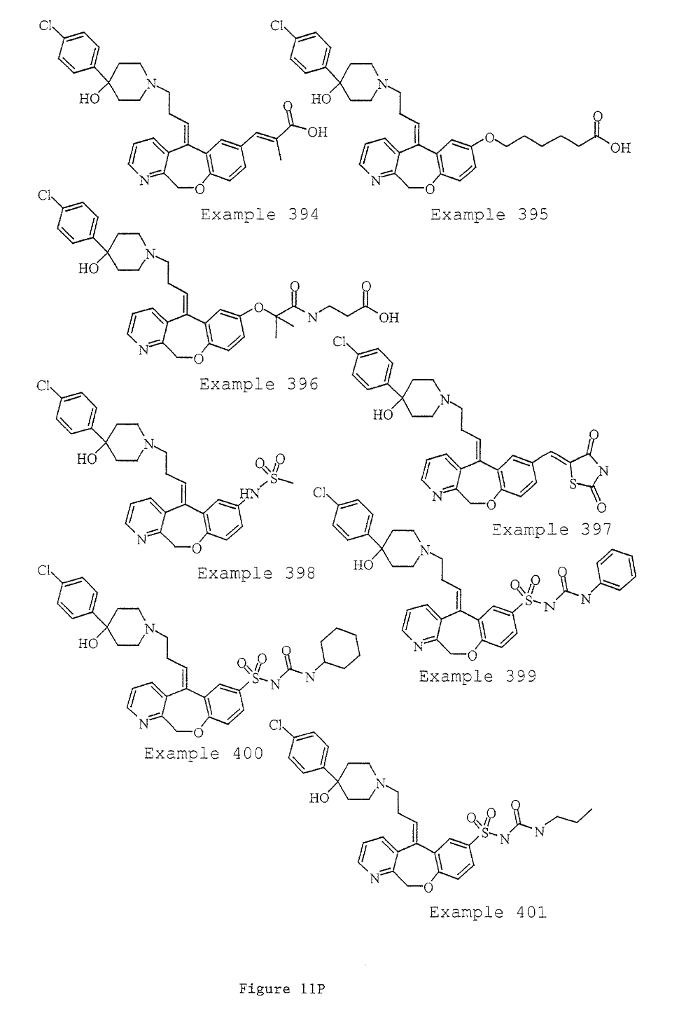

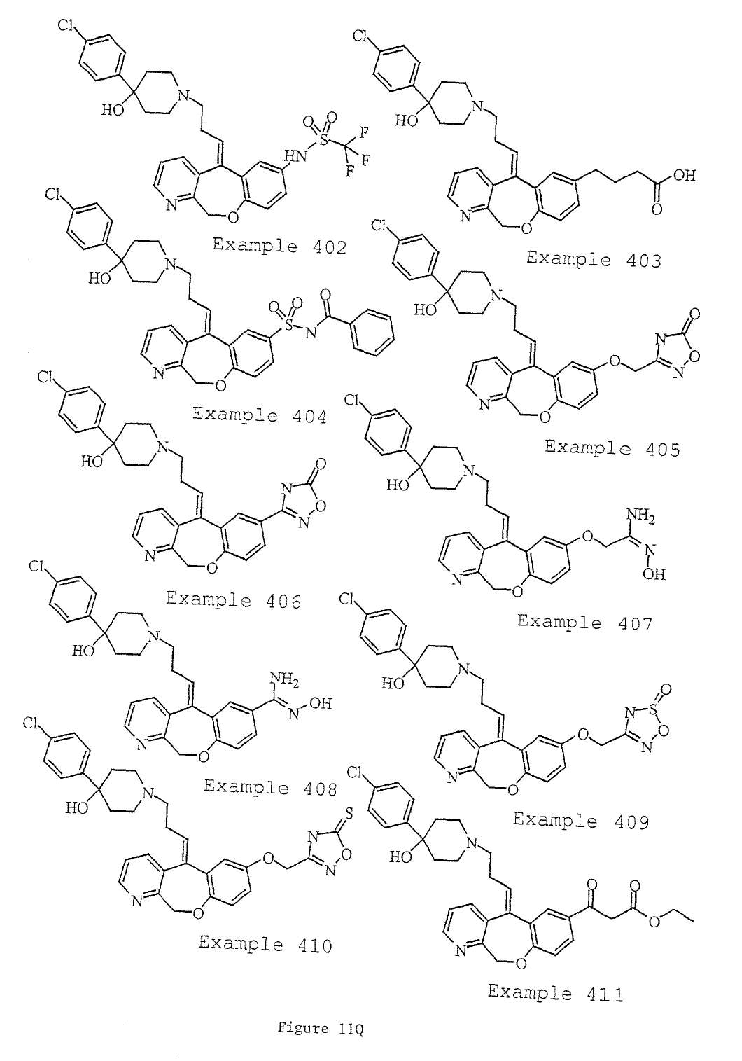

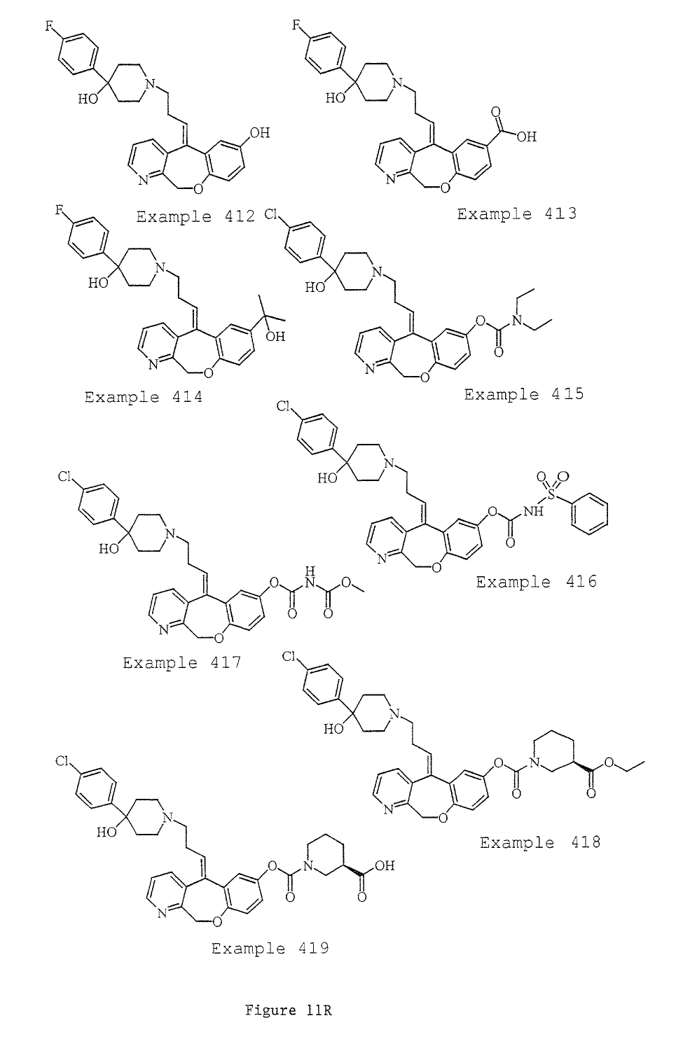

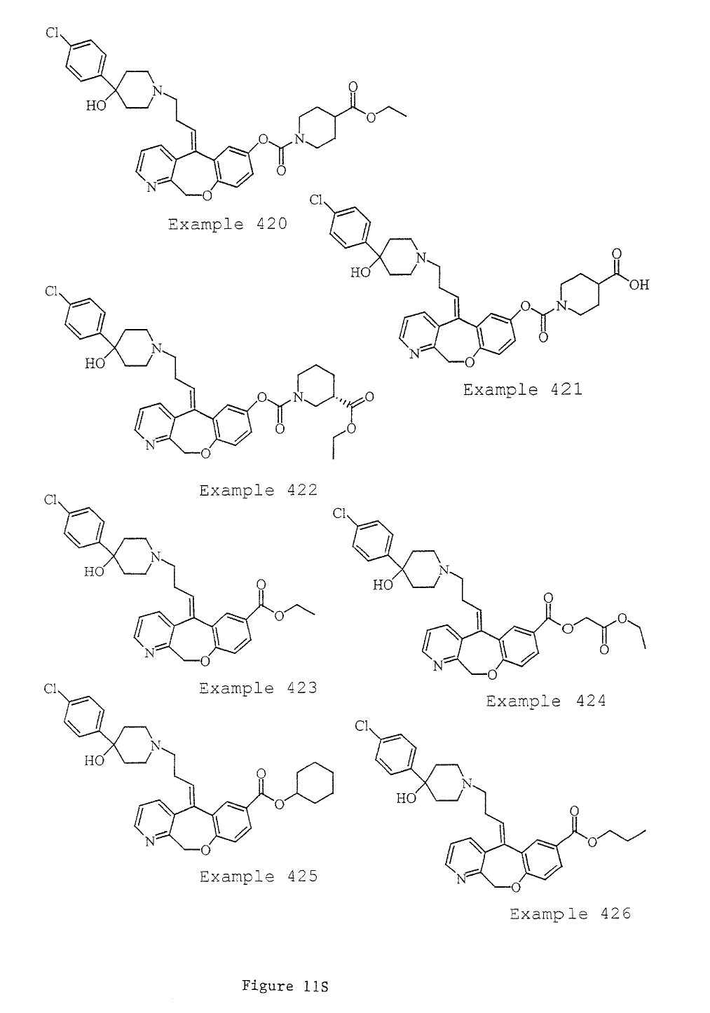

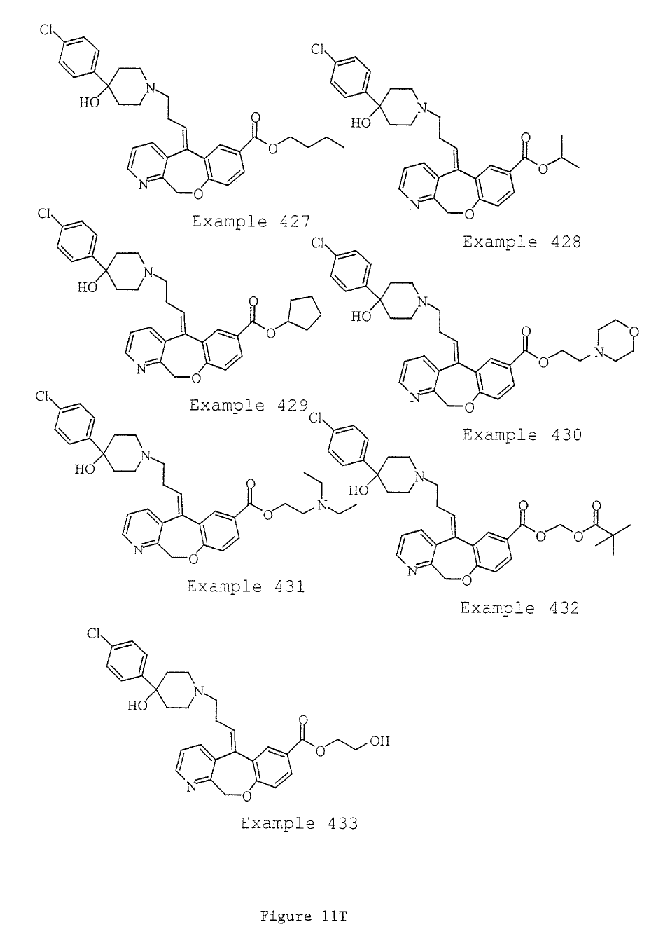

FIG. 10C shows three procedures for the preparation of compounds represented by Structural Formulas (I),(VII), (VIII) and (IX), wherein Z is represented by Structural Formula (III) and wherein Ring A or Ring B in Z is substituted with R₄₀. In FIG. 10C, R⁴⁰ is represented by —(O)ᵤ—(CH₂)ₜ—C(O)—NR²¹R²², u is one, t is zero. In FIG. 10C a compound containing a phenol can be reacted with a carbonate equivalent, such as a carbamoyl chloride (method A), an isocyanate (method B) or an acylimidazole (method C), in the presence of a base such as sodium hydroxide, potassium carbonate or sodium carbonate in a solvent such as dimethylformamide or tetrahydrofuran, at a temperature from 0° C. to reflux temperature for a period of about 5 minutes to about 72 hours.

Compounds represented by Structural Formula (I), wherein Z is represented by Structural Formulas (III) or (IV), X is —CO—NR_{c}— and R_{c} is —(CH₂)ₛ—COOR³⁰, —(CH₂)ₛ—C(O)—NR³¹R³² or —(CH₂)ₛ—NHC(O)—O—R³⁰, can be prepared by suitable modification of the scheme shown in FIG. 1-5 and 7. One modification utilizes the starting material shown in FIG. 1, wherein X is —CO—NH—. The amide is then alkylated with L³—(CH₂)ₛ—COOR³⁰, wherein L³ is a suitable leaving group, using the alkylation procedures described above. The remainder of the synthesis is as described in FIGS. 1-5 and 7.

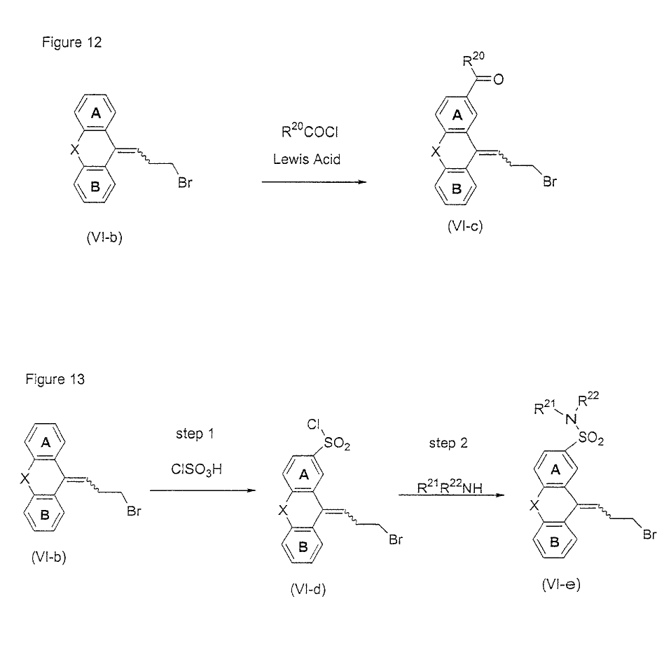

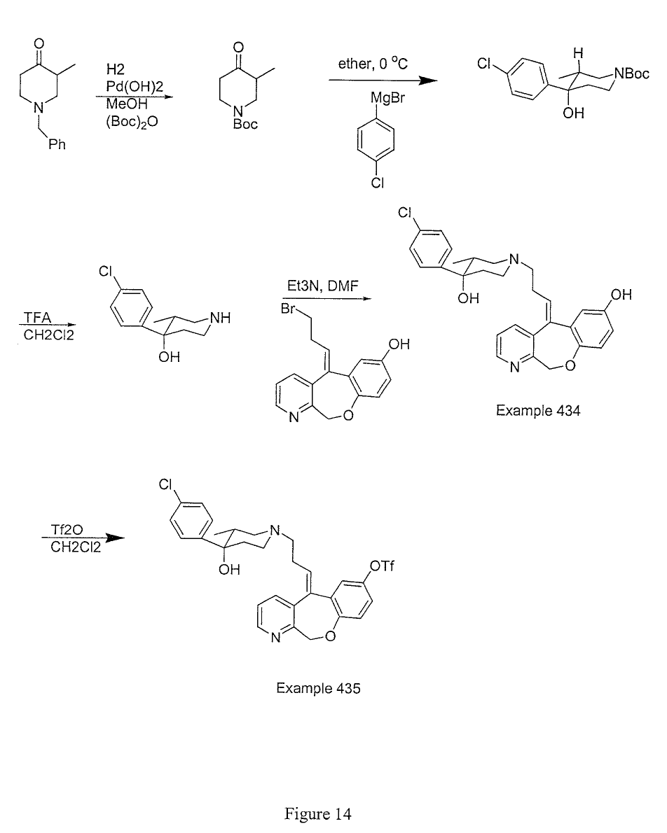

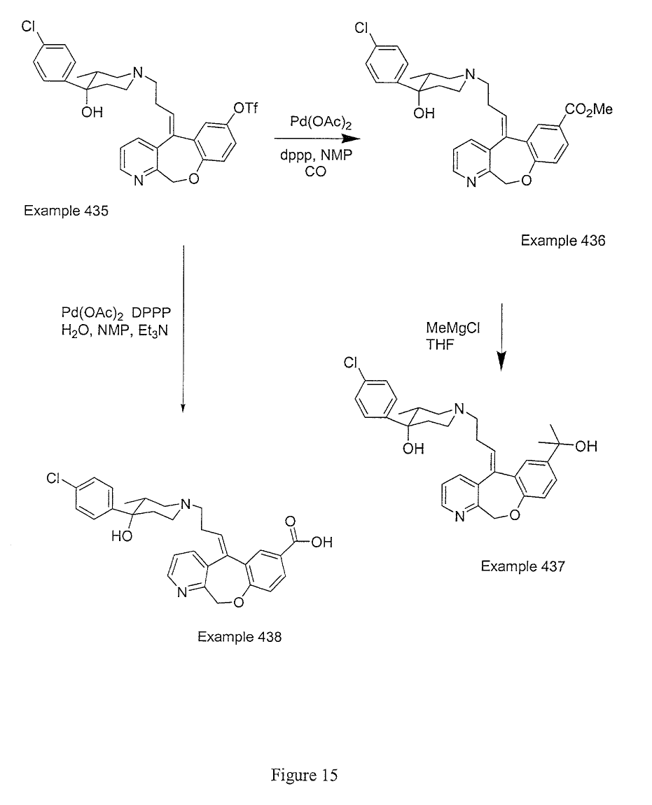

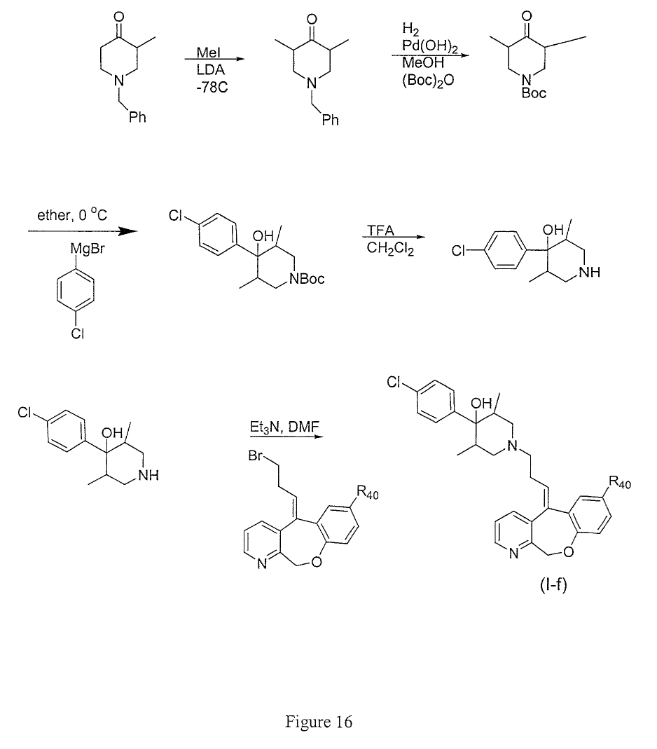

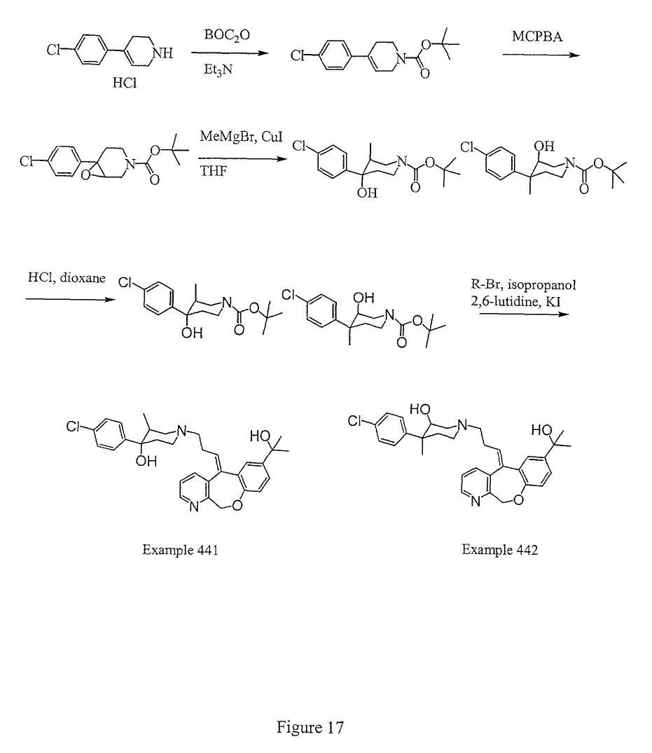

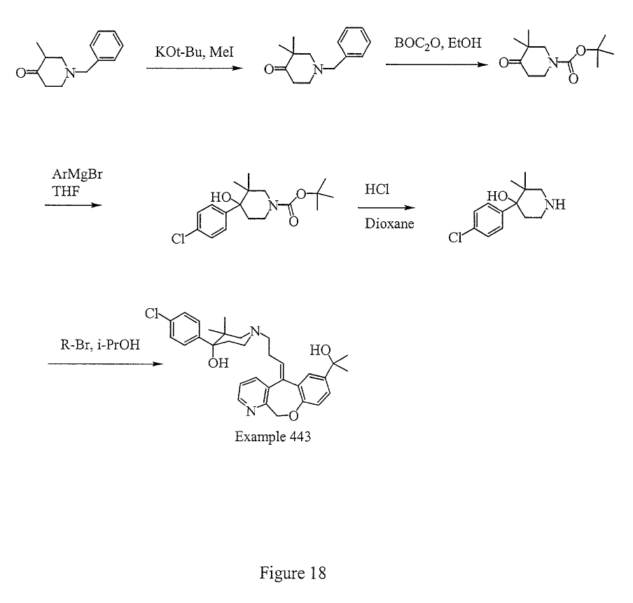

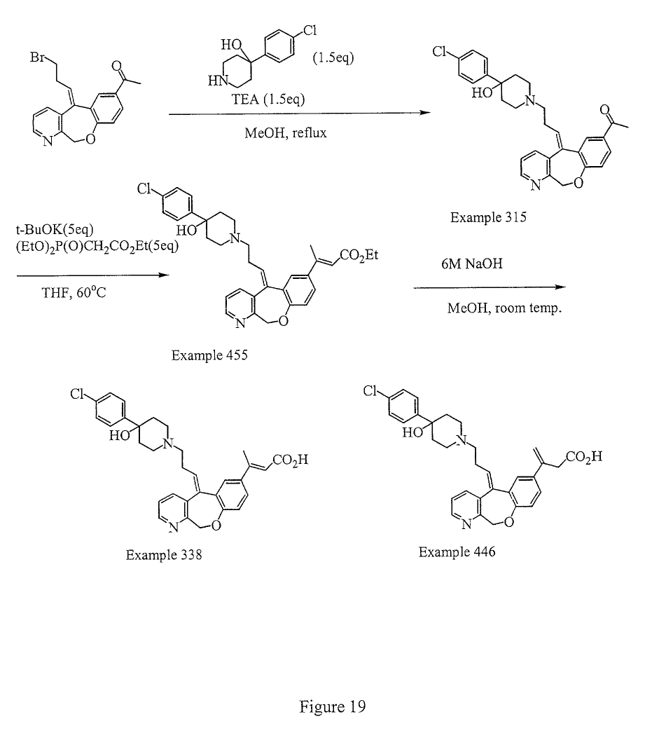

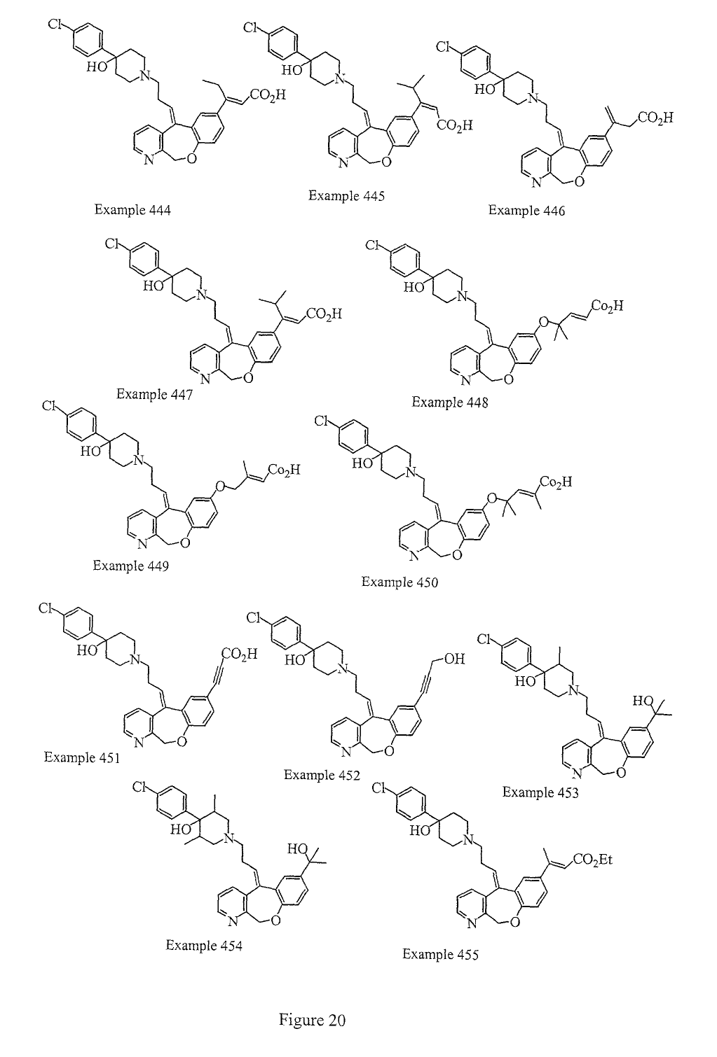

FIG. 12 shows the preparation of compounds of formula (VI-c). The Friedel-Crafts acylation can be carried out using an acid chloride in the presence of a Lewis acid, such as aluminum trichloride or titanium tetrachloride, in a solvent such as dichloromethane, dichloroethane, nitrobenzene or carbon disulfide. The acylation reaction can be run at a temperature of about room temperature up to the reflux temperature of the chosen solvent, and for a period of about 5 minutes to about 72 hours.

FIG. 13 shows the preparation of compounds of formula (VI-e). In Step 1 of FIG. 13, a chlorosulfonylation can be carried out using chlorosulfonic acid in a solvent, such as dichloromethane, or in the absence of a solvent at a temperature of about 0° C. to about 60° C. for a period of about 5 minutes to about 72 hours. In Step 2 of FIG. 12, a coupling reaction can be carried out using an amine in the presence of a base, such as triethylamine, in a solvent such as dichloromethane, acetone, ethanol, THF or DMF. The reaction can be carried out at a temperature of about room temperature up to the reflux temperature of the selected solvent, and for a period of about 5 minutes to about 72 hours.

Although FIGS. 1-5, 7, 12 and 13 show the preparation of compounds in which Rings A and B are phenyl rings, analogous compounds with heteroaryl groups for Rings A and B can be prepared by using starting materials with heteroaryl groups in the corresponding positions. These starting materials can be prepared according to methods disclosed in JP 61/152673, U.S. Pat. No. 5,089,496, WO 89/10369, WO 92/20681 and WO 93/02081.

The invention is illustrated by the following examples which are not intended to be limiting in any way.

### Exemplification

### EXAMPLE 1

### 4-(4-Chlorophenyl)-1-[3-(10,11-dihydro-5H-dibenzo[a,d]cycloheptene-5-ylidene)propyl]piperidin-4-ol

To a solution of 5-(3-bromopropylidene)-10,11-dihydro-5H-dibenzo[a,d]cycloheptene (described in JP 48-030064)(200 mg) in DMF (10 ml) were added 4-(4-chlorophenyl)-4-hydroxypiperidine (230 mg), potassium carbonate (360 mg), and potassium iodide (50 mg). The mixture was stirred at 70° C. for 24 hours. Water and ethyl acetate were added to the reaction mixture, the organic layer was separated and washed with saturated aqueous sodium chloride, and dried with magnesium sulfate. The solvent was distilled off under reduced pressure. The residue was purified by silica gel chromatography eluting with ethyl acetate-hexane (1:1) to give the titled compound (250 mg). ¹H-NMR (CDCl₃) d: 1.65-2.11 (5H, m), 2.32-3.10(8H, m), 3.22-3.67(4H, m), 5.87(1H, t), 7.03-7.44(12H, m). MS m/z: 444(M+1).

### EXAMPLE 2

### 4-(4-Chlorophenyl)-1-[3-(6,11-dihydrodibenz[b,e]oxepin-11-ylidene)propyl]piperidin-4-ol

The titled compound was prepared by following the procedure of Example 1, but replacing 5-(3-bromopropylidene)-10,11-dihydro-5H-dibenzo[a,d]cycloheptene with 11-(3-bromopropylidene)-6,11-dihydrodibenz[b,e]oxepine. ¹H-NMR (CDCl₃) d: 1.61-2.16(5H, m), 2.37-2.80(8H, m), 5.22(2H, brs), 5.70(0.6×1H, t), 6.03(0.4×1H, t), 6.73-6.90(2H, m), 7.09-7.45(10H, m). MS m/z: 446(M+1)

### EXAMPLE 3

### Membrane Preparations for Chemokine Binding and Binding Assays

Membranes were prepared from THP-1 cells (ATCC #TIB202). Cells were harvested by centrifugation, washed twice with PBS (phosphate-buffered saline), and the cell pellets were frozen at -70 to -85° C. The frozen pellet was thawed in ice-cold lysis buffer consisting of 5 mM HEPES (N-2-hydroxyethylpiperazine-N′-2-ethane-sulfonic acid) pH 7.5, 2 mM EDTA (ethylenediaminetetraacetic acid), 5 μg/ml each aprotinin, leupeptin, and chymostatin (protease inhibitors), and 100 μg/ml PMSF (phenyl methane sulfonyl fluoride—also a protease inhibitor), at a concentration of 1 to 5×10⁷ cells/ml. This procedure results in cell lysis. The suspension was mixed well to resuspend all of the frozen cell pellet. Nuclei and cell debris were removed by centrifugation of 400× g for 10 minutes at 4° C. The supernatant was transferred to a fresh tube and the membrane fragments were collected by centrifugation at 25,000× g for 30 minutes at 4° C. The supernatant was aspirated and the pellet was resuspended in freezing buffer consisting of 10 mM HEPES pH 7.5, 300 mM sucrose, 1 μg/ml each aprotinin, leupeptin, and chymostatin, and 10 μg/ml PMSF (approximately 0.1 ml per each 10⁸ cells). All clumps were resolved using a minihomogenizer, and the total protein concentration was determined using a protein assay kit (Bio-Rad, Hercules, Calif., cat #500-0002). The membrane solution was then aliquoted and frozen at -70 to -85° C. until needed.

Binding Assays utilized the membranes described above. Membrane protein (2 to 20 μg total membrane protein) was incubated with 0.1 to 0.2 nM ¹²⁵I-labeled RANTES or MIP-1α with or without unlabeled competitor (RANTES or MIP-1α) or various concentrations of compounds. The binding reactions were performed in 60 to 100 μl of a binding buffer consisting of 10 mM HEPES pH 7.2, 1 mM CaCl₂, 5 mM MgCl₂, and 0.5% BSA (bovine serum albumin), for 60 min at room temperature. The binding reactions were terminated by harvesting the membranes by rapid filtration through glass fiber filters (GF/B or GF/C, Packard) which were presoaked in 0.3% polyethyleneimine. The filters were rinsed with approximately 600 μl of binding buffer containing 0.5 M NaCl, dried, and the amount of bound radioactivity was determined by scintillation counting in a Topcount beta-plate counter.

The activities of test compounds are reported in the Table below as IC₅₀ values or the inhibitor concentration required for 50% inhibition of specific binding in receptor binding assays using ¹²⁵I-RANTES or ¹²⁵I-MIP-1α as ligand and THP-1 cell membranes. Specific binding is defined as the total binding minus the non-specific binding; non-specific binding is the amount of cpm still detected in the presence of excess unlabeled Rantes or MIP-1α,

TABLE
BIOLOGICAL DATA

| Example | IC₅₀ (μM) |
|---|---|
| 1 | <1 |
| 2 | <1 |
| 8 | <1 |
| 12 | <1 |
| 17 | <10 |
| 18 | <1 |
| 19 | <1 |
| 21 | <1 |
| 22 | <1 |
| 23 | <1 |
| 24 | <10 |
| 25 | <1 |
| 26 | <1 |
| 27 | <1 |
| 28 | <1 |
| 29 | <1 |
| 30 | <1 |
| 31 | <1 |
| 32 | <1 |
| 33 | <1 |
| 34 | <1 |
| 35 | <1 |
| 36 | <1 |
| 38 | <1 |
| 39 | <10 |
| 40 | <1 |
| 41 | <1 |
| 42 | <1 |
| 43 | <10 |
| 44 | <1 |
| 45 | <1 |
| 46 | <1 |
| 47 | <1 |
| 48 | <1 |
| 49 | <1 |
| 51 | <1 |
| 52 | <1 |
| 53 | <1 |
| 54 | <1 |
| 55 | <1 |
| 56 | <1 |
| 57 | <10 |
| 59 | <1 |
| 60 | <1 |
| 61 | <10 |
| 62 | <10 |
| 63 | <10 |
| 64 | <1 |
| 65 | <1 |
| 66 | <1000 |
| 67 | <1 |
| 68 | <10 |
| 69 | <1 |
| 71 | <1 |
| 72 | <10 |
| 73 | <10 |
| 74 | <1000 |
| 75 | <10 |
| 76 | <10 |
| 77 | <1 |
| 78 | <1 |
| 79 | <1 |
| 83 | <1000 |
| 85 | <1 |
| 86 | >10 |
| 89 | >10 |
| 90 | <1 |
| 91 | <1 |
| 111 | <1 |
| 114 | <1 |
| 117 | <1 |
| 118 | <1 |
| 120 | <1 |
| 122 | <1 |
| 123 | <1 |
| 128 | <1 |
| 130 | <1 |
| 131 | <1 |
| 132 | <1 |
| 133 | <1 |
| 134 | <1 |
| 135 | <1 |
| 138 | <1 |
| 139 | <1 |
| 140 | >10 |
| 141 | <1 |
| 142 | <10 |
| 143 | <1 |
| 144 | <1 |
| 145 | <10 |
| 146 | >10 |
| 147 | <10 |
| 148 | <10 |
| 149 | <1000 |
| 150 | <10 |
| 151 | <1 |
| 152 | <1 |
| 153 | <1 |
| 154 | <1 |
| 155 | <1 |
| 158 | <1 |
| 159 | <1 |
| 160 | <1 |
| 161 | <10 |
| 162 | <1 |
| 163 | <1 |
| 166 | <10 |
| 167 | >1 |
| 168 | 1 |
| 172 | <1 |
| 173 | <1 |
| 174 | <1 |
| 175 | <1 |
| 176 | <1 |
| 178 | <1 |
| 180 | <1 |
| 181 | <1 |
| 182 | <1 |
| 183 | <1 |
| 184 | <10 |
| 185 | <1000 |
| 186 | <1 |
| 187 | <1 |
| 188 | >10 |
| 190 | >10 |
| 191 | >10 |
| 192 | >10 |
| 193 | <1 |
| 194 | <1 |
| 195 | <10 |
| 197 | <1 |
| 198 | <1 |
| 199 | <1 |
| 200 | <1 |
| 201 | <1 |
| 203 | <1 |
| 204 | <1 |
| 205 | <1 |
| 211 | <1 |
| 212 | <1 |
| 215 | <1 |
| 216 | <1 |
| 218 | <1 |
| 242 | <1 |
| 248 | <10 |
| 249 | <1 |
| 262 | <1 |
| 263 | <1 |
| 264 | <1 |
| 265 | <1 |
| 266 | <1 |
| 267 | <1 |
| 268 | <1 |
| 269 | <1 |
| 270 | <1 |
| 271 | <1 |
| 272 | <1 |
| 273 | <1 |
| 277 | <1 |
| 278 | <1 |
| 279 | <1 |
| 280 | <1 |
| 281 | <1 |
| 282 | <1 |
| 283 | <1 |
| 284 | <1 |
| 285 | <1 |
| 286 | <1 |
| 287 | <1 |
| 288 | <1 |
| 289 | <1 |
| 290 | <1 |
| 291 | <1 |
| 292 | <1 |
| 306 | <1 |
| 422 | <1 |
| 423 | <1 |
| 424 | <1 |
| 425 | <1 |
| 426 | <1 |
| 427 | <1 |
| 428 | <1 |
| 429 | <1 |
| 430 | <1 |
| 431 | <1 |
| 432 | <1 |

### EXAMPLE 8

### 4-(4-Chlorophenyl)-1-[3-(6,11-dihydro-dibenz[b,e]thiepin-11-ylidene)propyl]piperidin-4-ol

Step 1

11-(3-Bromopropylidene)-6,11-dihydrodibenz[b,e]thiepine was prepared by following the procedure of example 45, step 1 and 2, but replacing 5,1 1-dihydro-7-methoxypyrido[2,3-c][1]benzoxepin-5-one with 6,11-dihydrodibenz[b,e]thiepin-11-one.

¹H-NMR (CDCl₃) δ: 2.50-2.64 (2H, m), 3.36-3.47 (3H, m), 4.99 (1H, d), 5.94 (1H, t), 6.98-7.31 (8H, m).

Step 2

The titled compound was prepared by following the procedure of example 45, step 3 but replacing 5-(3-bromopropylidene)-10,11-dihydro-5H-dibenzo[a,d]cycloheptene with the product of step 1.

¹H-NMR (CDCl₃) δ: 1.65-1.80 (3H, m), 1.95-2.70 (10H, m), 3.35 (1H, d), 4.98 (1H, d), 5.96 (1H, t), 7.09-7.43 (12H, m).

MS m/z: 462(M+1)

### EXAMPLE 12

### 1-[3-(5-Benzyl-6, 11-dihydro-6-oxo-5H-dibenz[b,e]azepin-11-ylidene)propyl]-4-(4-chlorophenyl)-piperidin-4-ol

To a solution 4-(4-chlorophenyl)-1-[3-(6,11-dihydro-6-oxo-5H-dibenz[b,e]azepin-11-ylidene)propyl]piperidin-4-ol hydrochloride (Example 39)(300 mg) in DMF (5 ml) were added sodium hydride (60% in oil, 200 mg), benzyl bromide (0.15 ml) and the mixture was stirred at room temperature for 1 hour. Water and ethyl acetate were added to the reaction mixture, the organic layer was separated and washed with saturated aqueous sodium chloride, and dried with magnesium sulfate. The solvent was distilled off under reduced pressure. The residue was purified by silica gel chromatography eluting with ethyl acetate to give the titled compound (1 80 mg).

¹H-NMR (CDCl₃) δ: 1.62-1.67 (2H, m), 1.99-2.20 (3H, m), 2.33-2.65 (8H, m), 5.10 (1H, d), 5.75 (1H, d), 5.94 (1H, t), 7.11-7.42 (16H, m), 7.91 (1H, dd).

MS m/z: 549(M+1)

### EXAMPLE 17

### 1-[3-(5-Carboxymethyl-6,11-dihydro-6-oxo-5H-dibenz[b,e]azepin-11-ylidene)propyl]-4-(4-chlorophenyl)-piperidin-4-ol

4-(4-Chlorophenyl)-1-[3-(6,11-dihydro-5-ethoxycarbonylmethyl-6-oxo-5H-dibenz[b,e]azepin-11-ylidene)propyl]piperidin-4-ol (Example 18)(1.0 g) was solved in 1M hydrogen chloride in diethyl ether and stirred at room temperature for 24 hours. Aqueous sodium hydroxide and ethyl acetate were added to the reaction mixture, the aqueous layer was separated and neutralized with dilute hydrochloric acid. The precipitation was filtered to give the titled compound (250 mg).

¹H-NMR (DMSO-d₆) δ: 1.44-1.61 (2H, m), 2.07-2.17 (1H, m), 2.35-3.01 (9H, m), 4.28 (1H, d), 4.59 (1H, d), 5.83 (1H, t), 7.18-7.71 (12H, m).

MS m/z: 517(M+1)

### EXAMPLE 18

### 4-(4-Chlorophenyl)-1-[3-(6,11-dihydro-5-ethoxycarbonymethyl-6-oxo-5H-dibenz[b,e]azepin-11-ylidene)propyl]piperidin-4-ol

The titled compound was prepared by following the procedure of example 1, but replacing 5-(3-bromopropylidene)-10,11-dihydro-5H-dibenzo[a,d]cycloheptene with 11-(3-bromopropylidene)-5-ethoxycarbonymetyl-6-oxo-5H-dibenz[b,e]azepine.

¹H-NMR (CDCl₃) δ: 1.30 (3H, t), 1.64-1.69 (2H, m), 1.97-2.10 (3H, m), 2.38-2.71 (8H, m), 4.27 (2H, q), 4.32 (1H, d), 4.84 (1H, d), 5.88 (1H, t), 7.16-7.45 (11H, m), 7.88 (1H, dd).

MS m/z: 545(M+1)

### EXAMPLE 19

### 4-(4-Chlorophenyl)-1-[3-(6,11-dihydro-5-methyl-6-oxo-5H-dibenz[b,e]azepin-11-ylidene)propyl]piperidin-4-ol

The titled compound was prepared by following the procedure of Example 1, but replacing 5-(3-bromopropylidene)-10,11-dihydro-5H-dibenzo[a,d]cycloheptene with 11-(3-bromopropylidene)-5-methyl-6-oxo-5H-dibenz[b,e]azepin.

¹H-NMR (CDCl₃) δ: 1.58-2.06 (5H, m), 2.39-2.75 (8H, m), 3.53 (3H, s), 5.84 (1H, t), 7.10-7.44 (11H, m), 7.85-7.89 (1H, m).

MS m/z: 473(M+1).

### EXAMPLE 21

### 4-(4-Chlorophenyl)-1-[3-(5H-dibenzo[a,d]cycloheptene-5-ylidene)propyl]piperidin-4-ol

The titled compound was prepared by following the procedure of example 1, but replacing 5-(3-bromopropylidene)-10,11-dihydro-5H-dibenzo[a,d]cycloheptene with 5-(3-bromopropylidene)-5H-dibenzo[a,d]cycloheptene.

¹H-NMR (CDCl₃) δ: 1.58-1.63 (2H, m), 2.00-2.05 (2H, m), 2.26-2.46 (6H, m), 2.62-2.66 (2H, m), 5.55 (1H, t), 6.85 (2H, s), 7.24-7.40 (12H, m).

MS m/z: 442 (M+1).

### EXAMPLE 22

### 4-(4-Chlorophenyl)-1-[3-(6,11-dihydro-2-methoxycarbonyldibenz[b,e]oxepin-11-ylidene)propyl]piperidin-4-ol

The titled compound was prepared by following the procedure of example 1, but replacing 5-(3-bromopropylidene)-10,11-dihydro-5H-dibenzo[a,d]cycloheptene with 11-(3-bromopropylidene)-6, 11-dihydro-2-methoxy-carbonyldibenz[b,e]oxepine.

¹H-NMR (CDCl₃) δ: 1.65-1.70 (2H, m), 2.01-2.13 (3H, m), 2.41-2.80 (7H, m), 3.85 (3H, s), 5.40 (2H, brs), 5.73 (0.6×1H, t), 6.09 (0.4×1H, t), 6.76 (0.6×1H, d), 6.82 (0.4×1H, d), 7.21 -7.43 (8H, m), 7.73 (1H, dd), 7.87 (0.6×1H, d), 7.97 (0.4×1H, d).

MS m/z: 504 (M+1).

### EXAMPLE 23

### 1-[3-(2-Butoxycarbonyl-6,11-dihydrodibenz[b,e]oxepin-11-ylidene)propyl]-4-(4-chlorophenyl)piperidin-4-ol

The titled compound was prepared by following the procedure of example 1, but replacing 5-(3-bromopropylidene)-10,11-dihydro-5H-dibenzo[a,d]cycloheptene with 11-(3-bromopropylidene)-2-butoxy-6,11-dihydrodibenz[b,e]oxepine.

¹H-NMR (CDCl₃) δ: 0.96 (3H, t), 1.53 (2H, q), 1.70-1.77 (3H, m), 2.02-2.14 (3H, m), 2.39-2.78 (5H, m), 4.27 (2H, t), 5.27 (2H, brs), 5.75 (0.8×1H, t), 6.10 (0.2×1H, t), 6.78 (1H, d), 7.27-7.43 (8H, m), 7.76 (1H, dd), 7.89 (0.8×1H, d), 7.98 (0.2×1H, d).

MS m/z: 546 (M+1).

### EXAMPLE 24

### 1-[3-(2-Carboxyl-6,11-dihydrodibenz[b,e]oxepin-11-ylidene)propyl]-4-(4-chlorophenyl)piperidin-4-ol

To a solution of 4-(4-Chlorophenyl)-1-[3-(6,11-dihydro-2-methoxycarbonyldibenz[b,e]oxepin-11-ylidene)propyl]piperidin-4-ol (Example 22)(100 mg) in ethanol (3 ml) were added 15% sodium hydroxide aqueous solution (0.6 ml) and the mixture was heated to reflux for 12 hours. The solvent was distilled off under reduced pressure. Water and ethyl acetate were added to the reaction mixture, the aqueous layer was separated and neutralized with dilute hydrochloric acid. The precipitation was filtered to give the titled compound (80 mg).

¹H-NMR (CD₃OD) δ: 1.73-1.79 (2H, m), 2.14-2.19 (2H, m), 2.80-2.93 (3H, m), 3.02-3.11 (3H, m), 3.24-3.29 (2H, m), 5.25 (2H, brs), 5.61 (0.7×1H, t), 6.05 (0.3×1H, t), 6.72 (1H, d),7.22-7.40 (8H, m), 7.52-7.65 (1H, m), 7.75(0.7×1H, d), 7.80 (0.3×1H, d).

MS m/z: 490 (M+1).

### EXAMPLE 25

### 4-(4-Chlorophenyl)-1-[3-(6,11-dihydro-2-dimethylaminocarbonyldibenz[b,e]oxepin-11-ylidene)propyl]piperidin-4-ol

The titled compound was prepared by following the procedure of example 1, but replacing 5-(3-bromopropylidene)-10,11-dihydro-5H-dibenzo[a,d]cycloheptene with 11-(3-bromopropylidene)-2-dimethylaminocarbonyl-6,11-dihydrodibenz[b,e]oxepine.

¹H-NMR (CDCl₃) δ: 1.62-1.67 (2H, m), 2.00-2.12 (2H, m), 2.37-2.47 (8H, m), 2.89 (6H, s), 5.25 (2H, brs), 5.68 (0.7×1H, t), 6.03 (0.3×1H, t), 6.71 (0.3×1H, d), 6.78 (0.7×1H, d), 7.13-7.40 (10H, m).

MS m/z: 517 (M+1).

### EXAMPLE 26

### 4-(4-Chlorophenyl)-1-[3-(6,11-dihydro-2-hydroxymethyldibenz[b,e]oxepin-11-ylidene)propyl]piperidin-4-ol

To a solution of (4-chlorophenyl)-1-[3-(6,11-dihydromethoxycarbonyldibenz[b,e]oxepin-11-ylidene)propyl]piperidin-4-ol (110 mg) in THF (8 ml) were added lithium aluminum hydride (10M, 0.42 ml) dropwise at 0° C., and the mixture was stirred at room temperature for 1 hour. Aqueous sodium hydroxide (1M) was added to the reaction mixture to stir for 30 minutes, then ethyl acetate and brine was added to the mixture. The organic layer was separated and washed with saturated aqueous sodium chloride, and dried with magnesium sulfate. The solvent was distilled off under reduced pressure. The residue was purified by silica gel chromatography eluting with dichloromethane-methanol (10:1) to give the titled compound (90 mg).

¹H-NMR (CDCl₃) δ: 1.61-1.66 (2H, m), 1.98-2.03 (2H, m), 2.39-2.48 (3H, m), 2.57-2.79 (6H, m), 4.52 (2H, s), 5.20 (2H, brs), 5.66 (0.8×1H, t), 6.01 (0.2×1H, t), 6.67 (0.2×1H, d), 6.79 (0.8×1H, d), 7.06 (1H, dd), 7.15-7.37 (9H, m).

MS m/z: 476 (M+1).

### EXAMPLE 27

### 4-(4-Chlorophenyl)-1-[3-(6,11-dihydro-2-(1-hydroxy-1-methyl)ethyldibenz[b,e]oxepin-11-ylidene)propyl]piperidin-4-ol

To a solution of 4-(4-chlorophenyl)-1-[3-(6,11-dihydro-2-methoxycarbonyldibenz[b,e]oxepin-11-ylidene)propyl]piperidin-4-ol (60 mg) in THF (6 ml) were added methylmagnesium chloride (3.0M, 0.16 ml) dropwise at 0° C., and the mixture was stirred at room temperature for 2 hour, the reaction mixture was quenched by saturated ammonium aqueous, then ethyl acetate and water was added to the mixture. The organic layer was separated and washed with saturated aqueous sodium chloride, and dried with magnesium sulfate. The solvent was distilled off under reduced pressure. The residue was purified by silica gel chromatography eluting with ethyl acetate-methanol (95:5) to give the titled compound (20 mg).

¹H-NMR (CDCl₃) δ: 1.54 (0.7×6H, s), 1.62 (0.3×6H, s), 1.63-1.70 (2H, m), 2.03-2.10 (3H, m), 2.38-2.49 (3H, m), 2.62-2.82 (4H, m), 5.17 (2H, brs), 5.68 (0.7×1H, t), 6.05 (0.3×1H, t), 6.75 (0.3×1H, d), 6.83 (0.7×1H, d), 7.18-7.43 (10H, m).

MS m/z: 504 (M+1).

### EXAMPLE 28

### 4-(4-Chlorophenyl)-1-[3-(2-cyano-6, 11-dihydrodibenz[b,e]oxepin-11-ylidene)propyl]piperidin-4-ol

The titled compound was prepared by following the procedure of example 1, but replacing 5-(3-bromopropylidene)-10,11-dihydro-SH-dibenzo[a,d]cycloheptene with 11-(3-bromopropylidene)-2-cyano-6, 11-dihydrodibenz[b,e]oxepine.

¹H-NMR (CDCl₃) δ: 1.67-1.72 (2H, m), 2.02-2.13 (2H, m), 2.37-2.77 (8H, m), 5.35 (2H, brs), 5.75 (0.7×1H, t), 6.07 (0.3×1H, t), 6.78 (0.3×1H, d), 6.82 (0.7×1H, d), 7.25 -7.51 (10H, m).

MS m/z: 471 (M+1).

### EXAMPLE 29

### 1-[3-(2-Aminomethyl-6,11-dihydrodibenz[b,e]oxepin-11-ylidene)propyl]-4-(4-chlorophenyl)piperidin-4-ol

To a solution of 4-(4-chlorophenyl)-1-[3-(2-cyano-6,11-dihydrodibenz[b,e]oxepin-11-ylidene)propyl]piperidin-4-ol (380 mg) in EtOH (20 ml) were added Raney nickel (50% slurry in water, 60 mg), and the mixture was hydrogenated at 15 psi for 2 hours. The mixture was filtered through the celite and distilled off under reduced pressure. The residue was purified by silica gel chromatography eluting with dichloromethane-methanol-aqueous ammonium (95:5:1) to give the titled compound (130 mg).

¹H-NMR (CDCl₃) δ: 1.76-1.94 (3H, m), 2.18-2.34 (2H, m), 2.85-3.10 (8H, m), 3.88 (2H, s), 5.30 (2H, brs), 5.59 (1H, t), 6.78 (1H, d), 7.13-7.40 (10H, m).

MS m/z: 475 (M+1).

### EXAMPLE 30

### 4-(4-Chlorophenyl)-1-[3-(6,11-dihydro-2-nitrodibenz[b,e]oxepin-11-ylidene)propyl]piperidin-4-ol

The titled compound was prepared by following the procedure of example 1, but replacing 5-(3-bromopropylidene)-10,11-dihydro-5H-dibenzo[a,d]cycloheptene with 11-(3-bromopropylidene)-6,11-dihydro-2-nitorodibenz[b,e]oxepine.

¹H-NMR (CDCl₃) δ: 1.62-1.67 (2H, m), 1.80-2.12 (3H, m), 2.28-2.78 (8H, m), 5.05 (0.3×2H, brs), 5.40 (0.7×2H, brs), 5.90 (0.7×1H, t), 6.17 (0.3×1H, t), 6.82 (0.3×1H, d), 6.92 (0.7×1H), 7.28-7.41 (8H, m), 7.82 (1H, dd), 8.15 (0.7×1H, d), 8.22 (0.3×1H, d).

MS m/z: 491 (M+1).

### EXAMPLE 31

### 1-[3-(2-Amino-6,11-dihydrodibenz[b,e]oxepin-11-ylidene)propyl]-4-(4-chlorophenyl)piperidin-4-ol

To a solution of 4-(4-chlorophenyl)-1-[3-(6,11-dihydro-2-nitrodibenz[b,e]oxepin-1 1-ylidene)propyl]piperidin-4-ol (120 mg) in EtOH (15 ml) were added tin (II) chloride (190 mg), and the mixture was heated to reflux for 1 hour. The solvent was distilled off under reduced pressure. To the residue was added ethyl acetate and sodium aqueous to neutralize. The organic layer was separated and washed with saturated aqueous sodium chloride, and dried with magnesium sulfate. The solvent was distilled off under reduced pressure. The residue was purified by silica gel chromatography eluting with dichloromethane-methanol (95:5) to give the titled compound (70 mg).

¹H-NMR (DMSO-d₆) δ: 1.54-1.60 (2H, m), 1.85-2.00 (2H, m), 2.30-2.80 (8H, m), 3.88 (2H, s).5.07 (2H, brs), 5.66 (1H, t), 6.41-6.46 (2H, m), 6.59 (1H, d), 7.24-7.49 (8H, m).

MS m/z: 461 (M+1).

### EXAMPLE 32

### 4-(4-Chlorophenyl)-1-[3-(6,11-dihydro-2-hydroxydibenz[b,e]oxepin-11-ylidene)propyl]piperidin-4-ol

Step 1

11-(3-Bromopropylidene)-6,11-dihydro-2-hydroxydibenz[b,e]oxepine was prepared by following the procedure of example 45, step 1 and 2, but replacing 5,11-dihydro-7-methoxypyrido[2,3-c][1]benzoxepin-5-one with 6,11-dihydro-2-hydroxydibenz[b,e]oxepin-11-one.

¹H-NMR (CDCl₃) δ: 2.69(2H, q), 3.39 (2H, t), 5.20(2H, brs), 5.92(1H, t), 6.50-6.81(4H, m), 7.17-7.37(4H, m).

Step 2

The titled compound was prepared by following the procedure of example 45, step 3, but replacing 5-(3-bromopropylidene)-10,11-dihydro-5H-dibenzo[a,d]cycloheptene with the product of step 1.

¹H-NMR (CDCl₃) δ: 1.60-1.75(3H, m), 1.95-2.10(2H, m), 2.35-2.80(8H, m), 5.10(2H, brs), 5.93(1H, t), 6.56(2H, brs), 6.71(1H, brs), 7.11-7.35(8H, m).

MS m/z: 462(M+1)

### EXAMPLE 33

### 4-(4-Chlorophenyl)-1-[3-(6,11-dihydro-2-methoxydibenz[b,e]oxepin-11-ylidene)propyl]piperidin-4-ol

Step 1

11-(3-Bromopropylidene)-6,11-dihydro-2-methoxydibenz[b,e]oxepine was prepared by following the procedure of example 45, step 1 and 2, but replacing 5,11-dihydro-7-methoxypyrido[2,3-c][1]benzoxepin-5-one with 6,11-dihydro-2-methoxydibenz[b,e]oxepin-11-one.

¹H-NMR (CDCl₃) δ: 2.74(2H, q), 3.43 (2H, t), 3.77(3H, s), 5.10(2H, brs), 6.02(1H, t), 6.70-6.83(3H, m), 7.21-7.38(4H, m).

Step 2

The titled compound was prepared by following the procedure of example 45, step 3, but replacing 5-(3-bromopropylidene)-10,11-dihydro-5H-dibenzo[a,d]cycloheptene with the product of step 1.

¹H-NMR (CDCl₃) δ: 1.59-1.65(2H, m), 1.95-2.66(1H, m), 3.75(3H, s), 5.10(2H, brs), 6.03(1H, t), 6.69(2H, brs), 6.82(1H, brs), 7.20-7.40(8H, m).

MS m/z: 476(M+1)

### EXAMPLE 34

### 4-(4-Chlorophenyl)-1-[3-(6,11-dihydro-2-ethoxydibenz[b,e]oxepin-11-ylidene)propyl]piperidin-4-ol

To a solution of 4-(4-chlorophenyl)-1-[3-(6,11-dihydro-2-hydroxydibenz[b,e]oxepin-11-ylidene)propyl]piperidin-4-ol (Example 32)(200 mg) in DMF (5 ml) were added sodium hydride (60% in oil, 25 mg), ethyl iodide (0.052 ml) and the mixture was stirred at room temperature for 1 hour. Water and ethyl acetate were added to the reaction mixture, the organic layer was separated and washed with saturated aqueous sodium chloride, and dried with magnesium sulfate. The solvent was distilled off under reduced pressure. The residue was purified by silica gel chromatography eluting with ethyl acetate-hexane (1:1) to give the titled compound (170 mg).

¹H-NMR (CDCl₃) δ: 1.37(3H, t), 1.60-1.65(2H, m), 1.95-2.08(3H, m), 2.28-75(8H, m), 3.96(2H, q), 5.15(2H, brs), 6.02(1H, t), 6.68(2H, brs), 6.82(1H, brs), 7.19-7.42(8H, m).

MS m/z: 490(M+1)

### EXAMPLE 35

### 1-[3-(3-Bromo-6,11-dihydrodibenz[b,e]oxepin-11-ylidene)propyl]-4-(4-chlorophenyl)piperidin-4-ol

Step 1

3-Bromo-11-(3-bromopropylidene)-6,11-dihydrodibenz[b,e]oxepine was prepared by following the procedure of example 45, step 1 and 2, but replacing 5,1 1-dihydro-7-methoxypyrido[2,3-c][1]benzoxepin-5-one with 3-bromo-6,11-dihydrodibenz[b,e]oxepin-11-one.

¹H-NMR (CDCl₃) δ: 2.74(2H, q), 3.43 (2H, t), 3.77(3H, s), 5.10(2H, brs), 6.02(1H, t), 6.70-6.83(3H, m), 7.21-7.38(4H, m).

Step 2

The titled compound was prepared by following the procedure of example 45, step 3, but replacing 5-(3-bromopropylidene)-10,11-dihydro-5H-dibenzo[a,d]cycloheptene with the product of step 1.

¹H-NMR (CDCl₃) δ: 1.63-1.70 (3H, m), 1.96-2.10 (2H, m), 2.32-2.69 (8H, m), 5.20(2H, brs), 6.00(1H, t), 6.92-7.00 (2H, m), 7.11-7.14 (1H, m), 7.24-7.42 (8H, m).

MS m/z: 524, 526(M+1)

### EXAMPLE 36

### 4-(4-Chlorophenyl)-1-[3-(6,11-dihydrodibenz[b,e]oxepin-11-ylidene)propyl]-4-methoxypiperidine

To a solution of 4-(4-chlorophenyl)-1-[3-(6,11-dihydro-2-methoxydibenz[b,e]oxepin-11-ylidene)propyl]piperidin-4-ol (Example 2)(400 mg) in DMF (5 ml) were added sodium hydride (60% in oil, 50 mg), methyl iodide (0.07 ml) and the mixture was stirred at room temperature for 1 hour. Water and ethyl acetate were added to the reaction mixture, the organic layer was separated and washed with saturated aqueous sodium chloride, and dried with magnesium sulfate. The solvent was distilled off under reduced pressure. The residue was purified by silica gel chromatography eluting with ethyl acetate-hexane (1:1) to give the titled compound (100 mg).

¹H-NMR (CDCl₃) δ: 1.90-2.04 (4H, m), 2.34-2.62 (8H, m), 2.93 (3H, s), 5.25 (2H, brs), 6.04 (1H, t), 6.75-6.91 (3H, m), 7.09-7.37 (9H, m).

MS m/z: 460 (M+1)

### EXAMPLE 37

### 4-Acetoxy-4-(4-chlorophenyl)-1-[3-(6,11-dihydrodibenz[b,e]oxepin-11-ylidene)propyl]piperidine

To a solution of 4-(4-chlorophenyl)-1-[3-(6,1 1-dihydro-2-methoxydibenz[b,e]oxepin-11-ylidene)propyl]piperidin-4-ol (Example 2)(200 mg) in dichloromethane (5 ml) were added acetyl chloride (0.06 ml), triethylamine (0.19 ml) and the mixture was stirred at room temperature for 1 hour. Aqueous sodium bicarbonate and ethyl acetate were added to the reaction mixture, the organic layer was separated and washed with saturated aqueous sodium chloride, and dried with magnesium sulfate. The solvent was distilled off under reduced pressure. The residue was purified by silica gel chromatography eluting with ethyl acetate-hexane (1:4) to give the titled compound (190 mg).

¹H-NMR (CDCl₃) δ: 1.98-2.85 (12H, m), 2.02 (3H, s), 2.93 (3H, s), 5.23 (2H, brs), 6.01 (1H, t), 6.73-6.90 (3H, m), 7.11-7.40 (9H, m).

MS m/z: 488 (M+1)

### EXAMPLE 38

### 1-[3-(8-Bromo-4,10-dihydrothieno[3,2-c][1]benzoxepin-10-ylidene)propyl]piperidin-4-(4-chlorophenyl)-4-ol

Step 1

8-Bromo-10-(3-bromopropylidene)-4,10-dihydrothieno[3,2-c][1]benzoxepine was prepared by following the procedure of example 45, step 1 and 2, but replacing 5,11-dihydro-7-methoxypyrido[2,3-c][1]benzoxepin-5-one with 4,10-dihydrothieno[3,2-c][1 ]benzoxepin-10-one.

¹H-NMR (CDCl₃) δ: 2.84 (2H, q), 3.45 (2H, t), 5.10 (2H, s), 6.11 (1H, t), 6.65 (1H, d), 7.03-7.08 (2H, m), 7.38-7.43 (2H, m).

Step 2

The titled compound was prepared by following the procedure of example 45, step 3, but replacing 5-(3-bromopropylidene)-10,11-dihydro-5H-dibenzo[a,d]cycloheptene with the product of step 1.

¹H-NMR (CDCl₃) δ: 1.66-1.75 (3H, m), 2.03-2.16 (2H, m), 2.40-2.86 (8H, m), 5.09 (0.7×2H, s),5.14 (0.3×2H, s), 5.90 (0.3×1H, t), 6.10 (0.7×1H, t), 6.64 (0.7×1H, d), 6.75 (0.3×1H, d), 6.90 (0.3×1H, d), 7.03-7.09 (2H, m), 7.21-7.45 (6H, m).

MS m/z: 532 (M+1)

### EXAMPLE 39

### 4-(4-Chlorophenyl)-1-[3-(6,11-dihydro-6-oxo-5H-dibenz[b,e]azepin-11-ylidene)propyl]piperidin-4-ol

Step 1

11-(3-Bromopropylidene)-6,11-dihydro-6-oxo-5H-dibenz[b,e]azepine was prepared by following the procedure of example 45, step 1 and 2, but replacing 5,1 1-dihydro-7-methoxypyrido[2,3-c][1]benzoxepin-5-one with 6,11-dihydro-6-5H-dibenz[b,e]azepin-6,1 1-dione.

¹H-NMR (CDCl₃) δ: 2.70-2.92 (2H, m), 3.45 (2H, t), 5.92 (1H, t), 7.08-7.58 (7H, m), 8.05 (1H, dd), 9.00 (1H, brs).

Step 2

The titled compound was prepared by following the procedure of example 45, step 3, but replacing 5-(3-bromopropylidene)-10,11-dihydro-5H-dibenzo[a,d]cycloheptene with the product of step 1.

¹H-NMR (CDCl₃) δ: 1.61-1.66 (2H, m), 1.97-2.20 (3H, m), 2.35-2.68 (8H, m), 5.80 (1H, t), 7.03-7.53 (11H, m), 8.02 (1H, dd), 9.27 (1H, brs). MS m/z: 459 (M+1)

### EXAMPLE 40

### 4-(4-Chlorophenyl)-1-[3-(6,11-dihydro-5-ethyl-6-oxo-5H-dibenz[b,e]azepin-11-ylidene)propyl]piperidin-4-ol

The titled compound was prepared by following the procedure of example 12, but replacing benzyl bromide with ethyl iodide.

¹H-NMR (CDCl₃) δ: 1.19-1.28 (3H, m), 1.63-1.69 (2H, m), 1.99-2.16 (3H, m), 2.37-2.70 (8H, m), 3.77-3.85 (1H, m), 4.40-4.48 (1H, m), 5.85 (1H, t), 7.12-7.45 (11H, m), 7.85 (1H, dd).

MS m/z: 487 (M+1)

### EXAMPLE 41

### 1-[3-(5-n-Butyl-6,11-dihydro-6-oxo-5H-dibenz[b,e]azepin-11-ylidene)propyl]-4-(4-chlorophenyl)-piperidin-4-ol

The titled compound was prepared by following the procedure of example 12, but replacing benzyl bromide with n-butyl iodide.

¹H-NMR (CDCl₃) δ: 0.90-0.98 (3H, m), 1.25-2.20 (9H, m), 2.40-2.87 (8H, m), 3.62-3.72 (1H, m), 4.52-4.64 (1H, m), 5.85 (1H, t), 7.16-7.45 (11H, m), 7.88 (11H, dd).

MS m/z: 515 (M+1)

### EXAMPLE 42

### 4-(4-Chlorophenyl)-1-[3-(6,11-dihydro-5-(3-hydroxypropyl)-6-oxo-5H-dibenz[b,e]azepin-11-ylidene)propyl]piperidin-4-ol

To a solution 4-(4-chlorophenyl)-1-[3-(6,11-dihydro-6-oxo-5H-dibenz[b,e]azepin-11-ylidene)propyl]piperidin-4-ol hydrochloride (Example 39)(500 mg) in DMF (8 ml) were added sodium hydride (60% in oil, 200 mg), 2-(3-bromopropoxy)tetrahydro-2H-pyran (0.5 ml) and the mixture was stirred at room temperature for 6 hours. Water and ethyl acetate were added to the reaction mixture, the organic layer was separated and washed with saturated aqueous sodium chloride, and dried with magnesium sulfate. The solvent was distilled off under reduced pressure. The residue was solved in 1M hydrogen chloride in diehyl ether and stirred at room temperature for 1 hour. Aqueous sodium bicarbonate and ethyl acetate were added to the reaction mixture, the organic layer was separated and washed with saturated aqueous sodium chloride, and dried with magnesium sulfate. The solvent was distilled off under reduced pressure. The residue was purified by silica gel chromatography eluting with ethyl acetate to give the titled compound (250 mg).

¹H-NMR (CDCl₃) δ: 1.25-2.87 (15H, m), 3.51-3.56 (2H, m), 3.76-3.82 (1H, m), 4.81-4.87 (1H, m), 5.86 (1H, t), 7.16-7.45 (1 1H, m), 7.82 (1H, dd).

MS m/z: 517 (M+1)

### EXAMPLE 43

### 1-[3-(5-tert-Butoxycarbonymethyl-6,11-dihydro-6-oxo-5H-dibenz[b,e]azepin-11-ylidene)propyl]-4-(4-chlorophenyl)-piperidin-4-ol

The titled compound was prepared by following the procedure of example 12, but replacing benzyl bromide with tert-butyl bromoacetate.

¹H-NMR (CDCl₃) δ: 1.50 (9H, s), 1.65-1.70 (2H, m), 1.95-2.10 (3H, m), 2.42-2.75 (8H, m), 4.24 (1H, d), 4.75 (1H, d), 5.88 (1H, t), 7.16-7.46 (1 1H, m), 7.90 (1H, dd).

MS m/z: 573 (M+1)

### EXAMPLE 44

### 4-(4-Chlorophenyl)-1-[3-(5,11-dihydro-7-hydroxy[1]benzoxepino[2,3-b]pyridin-5-ylidene)propyl]piperidin-4-ol

Step 1

To a solution of the product of example 45, step 1 (4.3 g) in dichloroethane (100 ml) was added boron tribromide-methyl sulfide complex (19.3 g) and the mixture was heated to reflux for 3 hour. Water and ethyl acetate were added to the reaction mixture and neutralized with dilute NaOH solution. The organic layer was separated and washed with saturated aqueous sodium chloride, and dried over magnesium sulfate. The solvent was distilled off under reduced pressure. The residue was purified by silica gel chromatography eluting with ethyl acetate-hexane (1:2) to give 5-(3-bromopropylidene)-5,11-dihydro-7-hydroxy[1 ]benzoxepino[2,3-b]pyridine (3.2 g).

¹H-NMR (CDCl₃) δ: 2.72 (2H, q), 3.45 (2H, t), 5.28 (2H, brs), 6.03 (1H, t), 6.66-6.80 (3H, m), 7.26 (1H, dd), 7.58 (1H, dd), 8.51 (1H, dd).

Step 2

The titled compound was prepared by following the procedure of example 45, step 3, but replacing 5-(3-bromopropylidene)-5,1 1-dihydro-7-methoxy[1]benzoxepino[2,3-b]pyridine with the product of step 1.

¹H-NMR (DMSO-d₆) δ: 1.46-1.51 (2H, m), 1.74-1.85 (2H, m), 2.29-2.51 (8H, m), 5.15 (2H, brs), 6.07 (1H, t), 6.61-6.70 (3H, m), 7.33-7.48 (5H, m), 7.73 (1H, dd), 8.47 (1H, dd), 9.06 (1H, s).

MS m/z: 463 (M+1)

### EXAMPLE 45

### 4-(4-Chlorophenyl)-1-[3-(5,11-dihydro-7-methoxy[1]benzoxepino[2,3-b]pyridin-5-ylidene)propyl]piperidin-4-ol

Step 1

To a solution of 5,1 1-dihydro-7-methoxy[1]benzoxepino[2,3-b]pyridin-5-one (5.0 g) in THF (50 ml) was added 1.1M cyclopropylmagnesium bromide THF solution (25 ml) at 0° C. The reaction mixture was warmed to room temperature, and stirred for 30 minutes. Aqueous ammonium chloride and ethyl acetate were added to the reaction mixture, the organic layer was separated and washed with saturated aqueous sodium chloride, and dried with magnesium sulfate. The solvent was distilled off under reduced pressure. The residue was filtered and washed with ethyl acetate-hexane (1:2) to give 5-cyclopropyl-5,11-dihydro-7-methoxy[1]benzoxepino[2,3-b]pyridin-5-ol (5.0 g).

Step 2

To a solution of the product of step 1 (4.3 g) in acetic acid (30 ml) was added 48% aqueous HBr (25 ml) at 1 0° C. The reaction mixture was warmed to room temperature, and stirred for 12 hours. Water and ethyl acetate were added to the reaction mixture and neutralized with dilute NaOH solution. The organic layer was separated and washed with saturated aqueous sodium chloride, and dried over magnesium sulfate. The solvent was distilled off under reduced pressure. The residue was purified by silica gel chromatography eluting with ethyl acetate-hexane (1:4) to give 5-(3-bromopropylidene)-5,11-dihydro-7-methoxy[1]benzoxepino[2,3-b]pyridine (5.6 g).

¹H-NMR (CDCl₃) δ: 2.74 (2H, q), 3.46 (2H, t), 3.78 (3H, s), 5.25 (2H, brs), 6.07 (1H, t), 6.72-6.82 (3H, m), 7.21-7.42 (5H, m), 7.56 (1H, dd), 8.45 (1H, dd).

Step 3

To a solution the product of step 2 (1.1 g) in DMF (15 ml) were added 4-(4-chlorophenyl)-4-hydroxypiperidine (0.81 g) and potassium carbonate (0.53 g) and the mixture was stirred at room temperature for 3 hours. Water and ethyl acetate were added to the reaction mixture, the organic layer was separated and washed with saturated aqueous sodium chloride, and dried with magnesium sulfate. The solvent was distilled off under reduced pressure. The residue was purified by silica gel chromatography eluting with methylene chloride-methanol (10:1) to give the titled compound as major regioisomer (0.86 g) and minor one (0.05 g). Major isomer

¹H-NMR (CDCl₃) _67 : 1.64-1.69 (2H, m), 1.91-2.08 (3H, m), 2.34-2.69 (8H, m), 3.77 (3H, s), 5.25 (2H, brs), 6.07 (1H, t), 6.72-6.82 (3H, m), 7.21-7.42 (5H, m), 7.56 (1H, dd), 8.45 (1H, dd).

MS m/z: 477 (M+1)

Minor Isomer

¹H-NMR (CDCl₃) d: 1.65-1.79 (3H, m), 2.01-2.13 (2H, m), 2.35-2.76 (8H, m), 3.76 (3H, s), 5.22 (2H, brs), 5.95 (1H, t), 6.72-6.80 (2H, m), 7.06 (1H, d), 7.16 (1H, dd), 7.28 (2H, d), 7.42 (2H, d), 7.66 (1H, dd), 8.39 (1H, dd).

MS m/z: 477 (M+1)

### EXAMPLE 46

### 4-(4-Chlorophenyl)-1-[3-(5,11-dihydro-7-ethoxy[1 ]benzoxepino[2,3-b]pyridin-5-ylidene)propyl]piperidin-4-ol

The titled compound was prepared by following the procedure of example 34, but replacing 4-(4-chlorophenyl)-1-[3-(6,11-dihydro-2-hydroxydibenz[b,e]oxepin-11-ylidene)propyl]piperidin-4-ol with 4-(4-chlorophenyl)-1-[3-(5,11-dihydro-7-hydroxy[1]benzoxepino[2,3-b]pyridin-5-ylidene)propyl]piperidin-4-ol (example 44).

¹H-NMR (CDCl₃) δ: 1.38 (3H, t), 1.67-1.72 (3H, m), 2.05-2.16 (2H, m), 2.40-2.80 (8H, m), 3.99 (2H, q), 5.26 (2H, brs), 6.05 (1H, t), 6.71-6.82 (3H, m), 7.23-7.43 (5H, m), 7.57 (1H, dd), 8.47 (1H, dd).

MS m/z: 491 (M+1)

### EXAMPLE 47

### 4-(4-Chlorophenyl)-1-[3-(5,11-dihydro-7-isopropoxy[1]benzoxepino[2,3-b]pyridin-5-ylidene)propyl]piperidin-4-ol

The titled compound was prepared by following the procedure of example 46, but replacing ethyl iodide with isopropyl bromide.

¹H-NMR (CDCl₃) δ: 1.30 (6H, d), 1.60-1.70 (3H, m), 1.99-2.09 (2H, m), 2.33-2.69 (8H, m), 4.37-4.48 (1H, m), 5.26 (2H, brs), 6.06 (1H, t), 6.73-6.82 (3H, m), 7.21-7.43 (5H, m), 7.55 (1H, dd), 8.47 (1H, dd).

MS m/z: 505 (M+1)

### EXAMPLE 48

### 4-(4-Chlorophenyl)-1-[3-(5,11-dihydro-7-ethoxycarbonylmethyloxy[1]benzoxepino[2,3-b]pyridin-5-ylidene)propyl]piperidin-4-ol

The titled compound was prepared by following the procedure of example 46, but replacing ethyl iodide with ethyl bromoacetate.

¹H-NMR (CDCl₃) δ: 1.28 (3H, t), 1.63-1.68 (2H, m), 1.97-2.02 (3H, m), 2.33-2.68 (8H, m), 4.24 (2H, q), 4.55 (2H, s), 5.26 (2H, brs), 6.06 (1H, t), 6.73-6.88 (3H, m), 7.21-7.42 (5H, m), 7.55 (1H, dd), 8.44 (1H, dd). MS m/z: 549 (M+1)

### EXAMPLE 49

### 4-(4-Chlorophenyl)-1-[3-(7-cyanomethyloxy-5,11-dihydro[1]benzoxepino[2,3-b]pyridin-5-ylidene)propyl]piperidin-4-ol

The titled compound was prepared by following the procedure of example 46, but replacing ethyl iodide with bromoacetonitrile.

¹H-NMR (CDCl₃) δ: 1.62-1.67 (2H, m), 1.94-2.06 (2H, m), 2.21 (1H, brs), 2.34-2.66 (8H, m), 4.70 (2H, s), 5.26 (2H, brs), 6.10 (1H, t), 6.80 (2H, brs), 6.92 (1H, brs), 7.22-7.41 (5H, m), 7.56 (1H, dd), 8.44 (1H, dd).

MS m/z: 502 (M+1)

### EXAMPLE 50

### 1-[3-(7-(2-Acetoxyethyl)oxy-5,11-dihydro[1]benzoxepino[2,3-b]pyridin-5-ylidene)propyl]-4-(4-chlorophenyl)piperidin-4-ol

The titled compound was prepared by following the procedure of example 46, but replacing ethyl iodide with 2-bromoethyl acetate.

¹H-NMR (CDCl₃) δ: 1.65-1.72 (3H, m), 1.97-2.09 (5H, m), 2.37-2.70 (8H, m), 4.11-4.14 (2H, m), 4.37-4.41 (2H, m), 5.25 (2H, brs), 6.07 (1H, t), 6.75-6.84 (3H, m), 7.23-7.43 (5H, m), 7.56 (1H, dd), 8.47 (1H, dd).

MS m/z: 549 (M+1)

### EXAMPLE 51

### 4-(4-Chlorophenyl)-1-[3-(5,11-dihydro-7-(2-hydroxyethyl)oxy[1]benzoxepino[2,3-b]pyridin-5-ylidene)propyl]piperidin-4-ol

To a solution of 1-[3-(7-(2-acetoxyethyl)oxy-5,11-dihydro[1]benzoxepino[2,3-b]pyridin-5-ylidene)propyl]-4-(4-chlorophenyl)piperidin-4-ol (Example 50)(140 mg) in ethanol (5 ml) were added 15% sodium hydroxide aqueous solution (2 ml) and the mixture was heated to reflux for 1 hour. Water and ethyl acetate were added to the reaction mixture, the organic layer was separated and washed with saturated aqueous sodium chloride, and dried with magnesium sulfate. The solvent was distilled off under reduced pressure. The residue was purified by silica gel chromatography eluting with methylene chloride-methanol (10:1) to give the titled compound (120 mg).

¹H-NMR (CDCl₃) δ: 1.64-1.69 (2H, m), 1.98-2.10 (3H, m), 2.36-2.79 (8H, m), 3.89-3.94 (2H, m), 3.99-4.04 (2H, m), 5.24 (2H, brs), 6.04 (1H, t), 6.71-6.84 (3H, m), 7.23-7.41 (5H, m), 7.54 (1H, dd), 8.43 (1H, dd).

MS m/z: 507 (M+1)

### EXAMPLE 52

### 4-(4-Chlorophenyl)-1-[3-(5,11-dihydro-7-(2-morpholinoethyl)oxy[1]benzoxepino[2,3-b]pyridin-5-ylidene)propyl]piperidin-4-ol

The titled compound was prepared by following the procedure of example 46, but replacing ethyl iodide with 4-(2-chloroethyl)morpholine hydrochloride. ¹H-NMR (CDCl₃) δ: 1.62-1.67 (2H, m), 1.95-2.08 (2H, m), 2.20-2.67 (13H, m), 2.74 (2H, t), 3.67-3.71 (4H, m), 4.04 (2H, t), 5.23 (2H, brs), 6.05 (1H, t), 6.73-6.82 (3H, m), 7.20-7.41 (5H, m), 7.53 (1H, dd), 8.42 (1H, dd).

MS m/z: 576 (M+1)

### EXAMPLE 53

### 4-(4-Chlorophenyl)-1-[3-(5,11-dihydro[1]benzoxepino[2,3-b]pyridin-5-ylidene)propyl]piperidin-4-ol

Step 1

5-(3-Bromopropylidene)-5,11-dihydro[1]benzoxepino[2,3-b]pyridine was prepared by following the procedure of example 45, step 1 and 2, but replacing 5,11-dihydro-7-methoxy[1]benzoxepino[2,3-b]pyridin-5-one with 5,11-dihydro[1]benzoxepino[2,3-b]pyridin-5-one.

¹H-NMR (CDCl₃) δ: 2.71 (2H, q), 3.46 (2H, t), 5.33 (2H, brs), 6.04 (1H, t), 7.01-7.17 (3H, m), 7.29 (1H, dd), 7.56 (1H, dd), 8.53 (1H, dd).

Step 2

The titled compound was prepared by following the procedure of example 45, step 3, but replacing 5-(3-bromopropylidene)-5,11-dihydro-7-methoxy[1]benzoxepino[2,3-b]pyridine with the product of step 1.

¹H-NMR (CDCl₃) δ: 1.66-1.71 (2H, m), 2.00-2.20 (3H, m), 2.36-2.69 (8H, m), 5.34 (2H, brs), 6.10 (1H, t), 6.83-6.96 (3H, m), 7.17-7.44 (6H, m), 7.60 (1H, dd), 8.46 (1H, dd).

MS m/z: 447 (M+1)

### EXAMPLE 54

### 1-[3-(8-Bromo-5,1 1-dihydro[1]benzoxepino[2,3-b]pyridin-5-ylidene)propyl]-4-(4-chlorophenyl)piperidin-4-ol

Step 1

8-Bromo-5-(3-bromopropylidene)-5,11-dihydro[1]benzoxepino[2,3-b]pyridine was prepared by following the procedure of example 45, step 1 and 2, but replacing 5,1 1-dihydro-7-methoxy[1]benzoxepino[2,3-b]pyridin-5-one with 8-bromo-5,11-dihydro[1]benzoxepino[2,3-b]pyridin-5-one.

¹H-NMR (CDCl₃) δ: 2.75 (2H, q), 3.50 (2H, t), 5.38 (2H, brs), 6.08 (1H, t), 6.85-6.98 (2H, m), 7.18-7.35 (3H, m), 7.59 (1H, dd), 8.54 (1H, dd).

Step 2

The titled compound was prepared by following the procedure of example 45, step 3, but replacing 5-(3-bromopropylidene)-5,11-dihydro-7-methoxy[1]benzoxepino[2,3-b]pyridine with the product of step 1.

¹H-NMR (CDCl₃) δ: 1.64-1.69 (2H, m), 1.90-2.07 (3H, m), 2.30-2.67 (8H, m), 5.30 (2H, brs), 6.08 (1H, t), 7.00-7.07 (2H, m), 7.13 (1H, d), 7.25-7.42 (5H, m), 7.56 (1H, dd), 8.47 (1H, dd).

MS m/z: 525, 527 (M+1)

### EXAMPLE 55

### 4-(4-Chlorophenyl) 1-[3-(10,11-dihydro-10-oxo-5H-pyrido[2,3-c][2]benzazepin-5-ylidene)propyl]piperidin-4-ol

Step 1

5-(3-Bromopropylidene)-10,11-dihydro-10-oxo-5H-pyrido[2,3-c][2]benzazepine was prepared by following the procedure of example 45, step 1 and 2, but replacing 5,11-dihydro-7-methoxy[1]benzoxepino[2,3-b]pyridin-5-one with 10,11-dihydro-5H-pyrido[2,3-c][2]benzazepin-5,10-dione.

¹H-NMR (CDCl₃) δ: 2.75-2.90 (2H, m), 3.45 (2H, t), 5.92 (1H, t), 7.04-7.70 (5H, m), 8.10 (1H, dd), 8.48 (1H, dd), 10.00 (1H, brs).

Step 2

The titled compound was prepared by following the procedure of example 45, step 3, but replacing 5-(3-bromopropylidene)-10,11-dihydro-5H-dibenzo[a,d]cycloheptene with the product of step 1.

¹H-NMR (CDCl₃) δ: 1.64-1.69 (3H, m), 2.00-2.12 (2H, m), 2.35-2.70 (8H, m), 5.82 (1H, t), 7.08 (1H, dd), 7.23-7.62 (8H, m), 8.04 (1H, dd), 8.32 (1H, dd), 8.76 (1H, brs).

MS m/z: 460 (M+1)

### EXAMPLE 56

### 4-(4-Chlorophenyl)-1-[3-(10,11-dihydro-11-methyl-10-oxo-5H-pyrido[2,3-c][2]benzazepin-5-ylidene)propyl]piperidin-4-ol

The titled compound was prepared by following the procedure of example 36, but replacing of 4-(4-chlorophenyl)-1-[3-(6,11-dihydro-2-methoxydibenz[b,e]oxepin-11-ylidene)propyl]piperidin-4-ol with 5-(3-bromopropylidene)-10,11-dihydro-10-oxo-5H-pyrido[2,3-c][2]benzazepine.

¹H-NMR (CDCl₃) δ: 1.64-1.70 (3H, m), 2.00-2.10 (2H, m), 2.41-2.69 (8H, m), 3.62 (3H, s), 5.82 (1H, t), 7.07 (1H, dd), 7.25-7.54 (8H, m), 7.91 (1H, dd), 8.34 (1H, dd).

MS m/z: 474 (M+1)

### EXAMPLE 57

### 4-(4-Chlorophenyl)-1-[3-(5,11-dihydro-7-methoxy[1]benzoxepino[2,3-b]pyridin-5-ylidene)ethyl]piperidin-4-ol

Step 1

To a solution of methyltriphenylphosphonium bromide (2.2 g) in THF (20 ml) was added 1.6M n-butyl lithium hexane solution (2.9 ml) at 0° C. for 30 minutes. To the reaction mixture cooled to 0° C. was added 5,11-dihydro-7-methoxy[1]benzoxepino[2,3-b]pyridin-5-one (1.0 g) dropwise as THF solution (5 ml), and the mixture was warmed to room temperature, and stirred for 3 hours. Aqueous ammonium chloride and ethyl acetate were added to the reaction mixture, the organic layer was separated and washed with saturated aqueous sodium chloride, and dried with magnesium sulfate. The solvent was distilled off under reduced pressure. The residue was purified by silica gel chromatography eluting with ethyl acetate-hexane (1:4) to give 5,11-dihydro-7-methoxy-5-methylenepyrido[2,3-c][1]benzoxepine (0.14 g).

Step 2

To a solution of DMF (0.54 ml) was added phosphorus oxychloride (0.41 ml) at 0° C. for 10 minutes. To the reaction mixture was added the product of step 1 (210 mg) in carbontetrachloride (5 ml) and the mixture was heated to reflux for 5 hours. Aqueous sodium bicarbonate and ethyl acetate were added to the reaction mixture, the organic layer was separated and washed with saturated aqueous sodium chloride, and dried with magnesium sulfate. The solvent was distilled off under reduced pressure. The residue was purified by silica gel chromatography eluting with ethyl acetate-hexane (1:4) to give 3-(5,11-dihydro-7-methoxy[1]benzoxepino[2,3-b]pyridin-5-ylidene)acetaldehyde (130 mg).

¹H-NMR (CDCl₃) δ: 3.77 (0.7×3H, s),3.79 (0.3×3H, s), 5.31 (2H, s), 6.46 (0.7×1H, d), 6.52 (0.3×1H, d), 6.78-7.40 (4H, m), 7.68 (0.3×1H, dd), 7.78 (0.7×1H, dd), 8.55 (0.7×1H, dd), 8.64 (0.3×1H, dd), 9.62 (0.3×1H, d), 9.79 (0.7×1H, d).

Step 3

The titled compound was prepared by following the procedure of example 58, step 2, but replacing of 3-(5,11-dihydro-7-methoxy[1]benzoxepino[2,3-b]pyridin-5-ylidene)propanaldehyde with product of step 2.

¹H-NMR (CDCl₃) δ: 1.64-1.82 (2H, m), 1.92-2.22 (3H, m), 2.43-2.58 (2H, m), 2.79-3.45 (6H, m), 3.68 (0.3×3H, s), 3.70 (0.7×3H, s), 5.24 (2H, brs), 6.18 (0.7×1H, t), 6.21 (0.3×1H, t), 6.72-7.42 (8H, m), 7.78 (0.3×1H, dd), 7.85 (0.7×1H, dd), 8.42 (0.7×1H, dd), 8.46 (0.3×1H, dd).

MS m/z: 463 (M+1).

### EXAMPLE 58

### 4-(4-Chlorophenyl)-1-[4-(5,11-dihydro-7-methoxy[1]benzoxepino[2,3-b]pyridin-5-ylidene)butyl]piperidin-4-ol

Step 1

3-(5,11-Dihydro-7-methoxy[1]benzoxepino[2,3-b]pyridin-5-ylidene)propenaldehyde was prepared by following the procedure of example 57, step 2, but replacing 5,1 1-dihydro-7-methoxy-5-methylene[1]benzoxepino[2,3-b]pyridine with 5,11-dihydro-7-methoxy-5-(propyl-1-ene) [1]benzoxepino[2,3-b]pyridine (by-product of example 45, step 3).

¹H-NMR (CDCl₃) δ: 3.78 (0.3×3H, s), 3.80 (0.7×3H, s), -5.32 (2H, brs), 6.34-6.39 (1H, m), 6.72-7.38 (6H, m), 7.58 (0.7×1H, dd), 7.77 (0.3×1H, dd), 8.49 (0.3×1H, dd), 8.60 (0.7×1H, dd), 9.51 (0.7×1H, d), 9.54 (0.3×1H, d).

Step 2

To a solution of the product of step 1 (90 mg) in dichloromethane (6 ml) were added sodium triacetoxyborohydride (170 mg), 4-(4-chlorophenyl)-4-hydroxypiperidine (70 mg) and acetic acid (0.02 ml) and the mixture stirred at room temperature for 24 hour. Water and ethyl acetate were added to the reaction mixture, the organic layer was separated and washed with saturated aqueous sodium chloride, and dried with magnesium sulfate. The solvent was distilled off under reduced pressure. The residue was purified by silica gel chromatography eluting with dichloromethane-methanol (95:5) to give 4-(4-chlorophenyl)-1-[4-(5,11-dihydro-7-methoxy[1]benzoxepino[2,3-b]pyridin-5-ylidene)buten-2-yl]piperidin-4-ol (110 mg).

¹H-NMR (CDCl₃) δ: 1.68-1.73 (2H, m), 2.04-2.16 (2H, m), 2.43-2.72 (3H, m), 2.77-2.81 (2H, m), 3.08-3.13 (2H, m), 3.73 (0.3×3H, s), 3.77 (0.7×3H, s), 5.20 (2H, brs), 5.98-6.05 (1H, m), 6.23-7.43 (10H, m), 7.58 (0.7×1H, dd), 7.65 (0.3×1H, dd), 8.37 (0.3×1H, dd), 8.45 (0.7×1H, dd).

MS m/z: 489 (M+1).

Step 3

To a solution of the product of step 2 (8 mg) in ethanol (2 ml) were added 10% Pd-C (2 mg) was stirred under hydrogen (under a balloon) at room temperature for 1 hour. The mixture was filtered through the celite and distilled off under reduced pressure to give the titled compound (6 mg).

¹H-NMR (CDCl₃) δ: 1.68-3.00 (15H, m), 3.77 (3H, s), 5.18-5.35 (2H, m), 5.94 (0.4H, t, E isomer), 6.06 (0.6H, t, Z isomer), 6.65-6.88 (3H, m), 7.05-7.73 (6H, m), 8.30-8.56 (1H, m).

MS m/z: 491 (M+1)

### EXAMPLE 59

### 1-[3-(5,11-Dihydro-7-methoxy[1]benzoxepino[2,3-b]pyridin-5-ylidene)propyl]piperidin-4-phenyl-4-ol

The titled compound was prepared by following the procedure of example 45, step 3, but replacing 4-(4-chlorophenyl)-4-hydroxypiperidine with 4-phenyl-4-hydroxypiperidine.

¹H-NMR (CDCl₃) δ: 1.68-1.73 (2H, m), 2.02-2.15 (3H, m), 2.38-2.72 (8H, m), 3.77 (3H, s), 5.26 (2H, brs), 6.08 (1H, t), 6.72-6.83 (3H, m), 7.21-7.36 (4H, m), 7.46-7.49 (2H, m), 7.58 (1H, dd), 8.46 (1H, dd).

MS m/z: 443 (M+1).

### EXAMPLE 60

### 4-(4-Bromophenyl)-1-[3-(5,11-dihydro-7-methoxy[1]benzoxepino[2,3-b]pyridin-5-ylidene)propyl]piperidin-4-ol

The titled compound was prepared by following the procedure of example 45, step 3, but replacing 4-(4-chlorophenyl)-4-hydroxypiperidine with 4-(4-bromophenyl)-4-hydroxypiperidine.

¹H-NMR (CDCl₃) δ: 1.65-1.69 (2H, m), 2.00-2.10 (3H, m), 2.37-2.71 (8H, m), 3.76 (3H, s), 5.24 (2H, brs), 6.05 (1H, t), 6.70-6.82 (3H, m), 7.24 (1H, dd), 7.38 (2H, d), 7.44 (2H, s), 7.52 (1H, dd), 8.44 (1H, dd).

MS m/z: 521,523 (M+1).

### EXAMPLE 61

### 1-[3-(5,11-Dihydro-7-methoxy[1]benzoxepino[2,3-b]pyridin-5-ylidene)propyl]piperidin-4-ol

The titled compound was prepared by following the procedure of example 45, step 3, but replacing 4-(4-chlorophenyl)-4-hydroxypiperidine with 4-hydroxypiperidine.

¹H-NMR (CDCl₃) δ: 1.43-1.60 (2H, m), 1.80-1.98 (2H, m), 2.00-2.18 (3H, m), 2.34-2.48 (4H, m), 2.63-2.76 (2H, m), 3.64-3.73 (1H, m), 3.70 (3H, s), 5.35 (2H, brs), 6.06 (1H, t), 6.74-6.84 (3H, m), 7.25 (1H, dd), 7.60 (1H, dd), 8.50 (1H, dd).

MS m/z: 367 (M+1).

### EXAMPLE 62

### 4-Benzyl-1-[3-(5,11-dihydro-7-methoxy[1]benzoxepino[2,3-b]pyridin-5-ylidene)propyl]piperidin-4-ol

The titled compound was prepared by following the procedure of example 45, step 3, but replacing 4-(4-chlorophenyl)-4-hydroxypiperidine with 4-benzyl-4-hydroxypiperidine.

¹H-NMR (CDCl₃) δ: 1.42-1.57 (3H, m), 1.62-1.75 (2H, m), 2.22-2.70 (8H, m), 2.79 (2H, s), 3.80 (3H, s), 5.25 (2H, brs), 6.08 (1H, t), 6.73-6.84 (3H, m), 7.18-7.24 (6H, m), 7.57 (1H, dd), 8.50 (1H, dd).

MS m/z: 457 (M+1).

### EXAMPLE 63

### 4-Cyano-1-[3-(5,11-dihydro-7-methoxy[1]benzoxepino[2,3-b]pyridin-5-ylidene)propyl]-4-phenylpiperidine

The titled compound was prepared by following the procedure of example 45, step 3, but replacing 4-(4-chlorophenyl)-4-hydroxypiperidine with 4-cyano-4-phenylpiperidine.

¹H-NMR (CDCl₃) δ: 1.97-2.06 (4H, m), 2.37-2.60 (6H, m), 2.85-2.90 (2H, m), 3.79 (3H, s), 5.27 (2H, brs), 6.08 (1H, t), 6.72-6.84 (3H, m), 7.24-7.58 (7H, m), 8.49 (1H, dd).

MS m/z: 452 (M+1).

### EXAMPLE 64

### 1-[3-(5,11-Dihydro-7-methoxy[1]benzoxepino[2,3-b]pyridin-5-ylidene)propyl]-4-phenylpiperidine

The titled compound was prepared by following the procedure of example 45, step 3, but replacing 4-(4-chlorophenyl)-4-hydroxypiperidine with 4-phenylpiperidine.

¹H-NMR (CDCl₃) δ: 1.73-1.79 (4H, m), 1.96-2.03 (2H, m), 2.37-2.52 (5H, m), 2.86-2.94 (2H, m), 3.77 (3H, s), 5.26 (2H, brs). 6.08 (1H, t), 6.72-6.83 (3H, m), 7.17-7.31 (6H, m), 7.56 (1H, dd), 8.49 (1H, dd).

MS m/z 426 (M+1).

### EXAMPLE 65

### 4-(4-Chlorophenyl)-1-[3-(5,11-dihydro-7-methoxy[1]benzoxepino[2,3-b]pyridin-5-ylidene)propyl]piperidine

The titled compound was prepared by following the procedure of example 45, step 3, but replacing 4-(4-chlorophenyl)-4-hydroxypiperidine with 4-(4-chlorophenyl)piperidine.

¹H-NMR (CDCl₃) δ: 1.68-1.74 (4H, m), 1.96-2.03 (2H, m), 2.36-2.48 (5H, m),2.89-2.94 (2H, m), 3.77 (3H, s), 5.27 (2H, brs), 6.07 (1H, t), 6.73-6.83 (3H, m), 7.10-7.27 (5H, m), 7.57 (1H, dd), 8.48 (1H, dd).

MS m/z: 461 (M+1).

### EXAMPLE 66

### 1-[3-(5,11-Dihydro-7-methoxy[1]benzoxepino[2,3-b]pyridin-5-ylidene)propyl]-4-piperidinopiperidine

The titled compound was prepared by following the procedure of example 45, step 3, but replacing 4-(4-chlorophenyl)-4-hydroxypiperidine with 4-piperidinopiperidine.

¹H-NMR (CDCl₃) δ: 1.40-2.00 (12H, m), 2.15-2.60 (9H, m), 2.80-2.92 (2H, m), 3.80 (3H, s), 5.28 (2H, brs), 6.05 (1H, t), 6.75-6.86 (3H, m), 7.30 (1H, dd), 7.55 (1H, dd), 8.46 (1H, dd).

MS m/z 434 (M+1).

### EXAMPLE 67

### 1-[3-(5,11-Dihydro-7-methoxy[1]benzoxepino[2,3-b]pyridin-5-ylidene)propyl]-4-(2-keto-1-benzimidazolinyl)piperidine

The titled compound was prepared by following the procedure of example 45, step 3, but replacing 4-(4-chlorophenyl)-4-hydroxypiperidine with 4-(2-keto-1-benzimidazolinyl)piperidine.

¹H-NMR (CDCl₃) δ: 1.75-1.79 (2H, m), 2.03-2.15 (2H, m), 2.38-2.52 (6H, m), 2.93-2.98 (2H, m), 3.78 (3H, s), 4.30-4.38 (1H, m), 5.30 (2H, brs), 6.10 (1H, t), 6.73-6.84 (3H, m), 7.01-7.03 (3H, m), 7.21-7.28 (2H, m), 7.59 (1H, dd), 8.48 (1H, dd).

MS m/z: 483 (M+1).

### EXAMPLE 68

### 1-[3-(5,11-Dihydro-7-methoxy[1]benzoxepino[2,3-b]pyridin-5-ylidene)propyl]-4-(2-keto-3-methyl-1-benzimidazolinyl)piperidine

The titled compound was prepared by following the procedure of example 36, but replacing of 4-(4-chlorophenyl)-1-[3-(6,11-dihydro-2-methoxydibenz[b,e]oxepin-11-ylidene)propyl]piperidin-4-ol with 1-[3-(5,11-dihydro-7-methoxy[1]benzoxepino[2,3-b]pyridin-5-ylidene)propyl]-4-(2-keto-1-benzimidazolinyl)piperidine.

¹H-NMR (CDCl₃) δ: 1.72-1.76 (2H, m), 2.09-2.14 (2H, m), 2.23-2.54 (6H, m), 2.91-2.96 (2H, m), 3.38 (3H, s), 3.77 (3H, s), 4.30-4.37 (1H, m), 5.27 (2H, brs), 6.08 (1H, t), 6.71-6.83 (3H, m), 6.93-7.06 (3H, m), 7.23-7.60 (2H, m), 8.08 (1H, dd), 8.48 (1H, dd). MS m/z: 497 (M+1).

### EXAMPLE 69

### 8-[3-(5,11-Dihydro-7-methoxy[1]benzoxepino[2,3-b]pyridin-5-ylidene)propyl]-1-phenyl-1,3,8-triazaspiro[4,5]decan-4-one

The titled compound was prepared by following the procedure of example 45, step 3, but replacing 4-(4-chlorophenyl)-4-hydroxypiperidine with 1-phenyl-1,3,8-triazaspiro[4,5]decan-4-one.

¹H-NMR (CDCl₃) δ: 1.65-1.70 (2H, m), 2.36-2.41 (2H, m), 2.53-2.79 (8H, m), 3.76 (3H, s), 4.70 (2H, s), 5.25 (2H, brs), 6.10 (1H, t), 6.71-6.88 (6H, m), 7.21-7.27 (3H, m), 7.58-7.61 (2H, m), 8.48 (1H, dd).

MS m/z: 497 (M+1).

### EXAMPLE 70

### 4-Anilino-4-carbamyl-1-[3-(5,11-dihydro-7-methoxy[1]benzoxepino[2,3-b]pyridin-5-ylidene)propyl]piperidine

The titled compound was prepared by following the procedure of example 45, step 3, but replacing 4-(4-chlorophenyl)-4-hydroxypiperidine with 4-anilino-4-carbamylpiperidine.

¹H-NMR (CDCl₃) d: 1.85-1.90 (2H, m), 2.03-2.08 (2H, m), 2.19-2.46 (6H, m), 2.62-2.67 (2H, m), 3.75 (3H, s), 3.97 (1H, brs), 5.27 (2H, brs), 5.53 (1H, brs), 6.03 (1H, t), 6.60 (2H, d), 6.70-6.85 (4H, m), 7.12-7.25 (4H, m), 7.53 (1H, dd), 8.46 (1H, dd).

MS m/z 485 (M+1).

### EXAMPLE 71

### 1-(4-Chlorophenyl)-4-[3-(5,11-dihydro-7-methoxy[1]benzoxepino[2,3-b]pyridin-5-ylidene)propyl]piperazine

The titled compound was prepared by following the procedure of example 45, step 3, but replacing 4-(4-chlorophenyl)-4-hydroxypiperidine with 1-(4-chlorophenyl)piperazine.

¹H-NMR (CDCl₃) δ: 2.36-2.53 (8H, m), 3.07-3.09 (4H, m), 3.76 (3H, s), 5.26 (2H, brs), 6.08 (1H, t), 6.72-6.81 (5H, m), 7.16-7.28 (3H, m), 7.56 (1H, dd), 8.49 (1H, dd).

MS m/z: 462 (M+1).

### EXAMPLE 72

### 1-[3-(5,11-Dihydro-7-methoxy[1]benzoxepino[2,3-b]pyridin-5-ylidene)propyl]-4-(2-pyrimidyl)piperazine

The titled compound was prepared by following the procedure of example 45, step 3, but replacing 4-(4-chlorophenyl)-4-hydroxypiperidine with 1-(2-pyrimidyl)piperazine.

¹H-NMR (CDCl₃) δ: 2.37-2.53 (8H, m), 3.74-3.83 (7H, m), 5.27 (2H, brs), 6.08 (1H, t), 6.45 (1H, t), 6.72-6.83 (3H, m), 7.25 (1H, dd), 7.56 (1H, dd), 8.27 (2H, d), 8.49 (1H, dd).

MS m/z: 430 (M+1).

### EXAMPLE 73

### 1-Cyclohexyl-4-[3-(5,11-dihydro-7-methoxy[1]benzoxepino[2,3-b]pyridin-5-ylidene)propyl]piperazine

The titled compound was prepared by following the procedure of example 45, step 3, but replacing 4-(4-chlorophenyl)-4-hydroxypiperidine with 1-cyclohexylpiperazine.

¹H-NMR (CDCl₃) δ: 1.12-1.27 (6H, m), 1.74-1.86 (6H, m), 2.18-2.52 (11H, m), 3.76 (3H, s), 5.26 (2H, brs), 6.04 (1H, t), 6.74-6.81 (3H, m), 7.23 (1H, dd), 7.55 (1H, dd), 8.48 (1H, dd).

MS m/z: 434 (M+1).

### EXAMPLE 74

### 1-[3-(5,11-Dihydro-7-methoxy[1]benzoxepino[2,3-b]pyridin-5-ylidene)propyl]-4-(2-furoyl)piperazine

The titled compound was prepared by following the procedure of example 45, step 3, but replacing 4-(4-chlorophenyl)-4-hydroxypiperidine with 1-(2-furoyl)piperazine.

¹H-NMR (CDCl₃) δ: 2.34-2.48 (8H, m), 3.71-3.74 (7H, s), 5.24 (2H, brs), 6.05 (1H, t), 6.42 (1H, dd), 6.70-6.80 (3H, m), 6.93 (1H, d), 7.23 (1H, dd), 7.42 (1H, d), 7.53 (1H, dd), 8.46 (1H, dd).

MS m/z: 446 (M+1).

### EXAMPLE 75

4-(3-Chlorophenyl)-1-[3-(5,11-dihydro-7-methoxy[1]benzoxepino[2,3-b]pyridin-5-ylidene)propyl]piperidin-4-ol

The titled compound was prepared by following the procedure of example 45, step 3, but replacing 4-(4-chlorophenyl)-4-hydroxypiperidine with 4-(3-chlorophenyl)-4-hydroxypiperidine.

¹H-NMR (CDCl₃) δ: 1.61-1.75 (2H, m), 1.98 (1H, brs), 1.99 (2H, dt), 2.25 (3H, s), 2.30-2.76 (8H, m), 3.73 (3H, s), 5.22 (2H, brs), 5.95 (0.1H, t, E isomer), 6.04 (0.9H, t, Z isomer), 6.71-6.89 (3H, m), 6.95 (1H, dd), 7.15-7.20 (0.3H, m, E isomer),7.21-7.35 (2.7H, m, Z isomer), 7.53 (0.9H, dd, Z isomer), 7.65 (0.1H, dd, E isomer), 8.35 (0.1H, dd, E isomer), 8.45 (0.9H, dd, Z isomer).

MS m/z: 477 (M+1)

### EXAMPLE 76

4-(2-Chlorophenyl)-1-[3-(5,11-dihydro-7-methoxy[1]benzoxepino[2,3-b]pyridin-5-ylidene)propyl]piperidin-4-ol

The titled compound was prepared by following the procedure of example 45, step 3, but replacing 4-(4-chlorophenyl)-4-hydroxypiperidine with ⁴-(²-chlorophenyl)-4-hydroxypiperidine.

¹H-NMR (CDCl₃) δ: 1.98-2.08 (2H, m), 2.24 (2H, dt), 2.38-2.78 (9H, m), 3.77 (3H, s), 5.27 (2H, brs), 6.08 (1H, t), 6.82-6.75 (3H, m), 7.28-7.19 (3H, m), 7.33 (1H, dd), 7.49 (1H, dd), 7.58 (1H, dd), 8.40 (0.1H, dd, Z isomer), 8.47 (0.9H, dd, E isomer).

MS m/z: 477 (M+1)

### EXAMPLE 77

1-[3-(5,11-Dihydro-7-methoxy[1]benzoxepino[2,3-b]pyridin-5-ylidene)propyl]-4-(4-fluorophenyl)piperidin-4-ol

The titled compound was prepared by following the procedure of example 45, step 3, but replacing 4-(4-chlorophenyl)-4-hydroxypiperidine with 4-(4-fluorophenyl)-4-hydroxypiperidine.

¹H-NMR (CDCl₃) δ: 1.58-1.72 (2H, m), 2.04 (2H, dt), 2.22-2.78 (9H, m), 3.75 (3H, s), 5.26 (2H, brs), 6.09 (1H, t), 6.70-6.88 (3H, m), 7.00 (2H, dd), 7.23 (1H, dd), 7.42 (2H, dd), 7.56 (1H, dd), 8.41 (1H, dd).

MS m/z: 461 (M+1)

### EXAMPLE 78

1-[3-(5,11-Dihydro-7-methoxy[1]benzoxepino[2,3-b]pyridin-5-ylidene)propyl]-4-(p-tolyl)piperidin-4-ol

The titled compound was prepared by following the procedure of example 45, step 3, but replacing 4-(4-chlorophenyl)-4-hydroxypiperidine with 4-(p-tolyl)-4-hydroxypiperidine.

¹H-NMR (CDCl₃) δ: 1.65-1.78 (2H, m), 2.02 (2H, dt), 2.31 (3H, s), 2.24-2.75 (9H, m), 3.75 (3H, s), 5.25 (2H, brs), 6.07 (1H, t), 6.72-6.84 (3H, m), 7.13 (2H, d), 7.23 (1H, dd), 7.34 (1H, d), 7.56 (1H, dd), 8.43 (1H, dd).

MS m/z: 457 (M+1)

### EXAMPLE 79

### 4-(3,4-Dichlorophenyl)-1-[3-(5,11-dihydro-7-methoxy[1]benzoxepino[2,3-b]pyridin-5-ylidene)propyl]piperidin-4-ol

The titled compound was prepared by following the procedure of example 45, step 3, but replacing 4-(4-chlorophenyl)-4-hydroxypiperidine with 4-(3,4-dichlorophenyl)-4-hydroxypiperidine.

¹HNMR (CDCl₃) d: 1.58-1.72 (2H, m), 1.84 (1H, brs), 2.02 (2H, td), 2.32-2.72 (8H, m), 3.76 (3H, s), 5.27 (2H, brs), 5.95 (0.1H, t, E isomer), 6.07 (0.9H, t, Z isomer), 6.72-6.85 (3H, M), 7.12-7.20 (0.2H, m, E isomer), 7.21-7.32 (0.18H, m, Z isomer), 7.32-7.45 (1H, m), 7.52-7.56 (2H, m), 8.37 (0.9H, dd, E siomer), 8.45 (0.1H, dd, Z isomer).

MS m/z: 512 (M+1)

### EXAMPLE 83

4-(5-Chloropyridin-2-yl)-1-[3-(5,11-dihydro-7-methoxy[1]benzoxepino[2,3-b]pyridin-5-ylidene)propyl]piperidin-4-ol

The titled compound was prepared by following the procedure of example 45, step 3, but replacing 4-(4-chlorophenyl)-4-hydroxypiperidine with 4-(5-chloropyridin-2-yl)-4-hydroxypiperidine.

¹H-NMR (CDCl₃) δ: 1.77-1.82 (2H, m), 2.36-2.94 (11H, m), 3.77 (3H, brs), 5.26 (2H, brs), 6.07 (1H, t), 6.76-6.84 (3H, m), 7.26 (1H, dd), 7.57 (1H, dd), 8.49-7.48 (1H, d), 8.42-8.53 (3H, m).

MS m/z: 478 (M+1)

### EXAMPLE 85

4-(5-Chloro-2-keto-1-benzimidazolinyl)-1-[3-(5,11-dihydro-7-methoxy[1]benzoxepino[2,3-b]pyridin-5-ylidene)propyl]piperidine

The titled compound was prepared by following the procedure of example 45, step 3, but replacing 4-(4-chlorophenyl)-4-hydroxypiperidine with 4-(5-chloro-2-keto-1-benzimidazolinyl)piperidine.

¹H-NMR (CDCl₃) δ: 1.68-1.72 (2H, m), 2.03-2.60 (8H, m), 2.90-3.02 (2H, m), 3.78 (3H, s), 4.32-4.21 (1H, m), 5.29 (2H, brs), 5.95 (0.1H, t, E isiomer), 6.08 (0.9H, t, Z isomer), 6.70-6.92 (3H, m), 7.02 (1H, dd), 7.08-7.20 (1H, m), 7.26 (1H, dd), 7.58 (0.9H, dd, Z isomer), 7.70 (0.1H, dd, E isomer), 8.42 (0.1H, dd, E isomer), 8.48 (0.9H, dd, Z isomer), 10.5 (1H, s). (NH is not observed in the spectrum)

MS m/z: 517 (M+1)

### EXAMPLE 86

4-(p-Chloroanilino)-1-[3-(5,11-dihydro-7-methoxy[1]benzoxepino[2,3-b]pyridin-5-ylidene)propyl]piperidine

The titled compound was prepared by following the procedure of example 45, step 3, but replacing 4-(4-chlorophenyl)-4-hydroxypiperidine with 4-(p-chloroanilino)piperidine.

¹H-NMR (CDCl₃) δ: 1.20-1.54 (2H, m), 1.85-2.20 (4H, m), 2.24-2.60 (4H, m), 2.73 (2H, m), 3.18 (1H, m), 3.77 (3H, s), 5.27 (2H, brs), 6.06 (1H, t), 6.47 (2H, m), 6.68-6.90 (3H, m), 7.07 (2H, m), 7.24 (1H, dd), 7.57 (1H, m), 8.48 (1Hdd). NH signal was not observed.

MS m/z: 476 (M+1)

### EXAMPLE 89

1-[3-(5,11-Dihydro-7-methoxy[1]benzoxepino[2,3-b]pyridin-5-ylidene)propyl]-4-(p-tosyl)piperazine

The titled compound was prepared by following the procedure of example 45, step 3, but replacing 4-(4-chlorophenyl)-4-hydroxypiperidine with -(p-tosyl)piperazine.

¹H-NMR (CDCl₃) δ: 2.20-2.54 (11H, m), 2.82-3.10 (4H, m), 3.73 (3H, s), 5.16 (2H, brs), 6.00 (1H, t), 6.66-6.85 (3H, m), 7.21 (1H, dd), 7.31 (2H, m), 7.51 (1H, dd), 7.61 (2H, m), 8.45 (1H, dd).

MS m/z: 506 (M+1)

### EXAMPLE 90

1′-[3-(5,11-Dihydro-7-methoxy[1]benzoxepino[2,3-b]pyridin-5-ylidene)propyl]spiro[isobenzofuran-1(3H),4′-piperidine]

The titled compound was prepared by following the procedure of example 45, step 3, but replacing 4-(4-chlorophenyl)-4-hydroxypiperidine with spiro[isobenzofuran-1(3H),4′-piperidine].

¹H-NMR (CDCl₃) δ: 1.62-1.82 (2H, m), 1.92 (2H, dt), 2.25-2.85 (8H, m), 3.76 (3H, s), 5.03 (2H, s), 5.30 (2H, brs), 6.11 (1H, t), 6.68-6.90 (3H, m), 7.02-7.34 (5H, m), 7.58 (1H, dd), 8.48 (1H, dd).

MS m/z: 455 (M+1)

### EXAMPLE 91

5-Chloro-1′-[3-(5,11-dihydro-7-methoxy[1]benzoxepino[2,3-b]pyridin-5-ylidene)propyl]spiro[isobenzofuran-1 (3H),4′-piperidine]

The titled compound was prepared by following the procedure of example 45, step 3, but replacing 4-(4-chlorophenyl)-4-hydroxypiperidine with 5-chlorospiro[isobenzofuran-1(3H),4′-piperidine].

1H-NMR (CDCl₃) δ: 1.69-1.74 (2H, m), 1.81-1.93 (2H, m), 2.30-2.44 (4H, m), 2.52-2.63 (2H, m), 2.71-2.75 (2H, m), 3.79 (3H, s), 5.00 (2H, s), 5.28 (2H, brs), 6.09 (1H, t), 6.73-6.84 (3H, m), 7.03 (1H, d), 7.17-7.28 (3H, m), 7.58 (1H, dd), 8.49 (1H, dd).

MS m/z: 489 (M+1)

### EXAMPLE 111

### 4-(4-Chlorophenyl)-1-[3-(5,11-dihydro[1]benzothiepino[2,3-b]pyridin-5-ylidene)propyl]piperidin-4-ol

The titled compound was prepared by following the procedure of example 45, but replacing 5,11-dihydro-7-methoxy[1]benzoxepino[2,3-b]pyridin-5-one with 5,11-dihydro[1]benzothiepino[2,3-b]pyridin-5-one.

1H-NMR (CDCl₃) d: 1.66-1.78 (3H, m), 2.04-2.65 (10H, m), 3.66 (1H, brd), 5.05 (1H, brd), 6.03 (1H, t), 7.04-7.46 (10H, m), 8.44 (1H, dd).

MS m/z: 463 (M+1)

### EXAMPLE 114

4-(4-Chlorophenyl)-1-[3-(5,11-dihydro-8-methoxy[1]benzoxepino[2,3-b]pyridin-5-ylidene)propyl]piperidin-4-ol

The titled compound was prepared by following the procedure of example 45, but replacing 5,11-dihydro-7-methoxy[1]benzoxepino[2,3-b]pyridin-5-one with 5,11-dihydro-8-methoxy[1]benzoxepino[2,3-b]pyridin-5-one.

1H-NMR (CDCl₃) d: 1.66-1.70 (3H, m), 1.98-2.09 (2H, m), 2.34-2.70 (8H, m), 3.75 (3H, s), 5.32 (2H, brs), 6.02 (1H, t), 6.39 (1H, d), 6.51 (1H, dd), 7.19-7.44 (6H, m), 7.57 (1H, dd), 8.49 (1H, dd).

MS m/z: 477 (M+1)

### EXAMPLE 115

4-(4-Chlorophenyl)-1-[3-(5,11-dihydro-7-methyl[1]benzoxepino[2,3-b]pyridin-5-ylidene)propyl]piperidin-4-ol

The titled compound was prepared by following the procedure of example 45, but replacing 5,11-dihydro-7-methoxy[1]benzoxepino[2,3-b]pyridin-5-one with 5,11-dihydro-7-methyl[1]benzoxepino[2,3-b]pyridin-5-one.

1H-NMR (CDCl₃) δ: 1.50 (1H, brs), 1.66-1.70 (2H, m), 1.98-2.10 (2H, m), 2.28 (3H, s), 2.34-2.42 (4H, m), 2.52-2.57 (2H, m), 2.66-2.70 (2H, m), 5.30 (2H, brs), 6.08 (1H, t), 6.76 (1H, d), 6.97 (1H, dd), 7.09 (1H, d), 7.24-7.44 (5H, m), 7.57 (1H, dd), 8.49 (1H, dd).

MS m/z: 461 (M+1)

### EXAMPLE 117

1-[3-(7-Chloro-5,11-dihydro[1]benzoxepino[2,3-b]pyridin-5-ylidene)propyl]-4-(4-chlorophenyl)piperidin-4-ol

The titled compound was prepared by following the procedure of example 45, but replacing 5,11-dihydro-7-methoxy[1]benzoxepino[2,3-b]pyridin-5-one with 7-chloro-5,11-dihydro[1]benzoxepino[2,3-b]pyridin-5-one.

1H-NMR (CDCl₃) δ: 1.66-1.71 (3H, m), 2.00-2.10 (2H, m), 2.36-2.44 (4H, m), 2.52-2.57 (2H, m), 2.66-2.70 (2H, m), 5.32 (2H, brs), 6.13 (1H, t), 6.78 (1H, d), 7.11 (1H, dd), 7.26-7.44 (5H, m), 7.58 (1H, dd), 8.51 (1H, dd).

MS m/z: 481 (M+1)

### EXAMPLE 118

1-[3-(7-Carboxy-5,11-dihydro[1]benzoxepino[2,3-b]pyridin-5-ylidene)propyl]-4-(4-chlorophenyl)piperidin-4-ol

A mixture of the product of example 169 (500 mg), potassium acetate (330 mg), palladium(II) diacetate (10 mg), 1,1′-bis(diphenylphosphino)ferrocene (93 mg), in dimethylsulfoxide (10 ml) was purged with carbon monoxide for 5 minutes and stirred under a carbon monoxide balloon at 60° C. for 3 hours. Water was added to the reaction mixture, the precipitation was filtered. The solid were dissolved with ethyl acetate and dilute sodium hydroxide solution. The aqueous layer was separated and neutralized with dilute hydrochloric acid. The precipitation was filtered to give the titled compound (250 mg).

1H-NMR (DMSO-d₆) δ: 1.45-1.55 (2H, m), 1.75-1.85 (2H, m), 2.36-2.62 (8H, m), 5.42 (2H, brs), 6.21 (1H, t), 6.90 (1H, d), 7.40-7.52 (5H, m), 7.75 (1H, dd), 7.83 (1H, dd), 7.95 (1H, d), 8.56 (1H, dd).

MS m/z: 491 (M+1)

### EXAMPLE 120

4-(4-Chlorophenyl)-1-[3-(7-carboxymethyl-5,11-dihydro[1]benzoxepino[2,3-b]pyridin-5-ylidene)propyl]piperidin-4-ol

To a solution of product of Example 290 (3.7 g) in methanol (74 ml), acetic acid (6 ml), and water (37 ml) were added sodium periodate (1.7 g) in water (15 ml) at 0° C., and the mixture was stirred at room temperature for 1 hour. To the reaction mixture were added amidosulfuric acid (1.2 g) and sodium chlorite (0.89 g) in water (10 ml), and the mixture was stirred at room temperature for 15 minutes. The reaction mixture was distilled off under reduced pressure into half volume. The residue was neutralized with 1N sodium hydroxide. The precipitation was filtered and washed with water to give the titled compound (2.6 g).

¹H-NMR (DMSO-d₆) δ: 1.45-1.50 (2H, m), 1.73-1.82 (2H, m), 2.24-2.50 (8H, m), 3.50 (2H, s), 4.84 (1H, brs), 5.24 (2H, brs), 6.13 (1H, t), 6.74 (1H, d), 7.06 (1H, dd), 7.21 (1H, d), 7.33-7.48 (5H, m), 7.74 (1H, dd), 8.50 (1H, dd).

### EXAMPLE 122

4-(4-Chlorophenyl)-1-[3-(7-dimethylaminocarbonylmethyl-5,11-dihydro[1]benzoxepino[2,3-b]pyridin-5-ylidene)propyl]piperidin-4-ol

The titled compound was prepared by following the procedure of example 134, but replacing the product of example 133 with the product of example 120.

¹H-NMR (CDCl₃) δ: 1.65-1.70 (2H, m), 1.95-2.06 (2H, m), 2.31-2.66 (9H, m), 2.93 (3H, s), 3.00 (3H, s), 3.61 (2H, s), 5.29 (2H, brs), 6.09 (1H, t), 6.78 (1H, d), 7.00 (1H, dd), 7.20-7.43 (6H, m), 7.56 (1H, dd), 8.42 (1H, dd).

MS m/z: 532 (M+1)

### EXAMPLE 123

1-[3-(7-(2-Carboxy)ethyl-5,11-dihydro[1]benzoxepino[2,3-b]pyridin-5-ylidene)propyl]-4-(4-chlorophenyl)-piperidin-4-ol

The titled compound was prepared by following the procedure of example 133, but replacing the product of example 48 with the product of example 288.

¹H-NMR (DMSO-d₆) δ: 1.44-1.49 (2H, m), 1.70-1.82 (2H, m), 2.22-2.48 (10H, m), 2.75 (2H, t), 4.82 (1H, brs), 5.23 (2H, brs), 6.14 (1H, t), 6.71 (1H, d), 7.04 (1H, dd), 7.17 (1H, d), 7.33-7.48 (5H, m), 7.72 (1H, dd), 8.49 (1H, dd).

MS m/z: 519 (M+1)

### EXAMPLE 128

4-(4-Chlorophenyl)-1-[3-(5,11-dihydro-7-propoxy[1]benzoxepino[2,3-b]pyridin-5-ylidene)propyl]piperidin-4-ol

The titled compound was prepared by following the procedure of example 46, but replacing ethyl iodide with propyl iodide.

¹H-NMR (CDCl₃) δ: 1.03 (3H, t), 1.65-1.70 (2H, m), 1.78 (2H, q), 1.98-2.09 (3H, m), 2.37-2.45 (4H, m), 2.51-2.56 (2H, m), 2.66-2.70 (2H, m), 3.88 (2H, t), 5.26 (2H, brs), 6.08 (1H, t), 6.72-6.84 (3H, m), 7.23-7.43 (5H, m), 7.58 (1H, dd), 8.43 (1H, dd).

MS m/z: 505 (M+1)

### EXAMPLE 130

4-(4-Chlorophenyl)-1-[3-(7-cyclopropylmethyloxy-5, 11-dihydro[1]benzoxepino[2,3-b]pyridin-5-ylidene)propyl]piperidin-4-ol

The titled compound was prepared by following the procedure of example 46, but replacing ethyl iodide with cyclopropylmethyl bromide.

¹H-NMR (CDCl₃) δ: 0.31-0.37 (2H, m), 0.60-0.67 (2H, m), 1.21-1.28 (1H, m), 1.66-1.72 (3H, m), 2.01-2.11 (2H, m), 2.37-2.71 (8H, m), 3.77 (2H, d), 5.27 (2H, brs), 6.08 (1H, t), 6.73-6.86 (3H, m), 7.23-7.44 (5H, m), 7.58 (1H, dd), 8.47 (1H, dd).

MS m/z: 517 (M+1)

### EXAMPLE 131

4-(4-Chlorophenyl)-1-[3-(5,11-dihydro-7-(2-dimetylaminoethyl)oxy)[1]benzoxepino[2, 3-b]pyridin-5-ylidene)propyl]piperidin-4-ol

The titled compound was prepared by following the procedure of example 46, but replacing ethyl iodide with 2-(dimethylamino)ethyl chloride hydrochloride.

¹H-NMR (CDCl₃) δ: 1.71-1.76 (2H, m), 2.12-2.21 (2H, m), 2.3 8 (6H, s), 2.40-2.79 (11H, m), 4.07 (2H, t), 5.28 (2H, brs), 6.07 (1H, t), 6.74-6.86 (3H, m), 7.27-7.46 (5H, m), 7.59 (1H, dd), 8.49 (1H, dd).

MS m/z: 534 (M+1)

### EXAMPLE 132

4-(4-Chlorophenyl)-1-[3-(5,11-dihydro-7-(tetrazol-5-yl)methyloxy)[1]benzoxepino[2,3-b]pyridin-5-ylidene)propyl]piperidin-4-ol

Step 1

4-(4-Chlorophenyl)-1-[3-(5,11-dihydro-7-(2-triphenylmethyltetrazol-5-yl)methyloxy)[1]benzoxepino[2,3-b]pyridin-5-ylidene)propyl]piperidin-4-ol was prepared by following the procedure of example 46, but replacing ethyl iodide with (2-triphenylmethyltetrazol-5-yl)methyl chloride.

¹H-NMR (CDCl₃) δ: 1.64-1.70 (3H, m), 2.02-2.15 (2H, m), 2.35-2.71 (8H, m),5.29 (2H, brs), 5.33 (2H, s), 6.03 (1H, t), 6.77 (1H, d), 6.83 (1H, dd), 6.96 (1H, d), 7.04-7.08 (6H, m), 7.23-7.45 (14H, m), 7.54 (1H, dd), 8.50 (1H, dd).

Step 2

A solution of the product of step 1 (530 mg) in acetone (2.5 ml), acetic acid (2.5 ml) and water (2.5 ml) was stirred at 55° C. for 30 minutes. The reaction mixture was distilled off under reduced pressure. The residue was washed with methanol to give the titled compound (280 mg).

¹H-NMR(DMSO-d₆) δ: 1.69-1.74 (2H, m), 1.99-2.09 (2H, m), 2.95-3.14 (8H, m), 5.18 (2H, brs), 5.20 (2H, s), 6.14 (1H, t), 6.76 (1H, d), 6.93 (1H, dd), 7.04 (1H, d), 7.39-7.48 (5H, m), 7.78 (1H, dd), 8.52 (1H, dd).

MS m/z: 545 (M+1)

### EXAMPLE 133

1-[3-(7-Carboxymethyloxy-5,11-dihydro[1]benzoxepino[2,3-b]pyridin-5-ylidene)propyl]-4-(4-chlorophenyl)piperidin-4-ol

To a solution of product of example 48 (3.0 g) in methanol (50 ml) was added IN sodium hydroxide solution (8 ml) and the mixture stirred at room temperature for 1 hour. The reaction mixture was distilled off under reduced pressure. The residue was dissolved with water and neutralized with 1N hydrochloric acid. The precipitation was filtered and washed with water to give the titled compound (2.6 g).

¹H-NMR (DMSO-d₆) δ: 1.48-1.53 (2H, m), 1.76-1.88 (2H, m), 2.32-2.60 (8H, m), 4.60 (2H, s), 5.18 (2H, brs), 6.16 (1H, t), 6.72-6.84 (3H, m), 7.34-7.48 (5H, m), 7.73 (1H, dd), 8.50 (1H, dd).

MS m/z: 521 (M+1)

### EXAMPLE 134

### 4-(4-Chlorophenyl)-1-[3-(5,11-dihydro-7-dimethylaminocarbonylmethyloxy[1]benzoxepino[2,3-b]pyridin-5-ylidene)propyl]piperidin-4-ol

To a solution of product of example 133 (420 mg) in dimethylformamide (17 ml) were added 1-hydroxybenzotriazol hydrate (250 mg), 1-(3-dimethylaminopropyl)-3-ethylcarbodiimide hydrochloride (310 mg), dimethylamine hydrochloride (270 mg) and triethylamine (0.45 ml), and the mixture stirred at room temperature for 12 hours. Water and chloroform were added to the reaction mixture, the organic layer was separated and washed with saturated aqueous sodium chloride, and dried with magnesium sulfate. The solvent was distilled off under reduced pressure to give the titled compound (380 mg).

¹H-NMR (CDCl₃) δ: 1.67-1.71 (2H, m), 1.95-2.11 (3H, m), 2.37-2.71 (8H, m), 2.97 (3H, s), 3.08 (3H, s), 4.64 (2H, s), 5.27 (2H, brs), 6.09 (1H, t), 6.74-6.82 (2H, m), 6.93 (1H, d), 7.24-7.44 (5H, m), 7.58 (1H, dd), 8.47 (1H, dd).

MS m/z: 548 (M+1)

### EXAMPLE 135

4-(4-Chlorophenyl)-1-[3-(5,11-dihydro-7-morpholinocarbonylmethyloxy[1]benzoxepino[2,3-b]pyridin-5-ylidene)propyl]piperidin-4-ol

The titled compound was prepared by following the procedure of example 134, but replacing dimethylamine hydrochloride with morpholine.

¹H-NMR (CDCl₃) δ: 1.67-1.71 (2H, m), 1.87 (1H, brs), 2.00-2.11 (2H, m), 2.38-2.71 (8H, m), 3.61-3.68 (8H, m), 4.65 (2H, s), 5.27 (2H, brs), 6.09 (1H, t), 6.74-6.83 (2H, m), 6.90 (1H, d), 7.25-7.44 (5H, m), 7.58 (1H, dd), 8.48 (1H, dd).

MS m/z: 590 (M+1)

### EXAMPLE 138

### 4-(4-Chlorophenyl)-1-[3-(5,11-dihydro-7-(1-ethoxycarbonyl-1-methylethyl)oxy[1]benzoxepino[2,3-b]pyridin-5-ylidene)propyl]piperidin-4-ol

The titled compound was prepared by following the procedure of example 46, but replacing ethyl iodide with ethyl 2-bromoisobutylate.

¹H-NMR (CDCl₃) δ: 1.27 (3H, t), 1.56 (6H, s), 1.63-1.71 (3H, m), 2.01-2.10 (2H, m), 2.35-2.70 (8H, m), 4.24 (2H, q), 5.28 (2H, brs), 6.05 (1H, t), 6.67-6.75 (2H, m), 6.87 (1H, d), 7.24-7.44 (5H, m), 7.56 (1H, dd), 8.49 (1H, dd).

MS m/z: 577 (M+1)

### EXAMPLE 139

1-[3-(7-(1-Carboxy-1-methylethyl)oxy-5,11-dihydro[1]benzoxepino[2,3-b]pyridin-5-ylidene)propyl]-4-(4-chlorophenyl)piperidin-4-ol

The titled compound was prepared by following the procedure of example 133, but replacing product of example 48 with product of example 138.

¹H-NMR (DMSO-d₆) δ: 1.45-1.52 (8H, m), 1.79-1.85 (2H, m), 2.28-2.53 (8H, m), 5.19 (2H, brs), 6.07 (1H, t), 6.69-6.73 (2H, m), 6.85 (1H, d), 7.33-7.47 (5H, m), 7.71 (1H, dd), 8.48 (1H, dd).

MS m/z: 549 (M+1)

### EXAMPLE 140

1-[3-(5,11-Dihydro-7-methoxy[1]benzoxepino[2,3-b]pyridin-5-ylidene)propyl]-4-(4-methoxyphenyl)piperidin-4-ol

The titled compound was prepared by following the procedure of example 45, step 3, but replacing 4-(4-chlorophenyl)-4-hydroxypiperidine with 4-(4-methoxyphenyl)-4-hydroxypiperidine.

¹H-NMR (CDCl₃) δ: 1.62-1.75 (2H, m), 2.08 (2H, dt), 2.41-2.76 (9H, m), 3.77 (3H, s), 3.78 (3H, s), 5.26 (2H, brs), 6.06 (1H, t), 6.75-6.871 (5H, m), 7.23 (1H, dd), 7.38 (2H, d), 7.57 (1H, dd), 8.45 (1H, dd).

MS m/z: 473 (M+1)

### EXAMPLE 141

4-(4-Cyanophenyl)-1-[3-(5,11-dihydro-7-methoxy[1]benzoxepino[2,3-b]pyridin-5-ylidene)propyl]piperidin-4-ol

The titled compound was prepared by following the procedure of example 45, step 3, but replacing 4-(4-chlorophenyl)-4-hydroxypiperidine with 4-(4-cyanophenyl)-4-hydroxypiperidine.

¹H-NMR (CDCl₃) δ: 1.58-1.70 (2H, m), 2.03 (2H, t), 2.31-2.64 (7H, m), 2.65-2.78 (2H, m), 3.75 (3H, s), 5.26 (2H, brs), 5.95 (0.1H, t, E isomer), 6.05 (0.9H, t, Z isomer), 6.70-6.80 (3H, m), 7.22 (1H, dd), 7.54-7.68 (5H, m), 8.31 (0.1H, dd, E iosmer), 8.39 (0.9H, dd, Z isomer).

MS m/z: 468 (M+1)

### EXAMPLE 142

1-[3-(5,11-Dihydro-7-methoxy[1]benzoxepino[2,3-b]pyridin-5-ylidene)propyl]-4-(4-hydroxyphenyl)piperidin-4-ol

The titled compound was prepared by following the procedure of example 45, step 3, but replacing 4-(4-chlorophenyl)-4-hydroxypiperidine with 4-(4-hydroxyphenyl)-4-hydroxypiperidine.

¹HNMR (CDCl₃) δ: 1.76-1.88 (2H, m). 2.08-2.22 (2H, m), 2.45-2.95 (9H, m), 3.76 (3H, s), 5.28 (2H, brs), 5.95 (0.3H, t, E isomer), 6.04 (0.7H, t, Z iosmer), 6.69-6.72 (3H, m), 6.90 (2H, d), 7.20-7.30 (3H, m), 7.56 (0.7H, dd, Z isomer), 7.67 (0.3H, dd, E isomer), 8.46 (0.7H, dd, Z isomer), 8.47 (0.3H, dd, E isomer). OH signal was not observed.

MS m/z: 473 (M+1)

### EXAMPLE 143

1-[3-(5,11-Dihydro-7-methoxy[1]benzoxepino[2,3-b]pyridin-5-ylidene)propyl]-4-(4-fluoro-3-methylphenyl)piperidin-4-ol

The titled compound was prepared by following the procedure of example 45, step 3, but replacing 4-(4-chlorophenyl)-4-hydroxypiperidine with 4-(4-fluoro-3-methylphenyl)-4-hydroxypiperidine.

¹H-NMR (CDCl₃) δ: 1.62-1.75 (2H, m), 2.05 (1H, brs), 2.09 (2H, dt), 2.25 (3H, s), 2.30-2.76 (8H, m), 3.76 (3H, s), 5.26 (2H, brs), 5.96 (0.1H, t, E isomer), 6.07 (0.9H, t, Z isomer), 6.75-6.89 (3H, m), 6.93 (1H, t), 7.11-7.20 (0.3H, m, E isomer), 7.21-7.35 (0.24H, m, Z isomer), 7.56 (0.9H, dd, E isomer), 7.67 (0.1H, dd, E isomer), 8.38 (0.1H, dd, E isomer), 8.45 (0.9H, dd, Z isomer).

MS m/z: 475 (M+1)

### EXAMPLE 144

4-(3,4-difluorophenyl)-1-[3-(5,11-dihydro-7-methoxy[1]benzoxepino[2,3-b]pyridin-5-ylidene)propyl]piperidin-4-ol

The titled compound was prepared by following the procedure of example 45, step 3, but replacing 4-(4-chlorophenyl)-4-hydroxypiperidine with 4-(3,4-difluorophenyl)-4-hydroxypiperidine.

¹H-NMR (CDCl₃) δ: 1.58-1.72 (2H, m), 1.96 (2H, dt), 2.33-2.71 (8H, m), 3.73 (3H, s), 5.23 (2H, brs), 5.94 (0.1H, t, E isomer), 6.04 (0.9H, t, Z isomer), 8.38-8.36 (0.9H, m, Z isomer), 6.68-6.79 (3H, m), 6.98-7.38 (4H, m), 7.50-7.62 (0.9H, m, Z isomer), 7.63-7.68 (0.1H, m, E isomer), 8.29-8.32 (0.1H, m, E isomer), 8.32-8.44 (0.9H, m, Z isomer). OH signal was not observed.

MS m/z: 479 (M+1)

### EXAMPLE 145

4-(4-Chloro-3-trifuluoromethylphenyl)-1-[3-(5,11-dihydro-7-methoxy[1]benzoxepino[2, 3-b]pyridin-5-ylidene)propyl]piperidin-4-ol

The titled compound was prepared by following the procedure of example 45, step 3, but replacing 4-(4-chlorophenyl)-4-hydroxypiperidine with 4-(4-chloro-3-trifluoromethylphenyl)-4-hydroxypiperidine.

¹H-NMR (CDCl₃) δ: 1.62-1.74 (2H, m), 2.10 (2H, dt), 2.35-2.80 (8H, m), 2.42 (1H, brs), 3.76 (3H, s), 5.26 (2H, brs), 6.07 (0.9H, t, Z isomer), 6.03 (0. 1H, t, E isomer), 6.82-6.71 (3H, m), 7.24 (1H, dd), 7.43 (1H, d), 7.56 (1.8H, dd, Z isomer), 7.65 (0.2H, dd, E isomer) 7.83 (1H, d), 8.36 (0.1H, dd, E isomer), 8.44 (0.9H, dd, Z iosmer),

MS m/z: 545 (M+1)

### EXAMPLE 146

4-(3,5-dichlorophenyl)-1-[3-(5,11-dihydro-7-methoxy[1]benzoxepino[2,3-b]pyridin-5-ylidene)propyl]piperidin-4-ol

The titled compound was prepared by following the procedure of example 45, step 3, but replacing 4-(4-chlorophenyl)-4-hydroxypiperidine with 4-(3,5-dichlorophenyl)-4-hydroxypiperidine.

¹H-NMR (CDCl₃) δ: 1.58-2.22 (5H, m), 2.38-2.77 (8H, m), 3.76 (3H, s), 5.26 (2H, brs), 5.92 (0.1H, t, E isomer), 6.07 (0.9H, t, Z isomer), 6.83-6.71 (3H, m), 7.19-7.42 (4H, m), 7.56 (0.9H, dd, Z isomer), 7.68 (0.1H, dd, E isomer), 8.38 (0.1H, dd, E isomer), 8.45 (0.9H, dd, Z isomer).

MS m/z: 512 (M+1)

### EXAMPLE 147

1-[3-(5,11-Dihydro-7-methoxy[1]benzoxepino[2,3-b]pyridin-5-ylidene)propyl]-4-(2-pyridyl)piperidin-4-ol

The titled compound was prepared by following the procedure of example 45, step 3, but replacing 4-(4-chlorophenyl)-4-hydroxypiperidine with 4-(2-pyridyl)-4-hydroxypiperidine

¹H-NMR (CDCl₃) d: 1.54-1.65 (2H, m), 2.06 (2H, dt), 2.07 (1H, brs), 2.35-2.62 (7H, m), 2.73-2.87 (2H, m), 3.78 (3H, s), 5.28 (2H, brs), 6.08 (1H, t), 6.72-6.85 (3H, m), 7.14-7.29 (2H, m), 7.57 (1H, d), 7.70 (1H, dd), 8.48 (2H, dd).

MS m/z: 444 (M+1)

### EXAMPLE 148

1-[3-(5,11-Dihydro-7-methoxy[1]benzoxepino[2,3-b]pyridin-5-ylidene)propyl]-4-(3-pyridyl)piperidin-4-ol

The titled compound was prepared by following the procedure of example 45, step 3, but replacing 4-(4-chlorophenyl)-4-hydroxypiperidine with 4-(3-pyridyl)-4-hydroxypiperidine.

¹H-NMR (CDCl₃) δ: 1.65-1.78 (2H, m), 2.08 (2H, dt), 2.37-2.88 (7H, m), 2.63-2.79 (2H, m), 3.78 (3H, s), 5.28 (2H, brs), 6.02 (0.1H, t, E isomer), 6.07 (0.9H, t, Z isomer), 6.70-6.84 (3H, m), 7.22-7.32 (3H, m), 7.56 (1H, dd), 7.77 (1H, dd), 8.46 (0.9H, d), 8.57 (0.1H, dd, E isomer), 8.73 (1H, dd).

MS m/z: 444 (M+1)

### EXAMPLE 149

1-[3-(5,11-Dihydro-7-methoxy[1]benzoxepino[2,3-b]pyridin-5-ylidene)propyl]-4-(4-pyridyl)piperidin-4-ol

The titled compound was prepared by following the procedure of example 45, step 3, but replacing 4-(4-chlorophenyl)-4-hydroxypiperidine with 4-(4-pyridyl)-4-hydroxypiperidine.

¹H-NMR (CDCl₃) δ: 1.58-1.72 (2H, m), 2.03 (2H, dt), 2.34-2.89 (8H, m), 2.96 (1H, brs), 3.76 (3H, s), 5.25 (2H, brs), 6.06 (1H, t), 6.72-6.83 (3H, m), 7.24 (1H, dd), 7.37 (2H, dd), 7.56 (1H, dd), 8.45 (1H, dd), 8.48 (2H, dd).

MS m/z: 444 (M+1)

### EXAMPLE 150

1-[3-(5,11-Dihydro-7-methoxy[1]benzoxepino[2,3-b]pyridin-5-ylidene)propyl]-4-(4-trifluoromethylphenyl)piperidin-4-ol

The titled compound was prepared by following the procedure of example 45, step 3, but replacing 4-(4-chlorophenyl)-4-hydroxypiperidine with 4-(4-trifluoromethylphenyl)-4-hydroxypiperidine.

¹H-NMR (CDCl₃) δ: 1.64-1.75 (2H, m), 2.01 (1H, brs), 2.16 (2H, dt), 2.38-2.86 (8H, m), 3.76 (3H, s), 5.26 (2H, brs), 6.04 (1H, t), 6.72-6.84 (3H, m), 7.23 (1H, dd), 7.56 (5H, m), 8.42 (1H, dd).

MS m/z: 511 (M+1)

### EXAMPLE 151

4-(4-Chlorophenyl)-1-[3-(5,11-dihydro-7-hydroxy[1]benzoxepino[2,3-b]pyridin-5-ylidene)propyl]piperidine

The titled compound was prepared by following the procedure of example 44, step 2, but replacing 4-(4-chlorophenyl)-4-hydroxypiperidine with 4-(4-chlorophenyl)piperidine.

¹H-NMR (CDCl₃) δ: 1.62-1.92 (4H, m), 1.94-2.18 (2H, m), 2.28-2.64 (5H, m), 2.99 (2H, m), 5.25 (2H, brs), 6.00 (1H, t), 6.60-6.82 (3H, m), 7.02-7.36 (5H, m), 7.50 (1H, dd), 8.47 (1H, dd). OH signal was not observed.

MS m/z: 447 (M+1)

### EXAMPLE 152

4-(4-Chlorophenyl)-1-[3-(5,11-dihydro-7-ethoxy[1]benzoxepino[2,3-b]pyridin-5-ylidene)propyl]piperidine

The titled compound was prepared by following the procedure of example 46, but replacing the product of example 44 with the product of example 151.

¹H-NMR (CDCl₃) δ: 1.40 (3H, t), 1.52-2.14 (6H, m), 2.30-2.57 (5H, m), 2.94 (2H, m), 4.00 (2H, q), 5.28 (2H, brs), 6.07 (1H, t), 6.68-6.86 (3H, m), 7.05-7.36 (5H, m), 7.58 (1H, m), 8.49 (1H, m).

MS m/z: 475 (M+1)

### EXAMPLE 153

4-(4-Chlorophenyl)-1-[3-(5,11-dihydro-7-ethoxycarbonylmethyloxy[1]benzoxepino[2,3 -b]pyridin-5-ylidene)propyl]piperidine

The titled compound was prepared by following the procedure of example 48, but replacing the product of example 44 with the product of example 151.

¹H-NMR (CDCl₃) δ: 1.29 (3H, t), 1.56-1.85 (4H, m), 1.99 (2H, dt), 2.28-2.55 (5H, m), 2.91 (2H, m), 4.27 (2H, q), 4.58 (2H, s), 5.28 (2H, brs), 6.09 (1H, t), 6.68-6.95 (3H, m), 7.07-7.32 (5H, m), 7.58 (1H, dd), 8.49 (1H, dd).

MS m/z: 533 (M+1)

### EXAMPLE 154

1-[3-(7-(Carboxymethyloxy-5,11-dihydro[1]benzoxepino[2,3-b]pyridin-5-ylidene)propyl]-4-(4-chlorophenyl)piperidine

The titled compound was prepared by following the procedure of example 133, but replacing the product of example 48 with the product of example 153.

¹H-NMR (CD₃OD) δ: 1.82-2.17 (4H, m), 2.69 (2H, m), 2.86 (1H, m), 3.07 (2H, m), 3.30 (2H, m), 3.57 (2H, m), 4.57 (2H, s), 5.21 (2H, brs), 6.10 (1H, t), 6.70-7.04 (3H, m), 7.16-7.38 (4H, m), 7.44 (1H, m), 7.77 (1H, m), 8.47 (1H, m). COOH signal was not observed.

MS m/z: 505 (M+1)

### EXAMPLE 155

4-(4-Chlorophenyl)-1-[3-(5,11-dihydro-7-dimethylaminocarbonylmethyloxy[1]benzoxe pino[2,3-b]pyridin-5-ylidene)propyl]piperidine

The titled compound was prepared by following the procedure of example 134, but replacing the product of example 133 with the product of example 154.

¹H-NMR (CDCl₃) δ: 1.58-1.92 (4H, m), 2.04 (2H, m), 2.30-2.68 (5H, m), 2.93 (2H, m), 2.98 (3H, s), 3.08 (3H, s), 4.65 (2H, s), 5.28 (2H, brs), 6.07 (1H, t), 6.70-6.98 (3H, m), 7.08-7.36 (5H, m), 7.60 (1H, m), 8.50 (1H, m).

MS m/z: 532 (M+1)

### EXAMPLE 156

1-[3-(7-(2-Acetoxyethyl)oxy-5,11-dihydro[1]benzoxepino[2,3-b]pyridin-5-ylidene)propyl]piperidine

The titled compound was prepared by following the procedure of example 50, but replacing the product of example 44 with the product of example 151.

¹H-NMR (CDCl₃) δ: 1.55-1.88 (4H, m), 1.90-2.32 (2H, m), 2.10 (3H, s), 2.28-2.60 (5H, m), 2.82-3.02 (2H, m), 4.14 (2H, dd), 4.41 (2H, dd), 5.29 (2H, brs), 6.08 (1H, t), 6.72-6.90 (3H, m), 7.18-7.34 (5H, m), 7.57 (1H, m), 8.50 (1H, m).

MS m/z: 533 (M+1)

### EXAMPLE 157

4-(4-Chlorophenyl)-1-[3-(5,11-dihydro-7-(2-hydroxyethyl)oxy[1]benzoxepino[2,3-b]pyridin-5-ylidene)propyl]piperidine

The titled compound was prepared by following the procedure of example 51, but replacing the product of example 50 with the product of example 156.

¹H-NMR (CD₃OD) δ: 1.66-1.98 (4H, m), 2.40-2.73 (5H, m), 2.82-2.94 (2H, m), 3.22 (2H, m), 3.84 (2H, dd), 4.01 (2H, dd), 5.23 (2H, brs), 6.13 (1H, t), 6.64-6.98 (3H, m), 7.13-7.34 (4H, m), 7.45 (1H, m), 7.77 (1H, m), 8.47 (1H, m). OH signal was not observed.

MS m/z: 491 (M+1)

### EXAMPLE 158

4-(4-Chlorophenyl)-1-[3-(5,11-dihydro-7-(1-ethoxycarbonyl-1-methylethyl)oxy[1]benzoxepino[2,3-b]pyridin-5-ylidene)propyl]piperidine The titled compound was prepared by following the procedure of example 138, but replacing the product of example 44 with the product of example 151.

¹H-NMR (CDCl₃) d: 1.28 (3H, t), 1.56 (6H, s), 1.56-1.85 (4H, m), 1.97 (2H, dt), 2.28-2.55 (5H, m), 2.93 (2H, m), 4.24 (2H, q), 5.28 (2H, brs), 6.04 (1H, t), 6.62-6.95 (3H, m), 7.07-7.32 (5H, m), 7.57 (1H, dd), 8.50 (1H, dd).

MS m/z: 561 (M+1)

### EXAMPLE 159

### 1-[3-(7-(1-Carboxy-1-methylethyl)oxy-5,11-dihydro[1]benzoxepino[2,3-b]pyridin-5-ylidene)propyl]-4-(4-chlorophenyl)piperidine

The titled compound was prepared by following the procedure of example 133, but replacing the product of example 48 with the product of example 158.

¹H-NMR (CD₃OD) d: 1.50 (6H, s), 1.82-2.18 (4H, m), 2.70 (2H, m), 2.87 (1H, m), 3.12 (2H, m), 3.30 (2H, m), 3.60 (2H, m), 5.25 (2H, brs), 6.07 (1H, t), 6.67-7.04 (3H, m), 7.16-7.38 (4H, m), 7.58 (1H, m), 7.96 (1H, m), 8.52 (1H, m). COOH signal was not observed.

MS m/z: 533 (M+1)

### EXAMPLE 160

1-[3-(8-Bromo-5,11-dihydro[1]benzoxepino[2,3-b]pyridin-5-ylidene)propyl]-4-(4-chlorophenyl)piperidine

The titled compound was prepared by following the procedure of example 65, but replacing the product of example 45, step 2 with the product of example 54, step 1.

¹H-NMR (CDCl₃) d: 1.50-1.86 (4H, m), 1.98 (2H, m), 2.26-2.60 (5H, m), 2.88 (2H, m), 5.30 (2H, brs), 6.09 (1H, t), 6.96-7.36 (8H, m), 7.57 (1H, dd), 8.51 (1H, dd).

MS m/z: 509, 511 (M+1)

### EXAMPLE 161

1-[3-(8-Carboxy-5,11-dihydro[1]benzoxepino[2,3-b]pyridin-5-ylidene)propyl]-4-(4-chlorophenyl)piperidine

To a solution of 1-[3-(8-Bromo-5,11-dihydro[1]benzoxepino[2,3-b]pyridin-5-ylidene)propyl]-4-(4-chlorophenyl)piperidine (Example 160) (130 mg) in THF (1.0 ml) was added 1.6M n-butyllithium hexane solution (0.17 ml) at −78° C. After stirring 10 minutes at the same temperature, CO₂ (dry-ice) was added to the mixture. After being warmed to ambient temperature, the mixture was stirred for 30 minutes at the same temperature. The mixture was concentrated in vacuo. The resulting oil was purified by silica gel chromatography eluted with dichloromethane -methanol (5:1) to give the titled compound

¹H-NMR (CD₃OD) δ: 1.55-1.95 (4H, m), 2.17 (2H, dt), 2.32-2.78 (5H, m), 3.00 (2H, m), 5.30 (2H, brs), 6.19 (1H, t), 7.08-7.54 (8H, m), 7.76 (1H, dd), 8.45 (1H, dd). COOH signal was not observed.

MS m/z: 475 (M+1)

### EXAMPLE 162

1-[3-(7-Bromo-5,11-dihydro[1]benzoxepino[2,3-b]pyridin-5-ylidene)propyl]-4-(4-chlorophenyl)piperidin-4-ol

The titled compound was prepared by following the procedure of example 45, but replacing 5,11-dihydro-7-methoxy[1]benzoxepino[2,3-b]pyridin-5-one with 8-bromo-5,11-dihydro[1]benzoxepino[2,3-b]pyridin-5-one.

1H-NMR (CDCl₃) δ: 1.60-1.71 (3H, m), 1.98-2.09 (2H, m), 2.34-2.69 (8H, m), 5.32 (2H, brs), 6.13 (1H, t), 6.73 (1H, d), 7.22-7.44 (7H, m), 7.57 (1H, dd), 8.52 (1H, dd).

MS m/z: 525, 527 (M+1)

### EXAMPLE 163

### 4-(4-Chlorophenyl)-1-[3-(5,11-dihydro-7-ethyl[1]benzoxepino[2,3-b]pyridin-5-ylidene)propyl]piperidin-4-ol

The titled compound was prepared by following the procedure of example 45, but replacing 5,11-dihydro-7-methoxy[1]benzoxepino[2,3-b]pyridin-5-one with 5,11-dihydro-7-ethyl[1]benzoxepino[2,3-b]pyridin-5-one.

1H-NMR (CDCl₃) d: 1.23 (3H, t), 1.52 (1H, brs), 1.66-1.71 (2H, m), 1.98-2.06 (2H, m), 2.35-2.70 (11H, m), 5.31 (2H, brs), 6.09 (1H, t), 6.79 (1H, d), 7.01 (1H, dd), 7.11 (1H, d), 7.25-7.44 (5H, m), 7.58 (1H, dd), 8.49 (1H, dd).

MS m/z: 475 (M+1)

### EXAMPLE 164

### 4-(4-Chlorophenyl)-1-[3-(5,11-dihydro-8-vinyl[1]benzoxepino[2,3-b]pyridin-5-ylidene)propyl]piperidin-4-ol

The titled compound was prepared by following the procedure of example 45, but replacing 5,11-dihydro-7-methoxy[1]benzoxepino[2,3-b]pyridin-5-one with 5,11-dihydro-8-vinyl[1]benzoxepino[2,3-b]pyridin-5-one.

1H-NMR (CDCl₃) d: 1.66-1.71 (3H, m), 2.00-2.10 (2H, m), 2.36-2.70 (8H, m), 5.22 (2H, d), 5.34 (2H, brs), 5.70 (1H, d), 6.11 (1H, t), 6.61 (1H, dd), 6.89 (1H, d), 6.99 (1H, dd), 7.24-7.44 (6H, m), 7.58 (1H, dd), 8.49 (1H, dd).

MS m/z: 473 (M+1)

### EXAMPLE 165

4-(4-Chlorophenyl)-1-[3-(5,11-dihydro-8-ethyl[1]benzoxepino[2,3-b]pyridin-5-ylidene)propyl]piperidin-4-ol

A mixture of the product of example 164 (100 mg) and Pd-C (20 mg) in ethanol(2 ml) stirred under a hydrogen balloon at room temperature for 1 hour. The mixture was filtered through the celite and distilled off under reduced pressure. The residue was purified by preparative thin layer chromatography eluting with chloroform-methanol (15:1) to give the titled compound (50 mg).

1H-NMR (CDCl₃) δ: 1.22 (3H, t), 1.55-1.77 (3H, m), 2.00-2.13 (2H, m), 2.33-2.74 (10H, m), 5.32 (2H, brs), 6.07 (1H, t), 6.70 (1H, d), 6.78 (1H, dd), 7.19-7.44 (6H, m), 7.57 (1H, dd), 8.49 (1H, dd).

MS m/z: 475 (M+1)

### EXAMPLE 166

4-(4-Chlorophenyl)-1-[3-(5,11-dihydro-9-methoxy[1]benzoxepino[2,3-b]pyridin-5-ylidene)propyl]piperidin-4-ol

The titled compound was prepared by following the procedure of example 45, but replacing 5,11-dihydro-7-methoxy[1]benzoxepino[2,3-b]pyridin-5-one with 5,11-dihydro-9-methoxy[1]benzoxepino[2,3-b]pyridin-5-one.

1H-NMR (CDCl₃) δ: 1.65-1.70 (2H, m), 1.95-2.06 (2H, m), 2.15 (1H, brs), 2.37-2.67 (8H, m), 3.83 (3H, s), 5.43 (2H, brs), 6.09 (1H, t), 6.79-6.91 (3H, m), 7.22-7.43 (5H, m), 7.57 (1H, dd), 8.44 (1H, dd).

MS m/z: 477 (M+1)

### EXAMPLE 167

4-(4-Chlorophenyl)-1-[3-(5,11-dihydro[1]benzoxepino[4,3-c]pyridin-5-ylidene)propyl]piperidin-4-ol

The titled compound was prepared by following the procedure of example 45, but replacing 5,11-dihydro-7-methoxy[1]benzoxepino[2,3-b]pyridin-5-one with 5,11-dihydro[1]benzoxepino[4,3-c]pyridin-5-one.

1H-NMR (CDCl₃) δ: 1.67-1.71 (2H, m), 1.97-2.08 (2H, m), 2.16 (1H, s), 2.40-2.69 (8H, m), 5.16 (2H, brs), 6.14 (1H, t), 6.80 (1H, dd), 6.91-6.97 (1H, m), 7.13-7.19 (1H, m), 7.26-7.44 (6H, m), 7.50-8.54 (2H, m).

MS m/z: 447 (M+1)

### EXAMPLE 168

4-(4-Chlorophenyl)-1-[3-(5,11-dihydro[1]benzoxepino[4,3-d]pyrimidin-5-ylidene)propyl]piperidin-4-ol

The titled compound was prepared by following the procedure of example 45, but replacing 5,11-dihydro-7-methoxy[1]benzoxepino[2,3-b]pyridin-5-one with 5,11-dihydro[1]benzoxepino[4,3-d]pyrimidin-5-one.

1H-NMR (CDCl₃) δ: 1.68-1.72 (2H, m), 1.90 (1H, brs), 2.06-2.19 (2H, m), 2.41-2.78 (8H, m), 5.20 (2H, s), 6.12 (1H, t), 7.14-7.45 (8H, m), 8.72 (1H, s), 8.97 (1H, s).

MS m/z: 448 (M+1)

### EXAMPLE 169

4-(4-Chlorophenyl)-1-[3-(5,11-dihydro-7-trifluoromethanesulfonyloxy[1]benzoxepino[2,3-b]pyridin-5-ylidene)propyl]piperidin-4-ol

To a solution of product of example 44 (1.0 g) in pyridine (10 ml) was added trifluoromethanesulfonic acid anhydride (0.55 ml) at 0° C., and the mixture was stirred at room temperature for 1 hour. Water and diethyl ether were added to the reaction mixture, the organic layer was separated and washed with saturated aqueous sodium chloride, and dried with magnesium sulfate. The solvent was distilled off under reduced pressure, and the residue was purified by silica gel chromatography eluting with ethyl acetate-methanol (10:1) to give the titled compound (1.1 g).

1H-NMR (CDCl₃) δ: 1.56 (1H, brs), 1.66-1.71 (2H, m), 1.97-2.09 (2H, m), 2.35-2.69 (8H, m), 5.35 (2H, brs) 6.15 (1H, t), 6.88 (1H, d), 7.05 (1H, dd), 7.21-7.44 (6H, m), 7.60 (1H, dd), 8.54 (1H, dd).

MS m/z: 595 (M+1)

### EXAMPLE 170

1-[3-(7-Allyl-5,11-dihydro[1]benzoxepino[2,3-b]pyridin-5-ylidene)propyl]-4-(4-chlorophenyl)piperidin-4-ol

A mixture of the product of example 169 (240 mg), allyltributyltin (0. 1 9 ml), dichlorobis(triphenylphosphine)palladium(II) (30 mg) and lithium chloride (76 mg), in dimethylformamide (3 ml) was heated under argon at 120° C. for 2 hours. Aqueous ammonium fluoride solution and ethyl acetate were added to the reaction mixture, the organic layer was separated and washed with saturated aqueous sodium chloride, and dried with magnesium sulfate. The solvent was distilled off under reduced pressure, and the residue was purified by silica gel chromatography eluting with chloroform-methanol (10:1) to give the titled compound (180 mg).

1H-NMR (CDCl3) δ: 1.62-1.72 (3H, m), 2.03-2.11 (2H, m), 2.39-2.73 (8H, m), 3.31 (2H, d), 5.04-5.11 (2H, m), 5.29 (2H, brs), 5.87-6.02 (1H, m), 6.06 (1H, t), 6.77 (1H, d), 6.99 (1H, dd), 7.10 (1H, d), 7.23-7.43 (5H, m), 7.57 (1H, dd), 8.40 (1H, dd).

### EXAMPLE 171

1-[3-(7-(2-t-Butoxycarboxy)ethenyl-5,11-dihydro[1]benzoxepino[2,3-b]pyridin-5-ylidene)propyl]-4-(4-chlorophenyl)piperidin-4-ol

A mixture of the product of example 169 (1.7 g), t-butyl acrylate (0.85 ml), triethylamine (2.5 ml), 1,1′-bis(diphenylphosphino)ferrocene (250 mg) and palladium(II) diacetate (33 mg) in dimethylformamide (3 ml) was heated under argon at 90° C. for 24 hours. Water ethyl acetate were added to the reaction mixture, the organic layer was separated and washed with saturated aqueous sodium chloride, and dried with magnesium sulfate. The solvent was distilled off under reduced pressure, and the residue was purified by silica gel chromatography eluting with ethyl acetate-methanol (30:1) to give the titled compound (780 mg).

1H-NMR (CDCl₃) δ: 1.45 (9H, s), 1.63-1.71 (3H, m), 1.98-2.10 (2H, m), 2.35-2.72 (8H, m), 5.35 (2H, brs), 6.15 (1H, t), 6.26 (1H, d), 6.83 (1H, d), 7.22-7.44 (7H, m), 7.53 (1H, d), 7.58 (1H, dd), 8.52 (1H, dd).

### EXAMPLE 172

1-[3-(7-(2-Carboxy)ethenyl-5,11-dihydro[1]benzoxepino[2,3-b]pyridin-5-ylidene)propyl]-4-(4-chlorophenyl)piperidin-4-ol

The product of example 171 (330 mg) was dissolved with 4N hydrochloric acid 1,4-dioxane solution (4 ml), and stirred at room temperature for 1 hour. The solvent was distilled off under reduced pressure. Water was added to the residue, and neutralized with sodium hydroxide solution. The precipitation was filtered to give the titled compound (190 mg).

1H-NMR (DMSO-d₆) δ: 1.45-1.52 (2H, m), 1.72-1.84 (2H, m), 2.25-2.58 (8H, m), 5.25 (2H, brs), 6.28 (1H, t), 6.43 (1H, d), 6.82 (1H, d), 7.34-7.60 (8H, m), 7.75 (1H, dd), 8.52 (1H, dd).

### EXAMPLE 173

4-(4-Chlorophenyl)-1-[3-(5,11-dihydro-7-propargyloxy[1]benzoxepino[2,3-b]pyridin-5-ylidene)propyl]piperidin-4-ol

The titled compound was prepared by following the procedure of example 46, but replacing ethyl iodide with propargyl chloride.

1H-NMR (CDCl₃) δ: 1.66-1.71 (2H, m), 1.79 (1H, brs), 1.99-2.10 (2H, m), 2.35-2.71 (9H, m), 4.66 (2H, d), 5.28 (2H, brs), 6.10 (1H, t), 6.80-6.93 (3H, m), 7.24-7.46 (5H, m), 7.59 (1H, dd), 8.48 (1H, dd).

MS m/z: 501 (M+1)

### EXAMPLE 174

4-(4-Chlorophenyl)-1-[3-(7-cyclopentoxy-5,11-dihydro[1 ]benzoxepino[2,3-b]pyridin-5-ylidene)propyl]piperidin-4-ol

The titled compound was prepared by following the procedure of example 46, but replacing ethyl iodide with cyclopentyl bromide.

1H-NMR (CDCl₃) δ: 1.54-2.18 (13H, m), 2.41-2.72 (8H, m), 4.66-4.73 (1H, m), 5.27 (2H, brs), 6.08 (1H, t), 6.70-6.87 (3H, m), 7.23-7.44 (5H, m), 7.58 (1H, dd), 8.49 (1H, dd).

MS m/z: 531 (M+1)

### EXAMPLE 175

4-(4-Chlorophenyl)-1-[3-(5,11-dihydro-7-(2-methoxyethyl)oxy)[1]benzoxepino[2,3-b]pyridin-5-ylidene)propyl]piperidin-4-ol

The titled compound was prepared by following the procedure of example 46, but replacing ethyl iodide with 2-methoxyethyl chloride.

1H-NMR (CDCl₃) δ: 1.66-1.75 (3H, m), 2.00-2.11 (2H, m), 2.36-2.71 (8H, m), 3.45 (3H, s), 3.71-3.75 (2H, m), 4.07-4.11 (2H, m), 5.27 (2H, brs), 6.09 (1H, t), 6.75-6.91 (3H, m), 7.23-7.44 (5H, m), 7.57 (1H, dd), 8.48 (1H, dd).

MS m/z: 521 (M+1)

### EXAMPLE 176

4-(4-Chlorophenyl)-1-[3-(7-(1-dimethyaminocarbonyl-1-methyl)ethyloxy-5,11-dihydro[1]benzoxepino[2,3-b]pyridin-5-ylidene)propyl]piperidin-4-ol

The titled compound was prepared by following the procedure of example 134, but replacing the product of example 133 with the product of example 139.

1H-NMR (CDCl₃) δ: 1.59 (6H, s), 1.67-1.72 (2H, m), 1.99-2.09 (2H, m), 2.36-2.70 (9H, m), 2.96 (3H, s), 3.21 (3H, s), 5.25 (2H, brs), 6.02 (1H, t), 6.60-6.77 (3H, m), 7.24-7.44 (5H, m), 7.58 (1H, dd), 8.44 (1H, dd).

MS m/z: 576 (M+1)

### EXAMPLE 177

4-(4-Chlorophenyl)-1-[3-(5,11-dihydro-7-(1-ethoxycarbonylethyl)oxy[1]benzoxepino[2,3-b]pyridin-5-ylidene)propyl]piperidin-4-ol

The titled compound was prepared by following the procedure of example 46, but replacing ethyl iodide with ethyl 2-bromopropionate.

1H-NMR (CDCl₃) δ: 1.25 (3H, t), 1.59 (3H, d), 1.65-1.70 (2H, m), 1.98-2.08 (2H, m), 2.35-2.68 (8H, m), 2.80 (1H, brs), 4.21 (2H, q), 4.68 (1H, q), 5.24 (2H, brs), 6.07 (1H, t), 6.68-6.79 (2H, m), 6.88 (1H, d), 7.22-7.44 (5H, m), 7.56 (1H, dd), 8.40 (1H, dd).

### EXAMPLE 178

1-[3-(7-(1-Carboxyethyl)oxy-5,11-dihydro[1]benzoxepino[2,3-b]pyridin-5-ylidene)prop yl]-4-(4-chlorophenyl)piperidin-4-ol

The titled compound was prepared by following the procedure of example 133, but replacing product of example 48 with product of example 177.

1H-NMR (DMSO-d₆) δ: 1.46 (3H, d), 1.58-1.63 (2H, m), 1.98-2.06 (2H, m), 2.41-2.45 (2H, m), 2.72-2.86 (6H, m), 4.74 (1H, q), 5.18 (2H, brs), 6.11 (11H, t), 6.73 (2H, s), 6.84 (1H, s), 7.36-7.47 (5H, m), 7.73 (1H, dd), 8.50 (1H, dd).

MS m/z: 535 (M+1)

### EXAMPLE 179

4-(4-Chlorophenyl)-1-[3-(5,11-dihydro-7-(1-ethoxycarbonyl)cyclobutoxy[1]benzoxepino[2,3-b]pyridin-5-ylidene)propyl]piperidin-4-ol

The titled compound was prepared by following the procedure of example 46, but replacing ethyl iodide with ethyl 2-bromocyclobutanecarboxylate.

1H-NMR (CDCl₃) δ: 1.19 (3H, t), 1.67-1.71 (2H, m), 1.92-2.11 (5H, m), 2.33-2.77 (12H, m), 4.21 (2H, q), 5.25 (2H, brs), 6.05 (1H, t), 6.47 (1H, dd), 6.70 (1H, d), 6.73 (1H, d), 7.23-7.44 (5H, m), 7.55 (1H, dd), 8.44 (1H, dd).

### EXAMPLE 180

1-[3-(7-(1-Carboxy)cyclbutoxy-5,11-dihydro[1]benzoxepino[2,3-b]pyridin-5-ylidene)propyl]-4-(4-chlorophenyl)piperidin-4-ol

The titled compound was prepared by following the procedure of example 133, but replacing product of example 48 with product of example 179.

1H-NMR (DMSO-d₆) δ: 1.60-1.65 (2H, m), 1.86-2.08 (4H, m), 2.24-2.90 (12H, m), 5.17 (2H, brs), 6.05 (1H, t), 6.50 (1H, dd), 6.66 (1H, d), 6.73 (1H, d), 7.37-7.48 (5H, m), 7.74 (1H, dd), 8.51 (1H, dd).

MS m/z: 561 (M+1)

### EXAMPLE 181

1-[3-(7-Carbamoylmethyloxy-5,11-dihydro[1]benzoxepino[2,3-b]pyridin-5-ylidene)propyl]-4-(4-chlorophenyl)piperidin-4-ol

The titled compound was prepared by following the procedure of example 134, but replacing dimethylamine hydrochloride with ammonium hydroxide.

1H-NMR (CDCl₃) δ: 1.66-1.71 (2H, m), 1.98-2.09 (2H, m), 2.21 (1H, brs), 2.38-2.70 (8H, m), 4.45 (2H, s), 5.28 (2H, brs), 6.09 (1H, t), 6.11 (1H, brs), 6.58 (1H, brs), 6.74-6.85 (3H, m), 7.24-7.44 (5H, m), 7.58 (1H, dd), 8.47 (1H, dd).

MS m/z: 520 (M+1)

### EXAMPLE 182

4-(4-Chlorophenyl)-1-[3-(5,11-dihydro-7-methylaminocarbonylmethyloxy[1]benzoxepino[2,3-b]pyridin-5-ylidene)propyl]piperidin-4-ol

The titled compound was prepared by following the procedure of example 134, but replacing dimethylamine hydrochloride with methylamine.

1H-NMR (CDCl₃) δ: 1.67-1.72 (2H, m), 1.99-2.10 (2H, m), 2.36-2.70 (9H, m), 2.89 (3H, d), 4.45 (2H, s), 5.28 (2H, brs), 6.08 (1H, t), 6.66 (1H, brs), 6.73-6.84 (3H, m), 7.25-7.45 (5H, m), 7.58 (1H, dd), 8.47 (1H, dd).

MS m/z: 534 (M+1)

### EXAMPLE 183

1-[3-(5,11-Dihydro-7-methoxy[1]benzoxepino[2,3-b]pyridin-5-ylidene)propyl]-4-(4-hydroxyphenyl)piperidine

The titled compound was prepared by following the procedure of example 45, step 3, but replacing 4-(4-chlorophenyl)-4-hydroxypiperidine with 4-(4-hydroxyphenyl)piperidine.

1H-NMR (CDCL3) δ: 1.52-1.88 (4H, m), 2.01 (2H, dt), 2.28-2.60 (5H, m), 2.93 (2H, m), 3.79 (3H, s), 5.28 (2H, brs), 6.08 (1H, t), 6.68-6.88 (3H, m), 7.05-7.36 (5H, m), 7.58 (1H, dd), 8.50 (1H, dd).

MS m/z: 461 (M+1)

### EXAMPLE 184

1-[3-(5,11-Dihydro-7-methoxy[1]benzoxepino[2,3-b]pyridin-5-ylidene)propyl]-4-(2-hydroxyphenyl)piperidine

The titled compound was prepared by following the procedure of example 45, step 3, but replacing 4-(4-chlorophenyl)-4-hydroxypiperidine with 4-(2-hydroxyphenyl)piperidine.

¹H-NMR (CDCl₃) δ: 1.78-1.92 (4H, m), 2.12-2.25 (2H, m), 2.32-2.70 (4H, m), 2.80-2.97 (1H, m), 3.01-3.15 (2H, m), 3.77 (3H, s), 3.78 (1H, brs), 5.28 (2H, brs), 6.03 (1H, t), 6.74-6.86 (4H, m), 7.05 (1H, dd), 7.1 1 (1H, dd), 7.23-7.28 (2H, m), 7.56 (1H, dd), 8.48 (1H, dd), OH signal was not observed.

MS m/z: 443 (M+1)

### EXAMPLE 185

4-(7-Chloro-1,2-benzisoxazol-3-yl)-1-[3-(5,11-dihydro-7-methoxy[1]benzoxepino[2,3-b]pyridin-5-ylidene)propyl]piperidine

The titled compound was prepared by following the procedure of example 45, step 3, but replacing 4-(4-chlorophenyl)-4-hydroxypiperidine with 4-(7-chloro-1,2-benzisoxazol-3-yl) piperidine. This tetrahydropyridine was prepared by the same method described in J Med. Chem. 28:761-769 (1985).

¹H-NMR (CDCl₃) δ: 1.94-2.20 (6H, m), 2.30-2.60 (4H, m), 2.86-3.14 (3H, m), 3.79 (3H, s), 5.29 (2H, brs), 6.10 (1H, t), 6.70-6.88 (3H, m), 7.22 (1H, t), 7.27 (1H, dd), 7.50 (1H, dd), 7.57-7.68 (2H, m), 8.49 (1H, dd).

### EXAMPLE 186

4-(7-Chloroindol-3-yl)-1-[3-(5,11-dihydro-7-methoxy[1]benzoxepino[2,3-b]pyridin-5-ylidene)propyl]piperidine

The titled compound was prepared by following the procedure of example 45, step 3, but replacing 4-(4-chlorophenyl)-4-hydroxypiperidine with 4-(7-chloroindol-3-yl)piperidine. This piperidine was prepared by the same method described in J. Med. Chem. 36:4006-4014 (1993) and following hydrogenation described in Example 58, step 3.

¹H-NMR(CDCl₃) δ: 1.66-1.88 (2H, m), 1.92-2.22 (4H, m), 2.32-2.63 (4H, m), 2.78 (1H, m), 2.97 (2H, m), 3.79 (3H, s), 5.29 (2H, brs), 6.09 (1H, t), 6.70-6.87 (3H, m), 6.97-7.07 (2H, m), 7.12-7.30 (2H, m), 7.52 (1H, m), 7.59 (1H, dd), 8.45 (1H, brs), 8.50 (1H, dd).

### EXAMPLE 187

4-Azido-4-(4-chlorophenyl)-1-[3-(5,11-dihydro-7-methoxy[1]benzoxepino[2,3-b]pyridin-5-ylidene)propyl]piperidine

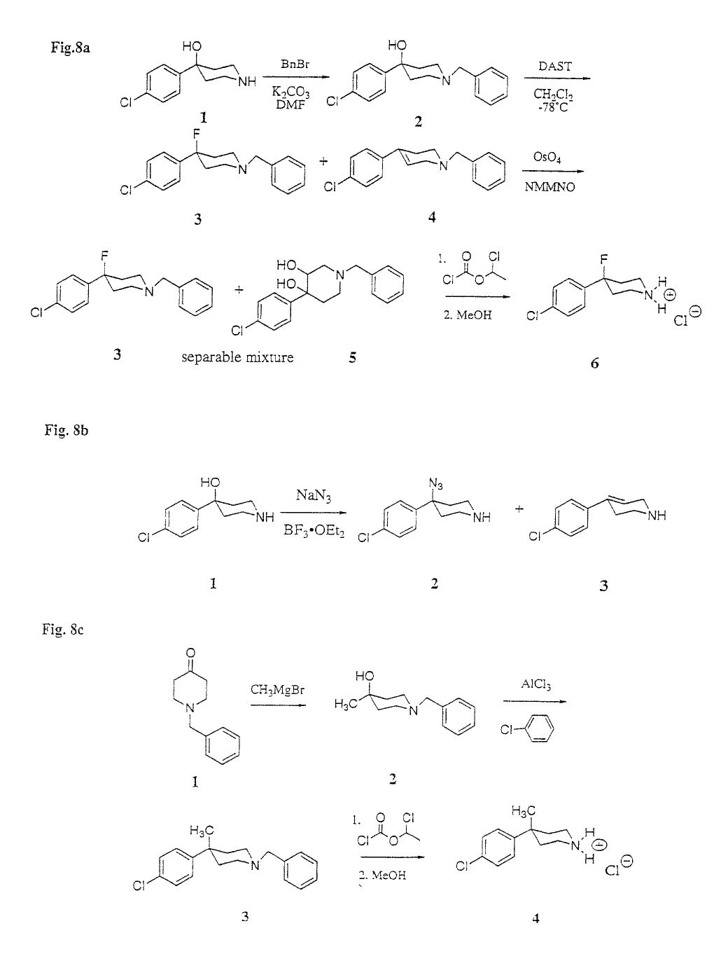

Step 1 4-azido-4-(4-chlorophenyl)piperidine (15): FIG. 8b

To a cold (0° C.) solution of 1 (3.0 g, 14 mmol) in anhydrous dioxane (15 mL) under an inert atmosphere was added NaN₃ (1.0 g, 15.4 mmol) followed by the slow dropwise addition of and BF₃.OEt (4.4 mL, 35 mmol). The reaction was stirred at 0° C. for 3 hrs and was quenched at 0° C. by the slow careful addition of saturated aqueous NaHCO₃ to basicity. The organic layer was separated and dried over Na₂SO₄. The reaction mixture was purified via silica gel flash chromatography eluting a 2 g 1:3 mixture of azidopiperidine 2 and olefin 3 with 2% MeOH/CH₂Cl₂. The mixture was taken directly on to the next reaction.

Step 2

The titled compound was prepared by then following the procedure of example 45, step 3, with the above reaction mixture (thereby replacing 4-(4-chlorophenyl)-4-hydroxypiperidine with 4-azido-4-(4-chlorophenyl)piperidine)), but limiting the amount of bromide to 0.25 equivalents.

¹H-NMR (CDCL₃) δ: 1.88 (2H, m), 2.55-2.85 (4H, m), 3.00-3.30 (6H, m). 3.75 (3H, s), 5.19 (2H, brs), 5.97 (1H, t), 6.68-6.65 (3H, m), 7.20-7.46 (5H, m), 7.63 (1H, dd), 8.35 (1H, dd).

MS m/z: 477 (M+1−N₂+H₂)

### EXAMPLE 188

Methyl 1-[3-(5,11-dihydro-7-methoxy[1]benzoxepino[2,3-b]pyridin-5-ylidene)propyl]-4-phenylpiperidin-4-carboxylate

The titled compound was prepared by following the procedure of example 45, step 3, but replacing 4-(4-chlorophenyl)-4-hydroxypiperidine with methyl 4-phenylpiperidin-4-carboxylate.

¹H-NMR (CDCl₃) δ: 1.82-2.15 (4H, m), 2.28-2.60 (6H, m), 2.78-2.82 (2H, m), 3.62 (3H, s), 3.68 (3H, s), 5.26 (2H, brs), 5.95 (0.1H, t, E isomer), 6.05 (0.9H, t, Z isomer), 6.82-6.70 (3H, m), 7.33-7.22 (6H, m), 7.65 (0.1H, dd, Z isomer), 7.55 (0.9H, dd, Z isomer), 8.39 (0.1H, dd, E isomer), 8.48 (0.9H, dd, Z isomer).

MS m/z: 485 (M+1)

### EXAMPLE 189

1-[3-(5,11-Dihydro-7-methoxy[1]benzoxepino[2,3-b]pyridin-5-ylidene)propyl]-4-phenylpiperidin-4-carboxylic Acid

The titled compound was prepared by following the procedure of example 133, but replacing product of example 48 with product of example 188.

¹H-NMR (CD₃OD) δ: 2.16-2.23 (2H, m), 2.69-2.91 (4H, m), 3.00-3.16 (2H, m), 3.37-3.25 (2H, m), 3.68-3.73 (2H, m), 3.76 (3H, s), 5.34 (2H, brs), 6.24 (1H, t), 6.70-7.04 (3H, m), 7.26-7.55 (5H, m), 7.79-7.89 (1H, m), 8.21-8.34 (1H, m), 8.56-8.62 (0.1H, m), 8.63-8.77 (0.9H, m),

MS m/z: 471 (M+1)

### EXAMPLE 190

1-(2-Chlorophenylsulfonyl)-4-[3-(5,11-Dihydro-7-methoxy[1]benzoxepino[2,3-b]pyridin-5-ylidene)propyl]piperazine

The titled compound was prepared by following the procedure of example 45, step 3, but replacing 4-(4-chlorophenyl)-4-hydroxypiperidine with 1-(2-chlorophenylsulfonyl)piperazine.

¹H-NMR (CDCl₃) δ: 2.20-2.58 (8H, m), 3.12-3.38 (4H, m), 3.76 (3H, s), 5.22 (2H, brs), 6.03 (1H, t), 6.64-6.90 (3H, m), 7.23 (1H, dd), 7.32-7.60 (4H, m), 8.01 (1H, dd), 8.48 (1H, dd).

MS m/z: 526 (M+1)

### EXAMPLE 191

1-(3-Chlorophenylsulfonyl)-4-[3-(5,11-Dihydro-7-methoxy[1]benzoxepino[2,3-b]pyridin-5-ylidene)propyl]piperazine

The titled compound was prepared by following the procedure of example 45, step 3, but replacing 4-(4-chlorophenyl)-4-hydroxypiperidine with 1-(3-chlorophenylsulfonyl)piperazine.

¹H-NMR (CDCl₃) δ: 2.20-2.60 (8H, m), 2.82-3.12 (4H, m), 3.76 (3H, s), 5.18 (2H, brs), 6.00 (1H, t), 6.64-6.90 (3H, m), 7.23 (1H, dd), 7.42-7.78 (5H, m), 8.48 (1H, dd).

MS m/z: 526 (M+1)

### EXAMPLE 192

1-(4-Chlorophenylsulfonyl)-4-[3-(5,11-Dihydro-7-methoxy[1]benzoxepino[2,3-b]pyridin-5-ylidene)propyl]piperazine

The titled compound was prepared by following the procedure of example 45, step 3, but replacing 4-(4-chlorophenyl)-4-hydroxypiperidine with 1-(4-chlorophenylsulfonyl)piperazine.

¹H-NMR (CDCl₃) δ: 2.20-2.56 (8H, m), 2.82-3.10 (4H, m), 3.76 (3H, s), 5.18 (2H, brs), 5.99 (1H, t), 6.62-6.92 (3H, m), 7.23 (1H, dd), 7.42-7.78 (5H, m), 8.48 (1H, dd).

MS m/z: 526 (M+1)

### EXAMPLE 193

4-(4-Chlorophenyl)-1-[3-(5,11-dihydro-7-hydroxy[1]benzoxepino[2,3-b]pyridin-5-ylidene)propyl]-1,2,3,6-tetrahydropyridine

The titled compound was prepared by following the procedure of example 44, step 2, but replacing 4-(4-chlorophenyl)-4-hydroxypiperidine with 4-(4-chlorophenyl)-1,2,3,6-tetrahydropyridine.

¹H-NMR (CDCl₃) d: 2.37-2.72 (8H, m), 3.07 (2H, m), 5.25 (2H, brs), 6.00 (1H, m), 6.07 (1H, t), 6.60-6.78 (3H, m), 7.18-7.47 (5H, m), 7.56 (1H, dd), 8.50 (1H, dd). OH signal was not observed.

MS m/z: 445 (M+1)

### EXAMPLE 194

4-(4-Chlorophenyl)-1-[3-(5,11-dihydro-7-methoxy[1]benzoxepino[2,3-b]pyridin-5-ylidene)propyl]-1,2,3,6-tetrahydropyridine

The titled compound was prepared by following the procedure of example 45, step 3, but replacing 4-(4-chlorophenyl)-4-hydroxypiperidine with 4-(4-chlorophenyl)-1,2,3,6-tetrahydropyridine.

¹H-NMR (CDCl₃) δ: 2.37-2.72 (8H, m), 3.06 (2H, m), 3.78 (3H, s), 5.27 (2H, brs), 5.99 (1H, m), 6.10 (1H, t), 6.72-6.90 (3H, m), 7.20-7.44 (5H, m), 7.60 (1H, dd), 8.50 (1H, dd).

MS m/z: 459 (M+1)

### EXAMPLE 195

4-(7-Chloroindol-3-yl)-1-[3-(5, 11-dihydro-7-methoxy[1]benzoxepino[2,3-b]pyridin-5-ylidene)propyl]-1,2,3,6-tetrahydropyridine.

The titled compound was prepared by following the procedure of example 45, step 3, but replacing 4-(4-chlorophenyl)-4-hydroxypiperidine with 4-(7-chloroindol-3-yl)-1,2,3,6-tetrahydropyridine. This piperidine was prepared by the same method described in J. Med. Chem. 36:4006-4014 (1993).

¹H-NMR (CDCl₃) δ: 2.37-2.76 (8H, m), 3.14 (2H, m), 3.78 (3H, s), 5.29 (2H, brs), 6.02-6.23 (2H, m), 6.67-6.90 (3H, m), 7.05 (1H, dd), 7.12-7.33 (3H, m), 7.60 (1H, dd), 7.77 (1H, m), 8.50 (1H, dd), 9.06 (1H, br s).

### EXAMPLE 196

5-Chloro-1′-[3-(5,11-dihydro-7-hydroxy[1]benzoxepino[2,3-b]pyridin-5-ylidene)propyl]spiro[isobenzofuran-1(3H),4′-piperidine]

The titled compound was prepared by following the procedure of example 44, step 2, but replacing4-(4-chlorophenyl)-4-hydroxypiperidine with 5-chlorospiro[isobenzofuran-1(3H),4′-piperidine].

1H-NMR (CDCl₃) δ: 1.66-1.71 (2H, m), 1.79-1.91 (2H, m), 2.26-2.73 (8H, m), 4.99 (2H, s), 5.22 (2H, brs), 6.07 (1H, t), 6.63-6.70 (2H, m), 6.76 (1H, d), 7.06 (1H, d), 7.19-7.32 (3H, m), 7.60 (1H, dd), 8.47 (1H, dd), 8.63 (1H, s).

MS m/z: 475 (M+1)

### EXAMPLE 197

5-Chloro-1′-[3-(5,11-dihydro-7-(2-methoxyethyl)oxy[1]benzoxepino[2,3-b]pyridin-5-ylidene)propyl]spiro[isobenzofuran-1(3H),4′-piperidine]

The titled compound was prepared by following the procedure of example 175, but replacing the product of example 44 with the product of example 196.

1H-NMR (CDCl₃) δ: 1.69-1.74 (2H, m), 1.83-1.94 (2H, m), 2.31-2.76 (8H, m), 3.45 (3H, s), 3.72-3.75 (2H, m), 4.08-4.11 (2H, m), 5.00 (2H, s), 5.28 (2H, brs), 6.09 (1H, t), 6.74-6.82 (2H, m), 6.89 (1H, d), 7.04 (1H, d), 7.17-7.28 (3H, m), 7.57 (1H, dd), 8.49 (1H, dd).

MS m/z: 531 (M+1)

### EXAMPLE 198

4-(4-Chlorophenyl)-1-[3-(7-dimethylaminocarbonyl-5,11-dihydro[1]benzoxepino[2,3-b]pyridin-5-ylidene)propyl]piperidin-4-ol

The titled compound was prepared by following the procedure of example 134, but replacing the product of example 133 with the product of example 118.

1H-NMR (CDCl₃) δ: 1.65-1.70 (2H, m), 1.99-2.09 (3H, m), 2.32-2.69 (8H, m), 2.17 (3H, s), 5.35 (2H, brs), 6.15 (1H, t), 6.82 (1H, d), 7.19 (1H, dd), 7.28-7.46 (6H, m), 7.58 (1H, dd), 8.49 (1H, dd).

### EXAMPLE 199

4-(4-Chlorophenyl)-1-[3-(7-(2-(1-hydroxy-2-methyl)propyl)oxy-5,11-dihydro[1]benzoxepino[2,3-b]pyridin-5-ylidene)propyl]piperidin-4-ol

To a solution of product of example 138 (500 mg) in methanol (5 ml) was added sodium borohydride (330 mg), and the mixture was heated to reflux for 1 hour. The mixture was distilled off under reduced pressure. Water and ethyl acetate were added to the residue, the organic layer was separated and washed with saturated aqueous sodium chloride, and dried with magnesium sulfate. The solvent was distilled off under reduced pressure, and the residue was purified by silica gel chromatography eluting with chloroform-methanol (10:1) to give the titled compound (440 mg).

1H-NMR (CDCl₃) δ: 1.26 (6H, s), 1.66-1.70 (2H, m), 1.79 (1H, brs), 2.00-2.08 (2H, m), 2.37-2.70 (9H, m), 3.58 (2H, s), 5.30 (2H, brs), 6.05 (1H, t), 6.75-6.84 (2H, m), 6.91 (1H, d), 7.26-7.44 (5H, m), 7.58 (1H, dd), 8.49 (1H, dd).

MS m/z: 535 (M+1)

### EXAMPLE 200

4-(4-Chlorophenyl)-1-[3-(7-(1-(2-methyl-2-hydroxy)propyl)oxy-5,11-dihydro[1]benzoxepino[2,3-b]pyridin-5-ylidene)propyl]piperidin-4-ol

To a solution of product of example 48 (500 mg) in tetrahydrofuran (5 ml) was added 0.95M methylmagnesium bromide tetrahydrofuran solution (3.8 ml) at 0° C., and the mixture was stirred at room temperature for 20 minutes. Aqueous ammonium chloride solution and ethyl acetate were added to the mixture, the organic layer was separated and washed with saturated aqueous sodium chloride, and dried with magnesium sulfate. The solvent was distilled off under reduced pressure, and the residue was purified by silica gel chromatography eluting with chloroform-methanol (10:1) to give the titled compound (360 mg).

1H-NMR (CDCl₃) δ: 1.34 (6H, s), 1.58 (1H, brs), 1.66-1.71 (2H, m), 1.99-2.10 (2H, m), 2.25 (1H, brs), 2.36-2.71 (8H, m), 3.77 (2H, s), 5.28 (2H, brs), 6.09 (1H, t), 6.74-6.86 (3H, m), 7.24-7.44 (5H, m), 7.57 (1H, dd), 8.49 (1H, dd).

MS m/z: 535 (M+1)

### EXAMPLE 203

4-(4-Chlorophenyl)-1-[3-(7-(2-ethoxy)ethyloxy)-5,11-dihydro[1]benzoxepino[2,3-b]pyridin-5-ylidene)propyl]piperidin-4-ol

The titled compound was prepared by following the procedure of example 46, but replacing ethyl iodide with 2-ethoxyethyl bromide.

¹H-NMR (CDCl₃) δ: 1.24 (3H, t), 1.66-1.75 (3H, m), 2.00-2.11 (2H, m), 2.36-2.71 (8H, m), 3.59 (2H, q), 3.71-.75 (2H, m), 4.07-4.11 (2H, m), 5.27 (2H, brs), 6.09 (1H, t), 6.75-6.91 (3H, m), 7.23-7.44 (5H, m), 7.57 (1H, dd), 8.48 (1H, dd).

MS m/z: 535 (M+1)

### EXAMPLE 205

4-(4-Chlorophenyl)-1-[3-(5,11-dihydro-7-(1-(2,3-dihydroxy)propyloxy)[1]benzoxepino[2,3-b]pyridin-5-ylidene)propyl]piperidin-4-ol

The titled compound was prepared by following the procedure of example 46, but replacing ethyl iodide with glycidol.

¹H-NMR (CDCl₃) δ: 1.66-1.75 (2H, m), 2.00-2.11 (2H, m), 2.36-2.71 (8H, m), 3.62-3.76 (2H, m), 3.94-4.02 (4H, m), 4.21 (2H, brs), 5.27 (2H, brs), 6.09 (1H, t), 6.76-6.86 (3H, m), 7.23-7.44 (5H, m), 7.57 (1H, dd), 8.48 (1H, dd).

MS m/z: 537 (M+1)

### EXAMPLE 211

1-[3-(7-(1-Carbamoyl-1-methyl)ethyloxy-5,11-dihydro[1]benzoxepino[2,3-b]pyridin-5-ylidene)propyl]-4-(4-chlorophenyl)piperidin-4-ol

The titled compound was prepared by following the procedure of example 176, but replacing dimethylamine hydrochloride with ammonium hydroxide.

¹H-NMR (CDCl₃) δ: 1.50 (6H, s), 1.67-1.72 (2H, m), 1.96-2.09 (3H, m), 2.36-2.70 (8H, m), 5.30 (2H, brs), 5.70 (1H, brs), 6.05 (1H, t), 6.75-6.90 (4H, m), 7.25-7.44 (5H, m), 7.58 (1H, dd), 8.49 (1H, dd).

MS m/z: 548 (M+1)

### EXAMPLE 212

4-(4-Chlorophenyl)-1-[3-(5,11-dihydro-7-(1-methylaminocarbonyl-1-methyl)ethyloxy[1]benzoxepino[2,3-b]pyridin-5-ylidene)propyl]piperidin-4-ol

The titled compound was prepared by following the procedure of example 176, but replacing dimethylamine hydrochloride with methylamine.

¹H-NMR (CDCl₃) δ: 1.47 (6H, s), 1.67-1.72 (2H, m), 1.96-2.09 (2H, m), 2.20 (1H, brs), 2.36-2.70 (8H, m), 2.87 (3H, d), 5.29 (2H, brs), 6.04 (1H, t), 6.72-6.86 (4H, m), 7.27-7.44 (5H, m), 7.58 (1H, dd), 8.47 (1H, dd).

MS m/z: 562 (M+1)

### EXAMPLE 215

4-(4-Chlorophenyl)-1-[3-(7-(2-dimethylaminocarboxy)ethenyl-5,11-dihydro[1]benzoxepino[2,3-b]pyridin-5-ylidene)propyl]piperidin-4-ol

The titled compound was prepared by following the procedure of example 134, but replacing the product of example 133 with the product of example 172.

¹H-NMR (CDCl3) δ: 1.63-1.71 (3H, m), 1.98-2.10 (2H, m), 2.35-2.72 (8H, m), 3.07 (3H, s), 3.17 (3H, s), 5.36 (2H, brs), 6.16 (1H, t), 6.76 (1H, d), 6.84 (1H, d), 7.28-7.45 (7H, m), 7.59-7.65 (2H, m), 8.52 (1H, dd).

MS m/z: 544 (M+1)

### EXAMPLE 218

1-[3-(7-(2-Carbamoyl)ethyl-5,1 1-dihydro[1]benzoxepino[2,3-b]pyridin-5-ylidene)propyl]-4-(4-chlorophenyl)-piperidin-4-ol

The titled compound was prepared by following the procedure of example 181, but replacing the product of example 133 with the product of example 123.

¹H-NMR (CDCl₃) δ: 1.65-1.90 (3H, m), 2.10-2.22 (2H, m), 2.40-2.80 (10H, m), 2.91 (2H, t), 5.31-5.46 (4H, m), 6.1 1 (1H, t), 6.78 (1H, d), 7.01 (1H, dd), 7.16 (1H, d), 7.28-7.46 (5H, m), 7.57 (1H, dd), 8.49 (1H, dd).

MS m/z: 518 (M+1)

### EXAMPLE 234

### 1-[3-(5,11-Dihydro-7-methoxy[1]benzoxepino[2,3-b]pyridin-5-ylidine)propyl]-4-(indol-3-yl)-piperidine

The titled compound was prepared by following the procedure of example 45, step 3, but replacing 4-(4-chlorophenyl)-4-hydroxypiperidine with 4-(indol-3-yl)-piperidine. This piperidine was prepared by the same method described in J. Med. Chem. 36:4006-4014 (1993) and follow hydrogenation described in Example 58, step 3.

¹H-NMR(CDCl₃) d: 1.65-1.93 (2H, m), 1.94-2.28 (4H, m), 2.34-2.70 (4H, m), 2.81 (1H, m), 2.96 (2H, m), 3.78 (3H, s), 5.28 (2H, brs), 6.09 (1H, t), 6.70-7.42 (8H, m), 7.53-7.72 (2H, m), 8.28 (1H, brs), 8.49 (1H, m).

### EXAMPLE 235

### 1-[3-(5,11-Dihydro-7-methoxy[1]benzoxepino[2,3-b]pyridin-5-ylidine)propyl]-4-(indol-3-yl)-1,2,3,6-tetrahydropyridine.

The titled compound was prepared by following the procedure of example 45, step 3, but replacing 4-(4-chlorophenyl)-4-hydroxypiperidine with 4-(indol-3-yl)-1,2,3,6-tetrahydropyridine. This piperidine was prepared by the same method described in J. Med. Chem. 36:4006-4014 (1993).

¹H-NMR (CDCl₃) d: 2.35-2.77 (8H, m), 3.06-3.26 (2H, m), 3.78 (3H, s), 5.29 (2H, brs), 6.05-6.22 (2H, m), 6.70-6.88 (3H, m), 7.07-7.38 (5H, m), 7.60 (1H, dd), 7.87 (1H, m), 8.42 (1H, brs), 8.50 (1H, m).

### EXAMPLE 236

### 4-(4-Chlorophenyl)-1-[3-(5,11-dihydro-7-(3-(ethoxycarbonyl)propyloxy[1]benzoxipino[2,3-b]pyridin-5-ylidine)propyl]piperidine

The titled compound was prepared by following the procedure of example 153, but replacing ethyl bromoacetate with ethyl 4-bromobutyrate.

¹H-NMR (CDCL₃) δ: 1.26 (3H, t), 1.56-1.85 (4H, m), 2.01 (2H, dt), 2.09 (2H, quint), 2.30-2.60 (7H, m), 2.93 (2H, m), 3.98 (2H, t), 4.15 (2H, q), 5.28 (2H, brs), 6.07 (1H, t), 6.68-6.86 (3H, m), 7.07-7.33 (5H, m),7.58 (1H, dd), 8.50 (1H, dd).

MS m/z: 561 (M+1)

### EXAMPLE 237

### 1-[3-(7-(3-Carboxypropyl)oxy-5,11-dihydro-[1]benzoxepino[2,3-b]pyridin-5-ylidine)propyl]-4-(4-chlorophenyl)-piperidine

The titled compound was prepared by following the procedure of example 133, but replacing the product of example 48 with the product of example 236.

¹H-NMR (CD₃OD) δ: 1.92-2.20 (6H, m), 2.48 (2H, t), 2.70-3.02 (3H, m), 3.06-3.45 (4H, m), 3.66 (2H, m), 4.01 (2H, t), 5.48 (2H, brs), 6.36 (1H, t), 6.85 (2H, s), 7.00 (1H, s), 7.20-7.40 (4H, m), 8.11 (1H, dd), 8.64 (1H, d), 8.81 (1H, d). COOH signal was not observed.

MS m/z: 533 (M+1)

### EXAMPLE 242

4-(4-Chlorophenyl)-1-[3-(5,11-dihydro-7-(1-hydroxy-1-methyl)ethyl[1]benzoxepino[2,3-b]pyridin-5-ylidene)propyl]piperidin-4-ol

The titled compound was prepared by following the procedure of example 200, but replacing the product of example 48 with the product of example 273.

¹H-NMR (CDCl₃) δ: 1.58 (6H, s), 1.65-1.70 (3H, m), 1.93-2.21 (2H, m), 2.28-2.73 (8H, m), 5.32 (2H, brs), 6.13 (1H, t), 6.82 (1H, d), 7.20-7.50 (7H, m), 7.59 (1H, dd), 8.50 (1H, dd)

MS m/z: 505 (M+1)

### EXAMPLE 243

1-[3-(7-(1-Carboxy-1-methyl)ethyl-5, 11-dihydro[1]benzoxepino[2,3-b]pyridin-5-ylidene)propyl]-4 (4-chlorophenyl)piperidin-4-ol

Step 1

To a solution of Example 363, step 2 (2.4 g) in toluene (30 ml) was added DIBAL (1 mol/L toluene solution, 9.2 ml) at −78° C., and the mixture stirred at 0° C. for 1 hour, and at room temperature for 30 minutes. The reaction mixture was added saturated aqueous ammonium chloride. 1 N aqueous hydrochloric acid, saturated sodium chloride and ethyl acetate were added to the mixture, the organic layer was separated and washed with saturated aqueous sodium chloride, and dried with magnesium sulfate. The solvent was distilled off under reduced pressure. The residue was purified by silica gel chromatography eluting with ethyl acetate-hexane (1:4) to give 5-(3-bromopropylidene)-5,11-dihydro-7-(1-hydroxy-1-methyl)ethyl[1]benzoxepino[2m30b]pyridine (2.0 g).

¹H-NMR (CDCl₃) δ: 1.45 (H, s), 2.75 (2H, q), 3.47 (1H, t), 5.33 (2H, brs), 6.04 (1H, t), 6.87 (1H, d), 7.09-7.14 (2H, m), 7.30 (1H, dd), 7.57 (1H, dd), 8.53 (1H, dd), 9.46 (1H, s).

Step 2

5-(3-bromopropylidene)-7-(1-carboxy-1-methyl)ethyl-5, 11-dihydro[1]benzoxepino [2,3-b]pyridine was prepared by following the procedure of Example 382, step 2, but replacing the product of Example 382, step 1 with the product of step 1 above.

Step 3

The titled compound was prepared by following the procedure of example 44, step 2, but replacing the product of example 44, step 1 with the product of step 2.

¹H-NMR (DMSO-d6) δ: 1.46 (6H, s), 1.63-1.84 (2H, m), 2.17-2.37 (4H, m), 2.37-2.53 (4H, m), 3.20-3.43 (2H, m), 4.83 (1H, s), 5.23 (2H, brs), 6.13 (1H, t), 6.76 (1H, d), 7.16 (1H, dd), 7.25 (1H, d), 7.35 (2H, d), 7.42-7.48 (3H, m), 7.76 (1H, dd), 8.50 (1H, dd).

MS m/z: 533 (M+1)

### EXAMPLE 248

### 1′-[3-(5,11-Dihydro-7-methoxy[1]benzoxepino[2,3-b]pyridin-5-ylidine)propyl]-6-methylspiro[4H-3,1-benzoxazine-4,4′-piperidine]-2(1H)-one

The titled compound was prepared by following the procedure of example 45, step 3, but replacing 4-(4-chlorophenyl)-4-hydroxypiperidine with 6-methylspiro[4H-3,1-benzoxazine-4,4′-piperidin]-2(1H)-one.

¹H-NMR (CDCl₃) δ: 1.99-2.06 (2H, m), 2.29 (3H, s), 2.32-2.69 (10H, m), 3.77 (3H, s), 5.27 (2H, brs), 6.08 (1H, t), 6.69-6.83 (4H, m), 6.94 (1H, s), 7.02 (1H, d), 7.25 (1H, dd), 7.55 (1H, dd), 8.48 (1H, dd), 8.56 (1H, s).

MS m/z: 498 (M+1)

### EXAMPLE 249

### 5-(4-Chlorophenyl)-1-[3-(5,11-dihydro-7-methoxy[1]benzoxepino[2,3-b]pyridin-5-ylidene)propyl]-4,6-dioxazacane.

5-(4-Chlorophenyl)-1-[3-(5,11-dihydro-7-methoxy[1]benzoxepino[2,3-b]pyridin-5-ylidene)propyl]-4,6-diazacyclooctylamine

Step 1

5-(3-(N,N′-Bis(2-hydroxyethyl)amino)propylidene)-5,11-dihydro-7-methoxy[1]benzoxepino[2,3-b]pyridine was prepared by following the procedure of example 45, step 3, but replacing 4-(4-chlorophenyl)-4-hydroxypiperidine with diethanolamine.

¹H-NMR (CD₃OD) δ: 2.46 (2H, m), 2.84 (4H, t), 2.98 (2H, m), 3.67 (4H, t), 3.75 (3H, s), 5.20 (2H, brs), 6.16 (1H, t), 6.68-6.80 (2H, m), 6.87 (1H, d), 7.46 (1H, dd), 7.81 (1H, dd), 8.45 (1H, dd).

Step 2

To a mixture of product of step I (78 mg) and 4-chlorobenzaldehyde dimethyl acetal (0.1 ml) in 1,2-dichloroethane (60 ml) was added p-toluenesulfonic acid monohydrate (5 mg) at room temperature, and the mixture was stirred at reflux for 12 hours. Dichloromethane and saturated aqueous sodium bicarbonate was added to the cooled reaction mixture, the organic layer was separated and washed with saturated aqueous sodium chloride, and dried with magnesium sulfate. The solvent was distilled off under reduced pressure, and the residue was purified by silica gel chromatography eluting with dichloromethane-methanol (20:1) to give the titled compound (40 mg).

¹H-NMR (CDCl₃) δ: 2.35 (2H, m), 2.64-2.94 (6H, m), 3.52-3.68 (2H, m), 3.78 (3H, s), 3.72-3.90 (2H, m), 5.27 (2H, brs), 5.66 (1H, s), 6.08 (1H, t), 6.68-6.88 (3H, m), 7.18-7.46 (5H, m), 7.58 (1H, dd), 8.50 (1H, dd).

### EXAMPLE 252

Step 1

To a cold (0° C.) stirred solution of 4-oxohomopiperidine·HCl (0.6 g, 4.05 mmol), K₂CO₃ (0.615 g, 4.46 mmol) in anhydrous THF (10 mL) will be ethyl chloroformate (0.44 mL, 4.05 mmol) dropwise. The reaction was warmed to RT for 2 hrs then quenched with H₂O, extracted with EtOAc, and the organic layer dried over Na₂SO₄. Pure 1-ethylcarbonyl-4-oxohomopiperidine will be isolated via silica gel flash chromatography

Step 2

To a cold (0° C.) stirred solution of 1-ethylcarbonyl-4-oxohomopiperidine (1.42 g, 6.07 mmol) in anhydrous THF (50 mL) under argon can be added dropwise 1.0 mM 4-chlorophenyhnagnesium bromide in diethyl ether (10 mL, 10 mmol). The reaction can be warmed to RT for 2 hrs then quenched with saturated aqueous NH₄Cl 95 mL). The reaction mixture can then be extracted with EtOAc (2×50 mL), the organic layers combined and dried over Na₂SO₄. Pure 1-ethoxycarbonyl-4-(4-chlorophenyl)-4-hydroxyhomopeperidine (2.1 g, 96%) can be isolated via silica gel flash chromatography eluting with 50% ETOAc/hexane. 4-(4-chlorophenyl)-4-hydroxyhomopiperidine can be prepared by reacting 1-ethoxycarbonyl-4-(4-chlorophenyl)-4-hydroxyhomopeperidine with a nucleophilic hydroxide equivalent such as LiOH in a solvent such as THF, methanol or ethanol. Removal of the solvent can afford 4-(4-chlorophenyl)-4-hydroxyhomopeperidine.

Step 4

The compound was prepared by following the procedure for Example 44, but replacing 4-(4-chlorophenyl)-4-hydroxypeperidine with 4-(4-chlorophenyl)-4-hydroxyhomopeperidine.

### EXAMPLES 253 AND 254

Step 1

To a stirred solution of 4-oxohomopiperidine·HCl (1.2 g, 8.05 mmol), NaOH (0.68 g, 16.9 mmol) in t-BuOH/H₂O (1:1, 10 mL) was added t-butyldicarbonate (1.93 mL, 8.9 mmol) drop-wise. The reaction was stirred at RT overnight, extracted with EtOAc (2×10 mL) and the organic layer separated. The organic layer was dried over Na₂SO₄ and concentrated under vacuo. Pure 1-t-butoxycarbonyl-4-oxohomopiperidine (1.42 g, 84%) was isolated via silica gel flash chromatography eluting with 50% EtOAc/hexane.

¹H NMR CDCl₃ δ: 44 (9H, s), 1.72-1.84 (2H, m), 2.60-2.65 (4H, m), 3.55-3.61 (4H, m).

Step 2

To a cold (0° C.) stirred solution of 1-t-butoxycarbonyl-4-oxohomopiperidine (1.42 g, 6.07 mmol) in anhydrous THF (50 mL) under argon was added dropwise 1.0 M 4-chlorophenylmagnesium bromide in diethyl ether (10 mL, 10 mmol). The reaction was warmed to RT for 2 hrs then quenched with sat'd aqueous NH₄Cl (5 mL). The reaction mixture was extracted with EtOAc (2×50 mL), the organic layers combined and dried over Na₂SO₄. Pure 1-t-butoxycarbonyl-4-(4-chlorophenyl)-4-hydroxyhomopiperidine (2.1 g, 96%) was isolated via silica gel flash chromatography eluting with 50% EtOAc/hexane. ¹H NMR CDCl₃ δ 1.43 (9H, s), 1.61-2.22 (6H, m), 3.21-3031 (2H, m), 3.48-3.82 (2H, m).

Step 3

To a stirred solution of 1-t-butoxycarbonyl-4-(4-chlorophenyl)-4-hydroxyhomopiperidine (2.1 g) at RT in CH₂Cl₂ (48 mL) was added TFA (2.0 mL). The reaction was stirred at RT for 2 hrs. Excess solvent and TFA was removed affording 2.0 g (92% yield) 1:1 mixture of 3-(4-chlorophenyl)-2,3-dehydrohomopiperidine and 3-(4-chlorophenyl)-3,4-dehydrohomopiperidine. ¹H NMR (MeOD, isomer A) δ 2.01-2.11 (2H, m, 4), 2.60-2.71 (2H, m, 5), 2.81-2.92 (2H, m, 4), 2.83-3.05 (2H, m, 5), 3.66-3.92 (4H, m, 5), 6.16-6.21 (1H, t, 5). ¹H NMR (MeOD, isomer B) 3.44-3.56 (2H, m, 4), 3.88-3.97 (2H, m, 4), 6.01-6.12 (1H, t, 4), 7.32-7.44 (1H, t, 4).

Step 4

The compounds can be prepared by following the procedure for Example 44 but replacing 4-(4-chlorophenyl)-4-hydroxypiperidine with 3-(4-chlorophenyl)-3,4-dehydrohomopiperidine and 3-(4-chlorophenyl)-4,5-dehydrohomopiperidine.

### EXAMPLE 255

1-(4-Chlorophenyl)-4-[3-(5,1 1-dihydro-7-hydroxy[1]benzoxepino[2,3-b]pyridin-5-ylidene)propyl]piperazinone

The titled compound was prepared by following the procedure of example 44, step 2, but replacing 4-(4-chlorophenyl)-4-hydroxypiperidine with 1-(4-chlorophenyl)piperazinone. ¹H-NMR (DMSO-d₆) δ: 2.30-2.34 (2H, m), 2,49-2.57 (2H, m), 2.68 (2H, t), 3.06 (2H, s), 3.58 (2H, t), 5,12 (2H, brs), 6.06 (2H, t), 6.57-6.69 (3H, m), 7.35-7.71 (5H, m), 7.72 (1H, dd), 8.48 (1H, dd).

### EXAMPLE 256

1-(4-Chlorophenyl)-4-[3-(5,11-dihydro-7-hydroxy[1]benzoxepino[2,3-b]pyridin-5-ylidene)propyl]homopiperazdine

The titled compound was prepared by following the procedure of example 44, step 2, but replacing 4-(4-chlorophenyl)-4-hydroxypiperidine with 1-(4-chlorophenyl)homopiperazdine.

¹H-NMR (CDCl₃) δ: 1.89 (2H, brs), 2.27-2.35 (2H, m), 2.51-2.70 (6H, m), 3.37-3.53 (4H, m), 5.23 (2H, brs), 5.98 (1H, t), 6.48-6.74 (6H, m), 7.05-7.26 (2H, m), 7.52 (1H, dd), 8.45 (1H, dd).

MS m/z: 462 (M+1)

### EXAMPLE 260

3-(4-Chlorophenyl)-8-[3-(5,11-dihydro-7-hydroxy[1]benzoxepino[2,3-b]pyridin-5-ylidene)propyl]-8-azabicyclo[3.2.1]octan-3-ol

The titled compound was prepared by following the procedure of example 44, step 2, but replacing 4-(4-chlorophenyl)-4-hydroxypiperidine with 3-(4-chlorophenyl)-8-azabicyclo[3.2.1]octan-3-ol

¹H-NMR(CDCl₃) δ: 1.65-2.10 (4H, m), 2.1-2.7 (8H, m), 3.32 (2H, bs), 3.78 (3H, s), 5.24 (2H, bs), 6.10 (1H, dd), 6.70-6.90 (3H, m), 7.15-7.31 (3H, m), 7.45 (bd, 2H), 7.64 (dd, 1H) 8.46 (dd, 1H)

MS m/z: 503 (M+1)

### EXAMPLE 261

1′-(4-Chlorophenyl)-1-[3-(5,11-dihydro-7-hydroxy[1]benzoxepino[2,3-b]pyridin-5-ylidene)propyl]spiro[5-chloro-1,3-benzodioxole-2,4′-piperidine]

The titled compound was prepared by following the procedure of example 44, step 2, but replacing 4-(4-chlorophenyl)-4-hydroxypiperidine with spiro[5-chloro-1,3-benzodioxole-2,4′-piperidine] (Journal of Medicinal Chemistry. 1995, 38, 2009-2017).

¹H-NMR(DMSO-d₆) δ: 1.78-2.02 (4H, m), 2.18-2.63 (8H, m), 4.97-5.27 (2H, brs), 6.06 (1H, t), 6.58-6.67 (3H, m), 6.79-6.87 (2H, m), 6.99 (1H, d), 7.42 (1H, dd), 7.72 (1H, dd), 8.49 (1H, dd), 9.07 (1H, s).

### EXAMPLE 262

1-[3-(7-(1-Carbamoyl-1-methyl)ethyloxy-5,11-dihydro[1]benzoxepino[2,3-b]pyridin-5-ylidene)propyl]-4-(4-chlorophenyl)-4-hydroxy-1-methylpiperidinium iodide

To a solution of the product of example 211 (330 mg) and in acetonitrile (1.2 ml) was added iodomethane (0.07 ml), and the reaction mixture was stirred at room temperature for 2 hours. The precipitation was filtered and washed with acetonitrile to give the titled compound (250 mg).

¹H-NMR (DMSO-d₆) δ: 1.39 (6H, s), 1.65-1.85 (2H, m), 2.20-2.64 (4H, m), 3.09 (3H, s), 3.30-3.65 (6H, m), 5.20 (2H, m), 5.61 (1H, s), 6.01 (1H, t), 6.75-6.92 (3H, m), 7.27 (1H, s), 7.38-7.64 (6H, m), 7.83 (1H, dd), 8.56 (1H, dd)

MS m/z: 562[(M−I)+]

### EXAMPLE 263

4-(4-Chlorophenyl)-1-[3-(7-diethylaminocarbonylmethyloxy-5,11-dihydro[1]benzoxepino[2,3-b]pyridin-5-ylidene)propyl]piperidin-4-ol

The titled compound was prepared by following the procedure of example 134, but replacing dimethylamine hydrochloride with diethylamine.

¹H-NMR (CDCl₃) δ: 1.67-1.72 (2H, m), 1.99-2.10 (2H, m), 2.36-2.70 (9H, m), 2.89 (3H, d), 4.45 (2H, s), 5.28 (2H, brs), 6.08 (1H, t), 6.66 (1H, brs), 6.73-6.84 (3H, m), 7.25-7.45 (5H, m), 7.58 (1H, dd), 8.47 (1H, dd).

MS m/z: 534 (M+1)

### EXAMPLE 268

4-(4-Chlorophenyl)-1-[3-(5, 11-dihydro-7-methylaminocarbonyl[1]benzoxepino[2,3-b]pyridin-5-ylidene)propyl]piperidin-4-ol

The titled compound was prepared by following the procedure of example 198, but replacing dimethylamine hydrochloride with methylamine.

¹H-NMR (DMSO-d6) δ: 1.75-1.80 (2H, m), 2.38-2.50 (2H, m), 2.63-2.73 (2H, m), 2.78 (3H, d), 3.17-3.50 (6H, m), 5.38 (2H, brs), 6.36 (1H, t), 6.87 (1H, d), 7.41-7.50 (4H, m), 7.55-7.99 (4H, m), 8.48-8.50 (1H, m), 8.61 (1H, dd).

MS m/z: 504 (M+1)

### EXAMPLE 269

1-[3-(7-Carbamoyl-5,11-dihydro[1]benzoxepino[2,3-b]pyridin-5-ylidene)propyl]-4-(4-chlorophenyl)piperidin-4-ol

The titled compound was prepared by following the procedure of example 198, but replacing dimethylamine hydrochloride with am onium hydroxide. ¹H-NMR (CDCl₃) δ: 1.67-1.79 (2H, m), 2.01-2.10 (2H, m), 2.17-2.71 (8H, m), 5.38 (2H, brs), 6.21 (1H, t), 6.85 (1H, d), 7.27-7.57 (9H, m), 7.90 (1H, dd), 8.50 (1H, dd).

MS m/z: 490 (M+1)

### EXAMPLE 270

4-(4-Chlorophenyl)-1-[3-(7-diethylaminocarbonyl-5,11-dihydro[1]benzoxepino[2,3-b]pyridin-5-ylidene)propyl]piperidin-4-ol

The titled compound was prepared by following the procedure of example 198, but replacing dimethylamine hydrochloride with diethylamine.

MS m/z: 546 (M+1)

### EXAMPLE 273

4-(4-Chlorophenyl)-1-[3-(5,11-dihydro-7-(methoxycarbonyl[1]benzoxepino[2,3-b]pyridin-5-ylidene)propyl]piperidin-4-ol

A mixture of the product of example 169 (15.0 g), palladium(II) diacetate (170 mg), 1,3-bis(diphenylphosphino)propane (310 mg), and triethylamine (7.0 ml) in methanol (100 ml) and dimethylformamide (150 ml) was purged with carbon monoxide for 5 minutes and stirred under a carbon monoxide balloon at 70° C. for 8 hours. The reaction mixture was evaporated under reduced pressure. The residue was added water and extracted with ethyl acetate. The extract was dried over magnesium sulfate, and the solvent was evaporated under reduced pressure. The residue was purified by silica gel column chromatography (ethyl acetate: methanol=10:1) to give the titled compound (13.1 g).

¹H-NMR(CDCl₃) δ: 1.45-1.80 (3H, m), 1.90-2.15 (2H, m), 2.28-2.48 (4H, m), 2.50-2.75 (4H, m), 3.89 (3H, s), 5.25-5.50 (2H, m), 6.20 (1H, dd), 6.85 (1H, d), 7.20-7.37 (3H, m), 7.42 (2H, d), 7.58 (1H, d), 7.80 (1H, dd), 8.01 (1H, dd), 8.52 (1H, dd)

MS m/z: 505 (M+1)

### EXAMPLE 274

4-(4-Chlorophenyl)-1-[3-(5,11-dihydro-7-hydroxymethyl[1]benzoxepino[2,3-b]pyridin-5-ylidene)propyl]piperidin-4-ol

To an ice-cooled solution of the product of example 273 (2.0 g) in tetrahydrofuran (100 ml) was added lithium aluminum hydride (300 mg), and the reaction mixture was stirred at room temperature for 12 hours. After the reaction mixture was cooled to 0C, water (0.3 ml), 15% sodium hydroxide aqueous solution (0.3 ml), and water (0.9 ml) were added. The reaction mixture was filtered, and the filtrate was dried over magnesium sulfate. The solvent was evaporated under reduced pressure and the residue was purified by silica gel column chromatography (chloroform: methanol: 28% ammonia in water=100:5:1) to give the titled compound (1.6 g).

¹H-NMR (CDCl₃) δ: 1.55-1.71 (3H, m), 1.95-2.25 (2H, m), 2.34-2.70 (8H, m), 4.62 (2H, s), 5.20-5.45 (2H, brs), 6.13 (1H, t), 6.84 (1H, d), 7.16 (1H, dd), 7.23-7.43 (6H, m), 7.58 (1H, dd), 8.51 (1H, dd)

MS m/z: 477 (M+1)

### EXAMPLE 275

4-(4-Chlorophenyl)-1-[3-(5,11-dihydro-7-(1-propylamino)methyl[1]benzoxepino[2,3-b]pyridin-5-ylidene)propyl]piperidin-4-ol

To a solution of the product of example 314 (300 mg) and 1-propylamine (0.26 ml) in tetrahydrofuran (6 ml) was added acetic acid (0.36 ml), and the reaction mixture was stirred at 60° C. for 30 minutes. Then the reaction mixture was added sodium triacetoxyborohydride (670 mg) at 0° C. , and stirred for 1.5 hours at room temperature. Sodium bicarbonate, water, and chloroform were added to the reaction mixture. The organic layer was extracted, and dried over potassium carbonate, and evaporated under reduced pressure. The residue was recrystallized with ethyl acetate to give titled compound (130 mg).

¹H-NMR (CDCl₃) δ: 0.92 (3H, t), 1.49-1.70 (6H, m), 1.98 (2H, m), 2.34-2.42 (4H, m), 2.51-2.70 (6H, m), 3.71 (2H, s), 5.32 (2H, brs), 6.12 (1H, t), 6.81 (1H, d), 7.11 (1H, dd), 7.25-7.45 (6H, m), 7.57 (1H, dd), 8.49 (1H, dd).

MS m/z: 518 (M+1)

### EXAMPLE 276

4-(4-Chlorophenyl)-1-[3-(5,11-dihydro-7-(3-hydroxy-1-propylamino)methyl[1]benzoxepino[2,3-b]pyridin-5-ylidene)propyl]piperidin-4-ol

The titled compound was prepared by following the procedure of Example 275, but replacing 1-propylamine with 3-amino-1-propanol.

MS m/z: 534 (M+1)

### EXAMPLE 277

4-(4-Chlorophenyl)-1-[3-(5,11-dihydro-7-(1-piperidino)methyl[1]benzoxepino[2,3-b]pyridin-5-ylidene)propyl]piperidin-4-ol

The titled compound was prepared by following the procedure of example 275, but replacing 1-propylamine with piperidine.

MS m/z: 544 (M+1)

### EXAMPLE 278

4-(4-Chlorophenyl)-1-[3-(5,11-dihydro-7-(4-morpholino)methyl[1]benzoxepino[2,3-b]pyridin-5-ylidene)propyl]piperidin-4-ol

The titled compound was prepared by following the procedure of example 275, but replacing 1-propylamine with morpholine.

MS m/z: 546 (M+1)

### EXAMPLE 279

4-(4-Chlorophenyl)-1-[3-(5,11-dihydro-7-(1-pyrrolidino)methyl[1 ]benzoxepino[2,3-b]pyridin-5-ylidene)propyl]piperidin-4-ol

The titled compound was prepared by following the procedure of Example 275, but replacing 1-propylamine with 4-aminobutyric acid.

H-NMR (CDCl₃) δ: 1.70-1.75 (2H, m), 1.98 (2H, m), 2.12-2.23 (2H, m), 2.40-2.86 (10H, m), 3.27 (2H, t), 4.36 (2H, s), 5.29 (2H, brs), 6.07 (1H, t), 6.80 (1H, d), 7.04 (1H, dd), 7.19 (1H, d), 7.28-7.32 (3H, m), 7.50 (1H, t), 7.61 (1H, dd), 8.51 (1H, dd).

MS m/z: 544 (M+1)

### EXAMPLE 280

4-(4-Chlorophenyl)-1-[3-(5,11-dihydro-7-(2-hydroxy)ethyl[1]benzoxepino[2,3-b]pyridin-5-ylidene)propyl]piperidin-4-ol

The titled compound was prepared by following the procedure of example 273, but replacing the product of example with the product of example 274.

¹H-NMR (CDCl₃) δ: 1.60-1.70 (4H, m), 2.01-2.12 (2H, m), 2.37-2.70 (8H, m), 2.81 (2H, t), 3.84 (2H, t), 5.31 (2H, brs), 6.09 (1H, t), 6.81 (1H, d), 7.03 (1H, dd), 7.15 (1H, d), 7.26-7.43 (5H, m), 7.57 (1H, dd), 8.49 (1H, dd).

MS m/z: 491 (M+1)

### EXAMPLE 281

1-[3-(7-Carbamoylmethyl-5,11-dihydro[1]benzoxepino[2,3-b]pyridin-5-ylidene)propyl]-4-(4-chlorophenyl)-piperidin-4-ol

The titled compound was prepared by following the procedure of example 122, but replacing dimethylamine hydrochloride with ammonium hydroxide.

¹H-NMR (CDCl₃) δ: 1.65-1.70 (2H, m), 1.98-2.06 (2H, m), 2.27-2.70 (9H, m), 3.46 (2H, s), 5.30 (2H, brs), 5.74 (1H, brs), 6.04 (1H, brs), 6.09 (1H, t), 6.79 (1H, d), 7.02 (1H, dd), 7.18-7.41 (6H, m), 7.54 (1H, dd), 8.43 (1H, dd).

MS m/z: 504 (M+1)

### EXAMPLE 288

4-(4-Chlorophenyl)-1-[3-(7-(2-ethoxycarboxy)ethyl-5,11-dihydro[1]benzoxepino[2,3-b]pyridin-5-ylidene)propyl]piperidin-4-ol

The titled compound was prepared by following the procedure of example 165, but replacing the product of example 164 with the product of example 310. ¹H-NMR (CDCl₃) δ: 1.23 (3H, t), 1.63-1.71 (3H, m), 1.98-2.10 (2H, m), 2.35-2.71 (10H, m), 2.89 (2H, t), 4.13 (2H, q), 5.31 (2H, brs), 6.08 (1H, t), 6.78 (1H, d), 7.00 (1H, dd), 7.12 (1H, d), 7.26-7.44 (5H, m), 7.57 (1H, dd), 8.49 (1H, dd).

MS m/z: 548 (M+1)

### EXAMPLE 289

4-(4-Chlorophenyl)-1-[3-(7-(1-(3-hydroxy)propyl)-5,11-dihydro[1]benzoxepino[2,3-b]pyridin-5-ylidene)propyl]piperidin-4-ol

The titled compound was prepared by following the procedure of example 133, but replacing the product of example 48 with the product of example 288.

¹H-NMR (DMSO-d₆) δ: 1.45-1.50 (2H, m), 1.66-1.80 (4H, m), 2.26-2.57 (10H, m), 3.41 (2H, q), 4.46 (1H, t), 4.83 (1H, s), 5.23 (2H, brs), 6.14 (1H, t), 6.71 (1H, d), 7.01 (1H, dd), 7.13 (1H, d), 7.34-7.48 (5H, m), 7.72 (1H, dd), 8.49 (1H, dd).

MS m/z: 505 (M+1)

### EXAMPLE 290

4-(4-Chlorophenyl)-1-[3-(5,11-dihydro-7-(2,3-dihydroxy)propyl[1]benzoxepino[2,3-b]pyridin-5-ylidene)propyl]piperidin-4-ol

To a solution of product of example 170 (6.9 g) in tetrahydrofuran (70 ml) and water (14 ml) were added N-methylmorpholine oxide(1.7 g) and osmium tetraoxide at 0° C., and the mixture was stirred at room temperature for 3 hours. Ethyl acetate was added to the mixture, the aqueous layer was separated. Chloroform-isopropanol (4:1) was added to the aqueous layer, the organic layer was extracted, and dried with magnesium sulfate. The solvent was distilled off under reduced pressure to give the titled compound (7.0 g).

¹H-NMR (CDCl₃) δ: 1.65-1.73 (2H, m), 1.95-2.10 (2H, m), 2.30-2.75 (13H, m), 3.45-3.50 (1H, m), 3.60-3.65 (1H, m), 3.83-3.90 (1H, m), 5.28 (2H, brs), 6.06 (1H, t), 6.84 (1H, d), 7.03 (1H, dd), 7.15 (1H, d), 7.26-7.43 (5H, m), 7.57 (1H, dd), 8.49 (1H, dd).

MS m/z: 521 (M+1)

### EXAMPLE 291

4-(4-Chlorophenyl)-1-[3-(5,11-dihydro-7-phenyl[1]benzoxepino[2,3-b]pyridin-5-ylidene)propyl]piperidin-4-ol

The titled compound was prepared by following the procedure of example 170, but replacing allyltributyltin with phenyltributyltin.

¹H-NMR (CDCl₃) δ: 1.84-1.92 (2H, m), 2.85-3.40 (10H, m), 5.33 (2H, brs), 6.05 (1H, t), 6.95 (1H, d), 7.30-7.58 (12H, m), 7.63-7.66 (1H, m), 8.56-8.58 (1H, m)

MS m/z: 523 (M+1)

### EXAMPLE 292

4-(4-Chlorophenyl)-1-[3-(7-(2-furyl)-5,11-dihydro[1]benzoxepino[2,3-b]pyridin-5-ylidene)propyl]piperidin-4-ol

The titled compound was prepared by following the procedure of example 170, but replacing allyltributyltin with ethyl (2-furyl)tributyltin.

¹H-NMR (CDCl₃) δ: 1.70-1.80 (3H, m), 1.97-2.16 (2H, m), 2.3-2.8 (8H, m), 5.36 (2H, m), 6.19 (1H, t), 6.45 (1H, dd), 6.55 (1H, d), 6.87 (1H, d), 7.20-7.50 (7H, m), 7.60-7.65 (2H, m), 8.52 (1H, dd)

MS m/z: 513 (M+1)

### EXAMPLE 293

### 4-(4-Chlorophenyl)-1-[3-(7-ethoxycarbonylamino-5,11-dihydro[1]benzoxepino[2,3-b]pyridin-5-ylidine)propyl]piperidin-4-ol

A mixture of product of example 118 (490 mg) and diphenylphosphonic azide (0.28 ml) was stirred at 110° C. for 30 minutes. After the mixture was cooled, and triethylamine (0.14 ml) and ethanol (5 ml) were added, and the mixture was heated to reflux for 8 hours. The reaction mixture was diluted with ethyl acetate and filterd through Celite. The filtrate was washed with saturated aqueous sodium bicarbonate, and dried over magnesium sulfate. The solvent was removed under reduced pressure and the residue was purified by silica gel column chromatography (chloroform:methanol=10:1) to give the titled compound (210 mg).

1H-NMR (CDCl₃) δ 1.31 (3H, t), 1.65-1.70 (2H, m), 2.01-2.09 (2H, m), 2.36-2.70 (8H, m), 4.21 (2H, q), 5.30 (2H, brs), 6.13 (1H, t), 6.46 (1H, brs), 6.80 (1H, d), 7.02 (1H, dd), 7.28-7.50 (6H, m), 7.57 (1H, dd), 8.50 (1H, dd).

MS m/z: 534 (M+H)

### EXAMPLE 294

1-[Bis(ethoxycarbonylmetyl)methoxy-5,11-dihydro[1]benzoxepino[2,3-b]pyridin-5-ylidene)propyl]-4-(4-chlorophenyl)-piperidin-4-ol

The titled compound was prepared by following the procedure of example 46, but replacing ethyl iodide with diethyl bromomalonate. ¹H-NMR (CDCl₃) δ: 1.30 (3H, t), 1.66-1.71 (2H, m), 1.98-2.09 (2H, m), 2.35-2.69 (9H, m), 4.30 (2H, q), 5.14 (1H, s), 5.26 (2H, brs), 6.10 (1H, t), 6.78 (2H, d), 7.00 (1H, t), 7.26-7.45 (5H, m), 7.57 (1H, dd), 8.43 (1H, dd).

MS m/z: 621 (M+1)

### EXAMPLE 295

1-[1,1-Bis(ethoxycarbonylmetyl)ethyloxy-5,11-dihydro[1]benzoxepino[2,3-b]pyridin-5-ylidene)propyl]-4-(4-chlorophenyl)-piperidin-4-ol

The titled compound was prepared by following the procedure of example 46, but replacing ethyl iodide with diethyl 2-bromo-2-methylmalonate.

¹H-NMR (CDCl₃) δ: 1.27 (6H, t), 1.65-1.70 (5H, m), 1.99-2.08 (3H, m), 2.31-2.69 (8H, m), 4.28 (4H, q), 5.27 (2H, brs), 6.06 (1H, t), 6.72 (1H, d), 6.80 (1H, dd), 7.00 (1H, d), 7.27-7.45 (5H, m), 7.56 (1H, dd), 8.46 (1H, dd).

### EXAMPLE 296

4-(4-Chlorophenyl)-1-[3-(5,11-dihydro-7-(2-hydroxy-1-hydroxymethyl)ethyloxy[1]benzoxepino[2,3-b]pyridin-5-ylidene)propyl]piperidin-4-ol

The titled compound was prepared by following the procedure of example 199, but replacing the product of example 138 with the product of example 294. ¹H-NMR (CDCl₃) δ: 1.70-1.75 (2H, m), 2.10-2.80 (11H, m), 3.90 (4H, d), 4.36 (1H, quint), 5.28 (2H, brs), 6.13 (1H, t), 6.71-6.87 (2H, m), 7.00 (1H, d), 7.29-7.45 (5H, m), 7.58 (1H, dd), 8.51 (1H, dd).

MS m/z: 537 (M+1)

### EXAMPLE 297

1-[1,1-Bis(hydroxymetyl)ethyloxy-5,11-dihydro[1]benzoxepino[2,3-b]pyridin-5-ylidene)propyl]-4-(4-chlorophenyl)-piperidin-4-ol

The titled compound was prepared by following the procedure of example 199, but replacing the product of example 138 with the product of example 295.

¹H-NMR (CDCl₃) δ: 1.09 (3H, s), 1.66-1.71 (2H, m), 1.90-2.10 (3H, m), 2.37-2.75(8H, m), 3.72-3.82(4H, m), 5.29(2H, brs), 6.05(1H, t), 6.77(1H, d), 6.88(1H, dd), 7.03(1H, d), 7.26-7.43(5H, m), 7.56(1H, dd), 8.48(1H, dd).

MS m/z: 551(M+1)

### EXAMPLE 299

4-(4-Chlorophenyl)-1-[3-(5,11-dihydro-7-(5-ethoxycarbonylpropyl)oxy[1]benzoxepino[2,3-b]pyridin-5-ylidene)propyl]piperidin-4-ol

The titled compound was prepared by following the procedure of example 46, but replacing ethyl iodide with ethyl 4-bromobutyrate.

¹H-NMR (CDCl₃) δ: 1.24(3H, t), 1.65-1.69(2H, m), 1.96-2.12(4H, m), 2.26-2.67(10H, m), 3.96(2H, t), 4.12(2H, q), 5.24(2H, brs), 6.08(1H, t), 6.70-6.83(3H, m), 7.21-7.59(6H, m), 8.39(1H, dd).

### EXAMPLE 300

1-[3-(7-(3-Carboxy-1-propyl)oxy-5,11-dihydro[1]benzoxepino[2,3-b]pyridin-5-ylidene)propyl]piperidin-4-ol

The titled compound was prepared by following the procedure of example 133, but replacing the product of example 48 with the product of example 299.

¹H-NMR (DMSO-d6) δ: 1.41-1.95(2H, m), 1.41-1.95(4H, m), 2.20-2.72(10H, m), 3.95(2H, t), 5.18(2H, brs), 6.17(1H, t), 6.72-6.84(3H, m), 7.36-7.48(5H, m), 7.77(1H, dd), 8.50(1H, dd).

MS m/z: 549(M+1)

### EXAMPLE 301

4-(4-Chlorophenyl)-1-[3-(5,11-dihydro-7-(4-methoxycarbonylphenyl)methoxy[1]benzoxepino[2,3-b]pyridin-5-ylidene)propyl]piperidin-4-ol

The titled compound was prepared by following the procedure of example 46, but replacing ethyl iodide with methyl 4-bromomethylbenzoate.

¹H-NMR (CDCl₃) δ: 1.66-1.70(2H, m), 1.93-2.09(3H, m), 2.37-2.70(8H, m), 3.91(3H, s), 5.09(2H, s), 5.27(2H, brs), 6.06(1H, t), 6.80-6.91(3H, m), 7.24-7.60(8H, m), 8.01-8.07(2H, m), 8.47 (1H, dd).

### EXAMPLE 302

1-[3-(7-(4-Carboxypheny)methoxy-5,11-dihydro[1]benzoxepino[2,3-b]pyridin-5-ylidene)propyl]-4-(4-chlorophenyl)piperidin-4-ol

The titled compound was prepared by following the procedure of example 133, but replacing the product of example 48 with the product of example 301.

¹H-NMR (DMSO-d6) δ: 1.44-1.49 (2H, m), 1.67-1.87 (2H, m), 2.26-2.56 (8H, m), 4.85 (1H, brs), 5.15-5.25 (4H, m), 6.17 (1H, t), 6.72-6.95 (3H, m), 7.30-7.75 (8H, m), 7.92-7.99 (2H, m), 8.48 (1H, dd).

MS m/z: 597 (M+1)

### EXAMPLE 303

4-(4-Chlorophenyl)-1-[3-(5,11-dihydro-7-((1-hydroxymethyl)cyclopropyl)methoxy[1]benzoxepino[2,3-b]pyridin-5-ylidene)propyl]piperidin-4-ol

Step 1

1-[3-(7-((1-Benzoyloxymethyl)cyclopropyl) methoxy-5,11-dihydro[1]benzoxepino [2,3-b]pyridin-5-ylidene)propyl]-4-(4-chlorophenyl)piperidin-4-ol was prepared by following the procedure of example 46, but replacing ethyl iodide with (1-benzoyloxymethyl)cyclopropylmethyl methanesulfonate.

¹H-NMR (CDCl₃) δ: 0.70-0.81 (4H, m), 1.65-1.70 (3H, m), 1.98-2.07 (2H, m), 2.35-2.70 (8H, m), 3.91 (2H, s), 4.39 (2H, s), 5.25 (2H, brs), 6.06 (1H, t), 6.72-6.84 (3H, m), 7.23-7.59 (9H, m), 8.02-8.06 (2H, m), 8.48 (1H, dd).

Step 2

The titled compound was prepared by following the procedure of example 133, but replacing the product of example 48 with the product of step 1.

¹H-NMR (CDCl₃) δ: 0.62 (4H, s), 1.67-1.72 (2H, m), 1.96-2.06 (2H, m), 2.34-2.69 (8H, m), 3.39 (1H, brs), 3.91 (2H, s), 3.91 (2H, s), 5.26 (2H, brs, 6.09 (1H, t), 6.72-6.86 (3H, M), 7.27-7.60 (6H, m), 8.48 (1H, dd).

MS m/z: 547 (M+1)

### EXAMPLE 305

1-[3-(5,11-dihydro-7-(2-hydroxyethyl)aminocarbonyl[1]benzoxepino[2,3-b]pyridin-5-ylidene)propyl]-4-(4-chlorophenyl)piperidin-4-ol

The titled compound was prepared by following the procedure of example 198, but replacing dimethylamine hydrochloride with 2-hydroxyehylamine.

¹H-NMR (CDCl₃) δ: 1.65-1.70 (2H, m), 2.03-2.06 (2H, m), 2.21 (1H, d), 2.32-2.68 (8H, m), 3.63 (2H, dt), 3.83 (2H, t), 5.37 (2H, brs), 6.18 (1H, t), 6.67 (1H, brs), 7.25-7.54 (7H, m), 7.86 (1H, dd), 8,50 (1H, dd).

MS m/z: 534 (M+1)

### EXAMPLE 306

4-(4-Chlorophenyl)-1-[3-(5,1 1-dihydro-7-(1-cyclohexyloxycarbonyloxy)ethyloxycarbonyl[1]benzoxepino[2,3-b]pyridin-5-ylidene)propyl]piperidin-4-ol Dihydrochloride

To a solution of product of example 118 (1.1 g) in dimethylformamide (15 ml) were added sodium iodide(0.17 g), potassium carbonate (0.38 g) and cyclohexyl 1-chloroethyl carbonate (J. Antibiotics, 1987, 40, 81.) (0.57 g) at room temperature. The mixture was stirred at 70° C. for 1 hour. Water and ethyl acetate were added to the reaction mixture, the organic layer was separated and washed with saturated aqueous sodium chloride, and dried with magnesium sulfate. The solvent was distilled off under reduced pressure and the residue was purified by silica gel column chromatography (ethyl acetate:methanol=100:3). The obtained oil was dissolved with ethyl acetate, and 4 N hydrochloric acid ethyl acetate solution (0.8 ml) was added. The precipitation was filtered to give the titled compound (0.96 g).

¹H-NMR (DMSO-d₆) δ: 1.22-1.47 (6H, m), 1.58 (3H, d), 1.63-1.81 (6H, m), 2.38-3.30 (10H, m), 4.07-4.59 (1H, m), 5.80 (2H, brs), 6.28 (1H, t), 6.87 (1H, q), 6.97 (1H, d), 7.40-7.49 (4H, m), 7.64 (1H, dd), 7.79 (1H, dd), 7.96 (1H, d), 8.03 (1H, dd), 8.65 (1H, dd), 11.07 (1H, brs).

MS m/z: 661[(M−2HCl)+1]

### EXAMPLE 307

4-(4-Chlorophenyl)-1-[3-(5,11-dihydro-7 (1-ethoxycarbonyloxy)ethyloxycarbonyl[1]benzoxepino[2,3-b]pyridin-5-ylidene)propyl]piperidin-4-ol

The titled compound was prepared by following the procedure of Example 307, but replacing cyclohexyl 1-chloroethyl carbonate with ethyl 1-chloroethyl carbonate.

MS m/z: 607 (M+1)

### EXAMPLE 308

4-(4-Chlorophenyl)-1-[3-(5,11-dihydro-7-(5-hydroxyfuran-2-yl)[1]benzoxepino[2,3-b]pyridin-5-ylidene)propyl]piperidin-4-ol

Step 1

4-(4-Chlorophenyl)-1-[3-(5,11-dihydro-7-(5-formylfuran-2-yl)[1]benzoxepino[2,3-b]pyridin-5-ylidene)propyl]piperidin-4-ol was prepared by following the procedure of example 170, but replacing allyltributyltin with (5-formylfuran-2-yl)tributyltin.

1H-NMR (CDCl₃) δ: 1.40-1.80 (2H, m), 1.89-2.12 (2H, m), 2.20-2.75 (8H, m), 5.28 (2H, brs), 6.16 (1H, t), 6.69 (1H, d), 6.84 (1H, d), 7.22-7.55 (8H, m), 7.76 (1H, d), 8.42 (1H, dd), 9.52 (1H, s).

Step 2

The titled compound was prepared by following the procedure of example 199, but replacing the product of example 138 with the product of step 1.

MS m/z: 543 (M+1)

### EXAMPLE 309

1-[3-(7-(5-Carboxyfuran-2-yl)-5,11-dihydro[1]benzoxepino[2,3-b]pyridin-5-ylidene)propyl]-4-(4-chlorophenyl)piperidin-4-ol

The titled compound was prepared by following the procedure of Example 382, step 2, butreplacing the product of Example 382, step 1 with the product of example 307, step 1.

MS m/z: 557 (M+1)

### EXAMPLE 310

4-(4-Chlorophenyl)-1-[3-(7-(2-ethoxycarboxy)ethenyl-5,11-dihydro[1]benzoxepino[2,3-b]pyridin-5-ylidene)propyl]piperidin-4-ol

The titled compound was prepared by following the procedure of example 171, but replacing t-butyl acrylate with ethyl acrylate.

¹H-NMR (CDCl₃) δ: 1.33 (3H, t), 1.63-1.71 (3H, m), 1.98-2.10 (2H, m), 2.35-2.72 (8H, m), 4.25 (2H, q), 5.36 (2H, brs), 6.10 (1H, t), 6.33 (1H, d), 6.85 (1H, d), 7.22-7.44 (7H, m), 7.58-7.65 (2H, m), 8.53 (1H, dd).

### EXAMPLE 311

4-(4-Chlorophenyl)-1-[3-(7-(1-(2-ethyl-2-hydroxy)butyl)oxy-5,11-dihydro[1]benzoxepino[2,3-b]pyridin-5-ylidene)propyl]piperidin-4-ol

The titled compound was prepared by following the procedure of example 200, but replacing ethylmagnesium bromide with methylmagnesium bromide.

¹H-NMR (CDCl₃) δ: 0.93 (6H, t), 1.60-1.70 (6H, m), 1.95-2.10 (3H, m), 2.36-2.70 (8H, m), 3.79 (2H, s), 5.28 (2H, brs), 6.09 (1H, t), 6.77-6.86 (3H, m), 7.24-7.43 (5H, m), 7.57 (1H, dd), 8.47 (1H, dd).

MS m/z: 563 (M+1)

### EXAMPLE 312

4-(4-Chlorophenyl)-1-[3-(7-(2-(2,3-dimethyl-3-hydroxy)butyl)oxy-5,11-dihydro[1]benzoxepino[2,3-b]pyridin-5-ylidene)propyl]piperidin-4-ol

The titled compound was prepared by following the procedure of example 200, but replacing the product of example 48 with the product of example 138.

¹H-NMR (CDCl₃) δ: 1.22 (6H, s), 1.32 (6H, s), 1.66-1.71 (2H, m), 1.99-2.10 (2H, m), 2.35-2.85 (9H, m), 3.77 (2H, s), 5.28 (2H, brs), 6.04 (1H, t), 6.74-6.89 (3H, m), 7.26-7.43 (5H, m), 7.57 (1H, dd), 8.44 (1H, dd).

MS m/z: 563 (M+1)

### EXAMPLE 313

4-(4-Chlorophenyl)-1-[3-(5,11-dihydro-7-(2-oxopropyl)oxy[1]benzoxepino[2,3-b]pyridin-5-ylidene)propl]piperidin-4-ol

The titled compound was prepared by following the procedure of example 146, but replacing ethyl iodide with chloracetone.

1H-NMR (CDCl₃) δ: 1.62-1.71 (3H, m), 1.99-2.10 (2H, m), 2.27 (3H, s), 2.3 5-2.70 (8H, m), 4.51 (2H, s), 5.28 (2H, brs), 6.08 (1H, t), 6.70-6.84 (3H, m), 7.25-7.32 (3H, m), 7.41-7.44 (2H, m), 7.58 (1H, dd), 8.50 (1H, dd).

MS m/z: 519 (M+1)

### EXAMPLE 314

4-(4-Chlorophenyl)-1-[3-(7-formyl-5,11-dihydro[1]benzoxepino[2,3-b]pyridin-5-ylidene)propyl]piperidin-4-ol

To a solution of the product of example 274 (1.0 g) in methylene chloride(200 ml) was added manganese(IV) oxide(3.0 g), and the suspension was stirred at ambient temperature for 12 hours. The reaction mixture was diluted with ethyl acetate and filtered through Celite. The solvent was evaporated under reduced pressure to give the titled compound (930 mg).

¹H-NMR (CDCl₃) δ: 1.71-1.80 (3H, m), 1.98-2.09 (2H, m), 2.35-2.43 (4H, m), 2.53-2.69 (4H, m), 5.30 (2H, brs), 6.24 (1H, t), 6.95 (1H, d), 7.27-7.44 (5H, m), 7.61 (1H, dd), 7.67 (1H, dd), 7.85 (1H, d), 8.54 (1H, dd), 9.88 (1H, s).

### EXAMPLE 315

1-[3-(7-Acetyl-5,11-dihydro[1]benzoxepino[2,3-b]pyridin-5-ylidene)propyl]-4-(4-chlorophenyl)piperidin-4-ol

Step 1

To a solution of example 53, step 1 (7.2 g) in dichloromethane (70 ml) was added aluminum chloride (9.1 g) and acetyl chloride (3.2 ml), and the mixture stirred at 0° C. for 10 minutes. The reaction mixture was poured into ice. The aqueous layer was extracted with ethyl acetate, and the organic layer was washed with saturated aqueous sodium chloride, and dried with magnesium sulfate. The solvent was distilled off under reduced pressure. The Residue was purified by silica gel chromatography, eluting with ethyl acetate-hexane (1:2) to give 7-acetyl-5-(3-bromopropylidene)-5,11-dihydro[1]benzoxepino[2,3-b]pyridine (7.9 g).

¹H-NMR (CDCl₃) δ: 2.57 (3H, s), 2.77 (2H, m), 3.49 (2H, t), 5.40 (2H, brs), 6.16 (1H, t),6.88 (1H, d), 8.33 (1H, dd), 7.58 (1H, dd), 7.77 (1H, dd), 7.96 (1H, d), 8.56 (1H, dd).

Step 2

The titled compound was prepared by following the procedure of example 44, step 2,but replacing the product of example 44, step 1 with the product of step 1.

¹H-NMR (CDCl₃) δ: 1.52-1.79 (2H, m), 1.93-2.11 (2H, m), 2.27-2.49 (4H, m), 2.49-2.60 (5H, m), 2.60-2.73 (2H, m), 5.40 (2H, brs), 6.22 (1H, t),6.87 (1H, d), 7.29-7.34 (3H, m), 7.42 (2H, d), 7.59 (1H, dd), 7.75 (1H, dd), 7.96 (1H, d), 8.53 (1H, dd).

MS m/z: 489 (M+1)

### EXAMPLE 316

To a stirred solution of phenol containing the product of Example 44 (1.0 mmol) and K₂CO₃ (1.5 mmol) in THF (10 mL) at RT was added N,N-dimethylcarbamoylchloride (1.2 mmol). The reaction was stirred at reflux for 24 hrs. Excess solvent was removed and pure compound was isolated via silica gel chromatography eluting with 5% MeOH/CH₂Cl₂. MS m/z: (M+535)

### EXAMPLE 317

To a stirred solution of phenol containing the product of Example 44 (1.0 mmol) and K₂CO₃ (1.5 mmol) in THF (10 mL) at RT was added morpholinocarbamoylchloride (1.2 mmol). The reaction was stirred at reflux for 24 hrs. Excess solvent was removed and pure compound was isolated via silica gel chromatography eluting with 5% MeOH/CH₂Cl₂. MS m/z: (M+577)

### EXAMPLE 318

To a stirred solution of phenol containing the product of Example 44 (1.0 mmol) in DMF at RT was added NaH (1.5 mmol) followed by the addition of N-isopropylisocyanate (1.5 mmol). The reaction was heated to 60° C. for 6 hrs. The reaction was quenched with 1.5 equivalents of H₂O and excess DMF was removed under reduced pressure. Residue was charged on a silica gel column and eluted off with 5% MeOH/CH₂Cl₂. MS m/z: (M+548)

### EXAMPLE 319

To a stirred solution of phenol containing the product of Example 44 (1.0 mmol) and K₂CO₃ (1.5 mmol) in THF (10 mL) at RT was added N-methyl-N-phenylcarbamoylchloride (1.2 mmol). The reaction was stirred at reflux for 24 hrs. Excess solvent was removed and pure compound was isolated via silica gel chromatography eluting with 5% MeOH/CH₂Cl₂. MS m/z: (M+597)

### EXAMPLE 320

To a stirred solution of phenol containing the product of Example 44 (1.0 mmol) in DMF at RT was added NaH (1.5 mmol) followed by the addition of N-phenylisocyanate (1.5 mmol). The reaction was heated to 60° C. for 6 hrs. The reaction was quenched with 1.5 equivalents of H₂O and excess DMF was removed under reduced pressure. Residue was charged on a silica gel column and eluted off with 5% MeOH/CH₂Cl₂. MS m/z: (M+583)

### EXAMPLE 321

To a stirred solution of phenol containing the product of Example 44 (1.0 mmol) in DMF at RT was added NaH (1.5 mmol) followed by the addition of N-(3-pyridyl)isocyanate(1.5 mmol). The reaction was heated to 60° C. for 6 hrs. The reaction was quenched with 1.5 equivalents of H₂O and excess DMF was removed under reduced pressure. Residue was charged on a silica gel column and eluted off with 5% MeOH/CH₂Cl₂. MS m/z: (M+584)

### EXAMPLE 322

To a stirred solution of phenol containing the product of Example 44 (1.0 mmol) and K₂CO₃ (1.5 mmol) in THF (10 mL) at RT was added pyrolidinylcarbamoylchloride (1.2 mmol). The reaction was stirred at reflux for 24 hrs. Excess solvent was removed and pure compound was isolated via silica gel chromatography eluting with 5% MeOH/CH₂Cl₂. MS m/z: (M+560)

### EXAMPLE 323

The compound was prepared by following the procedure for example 45, step 3, but replacing 4-(4-chlorophenyl)-4-hydroxypiperidine with 4-(4-chlorophenyl)-4-cyanopiperidine. MS m/z: (M+486).

### EXAMPLE 324

To a cold (0° C.) stirred solution of Example 323 (0.50 g, 0.104 mmol) in anhydrous THF (5 mL) was added lithium aluminum hydride (8 mg, 0.21 mmol). The reaction was stirred at RT for 2 hrs. The reaction was then quenched by the careful addition of H₂O (0.21 mL), 15% aqueous KOH (0.21 mL), then H₂O (0.21 mL). The organic layer was separated and dried over NaSO₄. The compound was purified via silica gel flash chromatography eluting with 10% methanol/methylene chloride. MS m/z: (M+490).

### EXAMPLE 325

The compound can be obtained by the reduction of the azido functionality of example 187 with a reducing agent, such as triphenyl phoshine, lithium aluminum hydride, sodium borohydride, in a solvent such as tetrahydrofuran or diethyl ether in reaction temperature ranges from 0° C. to reflux with a reaction time between 5 minutes and 72 hours.

### EXAMPLE 326

The compound was prepared by following the procedure for example 45, step 3, but replacing 4-(4-chlorophenyl)-4-hydroxypiperidine with 4-(4-chlorophenyl)-4-methylpiperidine provide in Example 329, steps 1-3. MS m/z: (M+475)

### EXAMPLE 328

Step 1

N-benzyl-4-(4-chlorophenyl)-4-hydroxypiperidine: FIG. 8a

To a stirred solution of commercially available 4-(4-chlorophenyl)-4-hydroxypiperidine (10 g, 47 mmol., 1) in anhydrous DMF (10 mL) was added benzyl bromide (5.6 mL, 47 mmol) and K₂CO₃ (7.4 g, 94 mmol.) and stirred at RT overnight. Excess solvent was removed under reduced pressure, brought up into CH₂Cl₂ (100 mL) washed with H₂O (2×50 mL). Organic layer separated, dried over Na₂SO₄ and charged on a silica gel flash column. Eluting off with 2% MeOH/CH₂Cl₂ 10 g 2 (80% yield) was obtained as a viscous liquid. MS m/z: (M+303)

Step 2

N-benzyl-4-(4-chlorophenyl)-4-fluoropiperidine: FIG. 8a

To a cold (−78° C.) solution of 2 (10 g, 33 mmol) in CH₂Cl₂ (20 mL) was slowly added DAST (diethylaminosulfur trifluoride, 5.3 mL, 39.8 mmol) under an inert atmosphere. The reaction was stirred at −78° C. for an additional 45 min. The reaction was quenched at −78° C. by the slow addition of enough saturated aqueous sodium bicarbonate solution to afford a pH >8. This reaction resulted a quantitative conversion of the starting material to a 1:1 mixture of fluoropiperidine 3 and 4-(4-chlorophenyl)tetrahydropyridine 4. The mixture of 3 and 4 (3.5 g, mixture, 35% yield) was purified via silica gel flash chromatography, eluting with 2% MeOH/CH₂Cl₂. This mixture proved to be inseparable by silica gel flash chromatography. In order to separate out the desired product, the mixture of 3 and 4 were subjected to osmium tetroxide oxidation.

To a stirred solution of the mixture of 3 and 4 (1.8 g) in acetone/H₂O (5:1, 10 mL) was added a catalytic amount of OSO₄ in isopropanol (2.5 mol %, 1 mL) and N-methylmorpholine-N-oxide (0.69 g, 6.56 mmol). The reaction was stirred at RT overnight. The reaction was then evaporated to dryness, brought up into CH₂Cl₂ and washed with NaHSO₃. This reaction resulted in the dihydroxylation of the undesired 4 to 5 and the clean separation of the desired fluoropiperidine 3 (1.0 g, 55% yield) from the byproduct by silica gel flash chromatography eluting with 2% MeOH/CH₂Cl₂. MS m/z: (M+306)

Step 3

4-(4-chlorophenyl)-4-fluoropiperidine: FIG. 8a

To a cold (0° C.) solution of 3 (1.07 g, 3.5 mmol) in 1,2-dichloroethane was added 1,1-chloroethylchloroformate (0.45 mL, 4.2 mmol). The reaction was then heated to reflux for 2 hrs. Excess solvent was removed and the residue was brought up into 5 mL methanol. The mixture was refluxed for 2 hrs and excess methanol was removed under reduced pressure. Precipitation of the hydrochloride salt of 6 by the addition of CH₂Cl₂/hexane (1:1) followed by filtration resulted in the quantitative isolation of the desired crystalline product 6 (80%, 0.70 g). MS m/z: (M+215)

Step 4

The compound was prepared by following the procedure for example 44, but replacing 4-(4-chlorophenyl)-4-hydroxypiperidine with 4-(4-chlorophenyl)-4-fluoropiperidine. MS m/z: (M+466).

### EXAMPLE 329

Step 1

N-benzyl-4-methylpiperidine: FIG. 8c

To a cold (−78° C.) stirred solution of 1.4 M methyllithium in THF (39 mL, 54 mmol) under an inert atmosphere was added N-benzyl-4-oxopiperidine (1, 5.1 g, 27 mmol). The reaction was stirred at -78° C. for 2hrs. The reaction was quenched by the slow addition of saturated aqueous NH₄Cl, the organic layer was separated and dried over Na₂SO₄. Pure methylpiperidine (2) was isolated via silica gel flash chromatography eluting with 5% MeOH/CH₂Cl₂. MS m/z: (M+206)

Step 2

N-benzyl-4-(4-chlorophenyl)-4-methylpiperidine: FIG. 8c

To a flask containing chlorobenzene (10 mL, excess) and methylpiperidine (0.42 g, 2.06 mmol, 2) was added aluminum trichloride (1.65 mL, 12.4 mmol). The reaction was heated to reflux for 24 hrs. Excess chlorobenzene was removed under reduced pressure and pure 3 was obtained via silica gel flash chromatography eluting with % EtOAc/hexane. MS m/z: (M+300)

Step 3

4-(4-chlorophenyl)-4-methylpiperidine: FIG. 8c

To a cold (0° C.) solution of N-benzyl-4-(4-chlorophenyl)-4-methylpiperidine (3) (0.41 g, 1.4 mmol) in CH₂Cl₂ was 1.1 equivalent of 1-chloroethylchloroformate. The reaction was then heated to reflux for 2 hrs. Excess solvent was removed and the residue was brought up into methanol. The mixture was refluxed for 2 hrs and excess methanol was removed under reduced pressure. Precipitation of the hydrochloride salt 4 by the addition of CH₂Cl₂ followed by filtration resulted in the quantitative isolation of the desired crystalline product 4 (100%, 0.34 g). MS m/z: (M+210)

Step 4

The compound was prepared by following the procedure for example 44, step 2, but replacing 4-(4-chlorophenyl)-4-hydroxypiperidine with 4-(4-chlorophenyl)-4-methylpiperidine. MS m/z: (M+461)

### EXAMPLE 330

The compound was prepared by following the procedure for example 199, but replacing the resultant compound of example 44 with the resultant compound of example 329. MS m/z: (M+533)

### EXAMPLE 331

Step 1

A mixture of epichlorohydrin (5.92 g, 64 mmol) and benzhydrylamine (11.7 g, 64 mmol) in MeOH (120 mL) was stirred under the protection of argon at room temperature for 48 hours. The mixture was then stirred at 50° C. for 72 hours. The reaction mixture was then stirred at room temperature for 72 hours. The reaction mixture was concentrated in vacuo and partitioned between EtOAc and H₂O. The aqueous layer was extracted with EtOAc (200 mL×3), dried over MgSO₄ and concentrated in vacuo. Chromatographic purification on silica gel (CH₂Cl₂/MeOH =95/5) provided 10.0 g (65%) of 1-benzhydril-3-hydroxyazetidine. m/z 240 (m+1)

Step 2

A mixture 1-benzhydril-3-hydroxyazetidine (2.6 g, 11 mmol) and palladium hydroxide on active carbon (0.26 g, w/w 20%) in EtOH (40 mL) was shaken in hydrogenation parr under 60 psi for 24 hours. The reaction mixture was filtered through celite and concentrated under vacuum. Concentration in vacuo provided 0.75 (95%) 3-hydroxyazetidine. ¹H NMR (250 MHz, CD3OD) 3.81-3.92 (2H, m), 4.14-4.25 (2H, m), 4.61-4.69 (1H, m).

Step 3

The compound 1-[3-(5,11-dihydro-7-(methoxy[1]benzoxepino[2,3-b]pyridin-5-ylidene)propyl]azetidin-3-ol was prepared by following the procedure for example 45, step 3, but replacing 4-(4-chlorophenyl)-4-hydroxypiperidine with 3-hydroxyazetidine. m/z 339 (m+1).

Step 4

To a mixture of morpholine N-oxide (0.028 g, 0.244 mmol), crushed molecular sieves (0.066 g) and Pr₄N+RO₄ (0.01 g, 0.024 mmol) in CH₂Cl₂ was added the 1-[3-(5,11-dihydro-7-(methoxy[1]benzoxepino[2,3-b]pyridin-5-ylidene)propyl]azetidin-3-ol (0.055 g, 0.16 mmol) under the protection of argon. The mixture was stirring over night at room temperature. The reaction mixture was filtered off through celite and concentrated under vacuum. Chromatographic purification on silica gel (CH₂Cl₂/MeOH=95/5 to 9/1) provided 0.033 g 1-[3-(5,1 1-dihydro-7-(methoxy[1]benzoxepino[2,3-b]pyridin-5-ylidene)propyl]azetidin-3-one (60%) of the desired product. m/z 337 (m+1)

Step 5

To a solution of 1-[3-(5,11-dihydro-7-(methoxy[1]benzoxepino[2,3-b]pyridin-5-ylidene)propyl]azetidin-3-one (0.06 g, 0.18 mmol) in THF (8 mL) was added dropwise a solution of 4-chlorophenyl magnesium bromide in diethyl ether (1.0 M, 0.27 mL) under the the protection of argon at 0° C. The reaction was stirred at room temperature for 1.5 hours and quenched by the addition of saturated aqueous NH₄OH (4 mL). The aqueous layer was extracted with EtOAc (10 mL×2), dried over MgSO₄ and concentrated in vacuo. Chromatographic purification on silica gel (CH₂Cl₂/MeOH =95/5) provided 0.048 g 3-(4-chlorophenyl)-1-[3-(5,11-dihydro-7-(methoxy[1]benzoxepino[2,3-b]pyridin-5-ylidene)propyl]azetidine (51%) m/z 449 (m+1)

### EXAMPLE 332

Step 1

tert-Butyl 3-(4-chlorobenzoyl)-1-(2-aminoethyl) carbamate: FIG. 10b

tert-Butyl N-(2-aminoethyl) carbamate (1, 0.50 g g, 3.12 mmol) was added to the mixture of 4-chlorobenzoic acid chloride (0.547 g, 3.12 mmol) and Et₃N (1.74 mL, 12.5 mmol) in CH₂Cl₂ (20 mL) under the protection of argon. Stirring at room temperature for 2 hours. The reaction mixture was diluted with H₂O (25 mL), extracted with CH₂Cl₂ (50 mL×2), dried over MgSO₄ and concentrated in vacuo. Chromatographic purification on silica gel (CH ₂Cl₂/MeOH=95/5) to provide 0.86 g (2, 93%) of the desired product tert-Butyl 3-(4-chlorobenzoyl)-1-(2-aminoethyl) carbamate. MS m/z: (M+299).

Step 2

1-(4-chlorobenzoyl)-1,2-ethylenediamine: FIG. 10b

Trifluoroacetic acid (7.5 mL) was added to the solution of tert-Butyl 3-(4-chlorobenzoyl)-1-(2-aminoethyl)carbamate (2, 0.86 g, 2.89 mmol) in CH₂Cl₂ (35 mL) at 0° C. Stirring at room temperature for 30 minutes. Concentration in vacuo provided 0.88 g (95%) of the desired product 1-(4-chlorobenzoyl)-1,2-ethylenediamine (3). MS m/z: (M+199).

Step 3

The compound was prepared by following the procedure for example 45, step 3, but replacing 4-(4-chlorophenyl)-4-hydroxypiperidine with 1-(4-chlorobenzoyl)-1,3-propylenediamine. MS m/z: (M+465).

### EXAMPLE 333

Step 1

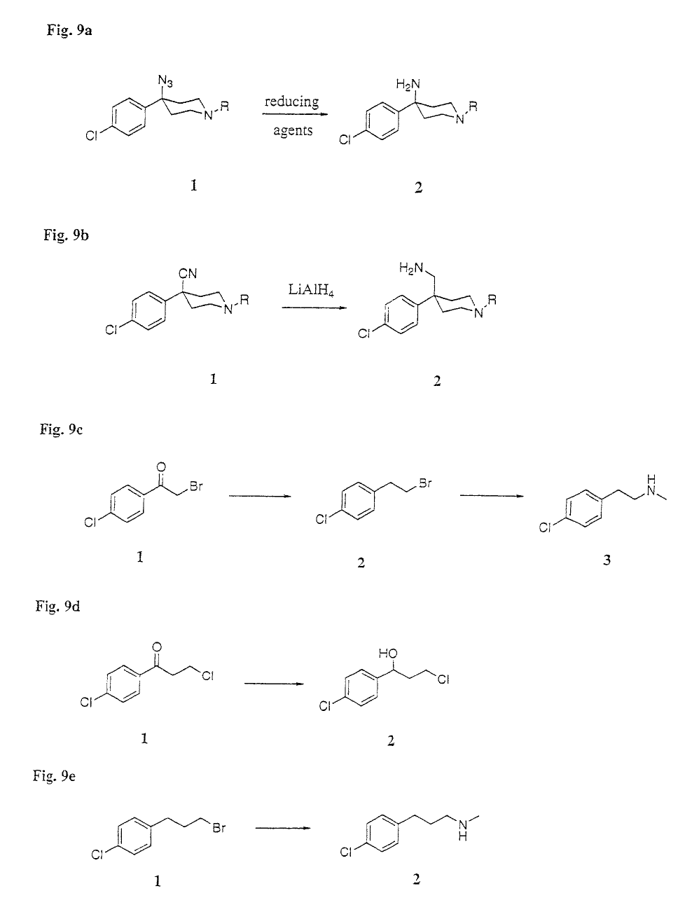

2-(4-Chlorophenyl)-1-bromoethylene: FIG. 9c

To a solution of AlCl₃ (1.96 g, 14.7 mmol) in anhydrous CH₂Cl₂ (50 mL), Borane-tert-butyl amine complex (2.57 g, 29.6 mmol) was added at 0° C. under argon protection, stirred for 10 minutes and clear solution was formed. 4-Chlorophenacyl bromide (1, 1.11 g, 4.91 mmol) in CH₂Cl₂ (5 mL) was added to the resulted mixture at 0° C. The reaction was stirred for 1.5 hours and then quenched by the addition of 0.1 N HCl (25 mL). The mixture was extracted with EtOAc (80 mL×3), dried over MgSO₄ and concentrated in vacuo. Chromatographic purification on silica gel (Hexane/EtOAc=9:1) provided 0.85 g (84%) of 2-(4-chlorophenyl)-1-bromoethylene (2). MS m/z: (M+219).

Step 2

2-(4-chlorophenyl)-1-(N-methyl)ethylamine: FIG. 9c

A mixture of 2-(4-chlorophenyl)-1-bromoethylene (2, 1.02 g, 4.62 mmol), EtOH (3 mL) and H₂NMe in H₂O (6 mL, 40% w/w) was heated at 135 0° C. over night. The mixture was cooled down to room temperature. The mixture was extracted with Et₂O (5 mL×2), dried over MgSO₄ and concentrated in vacuo. Chromatographic purification on silica gel (CH₂Cl₂/MeOH/NH₄OH 9/1/0.1) provided 0.61 g 2-(4-chlorophenyl)-1-(N-methyl)ethylamine (3, 79%). MS m/z: (M+170).

Step 3

The compound was prepared by following the procedure for example 45, step 3, but replacing 4-(4-chlorophenyl)-4-hydroxypiperidine with 2-(4-chlorophenyl)-1-(N-methyl)ethylamine. MS m/z: (M+451).

### EXAMPLE 334

Step 1

3-(4-chlorophenyl)-1-N-methylaminopropane: FIG. 9e

A mixture of 3-(4-chlorophenyl)-1-bromoropane (1, 0.70 g, 3.73 mmol), EtOH (3 mL) and H₂NMe in H₂O (6 mL, 40% w/w) was heated at 135 0° C. overnight. The mixture was then cooled down to room temperature. The mixture was extracted with Et2O (5 mL×2), dried over MgSO₄ and concentrated in vacuo. Chromatographic purification on silica gel (CH₂Cl₂/MeOH/NH₄OH=9/1/0.1) provided 0.5 g (76%) of 3-(4-chlorophenyl)-1-N-methylaminopropane (2). MS m/z: (M+189).

Step 2

The compound was prepared by following the procedure for example 45, step 3, 5 but replacing 4-(4-chlorophenyl)-4-hydroxypiperidine with 3-(4-chlorophenyl)-1-N-methylaminopropane. MS m/z: (M+450).

### EXAMPLE 335

Step 1

3-(4-chlorophenyl)-3-chloro-1-hydroxypropane: FIG. 9d

To 3,4′-Dichloropropylphenone (0.52 g, 2.53 mmol) in anhydrous MeOH (10 mL) at 0° C. under the protection of argon, NaBH₄ (0.23 g, 3.03 mmol) was added to the solution by several portions. The reaction was stirred under the same condition for 15 minutes. The mixture was warmed up to room temperature, stirred an additional 30 minutes, then concentration in vacuo. The residue was partitioned between EtOAc and H₂O. The aqueous layer was re-extracted with EtOAc (30 mL×2), dried over MgSO₄ and concentrated in vacuo. Chromatographic purification on silica gel (Hexane/EtOAc =(1/1) provided 0.52 g (99%) of 3-(4-chlorophenyl)-3-chloro-1-hydroxypropane. MS m/z: (M+205).

Step 2

The compound was prepared by following the procedure for example 45, step 3, but replacing 4-(4-chlorophenyl)-4-hydroxypiperidine with 3-(4-chlorophenyl)-3-chloro-1-hydroxypropane. MS m/z: (M+481).

### EXAMPLE 336

Step 1

3-(4-chlorophenyl)-3-hydroxy-3-methyl-1-chloropropane: FIG. 10a

To 3,4′-Dichloropropylphenone (1, 1.10 g, 5.40 mmol) in anhydrous THF at 0° C. under the protection of argon, was added MeMgBr (2.50 mL, 7.35 mmol) dropwise at 0° C. The reaction was stirred at room temperature for an additional hour. The reaction was quenched by adding saturated aqueous NH₄Cl. The reaction was then extracted with Et₂O (60 mL×2), dried over MgSO₄ and concentrated in vacuo. Chromatographic purification on silica gel (Hexane/EtOAc=10/1) provided 1.0 g (85%) of 3-(4-chlorophenyl)-3-hydroxy-3-methyl-1-bromoropane (2). MS m/z: (M+219).

Step 2

3-(4-chlorophenyl)-3-hydroxyl-3-methyl-1-N-methylaminopropane: FIG. 10a

A mixture of 3,3,3-(4-Chlorophenyl)-hydroxylmethyl-1-bromoropane (2, 1.04 g, 4.74 mmol), EtOH (5 mL) and H₂NMe in H₂O (10 mL, 40% w/w) was heated at 135 0° C. for 3 hours. The mixture was cooled down to room temperature. The mixture was extracted with Et₂O (5 mL×2), dried over MgSO₄ and concentrated in vauco. Chromatographic purification on silica gel (CH₂Cl₂/MeOH/NH₂OH=9/1/0.1) provided 1.01 g 3-(4-chlorophenyl)-3-hydroxyl-3-methyl-1-N-methylaminopropane (3, 99%). MS m/z: (M+214).

Step 3

The compound was prepared by following the procedure for example 45, step 3, but replacing 4-(4-chlorophenyl)-4-hydroxypiperidine with 3-(4-chlorophenyl)-3-hydroxyl-3-methyl-1-N-methylaminopropane. MS m/z: (M+480).

### EXAMPLE 345

Using the procedure of Example 45, but replacing 5,1 1-dihydro-7-methoxy[1]benzoxepino[2,3-b]pyridin-5-one with 1-azaxanthone, gives the desired compound.

### EXAMPLE 346

Using the procedure of Example 45, but replacing 5,11-dihydro-7-methoxy[1]benzoxepino[2,3-b]pyridin-5-one with 1-4-azafluorene, gives the desired compound.

### EXAMPLE 347

Using the procedure of Example 45, but replacing 5,11-dihydro-7-methoxy[1]benzoxepino[2,3-b]pyridin-5-one with 7-amino-1-azaxanthone, gives the desired compound.

### EXAMPLE 348

Using the procedure of Example 45, but replacing 5,1 1-dihydro-7-methoxy[1]benzoxepino[2,3-b]pyridin-5-one with 4,5-diazafluorene, gives the desired compound.

### EXAMPLE 349

Using the procedure of Example 45, but replacing 5,11-dihydro-7-methoxy[1]benzoxepino[2,3-b]pyridin-5-one with 1-aza-7-nitroxanthone, gives the desired compound.

### EXAMPLE 350

3-(4-chlorophenyl)-1-[3-(5,11-dihydro-7-(methoxy[1]benzoxepino[2,3-b]pyridin-5-ylidene)propyl]pyrrolidine

Step 1

A mixture of 1-benzyl-3-pyrrolidinone (10.0 g, 57 mmol), di-tert-butyl dicarbonate (13.7 g, 63 mmol) and palladium on active carbon (2.5 g, w/w 20%) in MeOH was shaken in a Parr hydrogenation vessel (50 psi H₂) for 48 hours. The reaction mixture was filtered through celite and concentrated in vacuo. Chromatographic purification on silica gel (Hexane/EtOAc=1/1) provided 6.21 g 1-t-butoxycarbonyl-3-pyrrolidinone (59%). ¹H NMR (250 MHz, CDCl3) δ: 1.46 (9H, s), 2.57 (2H, t, J=7.8 Hz), 3.71-3.75 (4H, m)

Step 2

To a stirred solution of 1-t-butoxycarbonyl-3-pyrrolidinone (0.57 g, 3.23 mmol) in THF (10 mL) was added 4-chlorophenyl magnesium bromide (1.0 M, 5.2 mL) under the protection of argon at 0° C. The reaction was stirred at room temperature for 1 hour then quenched by the addition of saturated aqueous NH₄OH (8 mL). The aqueous layer was extracted with EtOAc (50 mL×2), dried over MgSO₄ and concentrated in vacuo. Chromatographic purification on silica gel (Hexane/EtOAc=3/1) provided 0.57 g 1-t-butoxycarbonyl-3-(4-chlorophenyl)-3-hydroxypyrrolidine (60%). m/z 298 (m+1)

Step 3

To a stirred solution of 1-t-butoxycarbonyl-3-(4-chlorophenyl)-3-hydroxypyrrolidine (0.335 g, 1.28 mmol) in CH₂Cl₂ (8 mL) was added trifluoroacetic acid (2 mL) at 0° C. slowly. The reaction was stirred at room temperature for 30 minutes and concentrated in vacuo. This provided 0.355 g 3-(4-chlorophenyl)-3-hydroxypyrrolidine (100%) the desired product. m/z 198 (m+1)

Step 4

The titled compound was prepared by following the procedure for example 44 but replacing 4-(4-chlorophenyl)-4-hydroxypiperidine with 3-(4-chlorophenyl)-3-hydroxypyrrolidine. m/z 432 (m+1).

### EXAMPLE 351

Step 1

4-(4-chlorophenyl)-4-pyridine: FIG. 10d

To a solution of 4-bromopyridine (1, 1.94 g, mmol), 4-chlorophenylboronic acid (2, 1.56 g, mmol) and K₂CO₃ (2.76 g, 2.0 equiv) in ethanol/toluene (5 mL/100 mL) was added Pd(PPh₃)₃. The reaction was refluxed for 1 hr, cooled back down to RT and quenched with H₂O (15 mL). The reaction mixture was extracted with EtOAc and the organic layer was dried over Na₂SO₄. Pure 4-(4-chlorophenyl)-4-pyridine 2 (1.3 g, 68% yield) was isolated after silica gel flash column purification eluting with 50% EtOAc/hexane. MS m/z: (M+191).

Step 2

The titled compound was prepared by following the procedure for example 45, step 3, but replacing 4-(4-chlorophenyl)-4-hydroxypiperidine with 4-(4-chlorophenyl)-4-pyridine. MS m/z: (M+456).

### EXAMPLE 352

The compound was prepared by following the procedure for example 44, but replacing 4-(4-chlorophenyl)-4-hydroxypiperidine with 4-(4-chlorophenyl)-4-pyridine. MS m/z: (M+442).

### EXAMPLE 353

5-(2-(N-(4-(4-Chlorophenyl)-4-hydroxycyclohexyl)-N-methyl)ethylidene)-5,11-dihydro-7-methoxy[1]benzoxepino[2,3-b]pyridine The compound was prepared by the procedure of Example 57, step 3, but replacing 4-(4-chlorophenyl)-4-hydroxypiperidine with 4-(4-N-methyl-(4-chlorophenyl)-4-hydroxycyclohexylamin. The starting material can be prepared according to methods disclosed in Journal of Medicinal Chemistry, Vol. 15, No. 12, pp.1239-1243 (1972).

### EXAMPLE 354

1-[3-(7-(4-Carboxyphenoxy)-5,11-dihydro[1]benzoxepino[2,3-b]pyridin-5-ylidene)propyl]-4-(4-chlorophenyl)piperidin-4-ol

Step 1

4-(4-Chlorophenyl)-1-[3-(5,11-dihydro-7-(4-ethoxycarbonylphenoxy)[1]benzoxepino[2,3-b]pyridin-5-ylidene)propyl]piperidin-4-ol was prepared by following the procedure of example 46, but replacing ethyl iodide with ethyl 4-fluorobenzoate

¹H-NMR (CDCl₃) δ: 1.36 (3H, t), 1.65-2.07 (4H, m), 2.32-2.63 (8H, m), 4.34 (2H, q),5.33 (2H, brs), 6.07 (1H, t). 6.88-7.10 (5H, m), 7.27-7.51 (5H, m), 7.58 (1H, dd), 7.97-8.00 (2H, m), 8.49 (1H, dd).

Step 2

The titled compound was prepared by following the procedure of example 133, but replacing the product of example 48 with the product of step 1.

¹H-NMR (DMSO-d6) δ: 1.44-1.49 (2H, m), 1.67-1.87 (2H, m), 2.26-2.56 (8H, m),4.85 (1H, brs), 5.29 (2H, brs), 6.17 (1H, t), 6.88-7.09 (5H, m), 7.33-7.48 (5H, m), 7.75 (1H, dd), 7.89-7.93 (2H, m), 8.52 (1H, dd).

MS m/z: 582 (M)

### EXAMPLE 355

4-(4-Chlorophenyl)-1-[3-(5,11-dihydro-7-(2-(hydroxyimino)propyl)oxy[1]benzoxepino[2,3-b]pyridin-5-ylidene)propyl]piperidin-4-ol

To a solution of the product of example 313 (300 mg) in ethanol (3, ml) was added hydroxylammonium chloride (80 mg) at room temperature, and the mixture was stirred for 1 hour. The precipitation was filtered and washed with ethanol to give the titled compound (300 mg).

¹H-NMR (DMSO-d6) δ: 1.75-1.80 (2H, m). 2.23-2.42 (2H, m), 2.53 (3H, s)3.16-3.48 (8H, m), 4.54 (2H, s), 5.19 (2H, brs), 5.57 (1H, s), 6.14 (1H, t), 6.76-6.98 (3H, m),7.41-7.48 (5H, m), 7.79 (1H, dd), 8.53 (1H, dd), 10.93 (1H, s).

MS m/z: 515 (M+1)

### EXAMPLE 356

1-[3-(7-(2-Carboxy-2-methyl-1-propyl)oxy-5,11-dihydro[1]benzoxepino[2,3-b]pyridin-5-ylidene)propyl]-4-(4-chlorophenyl)piperidin-4-ol

Step 1

4-(4-Chlorophenyl)-1-[3-(5,11-dihydro-7-(2-ethoxycarbonyl-2-methylproyl)oxy)[1]benzoxepino[2,3-b]pyridin-5-ylidene)propyl]piperidin-4-ol was prepared by following the procedure of example 46, but replacing ethyl iodide with ethyl 2-bromo-1,1-dimethyl propionate.

¹H-NMR (CDCl₃) δ: 1.31 (6H, s), 1.67-1.72 (2H, m), 1.96-2.15 (2H, m), 2.39-2.78 (8H, m), 3.69 (3H, s), 3.93 (2H, s), 5.27 (2H, brs), 6.09 (1H, t), 6.70-6.83 (3H, m), 7.23-7.59 (6H, m), 8.46 (1H, dd).

Step 2

The titled compound was prepared by following the procedure of example 133, but replacing the product of example 48 with the product of step 1.

¹H-NMR (DMSO-d6) δ: 1.46-1.50 (2H, m), 1.74-1.85 (2H, m), 2.22-2.38 (8H, m),3.92 (2H, s), 4.58 (1H, brs), 5.19 (2H, brs), 6.18 (1H, t), 6.71-6.83 (3H, m), 7.33-7.48 (5H, m), 7.72 (1H, dd), 8.49 (1H, dd).

MS m/z: 514 (M+1)

### EXAMPLE 357

4-(4-Chlorophenyl)-1-[3-(5,1 1-dihydro-7-(2-(hydroxyimino)propyl)[1]benzoxepino[2,3-b]pyridin-5-ylidene)propyl]piperidin-4-ol

The titled compound was prepared by following the procedure of Example 354, but replacing the product of example 313 with the product of example 315.

¹H-NMR (DMSO-d6) δ: 1.39-1.54 (2H, m), 1.64-1.86 (2H, m), 2.13 (3H, s), 2.19-2.36 (4H, m), 2.36-2.52 (4H, m), 4.83 (1H, s), 5.28 (2H, brs), 6.20 (1H, t), 6.80 (1H, d),7.35 (2H, d), 7.43-7.49 (4H, m), 7.58 (1H, d), 7.76 (1H, d), 8.51 (1H, dd), 11.04 (1H, s).

MS m/z: 504 (M+1)

### EXAMPLE 358

4-(4-Chlorophenyl)-1-[3-(5,11-dihydro-7-propionyl[1]benzoxepino[2,3-b]pyridin-5-ylidene)propyl]piperidin-4-ol

The titled compound was prepared by following the procedure of example 315, but replacing acetyl chloride with propionyl chloride.

¹H-NMR (CDCl₃) δ: 1.22 (3H, t), 1.63-1.77 (2H, m), 1.97-2.13 (2H, m), 2.25-2.48 (4H, m), 2.48-2.60 (2H, m). 2.60-2.73 (2H, m), 2.96 (2H, q), 5.41 (2H, brs), 6.21 (1H, t),6.86 (1H, d), 7.30-7.34 (3H, m), 7.43 (2H, d), 7.59 (1H, d), 7.75 (1H, dd), 7.97 (1H, d),8.53 (1H, d).

MS m/z: 503 (M+1)

### EXAMPLE 359

4-(4-Chlorophenyl)-1-[3-(5,11-dihydro-7-isobutyry[1]benzoxepino[2,3-b]pyridin-5-ylidene)propyl]piperidin-4-ol

The titled compound was prepared by following the procedure of example 315, but replacing acetyl chloride with isobutyryl chloride.

1H-NMR (CDCL₃) δ: 1.21-1.33 (2H, m), 1.76-2.00 (2H, m), 2.46-3.47 (8H, m), 3.53 (1H, m), 5.47 (2H, brs), 6.09 (1H, t), 6.89 (1H, d), 7.32-7.45 (6H, m), 7.64 (1H, d),7.79 (1H, dd), 7.94 (1H, d), 8.57 (1H, d).

MS m/z: 517 (M+1)

### EXAMPLE 360

4-(4-Chlorophenyl)-1-[3-(7-cyclopropylacetyl-5,11-dihydro[1]benzoxepino[2,3-b]pyridin-5-ylidene)propyl]piperidin-4-ol

The titled compound was prepared by following the procedure of Example 315, but replacing acetyl chloride with cyclopropylacetyl chloride.

¹H-NMR (CDCl₃) δ: 0.98-1.05 (2H, m), 1.20-1.24 (2H, m), 1.58-1.70 (2H, m), 1.99-2.09 (2H, m), 2.34-2.55 (4H, m), 2.58-2.68 (5H, m), 5.40 (2H, brs), 6.23 (1H, t), 6.89 (1H, d), 7.30-7.34 (3H, m), 7.43 (2H, d), 7.59 (1H, dd), 7.86 (1H, dd), 8.00 (1H, d), 8.53 (1H, dd).

MS m/z: 515 (M+1)

### EXAMPLE 361

1-[3-(7-(3-Carboxypropionyl)-5,11-dihydro[1]benzoxepino[2,3-b]pyridin-5-ylidene)propyl]-4-(4-chlorophenyl)piperidin-4-ol

Step 1

4-(4-Chlorophenyl)-1-[3-(5, 11-dihydro-7-(3-methoxycarbonylpropionyl)[1]benzoxepino[2,3-b]pyridin-5-ylidene)piperidin-4-ol was prepared by following the procedure of Example 315, but replacing acetyl chloride with methyl succinyl chloride.

¹H-NMR (CDCl₃) δ: 1.57-1.77 (4H, m), 1.94-2.14 (4H, m), 2.27-2.61 (6H, m) 2.61-2.73 (2H, m), 3.67 (3H, s), 4.70 (1H, t), 5.30 (2H, brs), 6.1 1 (1H, t), 6.83 (1H, d), 7.14 (1H, d), 7.29-7.32 (4H, m), 7.42 (2H, d), 7.58 (1H, d), 8.50 (1H, d).

Step 2

The titled compound was prepared by following the procedure of Example 133, but replacing the product of example 48 with the product of step 1.

¹H-NMR (DMSO-d6) δ: 1.37-1.57 (2H, m), 1.63-1.86 (2H, m), 2.13-2.37 (4H, m), 2.45-2.63 (4H, m), 3.17-3.28 (4H, m), 4.85 (1H, brs), 5.36 (2H, brs), 6.30 (1H, t), 6.91 (1H, d),7.35 (2H, d), 7.46-7.50 (3H, m), 7.78-7.83 (2H, m), 7.95 (1H, d), 8.53 (1H, dd).

MS m/z: 547 (M+1)

### EXAMPLE 362

4-(4-Chlorophenyl)-1-[3-(5,11-dihydro-7-(1-ethyl-1-hydroxy)propyl[1]benzoxepino[2,3-b]pyridin-5-ylidene)propyl]piperidin-4-ol

The titled compound was prepared by flowing the procedure of example 242, but replacing methylmagnesium bromide with ethylmagnesium bromide.

¹H-NMR (CDCl₃) δ: 0.79 (6H, t), 1.65-2.04 (9H, m), 2.35-2.66 (8H, m), 5.37 (2H, brs),6.09 (1H, t), 6.81 (1H, d), 7.10 (1H, dd), 7.26-7.51 (6H, m), 7.59 (1H, dd), 8.49 (1H, dd).

MS m/z: 533 (M+1)

### EXAMPLE 363

4-(4-Chlorophenyl)-1-[3-(7-(1-cyano-1-methyl)ethyl-5,11-dihydro[1]benzoxepino[2,3-b]pyridin-5-ylidene)propyl]piperidin-4-ol

Step 1

5-(3-bromopropylidene)-7-(1-hydroxy-1-methyl)ethyl-5,11-dihydro[1]benzoxepino[2,3-b]pyridine was prepared by following the procedure of example 200, but replacing the product of example 48 with the product of example 315,

step 1.

¹H-NMR (CDCl₃) δ: 1.58 (6H, s), 2.74 (2H, q), 3.47 (2H, t), 5.34 (2H, brs), 6.09 (1H, t), 6.82 (1H, d), 7.25-7.31 (2H, m), 7.45 (1H, d), 7.57 (1H, dd), 8.52 (1H, dd).

Step 2

To a solution of the product of step 1 (3.8 g) in dichloromethane (40 ml) was added trimethylsilyl cyanide (4.1 ml) and boron trifluoride diethyl etherate (2.5 ml) at 0° C., and the mixture stirred at room temperature for 10 minutes. The reaction mixture was poured into saturated aqueous sodium bicarbonate. The aqueous layer was extracted with ethyl acetate, and the organic layer was washed with saturated aqueous sodium chloride, and dried with magnesium sulfate. The solvent was distilled off under reduced pressure. The residue was purified by silica gel chromatography eluting with ethyl acetate-hexane (1:3) to give 5-(3-bromopropylidene)-7-(-1-cyano-1-methyl)ethyl-5,11-dihydro[1]benzoxepino[2,3-b]pyridine (3.4 g).

¹H-NMR (CDCl₃) δ: 1.58 (6H, s), 2.76 (2H, m), 3.48 (2H, t), 5.34 (2H, brs), 6.09 (1H, t),6.87 (1H, d), 7.22 (1H, dd), 7.32 (1H, dd), 7.42 (1H, d), 7.58 (1H, dd), 8.55 (1H, dd).

Step 3

The titled compound was prepared by following the procedure of example 44, step 2,but replacing the product of example 44, step 1 with the product of step 2.

¹H-NMR (CDCl₃) δ: 1.58 (6H, s), 1.60-1.70 (2H, m), 1.93-2.12 (2H, m), 2.30-2.47 (4H, m), 2.50-2.74 (4H, m), 5.31 (2H, brs, 6.15 (1H, t), 6.86 (1H, d), 7.19 (1H, dd), 7.28-7.32 (3H, m), 7.41-7.43 (3H, m), 7.61 (1H, d), 8.53 (1H, dd).

MS m/z: 514 (M+1)

### EXAMPLE 364

4-(4-Chlorophenyl)-1-[3-(7-cyano-5,11-dihydro[1]benzoxepino[2,3-b]pyridin-5-ylidene)propyl]piperidin-4-ol

The titled compound was prepared by following the procedure of example 44, step 2,but replacing the product of example 44, step 1 with 5-(3-bromopropylidene)-7-cyano-5,1 1-dihydro[1]benzoxepino[2,3-b]pyridine.

¹H-NMR (CDCL₃) δ: 1.62-1.75 (2H, m), 1.98-2.09 (2H, m), 2.36-2.69 (8H, m), 5.36 (2H, brs), 6.19 (1H, t), 6.89 (1H, d), 7.29-7.62 (8H, m), 8.55 (1H, d).

MS m/z: 472 (M+1)

### EXAMPLE 365

4-(4-Chlorophenyl)-1-[3-(5,11-dihydro-7-(tetrazol-5-yl)[1]benzoxepino[2,3-b]pyridin-5-ylidene)propyl]piperidin-4-ol

To a solution of the product of Example 364 (1.0 g) in DMF (10 ml) were added sodium azide (0.69 g) and ammonium chloride (0.56 g) and the mixture stirred at 100° C. for 36 hour. Water was added to the reaction mixture, and the precipitate was filtered and washed with ethanol to give the titled compound (800 mg).

¹H-NMR (DMSO-d6) δ: 1.66-1.71 (2H, m), 1.91-2.01 (2H, m), 2.86-3.09 (8H, m),5.33 (2H, brs), 6.22 (1H, t), 6.91 (1H, d), 7.39-7.51 (5H, m), 7.79-7.84 (2H, m), 8.03 (1H, d), 8.55 (1H, dd).

MS m/z: 515 (M+1)

### EXAMPLE 366

4-(4-Chlorophenyl)-1-[3-(5,11-dihydro-7-(hydroxyiminomethyl) [1]benzoxepino[2,3-b]pyridin-5-ylidene)propyl]piperidin-4-ol

The titled compound was prepared by following the procedure of Example 357, but replacing the product of example 315, step 2 with the product of example 314.

¹H-NMR (DMSO-d6) δ: 141-1.52 (2H, m), 1.70-1.82 (2H, m), 2.27-2.46 (8H, m),4.83 (1H, s), 5.37 (2H, brs), 6.20 (1H, t), 6.83 (1H, d), 7.34-7.53 (7H, m), 7.76 (2H, dd),

MS m/z: 490 (M+1)

### EXAMPLE 367

1-(4-Chlorophenyl)-4-[3-(5,11-dihydro-7-(1-hydroxy-1-methyl)ethyl[1]benzoxepino[2,3-b]pyridin-5-ylidene)propyl]piperazine The titled compound was prepared by following the procedure of example 71, but replacing the product of example 45, step 2 with the product of Example 363, step 1.

¹H-NMR (CDCl₃) δ: 1.58 (6H, s), 2.31-2.63 (8H, m), 3.02-3.20 (4H, m), 5.32 (2H, brs),6.12 (1H, t), 6.79-6.83 (3H, m), 7.17-7.31 (6H, m), 7.45 (1H, d), 7.58 (1H, dd), 8.51 (1H, dd).

MS m/z: 490 (M+1)

### EXAMPLE 368

4-(4-Chlorophenyl)-1-[3-(5,11-dihydro-7-sulfamoyl[1]benzoxepino[2,3-b]pyridin-5-ylidene)propyl]piperidin-4-ol

Step 1

To the product of example 53, step 1 (5.4 g) was added chlorosulfonic acid (50 ml) and the mixture stirred at 0° C. for 1 hour. The reaction mixture was poured to ice, and ethyl acetate was added to the mixture, the organic layer was separated and washed with saturated aqueous sodium chloride, and dried with magnesium sulfate. The solvent was distilled off under reduced pressure. To the residue were added THF (250 ml) and ammonium hydroxide (30 ml) and the mixture stirred at room temperature for 10 minutes. Ethyl acetate and water were added to the mixture, the organic layer was separated and washed with saturated aqueous sodium chloride, and dried with magnesium sulfate. The solvent was distilled off under reduced pressure. The residue was purified by silica gel chromatography eluting with ethyl acetate-hexane (1:1) to give 5-(3-bromopropylidene)-5,11-dihydro-7-sulfamoyl[1]benzoxepino[2,3-b]pyridine(5.0 g).

¹H-NMR (CDCl₃) δ: 2.70-2.75 (2H, m), 3.48 (2H, t), 5.39-5.49 (4H, m), 6.16 (1H, t),6.88 (1H, d), 7.25-7.34 (2H, m), 7.53 (1H, dd), 7.68 (1H, dd), 7.93 (1H, d), 8.53 (1H, dd).

Step 2

The titled compound was prepared by following the procedure of example 44, step 2,but replacing the product of example 44, step 1 with the product of step 1.

¹H-NMR (DMSO-d6) δ: 1.65-1.70 (3H, m), 1.98-2.07 (2H, m), 2.35-2.64 (8H, m),4.98 (2H, brs), 5.39 (2H, brs), 6.22 (1H, t), 6.92 (1H, d) 7.26-7.43 (5H, m), 7.55-7.69 (2H, m), 7.91 (1H, d), 8.53 (1H, dd).

MS m/z: 526 (M+1)

### EXAMPLE 369

1-[-3-(7-(2-Aminothiazol-4-yl)-5,11-dihydro[1]benzoxepino[2,3-b]pyridin-5-ylidene)propyl]-4-(4-chlorophenyl)piperidin-4-ol

Step 1

7-bromoacetyl-5-(3-bromopropylidene)-5,11-dihydro[1]benzoxepino[2,3-b]pyridine was prepared by following the procedure of example 315, step 1, but replacing acetyl chloride with bromoacetyl chloride.

¹H-NMR (CDCl₃) δ: 2.77 (2H, m), 3.50 (2H, m), 4.40 (2H, s), 5.45 (2H, brs), 6.17 (1H, t),6.90 (1H, d), 7.35 (1H, dd), 7.60 (1H, dd), 7.79 (1H, dd), 8.01 (1H, d), 8.57 (1H, dd).

Step 2

To a solution of the product of step 1 (1.1 g) in ethanol (11 ml) was added thiourea (193 mg) at room temperature, and the mixture stirred at 70° C. for 30 minutes. The reaction mixture was cooled to room temperature and poured into saturated aqueous sodiumbicarbonate. The aqueous layer was extracted with ethyl acetate, and the organic layer was washed with saturated aqueous sodium chloride, and dried with magnesium sulfate. The solvent was distilled off under reduced pressure. The residue was purified by silica gel chromatography eluting with ethyl acetate to give 7-(2-aminothiazol-4-yl)-5-(3-bromopropylidene)-5,11-dihydro[1]benzoxepino[2,3-b]pyridine (749 mg).

¹H-NMR (CDCl₃) δ: 2.74 (2H, m), 3.47 (2H, t), 5.02 (2H, brs), 5.39 (2H, brs), 6.16 (1H, t), 6.62 (1H, s), 6.85 (1H, d), 7.30 (1H, dd), 7.54-7.57 (2H, m), 7.77 (1H, d), 8.53 (1H, dd).

Step 3

The titled compound was prepared by following the procedure of example 44, step 2, but replacing the product of example 44, step 1 with the product of step 2.

¹H-NMR (CDCl₃) δ: 1.57-1.70 (2H, m), 1.83-2.13 (2H, m), 2.30-2.46 (4H, m), 2.46-2.60 (2H, m), 2.60-2.73 (2H, M), 5.02 (2H, s), 5.37 (2H, brs), 6.20 (1H, t), 6.61 (1H, s), 6.85 (1H, d), 7.27-7.32 (3H, m), 7.42 (2H, d), 7.50-7.58 (2H, m), 7.76 (1H, d), 8.50 (1H, dd).

MS m/z: 545 (M+1)

### EXAMPLE 370

1-[3-(7-(3-Carboxy-1-hydroxy)propyl-5,11-dihydro[1]benzoxepino[2,3-b]pyridin-5-ylidene)propyl]-4-(4-chlorophenyl)piperidin-4-ol

Step 1

4-(4-Chlorophenyl)-1-[3-(5,11-dihydro-7-(3-methoxycarbony-1-hydroxy)propyl[1]benzoxepino[2,3-b]pyridin-5-ylidene)propyl]piperidin-4-ol was prepared by following the procedure of example 199, but replacing the product of example 138 with the product of Example 361, step 1.

¹H-NMR (CDCl₃) δ: 1.57-1.77 (4H, m), 1.94-2.14 (4H, m), 2.27-2.61 (6H, m), 2.61-2.73 (2H, m), 3.67 (3H, s), 4.70 (1H, t), 5.30 (2H, brs), 6.11 (1H, t), 6.83 (1H, d), 7.14 (1H, d), 7.29-7.32 (4H, m), 7.42 (2H, d), 7.58 (1H, d), 8.50 (1H, d).

Step 2

The titled compound was prepared by following the procedure of example 133, but replacing the product of example 48 with the product step 1.

¹H-NMR (DMSO-d6) δ: 1.44-1.63 (2H, m), 1.69-1.90 (2H, m), 2.17-2.29 (2H, m), 2.29-2.82 (6H, m), 3.24-3.53 (4H, m), 4.49 (1H, t), 5.03 (1H, brs), 5.20 (2H, brs), 6.13 (1H, t),6.76 (1H, d), 7.12 (1H, dd), 7.27 (1H, d), 7.37 (2H, d), 7.43-7.48 (3H, m), 7.76 (1H, d), 8.32 (1H, s), 8.51 (1H, dd).

MS m/z: 549 (M+1)

### EXAMPLE 371

4-(4-Chlorophenyl)-1-[3-(5,11-dihydro-7-(2-fluoroethylamino)carbonylmethyloxy[1]benzoxepino[2,3-b]pyridin-5-ylidene)propyl]piperidin-4-ol

The titled compound was prepared by following the procedure of example 134, but replacing dimethylamine hydrochloride with 2-fluoroethylamine.

¹H-NMR (CDCl₃) δ: 1.62-1.71 (3H, m), 1.98-2.10 (2H, m), 2.36-2.71 (8H, m), 3.63 (1H, q), 3.73 (1H, q), 4.46 (1H, t), 4.49 (2H, s), 4.63 (1H, t), 5.29 (2H, brs), 6.10 (1H, t), 6.75-6.96 (4H, m), 7.28-7.44 (5H, m), 7.60 (1H, dd), 8.51 (1H, dd).

MS m/z: 566 (M+1)

### EXAMPLE 372

4-(4-Chlorophenyl)-1-[3-(5,11-dihydro-7-(N-methylsulfamoyl)[1]benzoxepino[2,3-b]pyridin-5-ylidene)propyl]piperidin-4-ol

The titled compound was prepared by following the procedure of Example 368, but replacing ammonium hydroxide with methylamine.

¹H-NMR (CDCl₃) δ: 1.57-1.70 (3H, m), 1.93-2.08 (2H, m), 2.34-2.73 (11H, m), 4.33 (1H, q), 5.36 (2H, brs), 6.21 (1H, t), 6.91 (1H, d), 7.29-7.45 (6H, m), 7.58-7.65 (2H, m), 7.83 (1H, dd), 8.53 (1H, dd).

MS m/z: 540 (M+1)

### EXAMPLE 373

4-(4-Chlorophenyl)-1-[3-(5,11-dihydro-7-(N,N-dimethylsulfamoyl)[1]benzoxepino[2,3-b]pyridin-5-ylidene)propyl]piperidin-4-ol

The titled compound was prepared by following the procedure of Example 368, but replacing ammonium hydroxide with dimethylamine.

¹H-NMR (CDCl₃) δ: 1.55-1.75 (3H, m), 1.96-2.07 (2H, m), 2.35-2.67 (8H, m), 2.71 (6H, s), 5.51 (2H, brs), 6.19 (1H, t), 6.92 (1H, d), 7.29-7.73 (8H, m), 8.55 (1H, dd).

MS m/z: 554 (M+1)

### EXAMPLE 374

1-[3-(7-(1-Carboxy-2-hydroxyethyl)oxy-5,11-dihydro[1]benzoxepino[2,3-b]pyridin-5-ylidene)propyl]-4-(4-chlorophenyl)piperidin-4-ol

Step 1

4-(4-Chlorophenyl-1-[3-(5,11-dihydro-7-(1-ethoxycarboxy-2-hydroxyethyl)oxy[1]benzoxepino[2,3-b]pyridin-5-ylidene)propyl]piperidin-4-ol was prepared by following the procedure of example 199, but replacing the product of example 138 with the product of example 294.

¹H-NMR (CDCl₃) δ: 1.65-1.70 (2H, m), 2.01-2.11 (2H, m), 2.35-2.70 (8H, m), 3.76 (3H, s), 3.97-4.08 (2H, m), 4.71 (1H, t), 5.25 (1H, brs) 6.02 (1H, t) 6.70-6.91 (3H, m), 7.23-7.56 (6H, m), 8.44 (1H, dd).

Step 2

The titled compound was prepared by following the procedure of example 133, but replacing the product of example 48 with the product of Step 1.

¹H-NMR (DMSO-d6) δ: 1.51-1.56 (2H, m), 1.86-1.94 (2H, m), 2.33-2.67 (8H, m), 3.65-3.82 (2H, m), 4.58 (1H, t), 5.17 (2H, brs), 6.10 (1H, t), 6.71-6.89 (3H, m), 7.34-7.47 (5H, m), 7.72 (1H, dd), 8.48 (1H, dd).

MS m/z: 551 (M+1)

### EXAMPLE 375

4-(4-Chlorophenyl)-1-[3-(5,11-dihydro-7-ureidomethy[1]benzoxepino[2,3-b]pyridin-5-ylidene)propyl]piperidin-4-ol

To a solution of the product of example 314 (800 mg) in acetic acid (20 ml) were added urea (2 g) and trimethylsilyl chloride (0.24 ml) at room temperature, and the mixture stirred for 2 hours. Sodium borohydride was added to the reaction mixture at room temperature, and the mixture was stirred for 1 hour. The solvent was distilled off under reduced pressure, and, chloroform, 2-propanol and water were added. The organic layer was extracted, and the solvent was distilled off under reduced pressure. The residue was purified by silica gel chromatography eluting with chloroform-methanol-ammonium hydroxide (100:10:1) to give the titled compound (250 mg).

¹H-NMR (CDCl₃) δ: 1.62-2.04 (5H, m), 2.35-2.69 (8H, m), 4.26 (2H, d), 4.40 (2H, s), 4.48 (1H, t), 5.32 (2H, brs), 6.12 (1H, t), 6.80 (1H, d), 7.07 (1H, dd), 7.23-7.58 (7H, m), 8.49 (1H, dd).

MS m/z: 519 (M+1)

### EXAMPLE 376

4-(4-Chlorophenyl)-1-[3-(5,11-dihydro-7-methylthio[1]benzoxepino[2,3-b]pyridin-5-ylidene)propyl]piperidin-4-ol

The titled compound was prepared by following the procedure of example 44, step 2, but replacing the product of example 44, step 1 with 5-(3-bromopropylidene)-5,11-dihydro-7-methylthio[1]benzoxepino[2,3-b]pyridine.

¹H-NMR (CDCl₃) δ: 1.53-1.70 (3H, m), 1.98-2.16 (2H, m), 2.17 (3H, s), 2.34-2.70 (8H, m), 5.32 (2H, brs), 6.12 (1H, t), 6.81 (1H, d), 7.11-7.44 (7H, m), 7.57 (1H, dd), 8.50 (1H, dd).

MS m/z: 493 (M+1)

### EXAMPLE 377

4-(4-Chlorophenyl)-1-[3-(5,11-dihydro-7-(2-furanon-3-yl)oxy[1]benzoxepino[2,3]b]pyridin-5-ylidene)propyl]piperidin-4-ol

The titled compound was prepared by following the procedure of example 46, but replacing ethyl iodide with 3-bromotetrahydro-2-franon.

¹H-NMR (CDCl₃) δ: 1.65-1.70 (2H, m), 1.97-2.13 (2H, m), 2.25-2.73 (10H, m), 4.25-4.53 (2H, m), 4.82 (1H, t), 5.27 (2H, brs), 6.09 (1H, t), 6.73-6.91 (2H, m), 7.03 (1H, d), 7.22-7.59 (6H, m), 8.43 (1H, dd).

MS m/z: 547 (M+1)

### EXAMPLE 378

4-(4-Chlorophenyl)-1-[3-(5,11-dihydro-7-(N-methoxycarbonylmethylsulfamoyl) [1]benzoxepino[2,3-b]pyridin-5-ylidene)propyl]piperidin-4-ol

The titled compound was prepared by following the procedure of Example 368, but replacing ammonium hydroxide with glycine methyl ester hydrochloride.

¹H-NMR (CDCl₃) δ: 1.66-1.74 (3H, m), 1.97-2.15 (2H, m), 2.37-2.80 (8H, m), 3.63 (3H, s), 3.78 (2H, s) 5.40 (2H, brs), 6.22 (1H, t), 6.92 (1H, d), 7.28-7.45 (5H, m), 7.62 (2H, dd), 7.83 (1H, d), 8.53 (1H, dd).

MS m/z: 598 (M+1)

### EXAMPLE 379

1-[3-(7-(N-Carboxymethylsulfamoyl-5,11-dihydro[1]benzoxepino[2,3-b]pyridin-5-ylidene)propyl]-4-(4-chlorophenyl)piperidin-4-ol

The titled compound was prepared by following the procedure of example 133, but replacing the product of example 48 with the product of Example 378.

¹H-NMR (DMSO-d6) δ: 1.60-1.65 (2H, m), 2.16-2.25 (2H, m), 2.43-3.03 (8H, m), 3.45 (2H, s), 5.33 (2H, brs), 6.39 (1H, t), 6.94 (1H, d), 7.41-7.57 (6H, m), 7.83 (1H, dd), 8.00 (1H, d), 8.54 (1H, dd).

### EXAMPLE 380

4-(4-Chlorophenyl)-1-[3-(5,11-dihydro-7-(2-furanon-5-yl)[1]benzoxepino[2,3-b]pyridin-5-ylidene)propyl]piperidin-4-ol

The titled compound was prepared by following the procedure of example 249, step 2, but replacing the product of example 249, step 1 with the product of Example 370, step 1.

¹H-NMR (CDCl₃) δ: 1.45-1.78 (4H, m), 1.93-2.12 (2H, m), 2.30-2.50 (4H, m), 2.50-2.78 (6H, m), 5.33 (2H, brs), 5.46 (1H, t), 6.12 (1H, t), 6.86 (1H, d), 7.09 (1H, dd), 7.27-7.32 (4H, m), 7.42 (2H, d), 7.58 (1H, dd), 8.51 (1H, dd).

MS m/z: 531 (M+1)

### EXAMPLE 381

1-[3-(7-Amino-5,11-dihydro[1]benzoxepino[2,3-b]pyridin-5-ylidene)propyl[-4-(4-chlorophenyl)piperidin-4-ol

To a solution of the produce of example 293 (3.7 g) in ethanol (130 ml) was added 5N sodium hydroxide solution (100 ml) and the mixture stirred at 90° C. for 1 hour. The reaction mixture was distilled off under reduced pressure. The residue was dissolved with water and neutralized with 1N hydrochloric acid. Ethyl acetate was added to the mixture, the organic layer was separated and washed with saturated aqueous sodium chloride, and dried with magnesium sulfate to give the titled compound (3.0 g).

¹H-NMR (CDCl₃) δ: 1.62-1.72 (2H, m), 1.96-2.08 (2H, m), 2.27-2.72 (8H, m), 3.48 (2H, brs), 5.23 (2H, brs), 6.01 (1H, t), 6.49-6.73 (3H, m), 7.18-7.59 (6H, m), 8.49 (1H, dd).

MS m/z: 462 (M+1)

### EXAMPLE 382

1-[3-(7-(2-Carboxyphenyl)-5,11-dihydro[1]benzoxepino[2,3-b]pyridin-5-ylidene)propyl]-4-(4-chlorophenyl)piperidin-4-ol

Step 1

4-(4-Chlorophenyl)-1-[3-(5,11-dihydro-7-(2-formylphenyl)[1]benzoxepino[2,3-b]pyridin-5-ylidene)propyl]piperidin-4-ol was prepared by following the similar procedure of example 170, but replacing allyltributyltin with 2-formylphenylboronic acid.

¹H-NMR (CDCl₃) δ: 1.65-1.91 (3H, m), 1.99-2.04 (2H, m), 2.37-2.65 (8H, m), 5.39 (2H, brs), 6.15 (1H, t), 6.95 (1H, d), 7.19-7.65 (10H, m), 7.97-8.05 (2H, m), 8.52 (1H, dd), 10.03 (1H, s).

Step 2

To a solution of the product of step 1 (270 mg) in acetic acid (2.2 ml) and water (0.5 ml) were added amidosulfuric acid (67 mg) and sodium chlorite (68 mg) in water (0.1 ml), and the mixture was stirred at room temperature for 15 minutes. The reaction mixture was distilled off under reduced pressure into half volume. The residue was neutralized with 1N sodium hydroxide. The precipitation was filtered and washed with water to give the titled compound (80 mg).

¹H-NMR (DMSO-d6) δ: 1.41-1.57 (2H, m), 1.74-1.92 (2H, m), 2.21-2.58 (8H, m), 5.32 (2H, brs), 6.20 (1H, t), 6.82 (1H, d), 7.15 (1H, dd), 7.31-7.78 (11H, m), 8.52 (1H, dd).

MS m/z: 567 (M+1)

### EXAMPLE 383

4-(4-Chlorophenyl)-1-[3-(5,11-dihydro-7-(N-(2,2,2-trifluoro ethyl) sulfamoyl)[1]benzoxepino[2,3-b]pyridin-5-ylidene)propyl]piperidin-4-ol

The titled compound was prepared by following the procedure of Example 368, but replacing ammonium hydroxide with 2,2,2-trifluoroethylamine hydrochloride.

¹H-NMR (CDCl₃) δ: 1.64-1.77 (2H, m), 1.97-2.18 (2H, m), 2.35-2.80 (8H, m), 3.63 (2H, q), 5.41 (2H, brs), 6.21 (1H, t), 6.91 (1H, d), 7.22-7.65 (7H, m), 7.84 (1H, d), 8.57 (1H, dd).

MS m/z: 608 (M+1)

### EXAMPLE 384

4-(4-Chlorophenyl)-1-[3-(5,11-dihydro-7-methylsulfonyl[1]benzoxepino[2,3-b]pyridin-5-ylidene)propyl]piperidin-4-ol

The titled compound was prepared by following the procedure of Example 44, step 2, but replacing the product of Example 44, step 1 with 5-(3-bromopropylidene)-5,11-dihydro-7-metylsulfonyl[1]benzoxepino[2,3-b]pyridine.

¹H-NMR (CDCl₃) δ: 1.54-1.71 (3H, m), 1.99-2.08 (2H, m), 2.34-2.68 (8H, m), 3.04 (3H, s), 5.43 (2H, brs), 6.24 (1H, t), 6.97 (1H, d), 7.22-7.70 (7H, m), 7.89 (1H, d), 8.55 (1H, dd).

MS m/z: 525 (M+1)

### EXAMPLE 385

4-(4-Chlorophenyl)-1-[3-(5,11-dihydro-7-ureido[1]benzoxepino[2,3-b]pyridin-5-ylidene)propyl]piperidin-4-ol

Step 1

4-(4-Chlorophenyl)-1-[3-(5,11-dihydro-7-pheoxycarbonylamino[1]benzoxepino[2,3-b]pyridin-5-ylidene)propyl]piperidin-4-ol

The titled compound was prepared by following the procedure of Example 293, but replacing ethanol with phenol.

¹H-NMR (CDCl₃) δ: 1.62-1.68 (2H, m), 1.96-2.08 (2H, m), 2.35-2.65 (8H, m), 5.28 (2H, brs), 6.10 (1H, t), 6.78 (1H, m), 7.08-7.40 (6H, m), 7.52 (1H, dd), 7.62 (1H, s), 8.44 (1H, dd).

MS m/z: 582 (M+1)

Step 2

To a solution of the product of Step 1 (300 mg) in DMF (3 ml) was added ammonium hydroxide (1.5 ml) and the mixture was stirred at room temperature for 2 hours. Ethyl acetate and water were added to the mixture, the organic layer was separated and washed with saturated aqueous sodium chloride, and dried with magnesium sulfate. The solvent was distilled off under reduced pressure. The residue was purified by silica gel chromatography eluting with (chloroform:methanol=10:1) to give the titled compound (140 mg).

¹H-NMR (DMSO-d6) δ: 1.45-1.50 (2H, m), 1.72-1.88 (2H, m), 2.28-2.51 (8H, m), 4.82 (1H, s), 5.19 (1H, brs), 5.74 (2H, brs), 6.09 (1H, t), 6.69 (1H, d), 7.12 (1H, dd), 7.32-7.48 (6H, m), 7.74 (1H, dd), 8.37 (1H, s), 8.50 (1H, dd).

MS m/z: 505 (M+1)

### EXAMPLE 386

4-(4-Chlorophenyl)-1-[3-(5,11-dihydro-7-(1-morpholinocarbonylamino[1]benzoxepino[2,3-b]pyridin-5-ylidene)propyl]piperidin-4-ol

The titled compound was prepared by following the procedure of Example 385, step 2, but replacing ammonium hydroxide with morpholine.

¹H-NMR (CDCl₃) δ: 1.62-1.67 (2H, m), 1.95-2.16 (2H, m), 2.28-2.64 (8H, m), 3.41 (4H, t), 3.69 (4H, t), 5.26 (2H, brs), 6.08 (1H, t), 6.69-6.76 (2H, m), 6.98 (1H, dd), 7.21-7.51 (7H, m), 8.42 (1H, dd).

MS m/z: 575 (M+1)

### EXAMPLE 387

4-(4-Chlorophenyl)-1-[3-(5,11-dihydro-7-(3-(2-ethoxy)carbonylethyl)ureido[1]benzoxepino[2,3-b]pyridin-5-ylidene)propyl]piperidin-4-ol

The titled compound was prepared by following the procedure of Example 385, step 2, but replacing ammonium hydroxide with beta-alanine ethyl ester hydrochloride.

¹H-NMR (CDCl₃) δ: 1.18-1.39 (3H, t), 1.62-1.66 (2H, m), 1.92-2.01 (2H, m), 2.21-2.62 (10H, m), 3.47-3.50 (2H, m), 4.08 (2H, q), 5.22 (2H, brs), 5.98-6.03 (2H, m), 6.68-6.92 (2H, m), 7.15-7.42 (7H, m), 7.62 (1H, s), 8.36 (1H, dd).

MS m/z: 605 (M+1)

### EXAMPLE 388

1-[3-(7-(E)-(2-Carboxy-1-methyl)ethenyl-5,11-dihydro[1]benzoxepino[2,3-b]pyridin-5-ylidene)propyl]-4-(4-chlorophenyl)piperidin-4-ol

Step 1

4-(4-Chlorophenyl)-1-[3-(7-(E)-(2-ethoxycarboxy-1-methyl)ethenyl-5,11-dihydro[1]benzoxepino[2,3-b]pyridin-5-ylidene)propyl]piperidin-4-ol was prepared by following the procedure of Example 411, but replacing ethyl cyanoformate with ethyl (trimethylsilyl)acetate.

¹H-NMR (CDCl₃) δ: 1.30 (3H, t), 1.67-1.72 (3H, m), 1.98-2.05 (2H, m), 2.42-2.67 (11H, m), 4.23 (2H, q), 5.36 (2H, brs), 6.14-6.19 (2H, m), 6.85 (1H, d), 7.20-7.61 (8H, m), 8.52 (1H, dd).

Step 2

The titled compound was prepared by following the procedure of Example 133, but replacing the product of Example 48 with the product of step 1.

¹H-NMR (DMSO-d6) δ: 1.50-1.55 (2H, m), 1.87-1.99 (2H, m), 2.34-2.61 (11H, m), 5.29 (2H, brs), 6.12 (1H, s), 6.31 (1H, t), 6.83 (1H, d), 7.35-7.49 (7H, m), 7.76 (1H, dd), 8.52 (1H, dd).

MS m/z: 530 (M+l)

### EXAMPLE 389

4-(4-Chlorophenyl)-1-[3-(5,11-dihydro-7-oxalo[1]benzoxepino[2,3-b]pyridin-5-ylidene)propyl]piperidin-4-ol

The titled compound was prepared by following the procedure of Example 361, but replacing methyl succinyl chloride with methyl oxalyl chloride.

¹H-NMR (DMSO-d6) δ: 1.66-1.86 (2H, m), 2.08-2.34 (2H, m), 2.46-2.77 (2H, m), 3.00-3.68 (6H, m), 5.10 (2H, brs), 5.53 (1H, s), 6.15 (1H, t), 6.89 (1H, d), 7.34-7.49 (5H, m), 7.68 (1H, dd), 7.75 (1H, dd), 7.87 (1H, d), 8.53 (1H, dd).

MS m/z: 519 (M+1)

### EXAMPLE 390

1-[3-(7-(3-(2-Carboxy)ethyl)ureido-5,11-dihydro[1]benzoxepino[2,3-b]pyridin-5-ylidene)propyl]-4-(4-chlorophenyl)piperidin-4-ol

The titled compound was prepared by following the procedure of Example 133, but replacing the product of Example 48 with the product of Example 387.

¹H-NMR (DMSO-d6) δ: 1.45-1.55 (2H, m), 1.72-1.85 (2H, m), 2.32-2.49 (1 OH, m), 3.29 (2H, q), 4.88 (1H, s), 5.19 (2H, brs), 6.06-6.14 (2H, m), 6.69 (1H, d), 7.07 (1H, dd), 7.33-7.48 (6H, m), 7.73 (1H, dd), 8.43 (1H, s), 8.49 (1H, dd).

MS m/z: 577 (M+1)

### EXAMPLE 391

1-[3-(7-(3-(2-Hydroxy)ethyl)ureido-5,11-dihydro[1]benzoxepino[2,3-b]pyridin-5-ylidene)propyl]-4-(4-chlorophenyl)piperidin-4-ol

The titled compound was prepared by following the procedure of Example 385, step 2, but replacing ammonium hydroxide with 2-aminoethanol.

¹H-NMR (DMSO-d6) δ: 1.45-1.51 (2H, m), 1.72-1.84 (2H, m), 2.24-2.51 (8H, m), 3.11-3.46 (4H, m), 4.71 (1H, t), 4.83 (1H, s), 5.19 (2H, brs), 6.08 (1H, t), 6.69 (1H, d), 7.08 (1H, dd), 7.33-7.48 (6H, m), 7.73 (1H, dd), 8.41 (1H, s), 8.50 (1H, dd).

MS m/z: 549 (M+1)

### EXAMPLE 392

1-[3-(5,11-Dihydro-7-(1-hydroxy-l -methyl)ethyl[1]benzoxepino[2,3-b]pyridin-5-ylidene)propyl]-4-(2-keto-1-imidazolinyl)piperidine

The titled compound was prepared by following the procedure of Example 67, but replacing the product of Example 45, step 2 with the product of Example 363, step 1.

¹H-NMR (CDCl₃) δ: 1.59 (6H, s), 1.71-1.87 (2H, m), 2.01-2.18 (2H, m), 2.28-2.61 (6H, m), 2.86-3.00 (2H, m), 4.32 (1H, m), 5.36 (2H, brs), 6.15 (1H, t), 6.84 (1H, d), 7.02-7.07 (3H, m), 7.24-7.31 (3H, m), 7.47 (1H, d), 7.60 (1H, dd), 8.51 (1H, dd), 8.97 (1H, s).

MS m/z: 511 (M+1)

### EXAMPLE 393

4-(4-Chlorophenyl)-1-[3-(7-(E)-(2-ethoxycarboxy-2-methyl)ethenyl-5,11-dihydro[1]benzoxepino[2,3-b]pyridin-5-ylidene)propyl]piperidin-4-ol

To a solution of sodium hydride (60% in oil, 100 mg) in THF (6 ml) were added triethyl 2-phosphonopropionate (0.3 ml) and the product of Example 314 (300 mg) at 0° C., and the mixture was stirred at room temperature for 30 minutes. Water and ethyl acetate were added to the reaction mixture. The organic layer was extracted, and the solvent was distilled off under reduced pressure. The residue was purified by silica gel chromatography eluting with chloroform-methanol (30:1) to give the titled compound (310 mg).

¹H-NMR (CDCl₃) δ: 1.34 (3H, t), 1.58-1.71 (3H, m), 1.98-2.15 (5H, m), 2.37-2.70 (8H, m), 2.27 (2H, q), 5.37 (2H, brs), 6.14 (1H, t), 6.86 (1H, d), 7.25-7.44 (7H, m) 7.58-7.63 (2H, m), 8.52 (1H, dd).

MS m/z: 559 (M+1)

### EXAMPLE 394

1-[3-(7-(E)-(2-Carboxy-2-methyl)ethenyl-5,11-dihydro[1]benzoxepino[2,3-b]pyridin-5-ylidene)propyl]-4-(4-chlorophenyl)piperidin-4-ol

The titled compound was prepared by following the procedure of Example 133, but replacing the product of Example 48 with the product of step 1.

¹H-NMR (DMSO-d6) δ: 1.62-1.67 (2H, m), 1.91-2.05 (5H, m), 2.50-2.94 (8H, m), 5.28 (2H, brs), 6.23 (1H, t), 6.87 (1H, d), 7.34-7.55 (8H, m), 7.79 (1H, dd), 8.54 (1H, dd).

MS m/z: 531 (M+1)

### EXAMPLE 395

1-[3-(7-(5-Carboxy-1-pentyl)oxy-5,1 1-dihydro[1]benzoxepino[2,3-b]pyridin-5-ylidene)propyl]-4-(4-chlorophenyl)piperidin-4-ol

Step 1

4-(4-Chlorophenyl)-1-[3-(7-(5-ethoxycarbonyl-1-pentyl)oxy-5,11-dihydro[1]benzoxepino[2,3-b]pyridin-5-ylidene)propyl]piperidin-4-ol was prepared by following the procedure of Example 46, but replacing ethyl iodide with ethyl 6-bromohexanoate.

¹H-NMR (CDCl₃) δ: 1.21 (3H, t), 1.42-1.79 (8H, m), 1.98-2.03 (2H, m), 2.26-2.67 (10H, m), 3.87 (2H, t), 4.16 (2H, q), 5.23 (2H, brs), 6.09 (1H, t), 6.67-6.81 (3H, m), 7.21-7.63 (6H, m), 8.16 (1H, dd).

Step 2

The titled compound was prepared by following the procedure of Example 133, but replacing the product of Example 48 with the product of step 1.

¹H-NMR (DMSO-d6) δ: 1.41-1.95 (10H, m), 2.20-2.72 (10H, m), 3.92 (2H, t), 5.18 (2H, brs), 6.17 (1H, t), 6.72-6.84 (3H, m), 7.36-7.48 (5H, m), 7.77 (1H, dd), 8.50 (1H, dd).

MS m/z: 577 (M+1)

### EXAMPLE 396

1-[3-(7-(1-(2-Carboxy)ethyl)aminocarbonyl-1-methyl)ethyloxy[1]benzoxepino[2,3-b]pyridin-5-ylidene)propyl]-4-(4-chlorophenyl)piperidin-4-ol

Step 1

4-(4-Chlorophenyl)-1-[3-(7-(1-(2-ethoxycarbonyl)ethyl)aminocarbonyl-1-methyl)ethyloxy[1]benzoxepino[2,3-b]pyridin-5-ylidene)propyl]piperidin-4-ol was prepared by following the procedure of Example 176, but replacing dimethylamine hydrochloride with beta-alanine ethyl ester hydrochloride.

¹H-NMR (CDCl₃) δ: 1.42 (3H, s), 1.62-1.67 (2H, m), 1.95-2.10 (3H, m), 2.35-2.59 (10H, m), 3.51-3.53 (2H, m), 4.00 (2H, q), 5.23 (2H, brs), 6.00 (1H, t), 6.68-6.81 (3H, m), 7.24-7.56 (6H, m), 8.39 (1H, dd).

Step 2

The title compound was prepared by following the procedure of Example 133, but replacing the product of Example 48 with the product of step 1.

¹H-NMR (DMSO-d6) δ: 1.37 (6H, s), 1.41-1.52 (2H, m), 1.79-1.87 (2H, m), 2.28-2.41 (10H, m), 3.33 (2H, q), 5.21 (2H, brs), 6.12 (1H, t), 6.70-6.87 (3H, m), 7.34-7.48 (5H, m), 7.74 (1H, dd), 8.08 (1H, t), 8.50 (1H, dd).

MS m/z: 620 (M+1)

### EXAMPLE 397

4-(4-Chlorophenyl)-1-[3-(5,11-dihydro-7-(thiazoline-2,4-dione-5-ylidene)methyl[1]benzoxepino[2,3-b]pyridin-5-ylidene)propyl]piperidin-4-ol

To a solution of the product of Example 314 (590 mg) in ethanol (6 ml) were added 2,4-thiazolinedione (440 mg) and piperidine (0.36 ml), and the mixture was heated to reflux for 3 hours. The solvent was distilled off under reduced pressure, and, chloroform, 2-propanol and water were added. The organic layer was extracted, and the solvent was distilled off under reduced pressure. The residue was purified by silica gel chromatography eluting with chloroform-methanol (5: 1) to give the titled compound (510 mg).

¹H-NMR (DMSO-d6) δ: 1.61-1.66 (2H, m), 1.97-2.12 (2H, m), 2.79-2.99 (8H, m), 5.21 (2H, brs), 6.25 (1H, t), 6.90 (1H, d), 7.34-7.52 (7H, m), 7.81 (1H, dd), 8.54 (1H, dd).

MS m/z: 574 (M+1)

### EXAMPLE 398:

### 4-(4-Chlorophenyl)-1-[3-(5,11-dihydro-7-methanesulfonamido[1]benzoxepino[2,3-b]pyridin-5-ylidene)propyl]piperidin-4-ol

The titled compound was prepared by following the procedure of Example 402, but replacing trifluoromethanesulfonic acid anhydride with methanesulfony chloride.

¹H-NMR (CDCl₃) δ: 1.64-1.69 (2H, m), 1.89-2.05 (2H, m), 2.24-2.77 (8H, m), 2.95 (3H, s), 5.29 (2H, brs), 6.10 (1H, t), 6.84 (1H, d), 7.06 (1H, dd), 7.18-7.40 (6H, m), 7.56 (1H, dd), 8.42 (1H, dd).

MS m/z: 540 (M+1)

### EXAMPLE 399

4-(4-Chlorophenyl)-1-[3-(5,11-dihydro-7-(3-phenylureido)sulfonyl[1]benzoxepino[2,3-b]pyridin-5-ylidene)propyl]piperidin-4-ol

The titled compound was prepared by following the procedure of Example 320, but replacing compound of Example 44, step 2 with compound of Example 368, step 2.

¹H-NMR (DMSO-db) δ: 1.65-1.69 (2H, m), 1.95-2.05 (2H, m), 2.89-3.06 (8H, m), 5.31 (2H, brs), 6.14 (1H, t), 6.74-6.85 (2H, m), 7.08-7.12 (2H, m), 7.37-7.64 (8H, m), 7.80-7.84 (2H, m), 8.44 (1H, s), 8.54 (1H, dd).

MS m/z: 645 (M+1)

### EXAMPLE 400

4-(4-Chlorophenyl)-1-[3-(7-(3-cyclohexylureido)sulfonyl-5,1 1-dihydro[1]benzoxepino[2,3-b]pyridin-5-ylidene)propyl]piperidin-4-ol

The titled compound was prepared by following the procedure of Example 399, but replacing phenyl isocyanate with cyclohexyl isocyanate.

¹H-NMR (DMSO-d6) δ: 1.07-1.81 (14H, m), 2.23-2.58 (8H, m), 3.22-3.35 (1H, m), 4.91 (1H, s), 5.38 (2H, brs), 6.17-6.29 (2H, m), 6.96 (1H, d), 7.34-7.51 (5H, m), 7.62-7.84 (3H, m), 8.53 (1H, dd).

MS m/z: 651 (M+1)

### EXAMPLE 401

4-(4-Chlorophenyl)-1-[3-(5,11-dihydro-7-(3-propylureido)sulfonyl[1]benzoxepino[2,3-b]pyridin-5-ylidene)propyl]piperidin-4-ol

The titled compound was prepared by following the procedure of Example 399, but replacing phenyl isocyanate with propyl isocyanate.

¹H-NMR (DMSIO-d6) δ: 0.74 (3H, t), 1.25-1.53 (4H, m), 1.81-1.91 (2H, m), 2.33-2.59 (10H, m), 2.89 (2H, q), 4.92 (1H, s), 5.35 (2H, brs), 6.20 (1H, t), 6.44 (1H, brs), 6.96 (1H, d), 7.34-7.51 (5H, m), 7.64 (1H, dd), 7.78-7.85 (2H, m), 8.54 (1H, dd).

MS m/z: 611 (M+1)

### EXAMPLE 402

4-(4-Chlorophenyl)-1-[3-(5,11-dihydro-7-trifluoromethanesulfonamido[1]benzoxepino[2,3-b]pyridin-5-ylidene)propyl]piperidin-4-ol

The title compound was prepared by following the procedure of Example 169, but replacing the product of Example 44, step 2 with the product of Example 381.

¹H-NMR (DMSO-d6) δ: 1.75-1.80 (2H, m), 2.02-2.07 (2H, m), 2.49-2.54 (2H, m), 3.10-3.40 (6H, m), 5.15 (2H, brs), 5.52 (1H, s), 5.97 (1H, t), 6.58 (1H, d), 6.80 (1H, dd), 6.96 (1H, d), 7.43-7.47 (5H, m), 7.78 (1H, dd), 8.51 (1H, dd).

MS m/z: 593 (M+1)

### EXAMPLE 403

1-[3-(7-(3-carboxy)propyl-5,11-dihydro[1]benzoxepino[2,3-b]pyridin-5-ylidene)propyl]-4-(4-chlorophenyl)piperidin-4-ol

Step 1

To a solution of the product of Example 361, step 1 (820 mg) in TFA (8.0 ml) was added triethyl silane (0.92 ml) at 0° C., and the mixture stirred at room temperature for 4 hour. The solvent was distilled off under reduced pressure. The residue was poured into saturated aqueous sodium bicarbonate, and the aqueous layer was extracted with ethyl acetate. The organic layer was washed with saturated aqueous sodium chloride, and dried with magnesium sulfate. The solvent was distilled off under reduced pressure. The residue was purified by silica gel chromatography eluting with ethyl acetate-hexane (1:4) to give 4-(4-Chlorophenyl)-1-[3-(5,11-dihydro-7-(3-methoxycarbonyl)propyl[1]benzoxepino[2,3-b]pyridin-5-ylidene)propyl]piperidin-4-ol (636 mg).

¹H-NMR (CDCl₃) δ: 1.93 (2H, m), 2.34 (2H, t), 2.59 (2H, t), 2.74 (2H, q), 3.47 (2H, t), 3.67 (3H, s), 5.33 (2H, brs), 6.05 (1H, t), 6.78 (1H, d), 7.00 (1H, dd), 7.09 (1H, d), 7.29 (1H, dd), 7.57 (1H, dd), 8.52 (1H, dd).

Step 2

The titled compound was prepared by following the procedure of Example 133, but replacing the product of Example 48 with the product of step 1.

¹H-NMR (DMSO-d6) δ: 1.37-1.57 (2H, m), 1.63-1.87 (4H, m), 2.10-2.36 (6H, m), 2.36-2.61 (6H, m), 4.83 (1H, brs), 5.24 (2H, brs), 6.14 (1H, t), 6.72 (1H, d), 7.00 (1H, dd), 7.12 (1H, d), 7.35 (2H, d), 7.41-7.48 (3H, m), 7.73 (1H, dd), 8.49 (1H, dd).

MS m/z: 533 (M+1)

### EXAMPLE 404

1-[3-(7-Benzoylsulfamoyl-5,11-dihydro[1]benzoxepino[2,3-b]pyridin-5-ylidene)propyl]-4-(4-chlorophenyl)piperidin-4-ol

The titled compound was prepared by following the procedure of Example 399, but replacing phenyl isocyanate with benzoyl chloride.

MS m/z: 630 (M+1)

### EXAMPLE 405

4-(4-Chlorophenyl)-1-[3-(5,11-dihydro-7-(2,5-dihydro-5-oxo-4H-1,2,4-oxadiazol-3-yl)methyloxy[1]benzoxepino[2,3-b]pyridin-5-ylidene)propyl]piperidin-4-ol

To a solution of the product of Example 407 (1.7 g) in DMF (20 ml) was added 2-ethyl Hexyl chloroformate (0.62 ml) and the mixture was stirred at 0° C. for 1 hour. Chloroform and water were added to the reaction mixture. The organic layer was extracted, and the solvent was distilled off under reduced pressure. The residue was purified by silica gel chromatography eluting with chloroform-methanol (30:1) and dissolved in xylene (50 ml). The solution was heated to reflux for 4 hours. The solvent was distilled off under reduced pressure. The residue was reslurried with ethanol to the titled compound (490 mg).

¹H-NMR (DMSO-d6) δ: 1.60-1.65 (2H, m), 1.91-1.99 (2H, m), 2.41-2.52 (2H, m), 2.70-2,89 (6H, m), 4.90 (2H, s), 5.19 (2H, brs), 6.16 (1H, t), 6.75-7.05 (3H, m), 7.37-7.48 (5H, m), 7.75 (1H, dd), 8.52 (1H, dd).

MS m/z: 561 (M+1)

### EXAMPLE 406

4-(4-Chlorophenyl)-1-[3-(5,11-dihydro-7-(2,5-dihydro-5-oxo-4H-1,2,4-oxadiazol-3-yl)[1]benzoxepino[2,3-b]pyridin-5-ylidene)propyl]piperidin-4-ol

The titled compound was prepared by following the procedure of Example 405, but replacing the product of Example 407 with the product of Example 408.

¹H-NMR (DMSO-d6) δ: 1.58-1.63 (2H, m), 1.87-1.96 (2H, m), 2.40-2.51 (2H, m), 2.63-2.85 (6H, m), 5.14 (2H, brs), 6.23 (1H, t), 6.92 (1H, d), 7.36-7.62 (6H, m), 7.77-7.81 (2H, m), 8.54 (1H, dd).

MS m/z: 531 (M+1)

### EXAMPLE 407

4-(4-Chlorophenyl)-1-[3-(7-hydroxyamidinomethoxy-5,11-dihydro[1]benzoxepino[2,3-b]pyridin-5-ylidene)propyl]piperidin-4-ol

The titled compound was prepared by following the procedure of Example 355, but replacing the product of Example 313 with the product of Example 49.

¹H-NMR (DMSO-d6) δ: 1.45-1.50 (2H, m), 1.70-1.82 (2H, m), 2.27-2.51 (8H, m), 4.37 (2H, s), 4.83 (1H, s), 5.20 (1H, brs), 5.57 (2H, brs), 6.17 (1H, t), 6.72-6.94 (3H, m), 7.33-7.48 (5H, m), 7.72 (1H, dd), 8.49 (1H, dd), 9.26 (1H, s).

MS m/z: 535 (M+1)

### EXAMPLE 408

4-(4-Chlorophenyl)-1-[3-(7-hydroxyamidino-5,11-dihydro[1]benzoxepino[2,3-b]pyridin-5-ylidene)propyl]piperidin-4-ol

The titled compound was prepared by following the procedure of Example 355, but replacing the product of Example 313 with the product of Example 364.

¹H-NMR (DMSO-d6) δ: 1.45-1.50 (2H, m), 1.73-1.81 (2H, m), 2.28-2.51 (8H, m), 4.83 (1H, s), 5.79 (2H, brs), 6.25 (1H, t), 6.81 (1H, d), 7.33-7.49 (6H, m), 7.63-7.76 (2H, m), 8.51 (1H, dd), 9.48 (1H, s).

MS m/z: 505 (M+1)

### EXAMPLE 409

4-(4-Chlorophenyl)-1-[3-(5,11-dihydro-7-(2-oxo-3H-1,2,3,5-oxathiadiazol-4-yl)methyloxy[1]benzoxepino[2,3-b]pyridin-5-ylidene)propyl]piperidin-4-ol

To a solution of the product of Example 407 (700 mg) in THF (20 ml) were added pyridine (0.21 ml) and thionyl chloride (0.1 ml) at 0° C., and the mixture was stirred at 0° C. for 1 hour and the mixture was stirred at room temperature for 30 minutes. Water, chloroform and 2-propanol were added to the reaction mixture. The organic layer was extracted and the solvent was distilled off under reduced pressure. The residue was purified by silica gel chromatography eluting with chloroform-methanol (5:1) to give the titled compound (170 mg).

MS m/z: 581 (M+1)

### EXAMPLE 410

4-(4-Chlorophenyl)-1-[3-(5,11-dihydro-7-(2,5-dihydro-5-oxo-4H-1,2,4-thiadiazol-3-yl)methyloxy[1]benzoxepino[2,3-b]pyridin-5-ylidene)propyl]piperidin-4-ol

To a solution of the product of Example 407 (700 mg) in THF (20 ml) was added thiocarbonyldiimidazole (280 mg) and the mixture was stirred at room temperature for 30 minutes. Water and ethyl acetate were added to the reaction mixture. The organic layer was extracted, and the solvent was distilled off under reduced pressure. To the residue were added THF (50 ml) and boron trifluoride diethyl etherate (0.8 ml), and the mixture was stirred at room temperature for 1 hour. Chloroform, 2-propanol and water were added to the reaction mixture. The organic layer was extracted, and the solvent was distilled off under reduced pressure. The residue was reslurried with acetone to the titled compound (180 mg).

MS m/z: 577 (M+1)

### EXAMPLE 411

4-(4-Chlorophenyl)-1-[3-(5,11-dihydro-7-ethoxycarbonylacetyl[1]benzoxepino[2,3-b]pyridin-5-ylidene)propyl]piperidin-4-ol

To a solution of the product of Example 315 (250 mg) in THF (3.0 ml) was added LDA (0.51 mol/L THF-hexane solution, 3.0 ml) at −78° C., and the mixture stirred at room temperature for 20 minutes. The reaction mixture was cooled to −78° C. again, and added ethyl cyanoformate (76 μl), stirred at room temperature for 1 hour. Saturated aqueous ammonium chloride and aqueous sodium chloride were added to the mixture, and the aqueous layer was extracted with ethyl acetate. The organic layer was washed with saturated aqueous sodium chloride, and dried with magnesium sulfate. The solvent was distilled off under reduced pressure. The residue was purified by silica gel chromatography eluting with chloroform-methanol (10:1) to give the titled compound (280 mg).

¹H-NMR (CDCl₃) δ: 1.26 (3H, t), 1.67-1.85 (2H, m), 1.93-2.13 (2H, m), 2.28-2.47 (4H, m), 2.47-2.60 (2H, m), 2.60-2.76 (2H, m), 3.94 (2H, s), 4.21 (2H, q), 5.60 (2H, brs), 6.22 (1H, t), 6.88 (1H, d), 7.29-7.34 (3H, m), 7.43 (2H, d), 7.59 (1H, d), 7.71 (1H, dd), 7.97 (1H, d), 8.53 (1H, d).

MS m/z: 561 (M+1)

### EXAMPLE 412

4-(4-fluorophenyl)-1-[3-(5,11-dihydro-7-hydroxy[1]benzoxepino[2,3-b]pyridin-5-ylidene) propyl]piperidine-4-ol

To a solution of 5-(3-bromopropylidene)-5,11-dihydro-7-hydroxy-[1]benzoxepino[2,3-b]pyridine (2.59 g) in DMF (10 ml) was added 4-(4-Fluorophenyl)-4-hydroxypiperidine (1.02 g) and triethylamine (835 EM). The solution was stirred at room temperature for 23 hours. The reaction was quenched with water, extracted with ethyl acetate, and evaporated in vacuo. The residue was purified by silica gel chromatography (87:10:3 ethyl acetate: methanol: triethylamine) to yield 0.9 g (39%) of the title compound. ¹H-NMR (DMSO) d: 1.64-1.69 (2H, m), 1.74-1.85 (2H, m), 2.27-2.52 (8H, m), 4.81 (1H, s), 5.16 (2H, brs), 6.08 (1H, t), 6.62-6.71 (3H, m), 7.12 (2H, t), 7.40-7.51 (3H, m), 7.72 (1H, dd), 8.48 (1H, dd), 9.09 (1H, s).

ESI-MS m/z: 447 (M+1).

### EXAMPLE 413

4-(4-fluorophenyl)-1-[3-(5,11-dihydro-7-carboxy[1]benzoxepino[2,3-b]pyridin-5-ylidene)propyl]piperidine-4-ol

The titled compound was prepared by following the procedure of example 118, but replacing the compound of Example 169 with the triflate derived from compound 412.

¹H-NMR (MeOD) δ: 1.78-1.85 (2H, m), 2.25-2.40 (2H, m), 2.57-2.70 (2H, m), 3.06-3.35 (7H, m), 5.06-5.81 (2H, brs), 6.23 (1H, t), 6.77 (1H, d), 7.00-7.11 (2H, m), 7.37-7.56 (3H, m), 7.65-7.80 (2H, m), 8.01 (1H, d), 8.48 (1H, dd).

MS m/z: 475

### EXAMPLE 414

4-(4-fluorophenyl)-1-[3-(5,11-dihydro-7-(1-hydroxy-1-methylethyl)-[1]benzoxepino[2,3-b]pyridin-5-ylidene)propyl]piperidine-4-ol

The titled compound was prepared by following the procedure of Example 27, but starting with the methyl ester of the compound of Example 413.

¹H-NMR (CDCl₃) δ: 1.57-2.14 (12H, m), 2.34-2.45 (4H, m), 2.50-2.61 (2H, m), 2.63-2.78 (2H, m), 5.22-5.43 (2H, brs), 6.14 (1H, t), 6.95-7.10 (2H, m), 7.25-7.35 92H, m), 7.40-7.60 (4H, m), 8.50 (1H, dd).

MS m/z: 489

### EXAMPLE 415

4-(4-Chlorophenyl)-1-[3-(5,11-dihydro-7-diethylcarbamoyl-[1]benzoxepino[2,3-b]pyridin-5-ylidene) propyl]piperidine-4-ol

The titled compound was prepared by following the procedure of Example 316, but replacing dimethylamine with diethylamine.

¹H-NMR (CDCl₃) δ: 1.18-1.30 (6H, m), 1.65 (2H, d), 1.80 (1H, s), 2.05 (2H, dt), 2.30-2.45 (4H, m), 2.50 (2H, t), 2.60-2.70 (2H, m), 3.35-3.50 (4H, m), 5.30 (2H, brs), 6.15 (1H, t), 6.83 (1H, d), 6.90 (1H, dd), 7.10 (1H, dd), 7.23-7.35 (3H, m), 7.40 (2H, d), 7.56 (1H, dd), 8.50 (1H, dd).

MS m/z: 563

### EXAMPLE 416

4-(4-Chlorophenyl)-1-[3-(5,11-dihydro-7-phenylsulfonylcarbamoyl-[1]benzoxepino[2,3-b]pyridin-5-ylidene) propyl]piperidine-4-ol

To a solution of the compound of Example 44 (0.511 g, 1.1 mmol) in dry THF (20 mL) was added sodium hydride (60% in mineral oil, 48 mg, 1.2 mmol), and the slurry heated at 40° C. under argon with stirring for 20 minutes. Phenylsulfonylisocyanate (160 μL, 1.2 mmol) was added and the mixture was stirred for 14 hours. The solvent was then removed by rotary evaporation to give the crude product. The solid material was washed twice with 20 mL CH₂Cl₂, and then twice with 20 mL MeoH: CH₂Cl₂ (1:1) to give the title compound (274 mg).

MS m/z: 647

### EXAMPLE 417

4-(4-Chlorophenyl)-1-[3-(5,11-dihydro-7-methoxycarbonyl-carbamoyl-[1]benzoxepono[2,3-b]pyridin-5-ylidene) propyl]piperidine-4-ol

To a solution of the compound of Example 44 (0.214 g, 0.46 mmol) in dry THF (5 mL) was added sodium hydride (60% in mineral oil, 28 mg, 0.7 mmol), and the slurry heated at 50° C. under argon with stirring for 20 minutes. Methyl isocyanatoformate (56 μl, 0.7 mmol) was added and the mixture was stirred for 14 hours. The solvent was then removed by rotary evaporation to give the crude product. The residue was purified by silica gel chromatography eluting with a dichloromethane/2.0 M ammonia in methanol gradient (0 to 4% MeOH over 1 hour) to give the title compound (102 mg).

¹H-NMR (CDCl₃) δ: 1.60-1.65 (2H, m), 1.80 (1H, s), 2.05 (2H, dt), 2.30-2.45 (4H, m), 2.50 (2H, t), 2.60-2.70 (2H, m), 3.35 (3H, s), 5.30 (2H, brs), 6.15 (1H, t), 6.83 (1H, d), 6.90 (1H, dd), 7.10 (1H, dd), 7.23-7.35 (3H, m), 7.40 (2H, d), 7.56 (1H, dd), 8.50 (1H, dd).

MS m/z: 565

### EXAMPLE 418

4-(4-Chlorophenyl)-1-[3-(5,11-dihydro-7-(R-3-ethoxycarbonyl-piperidine-1-yl)-carbamoyl-[1]benzoxepino[2,3-b]pyridin-5-ylidene) propyl]piperidine-4-ol

Step 1

R-ethyl nipecotate-L-tartrate (1.53 g) was freebased with aqueous sodium hydroxide and ethyl acetate. The organic layers were evaporated, and the resulting amine was redissolved in THF (10 mL) and treated with carbonyl-diimidazole (0.81 g). The resulting solution was stirred at room temperature for 23 hours, concentrated in vacuo, and redissolved in acetonitrile (5 mL). This solution was treated with methyl iodide (0.347 mL) and stirred for 18 hours at room temperature.

Step 2

The compound of Example 44 (0.7 g) was suspended in THF (25 mL) and treated with sodium hydride (0.036 g) and stirred at room temperature for one hour. The resulting anion was added to the imidazolium salt prepared in Step 1, and the solution was heated to reflux for 18 hr. The crude material was then loaded on silica gel and purified by silica gel chromatography (87:10:3 ethyl acetate:methanol:triethylamine) to yield 0.278 g (64%) of the title compound.

¹H-NMR (DMSO) δ: 1.11-1.21 (3H, m), 1.45-2.0 (8H, m), 2.15-2.40 (6H, m), 3.05-3.15 (2H, m), 3.31 (2H, m), 3.95-4.15 (3H, m), 5.31 (2H, brs), 6.14 (1H, t), 6.78 (1H, d), 6.92 (1H, dd), 7.05 (1H, d), 7.33 (2H, d), 7.42-7.47 (3H, m), 7.72 (1H, dd), 8.50 (1H, dd).

ESI-MS m/z: 646 (M+1).

### EXAMPLE 419

4-(4-Chlorophenyl)-1-[3-(5,11-dihydro-7-(R-3-ethoxycarbonyl-piperidine-1-yl)-carbamoyl-[1]benzoxepino[2,3-b]pyridin-5-ylidene) propyl]piperidine-4-ol

The compound of Example 418 (0.195 g) was dissolved in THF (1 mL) and treated with aqueous lithium hydroxide (0.0084 g) and stirred at room temperature for 18 hours. The resulting solution was concentrated in vacuo, and the residue was purified by chromatography on a reverse-phase solid-phase-extraction column, eluting with water-acetonitrile, 0.1% formic acid, to yield 0.153 g (77%) of the title compound.

¹H-NMR (DMSO) δ: 1.55-2.25 (8H, m), 2.30-2.80 (1OH, m), 3.22 (1H, m), 4.15-4.35 (2H, m), 5.41 (2H, brs), 6.35 (1H, t), 6.98 (1H, d), 7.13 (1H, dd), 7.25 (1H, d), 7.54 (2H, d), 7.64 (3H, m), 7.90 (1H, dd), 8.50 (1H, s), 8.70 (1H, dd).

ESI-MS m/z: 618 (M+1).

### EXAMPLE 420

4-(4-Chlorophenyl)-1-[3-(5,11-dihydro-7-(4-ethoxycarbonyl-piperidine-1-yl)-carbamoyl-[1]benzoxepino[2,3-b]pyridin-5-ylidene) propyl]piperidine-4-ol

The titled compound was prepared by following the procedure of Example 418, but replacing R-ethyl nipecotate-L-tartrate with ethyl isonipecotate.

¹H-NMR (CDCl₃) δ: 1.25 (3H, t), 1.60-1.80 (4H, m), 1.90-2.05 (4H, m), 2.25-2.65 (10H, m), 2.90-3.15 (2H, m), 4.05-4.25 (4H, m), 5.30 (2H, brs), 6.15 (1H, t), 6.75-6.90 (2H, m), 7.05 (1H, d), 7.20-7.40 (3H, m), 7.40 (2H, d), 7.56 (1H, dd), 8.45 (1H, dd).

MS m/z: 647

### EXAMPLE 421

4-(4-Chlorophenyl)-1-[3-(5,11-dihydro-7-(4-carboxy-piperidine-1-yl)-carbamoyl-[1]benzoxepino[2,3-b]pyridin-5-ylidene) propyl]piperidine-4-ol

A solution of the compound of Example 420 (91 mg, 0.14 mmol) in MeOH (5 mL) was treated with a 0.4 M solution of lithium hydroxide (5 mL, 2 mmol) and stirred for 3 hours. After addition of 5 mL of 0.4 N HCl, the solvent was removed under reduced pressure to give the crude product. The residue was purified using silica gel chromatography eluting with a dichloromethane:methanol gradient (0 to 50% MeOH over 1 hour) to give the title compound (48 mg).

¹H-NMR (MeOD) δ: 1.60-1.65 (2H, m) 2.10-2.70 (10H, m), 5.30 (2H, brs), 6.15 (1H, t), 6.80-6.90 (2H, m), 7.20-7.50 (6H, m), 7.62 (1H, dd), 8.48 (1H, dd).

MS m/z: 619

### EXAMPLE 422

### 4-(4-Chlorophenyl)-1-[3-(5,11-dihydro-7-(S-3-ethoxycarbonyl-piperidine-1-yl)-carbamoyl-[1]benzoxepino[2,3-b]pyridin-5-ylidene) propyl]piperidine-4-ol

The titled compound was prepared by following the procedure of Example 418, but replacing R-ethyl nipecotate-L-tartrate with ethyl (S)-nipecotate-D-tartrate.

¹H-NMR (CDCl₃) δ: 1.25 (3H, t), 1.30-1.70 (5H, m), 1.94-2.05 (3H, m), 2.25-2.65 (11H, m), 3.05-3.15 (1H, m), 4.05-4.25 (4H, m), 5.30 (2H, brs), 6.15 (1H, t), 6.75-6.90 (2H, m), 7.05 (1H, d), 7.20-7.40 (3H, m), 7.40 (2H, d), 7.56 (1H, dd), 8.45 (1H, dd).

MS m/z: 647

### EXAMPLE 423

4-(4-Chlorophenyl)-1-[3-(5,11-dihydro-7-ethoxycarbonyl-[1]benzoxepino[2,3-b]pyridin-5-ylidene) propyl]piperidine-4-ol

The compound of Example 169 (0.166 g) was dissolved in DMF (1 mL) and treated with palladium (II) acetate (0.007 g), 1,3-bis-diphenylphosphinopropane (0.012 g), triethylamine (0.1 mL) and ethanol (1 mL), and stirred at 60° C. for 18 hours under a CO balloon. The resulting solution was quenched with water, extracted with ethyl acetate, concentrated in vacuo, and purified by silica gel chromatography (87:10:3 ethyl acetate:methanol:triethylamine). The residue was further purified by chromatography on a reverse-phase solid-phase-extraction column, eluting with water-acetonitrile, 0.1% formic acid, to yield 0.114 g (73%) of the title compound.

¹H-NMR (DMSO) δ: 1.28 (3H, t), 1.40-1.55 (2H, m), 1.71-1.85 (2H, m), 2.20-2.60 (6H, m), 3.22 (2H, m), 4.28 (2H, q), 5.00-5.60 (2H, brs), 6.21 (1H, t), 6.92 (1H, d), 7.40-7.80 (8H, m), 8.50 (1H, d).

ESI-MS m/z: 519 (M+1).

### EXAMPLE 424

4-(4-Chlorophenyl)-1-[3-(5,11-dihydro-7-(ethoxycarbonylmethyl)-oxycarbonyl-[1]benzoxepino[2,3-b]pyridin-5-ylidene) propyl]piperidine-4-ol

The procedure of Example 423 was followed, but replacing ethanol with ethyl glyoxylate to yield 0.041 g (26%) of the title compound.

¹H-NMR (DMSO) δ: 1.10-1.30 (3H, m), 1.35-1.55 (2H, m), 1.60-1.85 (2H, m), 2.20-2.60 (6H, m), 3.32 (2H, m), 4.05-4.25 (2H, m), 4.87 (2H, s), 5.00-5.60 (2H, brs), 6.21 (1H, t), 6.92 (1H, d), 7.2-7.90 (8H, m), 8.50 (1H, d).

ESI-MS m/z: 577 (M+1).

### EXAMPLE 425

4-(4-Chlorophenyl)-1-[3-(5,11-dihydro-7-cyclohexyloxycarbonyl-[1]benzoxepino[2,3-b]pyridin-5-ylidene) propyl]piperidine-4-ol

The procedure of Example 423 was followed, but replacing ethanol with cyclohexanol to yield 0.050 g (32%) of the title compound.

¹H-NMR (MeOD) δ: 1.30-2.20 (14H, m), 2.53-2.60 (2H, m), 2.95-3.32 (6H, m), 5.00 (1H, m), 5.00-5.60 (2H, brs), 6.28 (1H, t), 6.92 (1H, d), 7.40-7.55 (8H, m), 7.95 (2H, m), 8.05 (1H, s), 8.50 (2H, m).

ESI-MS m/z: 573 (M+1).

### EXAMPLE 426

4-(4-Chlorophenyl)-1-[3-(5,11-dihydro-7-(1-propoxy)carbonyl-[1]benzoxepino[2,3-b]pyridin-5-ylidene) propyl]piperidine-4-ol

To a solution of the compound of Example 118 (109 mg, 0.22 mmol) in dry DMF (5 mL) was added potassium carbonate (91 mg) followed by propyl iodide (24 μL, 0.66 mmol). The mixture was heated to 55° C. for 14 hours. The mixture was diluted with ethyl acetate (200 mL), washed twice with water (200 mL) and then with brine (100 mL), and dried with sodium sulfate. The organic solvent was removed under reduced pressure and the residue subjected to silica gel chromatography using a dichloromethane : methanol gradient (0 to 5% MeOH over 1 hour) to give the title compound (103 mg).

¹H-NMR (CDCl₃) δ: 1.06 (3H, t), 1.50-2.10 (4H, m), 2.14-2.25 (2H, m), 2.31-2.75 (10H, m), 4.28 (2H, t), 6.15 (1H, t), 6.83 (1H, d), 7.24-7.38 (3H, m), 7.42 (2H, d), 7.59 (1H, dd), 7.78 (1H, dd), 8.00 (1H, d), 8.50 (1H, dd).

MS m/z: 533

### EXAMPLE 427

4-(4-Chlorophenyl)-1-[3-(5,11-dihydro-7-(1-butoxy)carbonyl-[1]benzoxepino[2,3-b]pyridin-5-ylidene) propyl]piperidine-4-ol

The procedure of Example 423 was followed, but replacing ethanol with n-butanol to yield 0.065 g (45%) of the title compound. ¹H-NMR (MeOD) δ: 0.85-0.91 (3H, m), 1.25-1.45 (2H, m), 1.55-1.70 (2H, m), 1.70-1.85 (2H, m), 2.10-2.28 (2H, m), 2.53-2.60 (2H, m), 3.15-3.38 (6H, m), 4.12-4.21 (2H, m), 5.00-5.60 (2H, brs), 6.10 (1H, t), 6.76 (1H, d), 7.22-7.40 (3H, m), 7.71 (1H, m), 7.95 (1H, m), 8.05 (1H, s), 8.30 (1H, s), 8.41 (1H, m).

ESI-MS m/z: 547 (M+1).

### EXAMPLE 428

4-(4-Chlorophenyl)-1-[3-(5,11-dihydro-7-(2-propoxy)carbonyl-[1]benzoxepino[2,3-b]pyridin-5-ylidene) propyl]piperidine-4-ol

The titled compound was prepared by following the procedure of Example 426, but replacing propyl iodide with 2-bromopropane.

¹H-NMR (CDCl₃) δ: 1.30-2.10 (8H, m), 2.14-2.25 (2H, m), 2.31-2.75 (10H, m), 5.15-5.60 (2H, m), 6.15 (1H, t), 6.83 (1H, d), 7.24-7.38 (3H, m), 7.44 (2H, d), 7.59 (1H, dd), 7.80 (1H, dd), 8.02 (1H, d), 8.50 (1H, dd).

MS m/z: 533

### EXAMPLE 429

4-(4-Chlorophenyl)-1-[3-(5,11-dihydro-7-cyclopentyl-oxycarbonyl-[1]benzoxepino[2,3-b]pyridin-5-ylidene) propyl]piperidine-4-ol

The titled compound was prepared by following the procedure of Example 426, but replacing propyl iodide with cyclopentyl bormide.

¹H-NMR (MeOD) δ: 1.23-1.33 (1H, m), 1.50-2.04 (10H, m), 2.27-2.41 (2H, m), 2.70-2.90 (2H, m), 3.30-3.62 (5H, m), 5.21-5.85 (3H, m), 6.15 (1H, t), 6.85 (1H, d), 7.38 (2H, d), 7.42 (2H, d), 7.60-7.82 (2H, m), 8.04 (1H, d), 8.61 (1H, dd).

MS m/z: 559

### EXAMPLE 430

4-(4-Chlorophenyl)-1-[3-(5,11-dihydro-7-(2-morpholinoethyl-1-yl)-oxycarbonyl-[1]benzoxepino[2,3-b]pyridin-5-ylidene) propyl]piperidine-4-ol

The titled compound was prepared by following the procedure of Example 426, but replacing propyl iodide with 2-morpholinoethyl chloride.

¹H-NMR (CDCl₃) δ: 1.62-1.70 (2H, m) 1.90-2.13 (2H, m), 2.30-2.80 (14 H, m), 3.62-3.75 (4H, m), 4.41 (2H, t), 5.11-5.62 (2H, brs), 6.19 (1H, t), 6.83 (1H, d), 7.23-7.38 (3H, m), 7.42 (2H, d), 7.59 (1H, dd), 7.78 (1H, dd), 8.00 (1H, d), 8.50 (1H, dd).

MS m/z: 604

### EXAMPLE 431

4-(4-Chlorophenyl)-1-[3-(5,11-dihydro-7-(2,2-diethylaminoethyl-1-yl)-oxycarbonyl-[1]benzoxepino[2,3-b]pyridin-5-ylidene) propyl]piperidine-4-ol

The titled compound was prepared by following the procedure of Example 426, but replacing propyl iodide with 2-(N,N-diethylamino)ethyl chloride.

¹H-NMR (CDCl₃) δ: 1.06 (6H, t), 1.62-1.71 (2H, m), 1.93-2.10 (2H, m), 2.30-2.75 (12H, m), 2.85 (2H, t), 4.38 (2H, t), 5.20-5.58 (2H, brs), 6.15 (1H, t), 6.83 (1H, d), 7.24-7.38 (3H, m), 7.42 (2H, d), 7.59 (1H, dd), 7.78 (1H, dd), 8.00 (1H, d), 8.50 (1H, dd).

MS m/z: 590

### EXAMPLE 432

4-(4-Chlorophenyl)-1-[3-(5,11-dihydro-7-(1-2,2-dimethylpropionyl-oxymethyl)-oxycarbonyl-[1]benzoxepino[2,3-b]pyridin-5-ylidene) propyl]piperidine-4-ol

The procedure of Example 426 was followed, but replacing with chloromethyl pivalate to yield 0.36 g (77%) of the title compound.

¹H-NMR (CDCl₃) δ: 1.18 (9H, s), 1.58-1.72 (2H, m), 1.85-2.85 (10H, m), 5.00-5.60 (2H, brs), 5.94 (2H, s), 6.17 (1H, t), 6.82 (1H, d), 7.22-7.42 (5H, m), 7.56 (1H, dd), 7.80 (1H, dd), 7.99 (1H, d), 8.05 (1H, d), 8.46 (1H, dd).

ESI-MS m/z: 605 (M+1).

### EXAMPLE 433

4-(4-Chlorophenyl)-1-[3-(5,11-dihydro-7-(2-hydroxyethyl-1-yl)-oxycarbonyl-[1]benzoxepino[2,3-b]pyridin-5-ylidene) propyl]piperidine-4-ol

The procedure of Example 423 was followed, but replacing ethanol with ethylene glycol to yield 0.076 g (42%) of the title compound.

¹H-NMR (MeOD) δ: 1.80-2.00 (4H, m), 2.25-2.35 (2H, m), 2.55-2.65 (2H, m), 3.15-3.45 (5H, m), 3.75 (2H, dd), 4.24 (2H, dd), 5.00-5.60 (2H, brs), 6.10 (1H, t), 6.76 (1H, d), 7.18-7.42 (5H, m), 7.71 (2H, m), 7.99 (1H, m), 8.05 (1H, s), 8.30 (1H, s), 8.41 (1H, m).

ESI-MS m/z: 535 (M+1).

### EXAMPLE 434

5-{3-[4-(4-Chloro-phenyl)-4-hydroxy-3-methyl-piperidin-1-yl]-propylidene} -5,11-dihydro-10-oxa-1-aza-dibenzo[a,d]cyclohepten-7-ol

Step 1: 3-Methyl-4-oxo-piperidine-1-carboxylic Acid Tert-Butyl Ester

In a Parr shaker flask, 1-benzyl-3-methyl-4-piperidone (19 g, 93 mmol), di-tert-butyl dicarbonate (27 g, 121 mmol) and palladium hydroxide (2.6 g) were suspended in methanol (75 mL) and then purged with argon. The reaction mixture was then purged with hydrogen and placed on a Parr shaker apparatus for about 16 hours at about 44 psi of hydrogen. The catalyst was filtered over celite and washed with methanol. The crude product was chromatographed on silica gel, eluting with EtOAc/hexane (1:5) to give a white crystalline solid.

¹H-NMR (CDCl₃) δ: 0.97 (3H, d), 1.45 (9H, s), 2.37-2.53 (3H, m), 2.80 (1H, brs), 3.16-3.26 (1H, m), 4.11-4.18 (2H, m).

ESI-MS m/z: 158 [M—CH═C(CH₃)₂+1].

Step 2: 4-(4-Chloro-phenyl)-4-hydroxy-3-methyl-piperidine-1-carboxylic Acid Tert-Butyl Ester

To 4-chlorophenyl magnesium bromide (49 mL, 49 mmol, IM in diethyl ether) at about 0° C. under argon, was added 3-Methyl-4-oxo-piperidine-1-carboxylic acid tert-butyl ester (7 g, 32.8 mmol) in THF (50 mL) over a period of about 1 hour. The reaction was allowed to warm to room temperature and stirred overnight. The reaction was quenched with saturated ammonium chloride solution and extracted with ethyl acetate. The organic layer was washed with water, then brine and dried over sodium sulfate. The solvent was evaporated and the crude residue was recrystallized in EtOAc to give a white solid.

¹H-NMR (CDCl₃) δ: 0.58 (3H, d), 1.45 (9H, s), 1.50-1.69 (2H, m), 1.76-2.13 (2H, m), 2.80 (1H, t), 3.10 (1H, t), 3.96 (2H, brs), 7.29 (4H, m).

ESI-MS m/z: 252 [M—CH═C(CH₃)₂—H₂O+1].

Step 3: 4-(4-Chloro-phenyl)-3-methyl-piperidin-4-ol

To a solution of 4-(4-Chloro-phenyl)-4-hydroxy-3-methyl-piperidine-1-carboxylic acid tert-butyl ester (1.5 g, 4.6 mmol) in dichloromethane (40 mL) at about 0° C. was added trifluoroacetic acid (10 mL). The solution was stirred for about 2 hours. The solvent was evaporated and the residue was dissolved in ethyl acetate. The solution was neutralized with saturated sodium bicarbonate, and washed with brine and dried over magnesium sulfate. The solvent was removed to give a yellow solid. No purification was needed.

¹H-NMR (CDCl₃) δ: 0.65 (3H, d), 1.85 (1H, d), 2.52 (2H, m), 3.05 (1H, t), 3.27 (1H, dd), 3.34 (2H, d), 7.40 (4H, m).

ESI-MS m/z: 240 [M+1].

5-{3-[4-(4-Chloro-phenyl)-4-hydroxy-3-methyl-piperidin-1-yl]-propylidene}-5,11-dihydro-10-oxa-1-aza-dibenzo[a,d]cyclohepten-7-ol

To a solution of 4-(4-Chloro-phenyl)-3-methyl-piperidin-4-ol (1.0 g, 4.6 mmol) in DMF (10 mL) with triethylamine (1.75 mL, 12.54 mmol), was added 5-(3-Bromo-propylidene)-5,11-dihydro-10-oxa-1-aza-dibenzo[a,d]cyclohepten-7-ol (1.38 g, 4.18 mmol) dropwise for over a period of about 1.5 hours at about 50° C. The reaction was stirred overnight at about 50° C. The reaction was quenched with water, extracted with ethyl acetate, and evaporated in vacuo. The residue was purified by silica gel chromatography (87:10:3 ethyl acetate:methanol:triethylamine) to yield a brown solid of the title compound.

¹H-NMR (CDCl₃) δ: 0.53 (3H, d), 1.60-1.69 (2H, m), 2.07-2.30 (3H, m), 2.34-2.51 (3H, m), 2.56-2.75 (3H, m), 2.76-2.87 (1H, m), 2.98-3.11 (1H, m), 5.25 (2H, brs), 6.12 (1H, t), 6.64-6.79 (3H, m), 7.20-7.40 (5H, m), 7.53 (1H, d), 8.50 (1H, d).

ESI-MS m/z: 477 [M+1].

### EXAMPLE 435

Trifluoro-methanesulfonic Acid 5-{3-[4-(4-chloro-phenyl)-4-hydroxy-3-methyl-piperidin-1-yl]-propylidene} -5,11-dihydro-10-oxa-1-aza-dibenzo[a,d]cyclohepten-7-yl Ester

The title compound was prepared according to the procedure of Example 169 and obtained as a brown solid.

¹H-NMR (CDCl₃) δ: 0.55 (3H, d), 1.55-1.68 (2H, m), 1.92-2.18 (2H, m), 2.24-2.69 (7H, m), 5.36 (2H, brs), 6.15 (1H, t), 6.88 (1H, d), 7.05 (1H, dd), 7.19-7.43 (6H, m), 7.60 (1H, d), 8.54 (1H, d).

ESI-MS m/z: 609 [M+1].

### EXAMPLE 436

5-{3-[4-(4-Chloro-phenyl)-4-hydroxy-3-methyl-piperidin-1-yl]-propylidene}-5,11-dihydro-10-oxa-1-aza-dibenzo[a,d]cycloheptene-7-carboxylic Acid Methyl Ester

The title compound was prepared according to the procedure of Example 423 and obtained as a brown solid (formate salt).

¹H-NMR (CDCl₃) δ: 0.62 (3H, d), 1.75 (1H, d), 2.41-2.71 (5H, m), 2.90-3.14 (4H, m), 3.27 (1H, d), 3.89 (3H, s), 5.00-5.70 (2H, brs), 6.08 (1H, t), 6.86 (1H, d), 7.26-7.39 (5H, m), 7.59 (1H, d), 7.83 (1H, d), 7.98 (1H, s), 8.34 (1H, s), 8.56 (1H, d).

ESI-MS m/z: 519 [M+1].

### EXAMPLE 437

4-(4-Chloro-phenyl)-1-{3-[7-(1-hydroxy-1-methyl-ethyl)-11H-10-oxa-1-aza-dibenzo[a,d]cyclohepten-5-ylidene]-propyl} -3-methyl-piperidin-4-ol

The title compound was prepared according to the procedure of Example 27. The racemic compound was resolved using preparative chiral HPLC (ChiralPak AD, 20 mm×250 mm, isocrati elution with 10% ethanol:90% hexane, 15 mL/min., 35 minute run time). The more active enantiomer eluted first, at 17 minutes. The less active enantiomer eluted second, at 23 minutes. white solid (formate salt).

¹H-NMR (CDCl₃) δ: 0.61 (3H, d), 1.57 (6H, s), 1.76 (1H, d), 2.43-2.72 (5H, m), 2.91-3.12 (4H, m), 3.25 (1H, d), 5.04-5.46 (2H, brs), 6.01 (1H, t), 6.82 (1H, d), 7.22-7.37 (5H, m), 7.46 (1H, s), 7.55 (1H, d), 8.30-8.40 (1H, brs), 8.52 (1H, d).

ESI-MS m/z: 519 [M+1].

### EXAMPLE 438

5-{3-[4-(4-Chloro-phenyl)-4-hydroxy-3-methyl-piperidin-1-yl]-propylidene} -5,11-dihydro-10-oxa-1-aza-dibenzo[a,d]cycloheptene-7-carboxylic Acid

The title compound was prepared according to the procedure of Example 118 and obtained as a brown solid (formate salt).

¹H-NMR (CDCl₃) δ: 0.49 (3H, d), 1.63 (1H, d), 2.29-2.43 (2H, m), 2.45-2.63 (3H, m), 2.78-3.18 (5H, m), 4.82-5.85 (2H, brs), 6.13 (1H, t), 6.67 (1H, d), 7.19-7.31 (5H, m), 7.55 (1H, d), 7.67 (1H, d), 7.87 (1H, s), 8.19 (1H, s), 8.40 (1H, d).

ESI-MS m/z: 505 [M+1].

### EXAMPLE 439

5-{3-[4-(4-Chloro-phenyl)-4-hydroxy-3,5-dimethyl-piperidin-1-yl]-propylidene}-5,11-dihydro-10-oxa-1-aza-dibenzo[a,d]cyclohepten-7-ol

Step 1: 1-Benzyl-3,5-dimethyl-piperidin-4-one

To 1-benzyl-3-methyl-4-piperidone (2.0 g, 9.8 mmol) in THF at about −78° C. under argon, was added Lithium diisopropylamide (7.35 mL, 14.7 mmol, 2 M in heptane/THF/ethylbenzene). After stirring for about 1 hour at about −78° C., iodomethane (0.73 mL, 11.8 mmol) was added. The reaction mixture was stirred for about 1 hour at about −78° C., then warmed to room temperature. Stirring was continued overnight at room temperature. The reaction was quenched with saturated ammonium chloride solution, and extracted with ethyl acetate. The organic layer was washed with water, then brine and dried over magnesium sulfate. The crude product was chromatographed on silica gel, eluting with EtOAc/hexane (3:10) to give a yellow oil of the title compound.

¹H-NMR (CDCl₃) δ: 0.96 (6H, d), 2.04 (2H, t), 2.62-2.78 (2H, m), 3.12-3.17 (2H, m), 3.59 (2H, s), 7.23-7.38 (5H, m).

ESI-MS m/z: 218 [M+1].

Step 2: 3,5-Dimethyl-4-oxo-piperidine-1-carboxylic Acid Tert-Butyl Ester

The title compound was prepared according to the procedure of Example 434, step 1 and obtained as a white crystalline solid.

¹H-NMR (CDCl₃) δ: 1.02 (6H, d), 1.5 (9H, s), 2.47-2.75 (4H, m), 4.25-4.54 (2H, brs).

ESI-MS m/z: 172 [M—CH═C(CH₃)₂+1].

Step 3: 4-(4-Chloro-phenyl)-4-hydroxy-3,5-dimethyl-piperidine-1-carboxylic Acid Tert-Butyl Ester

The title compound was prepared according to the procedure of Example 434, step 2 and obtained as a white solid.

¹H-NMR (CDCl₃) δ: 0.57 (6H, d), 1.49 (9H, s), 1.98-2.08 (2H, m), 2.71-2.98 (2H, m), 3.79-4.10 (2H, m), 7.29-7.36 (4H, m).

ESI-MS m/z: 266 [M—CH═C(CH₃)₂—H₂O+1].

Step 4: 4-(4-Chloro-phenyl)-3,5-dimethyl-piperidin-4-ol

The title compound was prepared according to the procedure of Example 434, step 3 and obtained as a light yellow solid.

¹H-NMR (CDCl₃) δ: 0.54 (6H, d), 1.97-2.11 (4H, m), 2.75 (2H, t), 2.88 (2H, d), 7.27-7.32 (4H, m).

ESI-MS m/z: 240 [M+1].

5-{3-[4-(4-Chloro-phenyl)-4-hydroxy-3,5-dimethyl-piperidin-1-yl]-propylidene}-5,11-dihydro-10-oxa-1-aza-dibenzo[a,d]cyclohepten-7-ol

The title compound was prepared according to the procedure of Example 434, and obtained as a yellow solid.

¹H-NMR (CDCl₃) δ: 0.58 (6H, d), 2.50-2.80 (6H, m), 2.94-3.06 (2H, m), 3.17 (2H, d), 5.14-5.29 (2H, brs), 5.87 (1H, t), 6.75-6.90 (2H, m), 7.10-7.45 (6H, m), 8.44 (1H, d), 8.52 (1H, s).

ESI-MS m/z: 491 [M+1].

### EXAMPLE 440

4-(4-Chloro-phenyl)-1-{3-[7-(1-hydroxy-1-methyl-ethyl)-1H-10-oxa-1-aza-dibenzo[a,d]cyclohepten-5-ylidene]-propyl}-3,5-dimethyl-piperidin-4-ol

The title compound was prepared according to the procedure of Example 439 but using 2-[5-(3-Bromo-propylidene)-5,11-dihydro-10-oxa-1-aza-dibenzo[a,d]cyclohepten-7-yl]-propan-2-ol, and was obtained as a white solid (formate salt).

¹H-NMR (CDCl₃) δ: 0.59 (6H, d), 1.58 (6H, s), 2.49-2.74 (6H, m), 2.97-3.10 (2H, m), 3.16 (2H, d), 5.20-5.45 (2H, brs), 6.06 (1H, t), 6.83 (1H, d), 7.12-7.43 (6H, m), 7.49 (1H, s), 7.60 (1H, d), 8.38 (1H, s), 8.54 (1H, m).

ESI-MS m/z: 533 [M+1].

### EXAMPLES 441 AND 442

4-(4-Chloro-phenyl)-1-{3-[7-(1-hydroxy-1-methyl-ethyl)-11H-10-oxa-1-aza-dibenzo[a,d]cyclohepten-5-ylidene]-propyl}-3-methyl-piperidin-4-ol and

4-(4-Chloro-phenyl)-1-{3-[7-(1-hydroxy-1-methyl-ethyl)-11H-10-oxa-1-azadibenzo[a,d]cyclohepten-5-ylidene]-propyl}-4-methyl-piperidin-3-ol

Step 1: 4-(4-Chloro-phenyl)-3,6-dihydro-2H-pyridine-1-carboxylic Acid Tert-Butyl Ester

To a solution of di-tert-butyl-dicarbonate (3.27 g, 15.0 mmol) in CH₂Cl₂ (50 mL) was added 4-(4-chlorophenyl)-1,2,3,6-tetrahydropiperidine hydrochloride (3.02 g, 13.1 mmol) and triethylamine (3.6 mL, 20 mmol). The solution was stirred at about room temperature for about 15 hours. Gas evolution was observed. The reaction was quenched with aqueous ammonium chloride, extracted with CH₂Cl₂, and the organic layers were evaporated in vacuo. The residue was purified by plug filtration through silica gel to yield the title compound as a colorless oil.

¹H-NMR (CDCL₃) d: 1.50 (9H, s), 2.46 (2H, br s), 3.62 (2H, br s), 4.05 (2H, br s), 6.01 (1H, br s), 7.25 (4H, s).

Step 2: 6-(4-Chloro-phenyl)-7-oxa-3-aza-bicyclo[4.1.0]heptane-3-carboxylic Acid Tert-Butyl Ester

To a solution of 4-(4-Chloro-phenyl)-3,6-dihydro-2H-pyridine-1-carboxylic acid tert-butyl ester (3.8 g, 13 mmol) in CH₂Cl₂ (50 mL) was added 3-chloroperbenzoic acid (3.8 g, 17.0 mmol) and triethylamine (3.6 mL, 20 mmol). The solution was stirred at room temperature for about 4 hours. A white precipitate was observed. The reaction was quenched with aqueous sodium bicarbonate, extracted with CH₂Cl₂, and the organic layers were evaporated in vacuo. The residue was purified by flash chromatography on silica gel (35 g SiO₂, gradient elution from 100% hexane to 100% ethyl acetate) to yield the title compound as a colorless oil.

¹H-NMR (CDCL₃) ∂: 1.50 (9H, s), 2.15 (1H, m), 3.15 (2H, m), 3.6-4.2 (4H, m), 7.31 (4H, s).

Step 3: 4-(4-Chloro-phenyl)-4-hydroxy-3-methyl-piperidine-1-carboxylic Acid Tert-Butyl Ester and 4-(4-Chloro-phenyl)-3-hydroxy-4-methyl-piperidine-1-carboxylic Acid Tert-Butyl Ester

A suspension of copper(I) iodide (0.38 g, 2 mmol) in THF (20 mL) was cooled to about 4° C., and treated with methylmagnesium bromide (6 mL of 3M solution in diethyl ether, 18 mmol). To the cooled suspension of cuprate was added 6-(4-Chloro-phenyl)-7-oxa-3-aza-bicyclo[4.1.0]heptane-3-carboxylic acid tert-butyl ester (1.9 g, 6.1 mmol) in THF (5 mL). The solution was stirred and allowed to warm to about room temperature for about 4 hours. The reaction was quenched with aqueous ammonium chloride, extracted with ethyl acetate, and the organic layers were evaporated in vacuo. The residue was purified by flash chromatography on silica gel (35 g SiO₂, gradient elution from 100% hexane to 50% ethyl acetate) to yield a mixture of the title compounds as a white foam. This mixture was carried on to the next step.

Step 4: Synthesis of 4-(4-Chloro-phenyl)-3-methyl-piperidin-4-ol and 4-(4-Chloro-phenyl)-4-methyl-piperidin-3-ol

The BOC-protected amino-alcohol mixture (0.8 g, 2.5 mmol) was dissolved in 4M HCl/Dioxane (5 mL, 20 mmol). The solution was stirred at room temperature for about 1 hour. The solvent was removed in vacuo. The residue was quenched with aqueous sodium hydroxide, extracted with ethyl acetate, and the organic layers were dried over sodium sulfate and evaporated in vacuo to yield the title compound as a brown solid.

ESI-MS m/z: 226 (M+1), 208 (M+1-H₂O). The mixture was carried on to the next step without further purification.

4-(4-Chloro-phenyl)-1-{3-[7-(1-hydroxy-1-methyl-ethyl)-11H-10-oxa-1-aza-dibenzo[a,d]cyclohepten-5-ylidene]-propyl}-3-methyl-piperidin-4-ol and

4-(4-Chloro-phenyl)-1-{3-[7-(1-hydroxy-1-methyl-ethyl)-11H-10-oxa-1-azadibenzo[a,d]cyclohepten-5-ylidene]-propyl}-4-methyl-piperidin-3-ol

To a solution of the amino alcohol mixture (0.53 g, 2.3 mmol) in isopropanol (10 mL) was added 2,6-lutidine (0.23 mL, 2.0 mmol) and catalytic potassium iodide. This mixture was heated to about 80° C., and treated with 2-[5-(3-Bromo-propylidene)-5,11-dihydro-10-oxa-1-aza-dibenzo[a,d]cyclohepten-7-yl]-propan-2-ol (0.37 g, 1.0 mmol), added in portions over about 2 hours. The solution was then stirred at about 80° C. for about an additional 2 hours. The reaction was concentrated in vacuo, then purified by flash chromatography on silica gel (35 g SiO₂, gradient elution from 100% ethyl acetate to 87% ethyl acetate: 10% methanol:3% triethylamine) to yield the title compounds.

The faster eluting isomer: 4-(4-Chloro-phenyl)-1-{3-[7-(1-hydroxy-1-methyl-ethyl)-11H-10-oxa-1-aza-dibenzo[a,d]cyclohepten-5-ylidene]-propyl}-3-methyl-piperidin-4-ol, a brown semisolid. ¹H-NMR (CDCL₃) ∂: 0.70 (3H, d, J=7.2 Hz), 1.53 (6H, s), 1.92 (3H, m), 2.28-2.69 (8H, m), 5.30 (2H, br s), 6.15 (1H, t, J=1.4 Hz), 6.79 (2H, d, J=8.4 Hz), 7.18-7.45 (7H, m), 7.59 (1H, d, J=8 Hz), 8.45 (1H, m). ESI-MS m/z: 519 (M+1).

The slower eluting isomer: 4-(4-Chloro-phenyl)-1-{3-[7-(1-hydroxy-1-methyl-ethyl)-, a brown semisolid. ¹H-NMR (CDCL₃) ∂: 1.16 (3H, s), 1.55 (6H, s), 2.28-2.69 (5H, m), 3.86 (1H, br s), 5.30 (2H, br s), 6.07 (1H, t, J=1.4 Hz), 6.79 (2H, d, J=8.4 Hz), 7.18-7.45 (7H, m), 8.45 (1H, m). ESI-MS m/z: 519 (M+1).

### EXAMPLE 443

4-(4-Chloro-phenyl)-1-{3-[7-(1-hydroxy-1-methyl-ethyl)-11H-10-oxa-1-aza-dibenzo[a,d]cyclohepten-5-ylidene]-propyl}-3,3-dimethyl-piperidin-4-ol

Step 1: 1-Benzyl-3,3-dimethyl-piperidin-4-one

To a solution of 1-Benzyl-3-methyl-piperidin-4-one (2.03 g, 10 mmol) in THF (10 mL) was added potassium t-Butoxide (1.1 g, 10 mmol) and methyl iodide (0.62 mL, 10 mmol). The solution was then stirred at room temperature for about 72 hours. The reaction was quenched with brine and extracted with ethyl acetate. The combined organic layers were concentrated in vacuo, then purified by flash chromatography on silica gel (35 g SiO₂, gradient elution from 100% hexane to 100% ethyl acetate) to yield the title compound as a colorless oil.

¹H-NMR (CDCL₃) ∂: 1.12 (6H, s), 2.41 (2H, s), 2.52 (2H, m), 2.73 (2H, m), 3.56 (2H, s), 7.20-7.40 (5H, m). ESI-MS m/z: 218 (M +1).

Step 2: 3,3-Dimethyl-4-oxo-piperidine-1-carboxylic Acid Tert-Butyl Ester

To a solution of 1-Benzyl-3,3-dimethyl-piperidin-4-one (2.48 g, 11 mmol) in ethanol (100 mL) was added di-tert-butyl dicarbonate (2.18, 10 mmol) and palladium hydroxide (0.10 g), The suspension was then shaken under a hydrogen atmosphere (40 PSI) at room temperature for about an additional 12 hours. The reaction was filtered through celite and evaporated in vacuo to yield the title compound as a white solid.

¹H-NMR (CDCL₃) ∂: 1.12 (6H, br s), 1.48 (9H, s), 2.45-2.80 (3H, m), 3.12 (1H, m), 3.42 (1H, br s), 3.70 (1H, m).

Step 3: 4-(4-Chloro-phenyl)-4-hydroxy-3,3-dimethyl-piperidine-1-carboxylic Acid Tert-Butyl Ester

To an ice-cooled solution 3,3-Dimethyl-4-oxo-piperidine-1-carboxylic acid tert-butyl ester (1.6 g, 7.2 mmol) in THF (20 mL) was added 4-chlorophenylmagnesium bromide (1 M in ether, 15 mL, 15 mmol). The solution was allowed to warm to about room temperature, then stirred at room temperature for about 22 hours. The reaction was quenched with aqueous ammonium chloride and extracted with ethyl acetate. The combined organic layers were concentrated in vacuo, then purified by flash chromatography on silica gel (35 g SiO₂, gradient elution from 100% hexane to 100% ethyl acetate) to yield the title compound as a colorless oil.

¹H-NMR (CDCL₃) ∂: 1.12 (6H, s), 1.50 (11H, m), 2.60 (1H, m), 3.20 (2H, m), 3.56 (1H, m), 4.20 (1H, m), 7.20-7.40 (4H, m). ESI-MS m/z: 218 (M+1).

Step 4: 4-(4-Chloro-phenyl)-3,3-dimethyl-piperidin-4-ol

4-(4-Chloro-phenyl)-4-hydroxy-3,3-dimethyl-piperidine-1-carboxylic acid tert-butyl ester (0.25 g, 0.73 mmol) was dissolved in 4M HCl/Dioxane (2 mL, 8 mmol). The solution was stirred at room temperature for about 4 hours. The solvent was removed in vacuo. The residue was quenched with aqueous sodium hydroxide, extracted with ethyl acetate, and the organic layers were dried over sodium sulfate and evaporated in vacuo to yield the title compound as a yellow solid. The mixture was carried on to the next step without further purification.

4-(4-Chloro-phenyl)-1-{3-[7-(1-hydroxy-1-methyl-ethyl)-11H-10-oxa-1-aza-dibenzo[a,d]cyclohepten-5-ylidene]-propyl}-3,3-dimethyl-piperidin-4-ol

To a solution of the amino alcohol mixture (0.17 g, 0.7 mmol) in isopropanol (5 mL) was added 2,6-lutidine (0.23 mL, 2.0 mmol) and catalytic potassium iodide. This mixture was heated to about 80° C., and treated with 2-[5-(3-Bromo-propylidene)-5,11-dihydro-10-oxa-1-aza-dibenzo[a,d]cyclohepten-7-yl]-propan-2-ol (0.19 g, 0.5 mmol), added in portions over about 2 hours. The solution was then stirred at about 80° C. for an additional 2 hours. The reaction was concentrated in vacuo, then purified by flash chromatography on silica gel (10 g SiO₂, gradient elution from 100% ethyl acetate to 87% ethyl acetate: 10% methanol:3% triethylamine) to yield the title compound as a brown semisolid.

¹H-NMR (CDCL₃) ∂: 0.78 (3H, s), 0.90 (3H, s), 1.45 (2H, m), 1.53 (6H, s), 2.28-2.80 (9H, m), 5.30 (2H, br s), 6.15 (1H, t, J=1.4 Hz), 6.79 (2H, d, J=8.4 Hz), 7.18-7.45 (7H, m), 7.59 (1H, d, J=8 Hz), 8.45 (1H, m). ESI-MS m/z: 533 (M+1).

Additional compounds of the invention can be prepared by the schemes set forth in FIGS. 1 -5, 7, 8A-8C, 9A-9E, 10A-10d and 12-19 and by the procedures described herein.

Those skilled in the art will be able to recognize, or be able to ascertain, using no more than routine experimentation, many equivalents to the specific embodiments of the invention described herein. Such equivalents are intended to be encompassed by the following claims.

What is claimed:

1. A compound having the formula: 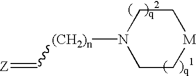
or physiologically acceptable salt thereof, wherein:
n is an integer from one to four;
M is >NR², >CR¹R², —O—CR¹R²—O— or —CH₂-CR¹R²—O—;
the ring containing M is substituted or unsubstituted;
q¹ is an integer from zero to three;
q² is zero or one;
R¹ is —H, —OH, —N₃, a halogen, an aliphatic group, a substituted aliphatic group, an aminoalkyl group, —O-(aliphatic group), —O-(substituted aliphatic group), —SH, —S-(aliphatic group), —S-(substituted aliphatic group), —OC(O)-(aliphatic group), —O-C(O)-(substituted aliphatic group), —C(O)O-(aliphatic group), —C(O)O-(substituted aliphatic group), —COOH, —CN, —CO—NR³R⁴, —NR³R⁴ or R¹ is a covalent bond between the ring atom at M and an adjacent carbon atom in the ring which contains M;
R² is —OH, a halogen, an acyl group, a substituted acyl group, —NR⁵R⁶, an aliphatic group, a substituted aliphatic group, an aromatic group, a substituted aromatic group, a benzyl group, a substituted benzyl group, a non-aromatic heterocyclic group, a substituted non-aromatic heterocyclic group, —O-(substituted or unsubstituted aromatic group) or —O-(substituted or unsubstituted aliphatic group);
R³, R⁴, R⁵ and R⁶ are independently —H, an acyl group, a substituted acyl group, an aliphatic group, a substituted aliphatic group, an aromatic group, a substituted aromatic group, a benzyl group, a substituted benzyl group, a non-aromatic heterocyclic group or a substituted non-aromatic heterocyclic group; or
R¹ and R², R³ and R⁴, or R⁵ and R⁶ taken together with the atom to which they are bonded, form a substituted or unsubstituted non-aromatic carbocyclic or heterocyclic ring;
Z is: 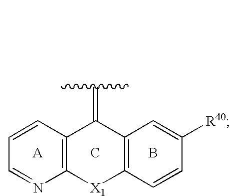
X₁ is —S—, —CH₂—, —CH₂—CH₂—, —CH₂—S—, —S—CH₂—, —O—CH₂—, —CH₂O—, —NR_{c}—CH₂—, —CH₂—NR_{c}—, —SO—CH₂—, —CH₂—SO—, —S(O)₂—CH₂—, —CH₂—S(O)₂—, —CH═CH—, —NR_{c}—CO—, a bond, —O—, or —CO—NR_{c}—;
R_{c} is —H, an aliphatic group, a substituted aliphatic group, an aromatic group, a substituted aromatic group, a benzyl group or a substituted benzyl group; and
R⁴⁰ is a substituted aliphatic group, a substituted aromatic group, —O-substituted aliphatic group or —O-substituted aromatic group, wherein the aliphatic or aromatic moiety of R⁴⁰ bears a substituent selected from the group consisting of —OH, —COOH, —C(O)O-aliphatic group and —C(O)O-aromatic group.

2. The compound of claim 1 wherein q¹ and q² are both one, and said compound has the formula: 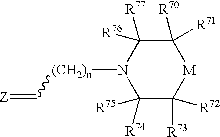
wherein:
n is an integer from one to four;
M is >NR² or >CR¹R²;
R⁷⁰, R⁷¹, R⁷², R⁷³, R⁷⁴, R⁷⁵, R⁷⁶ and R⁷⁷ are independently —H, —OH, —N₃, a halogen, an aliphatic group, a substituted aliphatic group, an aminoalkyl group, —O-(aliphatic group), —O-(substituted aliphatic group), —SH, —S-(aliphatic group), —S-(substituted aliphatic group), —OC(O)-(aliphatic group), —O—C(O)-(substituted aliphatic group), —C(O)O-(aliphatic group), —C(O)O-(substituted aliphatic group), —COOH, —CN, —CO—NR³R⁴, —NR³R⁴, an acyl group, a substituted acyl group, a benzyl group, a substituted benzyl group, a non-aromatic heterocyclic group, a substituted non-aromatic heterocyclic group, —O-(substituted or unsubstituted aromatic group), or
any two of R⁷⁰, R⁷¹, R⁷², R⁷³, R⁷⁴, R⁷⁵, R⁷⁶ and R⁷⁷ taken together with the atoms to which they are bonded form a three to eight membered ring;
R³ and R⁴ are independently -H, an acyl group, a substituted acyl group, an aliphatic group, a substituted aliphatic group, an aromatic group, a substituted aromatic group, a benzyl group, a substituted benzyl group, a non-aromatic heterocyclic group or a substituted non-aromatic heterocyclic group; or
R³ and R⁴ taken together with the atom to which they are bonded, form a substituted or unsubstituted non-aromatic heterocyclic ring.

3. The compound of claim 2 wherein R⁷⁰, R⁷³, R⁷⁴, R⁷⁵, R⁷⁶ and R⁷⁷ are —H.

4. The compound of claim 3 wherein R⁷¹ is —H, and R⁷² is an aliphatic group selected from the group consisting of a C₁-C₆ alkyl group, a C₂-C₆ alkenyl or C₂-C₆ alkynyl group.

5. The compound of claim 4 wherein R⁷² is —CH₃.

6. The compound of claim 3 wherein is R⁷¹ is an aliphatic group selected from the group consisting of a C₁-C₆ alkyl group, a C₂-C₆ alkenyl or C₂-C₆ alkynyl group, and R⁷² is —H.

7. The compound of claim 6 wherein R⁷¹ is —CH₃.

8. The compound of claim 3 wherein R⁷¹ and R⁷² are —CH₃.

9. The compound of claim 3 wherein X₁ is —CH₂—O—.

10. The compound of claim 3 wherein X₁ is —CH₂—S— or —CH₂—CH₂—.

11. The compound of claim 3 wherein
R¹ is a substituted aromatic group; and
R² is —H or —OH.

12. The compound of claim 3 wherein
R¹ is 4-chlorophenyl; and
R² is —OH.

13. The compound of claim 2 wherein R⁴⁰ is a substituted aliphatic group bearing a —OH or —COOH substituent, wherein the aliphatic moiety of said substituted aliphatic group is a linear, branched or cyclic alkyl, alkenyl or alkynyl.

14. The compound of claim 13 wherein aliphatic moiety is a linear or branched C₁-C₆ alkyl, a linear or branched C₂-C₆ alkenyl or a linear or branched C₂-C₆ alkynyl.

15. The compound of claim 2 wherein R⁴⁰ is —O-substituted aliphatic group bearing a —OH or —COOH substituent., wherein the aliphatic moiety of said —O-substituted aliphatic group is a linear, branched or cyclic alkyl, alkenyl or alkynyl.

16. The compound of claim 15 wherein said aliphatic moiety is a linear or branched C₁-C₆ alkyl, a linear or branched C₂-C₆ alkenyl or a linear or branched C₂-C₆ alkynyl.

17. The compound of claim 2 wherein R⁴⁰ is an aromatic group that bears a —OH or —COOH substituent.

18. The compound of claim 17 wherein said aromatic group is phenyl.

19. The compound of claim 2 wherein R⁴⁰ is a substituted aliphatic or substituted aromatic group bearing a substituent selected from the group consisting of —C(O)O—(C₁-C₆ alkyl), —C(O)O—(C₂-C₆ alkenyl), —C(O)O—(C₂-C₆ alkynyl) and —C(O)O—(aromatic group), wherein the aliphatic moiety of said substituted aliphatic group is a linear, branched or cyclic alkyl, alkenyl or alkynyl.

20. A compound having the formula: 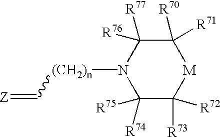
or physiologically acceptable salt thereof, wherein:
n is an integer from one to four;
M is >NR² or >CR¹R²;
R¹ is —H, —OH, —N₃, a halogen, an aliphatic group, a substituted aliphatic group, an aminoalkyl group, —O-(aliphatic group), —O-(substituted aliphatic group), —SH, —S-(aliphatic group), —S-(substituted aliphatic group), —OC(O)-(aliphatic group), —O—C(O)-(substituted aliphatic group), —C(O)O-(aliphatic group), —C(O)O-(substituted aliphatic group), —COOH, —CN, —CO—NR³R⁴, —NR³R⁴ or R¹ is a covalent bond between the ring atom at M and an adjacent carbon atom in the ring which contains M;
R² is —OH, a halogen, an acyl group, a substituted acyl group, —NR⁵R⁶, an aliphatic group, a substituted aliphatic group, an aromatic group, a substituted aromatic group, a benzyl group, a substituted benzyl group, a non-aromatic heterocyclic group, a substituted non-aromatic heterocyclic group, —O-(substituted or unsubstituted aromatic group) or —O-(substituted or unsubstituted aliphatic group);
R⁷⁰, R⁷¹, R⁷², R⁷³, R⁷⁴, R⁷⁵, R⁷⁶ and R⁷⁷ are independently —H, —OH, —N₃, a halogen, an aliphatic group, a substituted aliphatic group, an aminoalkyl group, —O-(aliphatic group), —O-(substituted aliphatic group), —SH, —S-(aliphatic group), —S-(substituted aliphatic group), —OC(O)-(aliphatic group), —O—C(O)-(substituted aliphatic group), —C(O)O-(aliphatic group), —C(O)O-(substituted aliphatic group), —COOH, —CN, —-CO-NR³R⁴, —NR³R⁴, an acyl group, a substituted acyl group, a benzyl group, a substituted benzyl group, a non-aromatic heterocyclic group, a substituted non-aromatic heterocyclic group, —O-(substituted or unsubstituted aromatic group), or
any two of R⁷⁰, R⁷¹, R⁷², R⁷, R⁷⁴, R⁷⁵, R⁷⁶ and R⁷⁷ taken together with the atoms to which they are bonded form a three to eight membered ring;
R³, R⁴, R⁵ and R⁶ are independently —H, an acyl group, a substituted acyl group, an aliphatic group, a substituted aliphatic group, an aromatic group, a substituted aromatic group, a benzyl group, a substituted benzyl group, a non-aromatic heterocyclic group or a substituted non-aromatic heterocyclic group; or
R¹ and R², R³ and R⁴, or R⁵ and R⁶ taken together with the atom to which they are bonded, form a substituted or unsubstituted non-aromatic carbocyclic or heterocyclic ring;
Z is 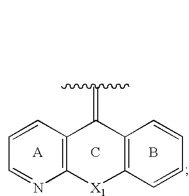
X₁ is —S—, —CH₂—, —CH₂—CH₂—, —CH₂—S—, —S—CH₂—, —O—CH₂—, —CH₂—O—, —NR_{c}—CH₂—, —CH₂—NR_{c}—, —SO—CH₂—, —CH₂—SO—, —S(O)₂—CH₂—, —CH₂—S(O)₂—, —CH═CH—, —NR_{c}—CO—, a bond, —O—, or —CO—NR_{c}—;
R_{c} is —H, an aliphatic group, a substituted aliphatic group, an aromatic group, a substituted aromatic group, a benzyl group or a substituted benzyl group; and
Ring A and Ring B are independently substituted or unsubstituted.

21. The compound of claim 20 wherein ring B is substituted para to the carbon atom of ring B that is bonded to X₁ in ring C, and Z is represented by the structural formula: 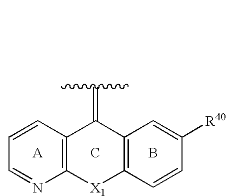
wherein R⁴⁰ is —OH, —COOH, —NO₂, halogen, aliphatic group, substituted aliphatic group, an aromatic group, a substituted aromatic group, —NR²⁴R²⁵, —CONR²⁴R²⁵, Q-(aliphatic group), Q-(substituted aliphatic group), —O-(aliphatic group), —O-(substituted aliphatic group), —O-(aromatic group), —O-(substituted aromatic group), an electron withdrawing group, —(O)ᵤ—(CH₂)ₜ—C(O)OR²⁰, —(O)ᵤ—(CH₂)ₜ—OC(O)R²⁰, —(O)ᵤ—(CH₂)ₜ—C(O)—NR²¹R²² or —(O)ᵤ—(CH₂)ₜ—NHC(O)O—R²⁰;
R²⁰, R²¹ or R²² are independently —H, an aliphatic group, a substituted aliphatic group, an aromatic group, a substituted aromatic group or a non-aromatic heterocyclic group; or
R²¹ and R²², taken together with the nitrogen atom to which they are bonded, form a non-aromatic heterocyclic ring;
Q is —NR²⁴C(O)—, —NR²⁴S(O)₂—, —C(O)O— or —C(O)—;
R²⁴ and R²⁵ are independently —H, —OH, an aliphatic group or a substituted aliphatic group;
u is zero or one; and
t is an integer from zero to 3.

22. The compound of claim 20 wherein X₁ is —CH₂—O—.

23. The compound of claim 20 wherein
R¹ is a substituted aromatic group; and
R² is —H or —OH.

24. A compound having the formula: 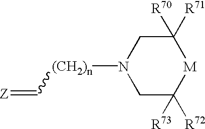
or physiologically acceptable salt thereof, wherein:
n is an integer from one to four;
R⁷⁰, R⁷¹, R²² and R⁷³ are independently —H, an aliphatic group or a substituted aliphatic group, with the proviso that at least one of R⁷⁰ and R⁷¹ is an aliphatic group or a substituted aliphatic group;
M is >NR² or >CR¹R²;
R¹ is —H, —OH, —N₃, a halogen, an aliphatic group, a substituted aliphatic group, an aminoalkyl group, —O-(aliphatic group), —O-(substituted aliphatic group), —SH, —S-(aliphatic group), —S-(substituted aliphatic group), —OC(O)-(aliphatic group), —O-C(O)-(substituted aliphatic group), —C(O)O-(aliphatic group), —C(O)O-(substituted aliphatic group), —COOH, —CN, —CO—NR³R⁴, —NR³R⁴ or R¹ is a covalent bond between the ring atom at M and an adjacent carbon atom in the ring which contains M;
R² is —OH, halogen, an acyl group, a substituted acyl group, —NR⁵R⁶, an aliphatic group, a substituted aliphatic group, an aromatic group, a substituted aromatic group, a benzyl group, a substituted benzyl group , a non-aromatic heterocyclic group, a substituted non-aromatic heterocyclic group, —O-(substituted or unsubstituted aromatic group) or —O-(substituted or unsubstituted aliphatic group);
R³, R⁴, R⁵ and R⁶ are independently -H, an acyl group, a substituted acyl group, an aliphatic group, a substituted aliphatic group, an aromatic group, a substituted aromatic group, a benzyl group, a substituted benzyl group, a non-aromatic heterocyclic group or a substituted non-aromatic heterocyclic group; or
R¹ and R², R³ and R⁴, or R⁵ and R⁶ taken together with the atom to which they are bonded, form a substituted or unsubstituted non-aromatic carbocyclic or heterocyclic ring;
Z is 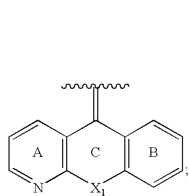
X₁ is —S—, —CH₂—, —CH₂—CH₂—, —CH₂—S—, —S—CH₂—, —O—CH₂—, —CH₂—O—, —NR_{c}—CH₂—, —CH₂—NR_{c}—, —SO—CH₂—, —CH₂—SO—, —S(O)₂—CH₂—, —CH₂—S(O)₂—, —CH═CH—, —NRC—CO—, a bond, —O—, or —CO—NR_{c}—;
R_{c} is —H, an aliphatic group, a substituted aliphatic group, an aromatic group, a substituted aromatic group, a benzyl group or a substituted benzyl group; and
Ring A and Ring B are independently substituted or unsubstituted.

25. The compound of claim 24 wherein X₁ is —CH₂—O—.

26. The compound of claim 24 wherein X₁ is —CH₂—S—or —CH₂—CH₂—.

27. The compound of claim 24 wherein
R¹ is a substituted aromatic group; and
R² is —H or —OH.

28. The compound of claim 24 wherein
R¹ is 4-chlorophenyl; and
R² is —OH.

29. The compound of claim 24 wherein ring B is substituted para to the carbon atom of ring B that is bonded to X₁ in ring C, and Z is represented by the structural formula: 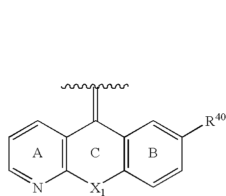
wherein R⁴⁰ is —OH, —COOH, —NO₂, halogen, aliphatic group, substituted aliphatic group, an aromatic group, a substituted aromatic group, —NR²⁴R²⁵, —CONR²⁴R²⁵, Q-(aliphatic group), Q-(substituted aliphatic group), —O-(aliphatic group), —O-(substituted aliphatic group), —O-(aromatic group), —O-(substituted aromatic group), an electron withdrawing group, —(Oᵤ—(CH₂)ₜ—C(O)OR²⁰, —(O)ᵤ—(CH₂)ₜ—OC(O)R²⁰, —(O)ᵤ—(CH₂)ₜ—C(O)—NR²¹R²² or —(O)ᵤ—(CH₂)ₜ—NHC(O)O—R²⁰;
R²⁰, R²¹ or R²² are independently —H, an aliphatic group, a substituted aliphatic group, an aromatic group, a substituted aromatic group or a non-aromatic heterocyclic group; or
R²¹ and R²², taken together with the nitrogen atom to which they are bonded, form a non-aromatic heterocyclic ring;
Q is —NR²⁴C(O)—, —NR²⁴S(O)₂—, —C(O)O— or —C(O)—;
R²⁴ and R²⁵ are independently —H, —OH, an aliphatic group or a substituted aliphatic group;
u is zero or one; and
t is an integer from zero to 3.

30. The compound of claim 29 wherein R⁴⁰ is —COOH.

31. The compound of claim 29 wherein R⁴⁰ is —CH(CH₃)(OH)CH₃.

32. The compound of claim 29 wherein R⁷⁰ and R⁷³ are —H.

33. The compound of claim 32 wherein at least one of R⁷¹ and R⁷² is C₁-C₆ alkyl.

34. The compound of claim 32 wherein at least one of R⁷¹ and R⁷² is —CH₃.

35. The compound of claim 32 wherein R⁷¹ and R⁷² are both —CH₃.

36. The compound of claim 32 wherein R⁷¹ is —H and R⁷² is C₁-C₆ alkyl.

37. The compound of claim 32 wherein R⁷¹ is C₁-C₆ alkyl and R⁷² is —H.

38. A compound having the formula: 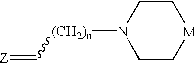
or physiologically acceptable salt thereof, wherein:
n is two;
M is >CR¹R²;
R¹ is —OH;
R² is 4-chlorophenyl;
Z is 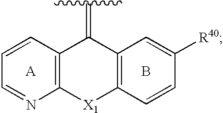
X₁ is —CH₂—O—; and
R⁴⁰ is selected from the group consisting of

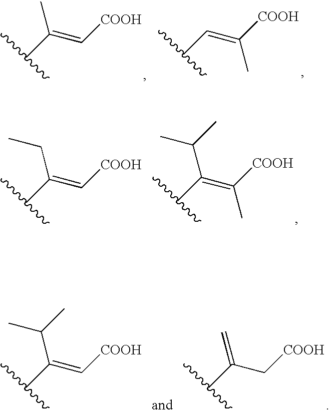

39. A compound having the formula: 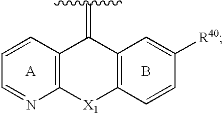
or physiologically acceptable salt thereof, wherein:
n is two;
M is >CR¹R²;
R¹ is —OH;
R² is 4-chlorophenyl;
Z is 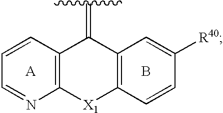
X₁ is —CH₂—O—; and
R⁴⁰ is selected from the group consisting of

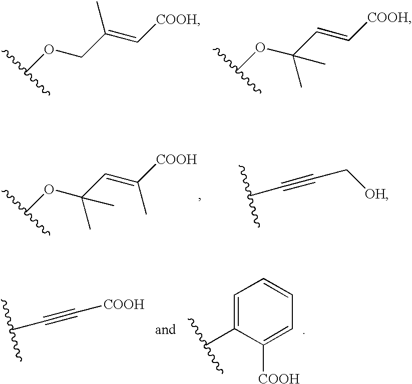

40. A compound having the formula: 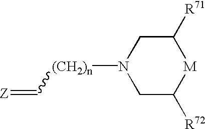
or physiologically acceptable salt thereof, wherein:
n is two;
R⁷¹ and R⁷² are independently selected from the group consisting of —H and C₁-C₆ alkyl, with the proviso that at least one of R⁷¹ and R⁷² is C₁-C₆ alkyl;
M is >CR¹R²;
R¹ is —OH;
R² is 4-chlorophenyl;
Z is 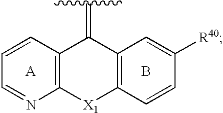
X₁ is —CH₂—O—; and
R⁴⁰ is —CH(CH₃)(OH)CH₃.

41. The compound of claim 40 wherein said C₁-C₆ alkyl is —CH₃.

42. A method of treating a disease associated with leukocyte recruitment and/or activation mediated by chemokine receptor function, comprising administering to a subject in need thereof an effective amount of a compound having the formula: 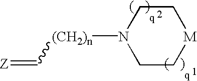
or physiologically acceptable salt thereof, wherein:
n is an integer from one to four;
M is >NR², >CR¹R², —O—CR¹R²—O— or —CH₂—CR¹R²—O—;
the ring containing M is substituted or unsubstituted;
q¹ is an integer from zero to three;
q² is zero or one;
R¹ is —H, —OH, —N₃, a halogen, an aliphatic group, a substituted aliphatic group, an aminoalkyl group, —O-(aliphatic group), —O-(substituted aliphatic group), —SH, —S-(aliphatic group), —S-(substituted aliphatic group), —OC(O)-(aliphatic group), —O—C(O)-(substituted aliphatic group), —C(O)O-(aliphatic group), —C(O)O-(substituted aliphatic group), —COOH, —CN, —CO—NR³R⁴, —NR³R⁴ or R¹ is a covalent bond between the ring atom at M and an adjacent carbon atom in the ring which contains M;
R² is —OH, a halogen, an acyl group, a substituted acyl group, —NR⁵R⁶, an aliphatic group, a substituted aliphatic group, an aromatic group, a substituted aromatic group, a benzyl group, a substituted benzyl group, a non-aromatic heterocyclic group, a substituted non-aromatic heterocyclic group, —O-(substituted or unsubstituted aromatic group) or —O-(substituted or unsubstituted aliphatic group);
R³, R⁴, R⁵ and R⁶ are independently —H, an acyl group, a substituted acyl group, an aliphatic group, a substituted aliphatic group, an aromatic group, a substituted aromatic group, a benzyl group, a substituted benzyl group, a non-aromatic heterocyclic group or a substituted non-aromatic heterocyclic group; or
R¹ and R², R³ and R⁴, or R⁵ and R⁶ taken together with the atom to which they are bonded, form a substituted or unsubstituted non-aromatic carbocyclic or heterocyclic ring;
Z is: 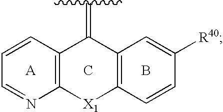
X₁ is —S—, —CH₂—, —CH₂—CH₂—, —CH₂—S—, —S—CH₂—, —O—CH₂—, —CH₂—O—, —NR_{c}—CH₂—, —CH₂—NR_{c}—, —SO—CH₂—, —CH₂—SO—, —S(O)₂—CH₂—, —CH₂—S(O)₂—, —CH═CH—, —NR_{c}—CO—, a bond, —O—, or —CO—NR_{c}—;
R_{c} is —H, an aliphatic group, a substituted aliphatic group, an aromatic group, a substituted aromatic group, a benzyl group or a substituted benzyl group; and
R⁴⁰ is a substituted aliphatic group, a substituted aromatic group, —O-substituted aliphatic group or —O-substituted aromatic group, wherein the aliphatic or aromatic moiety of R⁴⁰ bears a substituent selected from the group consisting of —OH, —COOH, —C(O)O-aliphatic group and —C(O)O-aromatic group.

43. The method of claim 42 wherein q¹ and q²are both one, and said compound has the formula: 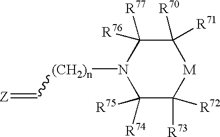
wherein:
n is an integer from one to four;
M is >NR² or >CR¹R²;
R⁷⁰, R⁷¹, R⁷², R⁷³, R⁷⁴, R⁷⁵, R⁷⁶ and R⁷⁷ are independently —H, —OH, —N₃, a halogen, an aliphatic group, a substituted aliphatic group, an aminoalkyl group, —O-(aliphatic group), —O-(substituted aliphatic group), -SH, —S-(aliphatic group), —S-(substituted aliphatic group), —OC(O)-(aliphatic group), —O—C(O)-(substituted aliphatic group), —C(O)O-(aliphatic group), —C(O)O-(substituted aliphatic group), —COOH, —CN, —CO—NR³R⁴, —NR³R⁴, an acyl group, a substituted acyl group, a benzyl group, a substituted benzyl group, a non-aromatic heterocyclic group, a substituted non-aromatic heterocyclic group, —O-(substituted or unsubstituted aromatic group), or
any two of R⁷⁰, R⁷¹, R⁷², R⁷³, R⁷⁴, R⁷⁵, R⁷⁶ and R⁷⁷ taken together with the atoms to which they are bonded form a three to eight membered ring;
R³ and R⁴ are independently —H, an acyl group, a substituted acyl group, an aliphatic group, a substituted aliphatic group, an aromatic group, a substituted aromatic group, a benzyl group, a substituted benzyl group, a non-aromatic heterocyclic group or a substituted non-aromatic heterocyclic group; or
R³ and R⁴ taken together with the atom to which they are bonded, form a substituted or unsubstituted non-aromatic heterocyclic ring.

44. The method of claim 43 wherein R⁷⁰, R⁷³, R⁷⁴, R⁷⁵, R⁷⁶ and R⁷⁷ are —H.

45. The method of claim 44 wherein R⁷¹ is —H, and R⁷² is an aliphatic group selected from the group consisting of a C₁-C₆ alkyl group, a C₂-C₆ alkenyl or C₂-C₆ alkynyl group.

46. The method of claim 45 wherein R⁷² is —CH₃.

47. The method of claim 44 wherein is R⁷¹ is an aliphatic group selected from the group consisting of a C₁-C₆ alkyl group, a C₂-C₆ alkenyl or C₂-C₆ alkynyl group, and R⁷² is —H.

48. The method of claim 47 wherein R⁷¹ is —CH₃.

49. The method of claim 44 wherein R⁷¹ and R⁷² are —CH₃.

50. The method of claim 44 wherein X₁ is —CH₂—O—.

51. The method of claim 44 wherein X₁ is —CH₂—S— or —CH₂—CH₂—.

52. The method of claim 44 wherein
R¹ s a substituted aromatic group; and
R² is —H or —OH.

53. The method of claim 44 wherein
R¹ is 4-chlorophenyl; and
R² is —OH.

54. The method of claim 43 wherein X₁ is a substituted aliphatic group bearing a —OH or —COOH substituent, wherein the aliphatic moiety of said substituted aliphatic group is a linear, branched or cyclic alkyl, alkenyl or alkynyl.

55. The method of claim 54 wherein aliphatic moiety is a linear or branched C₁-C₆ alkyl, a linear or branched C₂-C₆ alkenyl or a linear or branched C₂-C₆ alkynyl.

56. The method of claim 43 wherein R⁴⁰ is —O-substituted aliphatic group bearing a —OH or —COOH substituent., wherein the aliphatic moiety of said —O-substituted aliphatic group is a linear, branched or cyclic alkyl, alkenyl or alkynyl.

57. The method of claim 56 wherein said aliphatic moiety is a linear or branched C₁-C₆ alkyl, a linear or branched C₂-C₆ alkenyl or a linear or branched C₂-C₆ alkynyl.

58. The method of claim 43 wherein R⁴⁰ is an aromatic group that bears a —OH or —COOH substituent.

59. The method of claim 58 wherein said aromatic group is phenyl.

60. The method of claim 43 wherein R⁴⁰ is a substituted aliphatic or substituted aromatic group bearing a substituent selected from the group consisting of —C(O)O—(C₁-C₆ alkyl), —C(O)O—(C₂-C₆ alkenyl), —C(O)O—(C₂-C₆ alkynyl) and —C(O)O-(aromatic group), wherein the aliphatic moiety of said substituted aliphatic group is a linear, branched or cyclic alkyl, alkenyl or alkynyl.

61. A method of treating a disease associated with leukocyte recruitment and/or activation mediated by chemokine receptor function, comprising administering to a subject in need thereof an effective amount of a compound having the formula: 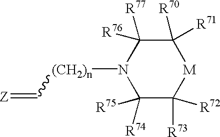
or physiologically acceptable salt thereof, wherein:
n is an integer from one to four;
M is >NR² or >CR¹R²;
R¹ is —H, —OH, —N₃, a halogen, an aliphatic group, a substituted aliphatic group, an aminoalkyl group, —O-(aliphatic group), —O-(substituted aliphatic group), —SH, —S-(aliphatic group), —S-(substituted aliphatic group), —OC(O)-(aliphatic group), —O—C(O)-(substituted aliphatic group), —C(O)O-(aliphatic group), —C(O)O-(substituted aliphatic group), —COOH, —CN, —CO—NR³R⁴, —NR³R⁴ or R¹ is a covalent bond between the ring atom at M and an adjacent carbon atom in the ring which contains M;
R² is —OH, a halogen, an acyl group, a substituted acyl group, —NR⁵R⁶, an aliphatic group, a substituted aliphatic group, an aromatic group, a substituted aromatic group, a benzyl group, a substituted benzyl group, a non-aromatic heterocyclic group, a substituted non-aromatic heterocyclic group, —O-(substituted or unsubstituted aromatic group) or —O-(substituted or unsubstituted aliphatic group);
R⁷⁰, R⁷¹, R⁷², R⁷³, R⁷⁴, R⁷⁵, R⁷⁶ and R⁷⁷ are independently —H, —OH, —N₃, a halogen, an aliphatic group, a substituted aliphatic group, an aminoalkyl group, —O-(aliphatic group), —O-(substituted aliphatic group), -SH, —S-(aliphatic group), —S-(substituted aliphatic group), —OC(O)-(aliphatic group), —O—C(O)-(substituted aliphatic group), —C(O)O-(aliphatic group), —C(O)O-(substituted aliphatic group), —COOH, —CN, —CO-NR³R⁴, —NR³R⁴, an acyl group, a substituted acyl group, a benzyl group, a substituted benzyl group, a non-aromatic heterocyclic group, a substituted non-aromatic heterocyclic group, —O-(substituted or unsubstituted aromatic group), or
any two of R⁷⁰, R⁷¹, R⁷², R⁷³, R⁷⁴, R⁷⁵, R⁷⁶ and R⁷⁷ taken together with the atoms to which they are bonded form a three to eight membered ring;
R³, R⁴, R⁵ and R⁶ are independently —H, an acyl group, a substituted acyl group, an aliphatic group, a substituted aliphatic group, an aromatic group, a substituted aromatic group, a benzyl group, a substituted benzyl group, a non-aromatic heterocyclic group or a substituted non-aromatic heterocyclic group; or
R¹ and R², R³ and R⁴, or R⁵ and R⁶ taken together with the atom to which they are bonded, form a substituted or unsubstituted non-aromatic carbocyclic or heterocyclic ring;
Z is 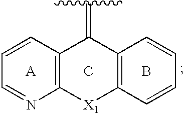
X₁ is —S—, —CH₂—, —CH₂—CH₂—, —CH₂—S—, —S—CH₂—, —O—CH₂—, —CH₂—O—, —NR_{c}—CH₂—, —CH₂—NR_{c}—, —SO—CH₂—, —CH₂—SO—, —S(O)₂—CH₂—, —CH₂—S(O)₂—, —CH═CH—, —NR_{c}—CO—, a bond, —O—, or —CO—NR_{c}—;
R_{c} is —H, an aliphatic group, a substituted aliphatic group, an aromatic group, a substituted aromatic group, a benzyl group or a substituted benzyl group; and
Ring A and Ring B are independently substituted or unsubstituted.

62. The method of claim 61 wherein ring B is substituted para to the carbon atom of ring B that is bonded to X₁ in ring C, and Z is represented by the structural formula: 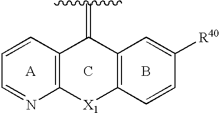
wherein R⁴⁰ is —OH, —COOH, —NO₂, halogen, aliphatic group, substituted aliphatic group, an aromatic group, a substituted aromatic group, —NR²⁴R²⁵, —CONR²⁴R²⁵, Q-(aliphatic group), Q-(substituted aliphatic group), —O-(aliphatic group), —O-(substituted aliphatic group), —O-(aromatic group), —O-(substituted aromatic group), an electron withdrawing group, —(O)ᵤ—(CH₂)ₜ—C(O)OR²⁰, —(O)ᵤ—(CH₂)ₜ—OC(O)R²⁰, —(O)ᵤ—(CH₂)ₜ—C(O)—NR²¹R²² or —(O)ᵤ—(CH₂)ₜ—NHC(O)O—R²⁰;
R²⁰, R²¹ or R²² are independently —H, an aliphatic group, a substituted aliphatic group, an aromatic group, a substituted aromatic group or a non-aromatic heterocyclic group; or
R²¹ and R²², taken together with the nitrogen atom to which they are bonded, form a non-aromatic heterocyclic ring;
Q is —NR²⁴C(O)—, —NR²⁴S(O)₂—, —C(O)O— or —C(O)—;
R²⁴ and R²⁵ are independently —H, —OH, an aliphatic group or a substituted aliphatic group;
u is zero or one; and
t is an integer from zero to 3.

63. The method of claim 61 wherein X₁ is —CH₂—O—.

64. The method of claim 61 wherein
R¹ is a substituted aromatic group; and
R² is —H or —OH.

65. A method of treating a disease associated with leukocyte recruitment and/or activation mediated by chemokine receptor function, comprising administering to a subject in need thereof an effective amount of a compound having the formula: 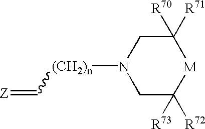
or physiologically acceptable salt thereof, wherein:
n is an integer from one to four;
R⁷⁰, R⁷¹, R⁷² and R⁷³ are independently —H, an aliphatic group or a substituted aliphatic group, with the proviso that at least one of R⁷⁰ and R⁷¹ is an aliphatic group or a substituted aliphatic group;
M is >NR² or >CR¹R²;
R¹ is —H, —OH, —N₃, a halogen, an aliphatic group, a substituted aliphatic group, an aminoalkyl group, —O-(aliphatic group), —O-(substituted aliphatic group), —SH, —S-(aliphatic group), —S-(substituted aliphatic group), —OC(O)-(aliphatic group), —O—C(O)-(substituted aliphatic group), —C(O)O-(aliphatic group), —C(O)O-(substituted aliphatic group), —COOH, —CN, —CO—NR³R⁴, —NR³R⁴ or R¹ is a covalent bond between the ring atom at M and an adjacent carbon atom in the ring which contains M;
R² is —OH, halogen, an acyl group, a substituted acyl group, —NR⁵R⁶, an aliphatic group, a substituted aliphatic group, an aromatic group, a substituted aromatic group, a benzyl group, a substituted benzyl group, a non-aromatic heterocyclic group, a substituted non-aromatic heterocyclic group, —O-(substituted or unsubstituted aromatic group) or —O-(substituted or unsubstituted aliphatic group);
R³, R⁴, R⁶ and R⁶ are independently -H, an acyl group, a substituted acyl group, an aliphatic group, a substituted aliphatic group, an aromatic group, a substituted aromatic group, a benzyl group, a substituted benzyl group, a non-aromatic heterocyclic group or a substituted non-aromatic heterocyclic group; or
R¹ and R², R³ and R⁴, or R⁶ and R⁶ taken together with the atom to which they are bonded, form a substituted or unsubstituted non-aromatic carbocyclic or heterocyclic ring;
Z is 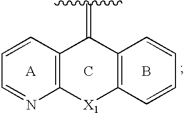
X₁ is —S—, —CH₂—, —CH₂—CH₂—, —CH₂—S—, —S—CH₂—, —O—CH₂—, —CH₂—O—, —NR_{c}—CH₂—, —CH₂—NR_{c}—, —SO—CH₂—, —CH₂—SO—, —S(O)₂—CH₂—, —CH₂—S(O)₂—, —CH═CH—, —NR_{c}—CO—, a bond, —O—, or —CO—NR_{c}—;
R_{c} is —H, an aliphatic group, a substituted aliphatic group, an aromatic group, a substituted aromatic group, a benzyl group or a substituted benzyl group; and
Ring A and Ring B are independently substituted or unsubstituted.

66. The method of claim 65 wherein X₁ is —CH₂—O—.

67. The method of claim 65 wherein X₁ is —CH₂—S— or —CH₂—CH₂—.

68. The method of claim 65 wherein
R¹ is a substituted aromatic group; and
R² is —H or —OH.

69. The method of claim 65 wherein
R¹ is 4-chlorophenyl; and
R² is —OH.

70. The method of claim 65 wherein ring B is substituted para to the carbon atom of ring B that is bonded to X₁ in ring C, and Z is represented by the structural formula: 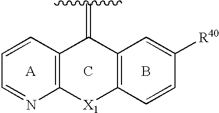
wherein R⁴⁰ is —OH, —COOH, —NO₂, halogen, aliphatic group, substituted aliphatic group, an aromatic group, a substituted aromatic group, —NR²⁴R²⁵, —CONR²⁴R²⁵, Q-(aliphatic group), Q-(substituted aliphatic group), —O-(aliphatic group), —O-(substituted aliphatic group), —O-(aromatic group), —O-(substituted aromatic group), an electron withdrawing group, —(O)ᵤ—(CH₂)ₜ—C(O)OR²⁰, —(O)ᵤ—(CH₂)ₜ—OC(O)R²⁰, —(O)ᵤ—(CH₂)ₜ—C(O)—NR²¹R²² or —(O)ᵤ—(CH₂)ₜ—NHC(O)O—R²⁰;
R²⁰, R²¹ or R²² are independently —H, an aliphatic group, a substituted aliphatic group, an aromatic group, a substituted aromatic group or a non-aromatic heterocyclic group; or
R²¹ and R²², taken together with the nitrogen atom to which they are bonded, form a non-aromatic heterocyclic ring;
Q is —NR²⁴C(O)—, —NR²⁴S(O)₂—, —C(O)O— or —C(O)—;
R²⁴ and R²⁵ are independently —H, —OH, an aliphatic group or a substituted aliphatic group;
u is zero or one; and
t is an integer from zero to 3.

71. The method of claim 70 wherein R⁴⁰ is —COOH.

72. The method of claim 70 wherein R⁴⁰ is —CH(CH₃)(OH)CH₃.

73. The method of claim 70 wherein R⁷⁰ and R⁷³ are —H.

74. The method of claim 73 wherein at least one of R⁷¹ and R⁷² is C₁-C₆ alkyl.

75. The method of claim 73 wherein at least one of R⁷¹ and R⁷² is —CH₃.

76. The method of claim 73 wherein R⁷¹ and R⁷² are both —CH₃.

77. The method of claim 73 wherein R⁷¹ is —H and R⁷² is C₁-C₆ alkyl.

78. The method of claim 73 wherein R⁷¹ is C₁-C₆ alkyl and R⁷² is —H.

79. A method of treating a disease associated with leukocyte recruitment and/or activation mediated by chemokine receptor function, comprising administering to a subject in need thereof an effective amount of a compound having the formula: 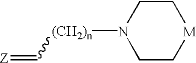
or physiologically acceptable salt thereof, wherein:
n is two;
M is >CR¹R²;
R¹ is —OH;
R² is 4-chlorophenyl;
Z is 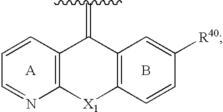
X₁ is —CH₂—O—; and
R⁴⁰ is selected from the group consisting of

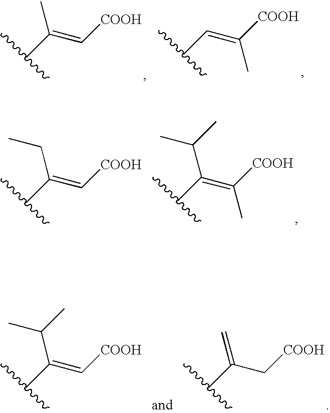

80. A method of treating a disease associated with leukocyte recruitment and/or activation mediated by chemokine receptor function, comprising administering to a subject in need thereof an effective amount of a compound having the formula: 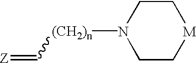
or physiologically acceptable salt thereof, wherein:
n is two;
M is >CR¹R²;
R¹ is —OH;
R² is 4-chlorophenyl;
Z is 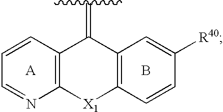
X¹ is —CH₂—O—; and
R⁴⁰ is selected from the group consisting of

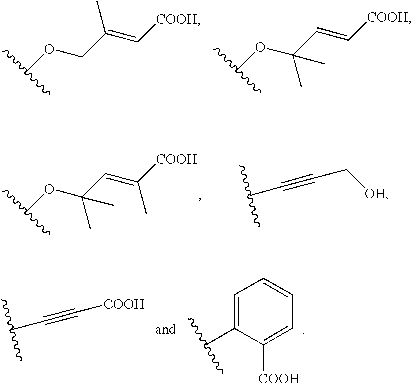

81. A method of treating a disease associated with leukocyte recruitment and/or activation mediated by chemokine receptor function, comprising administering to a subject in need thereof an effective amount of a compound having the formula: 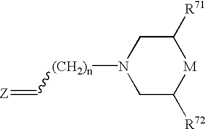
or physiologically acceptable salt thereof, wherein:
n is two;
R⁷¹ and R⁷² are independently selected from the group consisting of —H and C₁-C₆ alkyl, with the proviso that at least one of R⁷¹ and R⁷² is C₁-C₆ alkyl;
M is >CR¹R²;
R¹ is —OH;
R² is 4-chlorophenyl;
Z is 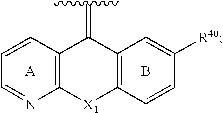
X₁ is —CH₂—O—; and
R⁴⁰ is —CH(CH₃)(OH)CH₃.

82. The method of claim 81 wherein said C₁-C₆ alkyl is —CH₃.
